SEQUENCE LISTING (369 sequences)

SEQ ID NO: 1 - DNA, 60 bp, Artificial Sequence
  Synthetic sequence
gccctcatcttcttccctgcgttctcaccaccctcaccaaggaagaagtgagggcttctc

SEQ ID NO: 2 - DNA, 60 bp, Artificial Sequence
  Synthetic sequence
gccctcatcttcttccctgcgttctcaccaccctcaccaaaaatcaaggtgaccagctcc

SEQ ID NO: 3 - DNA, 62 bp, Artificial Sequence
  Synthetic sequence
gccctcatcttcttccctgcgttctcaccaccctcaccaatcatctgccaagacagaagttc

SEQ ID NO: 4 - DNA, 60 bp, Artificial Sequence
  Synthetic sequence
gccctcatcttcttccctgcgttctcaccaccctcaccaagcaggagagtcaaaggtctg

SEQ ID NO: 5 - DNA, 60 bp, Artificial Sequence
  Synthetic sequence
gccctcatcttcttccctgcgttctcaccaccctcaccaagttgccatggagattgttgc

SEQ ID NO: 6 - DNA, 60 bp, Artificial Sequence
  Synthetic sequence
gccctcatcttcttccctgcgttctcaccaccctcaccaacagctcagtgatgtcattgc

SEQ ID NO: 7 - DNA, 61 bp, Artificial Sequence
  Synthetic sequence
gccctcatcttcttccctgcgttctcaccaccctcaccaaccttgacctctgctaatgtgg

SEQ ID NO: 8 - DNA, 60 bp, Artificial Sequence
  Synthetic sequence
gccctcatcttcttccctgcgttctcaccaccctcaccaacacctgtccaacagctacag

SEQ ID NO: 9 - DNA, 61 bp, Artificial Sequence
  Synthetic sequence
gccctcatcttcttccctgcgttctcaccaccctcaccaaagaatgtatcttcaggcctgc

SEQ ID NO: 10 - DNA, 60 bp, Artificial Sequence
  Synthetic sequence
gccctcatcttcttccctgcgttctcaccaccctcaccaaaagtaatcactctgggtggc

SEQ ID NO: 11 - DNA, 60 bp, Artificial Sequence
  Synthetic sequence
gccctcatcttcttccctgcgttctcaccaccctcaccaaagctcacagacaaccttgtg

SEQ ID NO: 12 - DNA, 60 bp, Artificial Sequence
  Synthetic sequence
gccctcatcttcttccctgcgttctcaccaccctcaccaagcaatagacacctacaggcg

SEQ ID NO: 13 - DNA, 60 bp, Artificial Sequence
  Synthetic sequence
gccctcatcttcttccctgcgttctcaccaccctcaccaagcacattatcaaaggccacg

SEQ ID NO: 14 - DNA, 60 bp, Artificial Sequence
  Synthetic sequence
gccctcatcttcttccctgcgttctcaccaccctcaccaacaacgacctaaagcatgtgc

SEQ ID NO: 15 - DNA, 60 bp, Artificial Sequence
  Synthetic sequence
gccctcatcttcttccctgcgttctcaccaccctcaccaagacatacatggctttggcag

SEQ ID NO: 16 - DNA, 62 bp, Artificial Sequence
  Synthetic sequence
gccctcatcttcttccctgcgttctcaccaccctcaccaagagatactgccacttatgcacg

SEQ ID NO: 17 - DNA, 40 bp, Artificial Sequence
  Synthetic sequence
cgtgctaatagtctcagggcttcctccaccgaacgtgtct

SEQ ID NO: 18 - DNA, 40 bp, Artificial Sequence
  Synthetic sequence
cgacgcttcattgcttcattttcctccaccgaacgtgtct

SEQ ID NO: 19 - DNA, 40 bp, Artificial Sequence
  Synthetic sequence
cttgcgccaaacaattgtccttcctccaccgaacgtgtct

SEQ ID NO: 20 - DNA, 40 bp, Artificial Sequence
  Synthetic sequence
gctgcagagtttgcattcatttcctccaccgaacgtgtct

SEQ ID NO: 21 - DNA, 41 bp, Artificial Sequence
  Synthetic sequence
catacacacagaccgagagtcttcctccaccgaacgtgtct

SEQ ID NO: 22 - DNA, 40 bp, Artificial Sequence
  Synthetic sequence
ggatgtcagccagcataagtttcctccaccgaacgtgtct

SEQ ID NO: 23 - DNA, 40 bp, Artificial Sequence
  Synthetic sequence
gcaagtgccaaacagttctcttcctccaccgaacgtgtct

SEQ ID NO: 24 - DNA, 41 bp, Artificial Sequence
  Synthetic sequence
gattccagcacacttgagtctttcctccaccgaacgtgtct

SEQ ID NO: 25 - DNA, 40 bp, Artificial Sequence
  Synthetic sequence
ccgttgcaggtttaaatggcgccctattgcaagccctctt

SEQ ID NO: 26 - DNA, 40 bp, Artificial Sequence
  Synthetic sequence
caagagtgctttatgggcctgccctattgcaagccctctt

SEQ ID NO: 27 - DNA, 41 bp, Artificial Sequence
  Synthetic sequence
gcactcaaggagatcagactggccctattgcaagccctctt

SEQ ID NO: 28 - DNA, 41 bp, Artificial Sequence
  Synthetic sequence
ggctatcgaactacaaccacagccctattgcaagccctctt

SEQ ID NO: 29 - DNA, 41 bp, Artificial Sequence
  Synthetic sequence
gtagctgtctgtggtgtgatcgccctattgcaagccctctt

SEQ ID NO: 30 - DNA, 42 bp, Artificial Sequence
  Synthetic sequence
caagaaacttcgagccttagcagccctattgcaagccctctt

SEQ ID NO: 31 - DNA, 40 bp, Artificial Sequence
  Synthetic sequence
gtgaaccagtccgagtgaaagccctattgcaagccctctt

SEQ ID NO: 32 - DNA, 41 bp, Artificial Sequence
  Synthetic sequence
gcaaatgatgttcagcaccacgccctattgcaagccctctt

SEQ ID NO: 33 - DNA, 20 bp, Artificial Sequence
  Synthetic sequence
gccctcatcttcttccctgc

SEQ ID NO: 34 - DNA, 20 bp, Artificial Sequence
  Synthetic sequence
gttctcaccaccctcaccaa

SEQ ID NO: 35 - DNA, 20 bp, Artificial Sequence
  Synthetic sequence
ggaagaagtgagggcttctc

SEQ ID NO: 36 - DNA, 20 bp, Artificial Sequence
  Synthetic sequence
aaatcaaggtgaccagctcc

SEQ ID NO: 37 - DNA, 22 bp, Artificial Sequence
  Synthetic sequence
tcatctgccaagacagaagttc

SEQ ID NO: 38 - DNA, 20 bp, Artificial Sequence
  Synthetic sequence
gcaggagagtcaaaggtctg

SEQ ID NO: 39 - DNA, 20 bp, Artificial Sequence
  Synthetic sequence
gttgccatggagattgttgc

SEQ ID NO: 40 - DNA, 20 bp, Artificial Sequence
  Synthetic sequence
cagctcagtgatgtcattgc

SEQ ID NO: 41 - DNA, 21 bp, Artificial Sequence
  Synthetic sequence
ccttgacctctgctaatgtgg

SEQ ID NO: 42 - DNA, 20 bp, Artificial Sequence
  Synthetic sequence
cacctgtccaacagctacag

SEQ ID NO: 43 - DNA, 21 bp, Artificial Sequence
  Synthetic sequence
agaatgtatcttcaggcctgc

SEQ ID NO: 44 - DNA, 20 bp, Artificial Sequence
  Synthetic sequence
aagtaatcactctgggtggc

SEQ ID NO: 45 - DNA, 20 bp, Artificial Sequence
  Synthetic sequence
agctcacagacaaccttgtg

SEQ ID NO: 46 - DNA, 20 bp, Artificial Sequence
  Synthetic sequence
gcaatagacacctacaggcg

SEQ ID NO: 47 - DNA, 20 bp, Artificial Sequence
  Synthetic sequence
gcacattatcaaaggccacg

SEQ ID NO: 48 - DNA, 20 bp, Artificial Sequence
  Synthetic sequence
caacgacctaaagcatgtgc

SEQ ID NO: 49 - DNA, 20 bp, Artificial Sequence
  Synthetic sequence
gacatacatggctttggcag

SEQ ID NO: 50 - DNA, 22 bp, Artificial Sequence
  Synthetic sequence
gagatactgccacttatgcacg

SEQ ID NO: 51 - DNA, 20 bp, Artificial Sequence
  Synthetic sequence
cgtgctaatagtctcagggc

SEQ ID NO: 52 - DNA, 20 bp, Artificial Sequence
  Synthetic sequence
cgacgcttcattgcttcatt

SEQ ID NO: 53 - DNA, 20 bp, Artificial Sequence
  Synthetic sequence
cttgcgccaaacaattgtcc

SEQ ID NO: 54 - DNA, 20 bp, Artificial Sequence
  Synthetic sequence
gctgcagagtttgcattcat

SEQ ID NO: 55 - DNA, 21 bp, Artificial Sequence
  Synthetic sequence
catacacacagaccgagagtc

SEQ ID NO: 56 - DNA, 20 bp, Artificial Sequence
  Synthetic sequence
ggatgtcagccagcataagt

SEQ ID NO: 57 - DNA, 20 bp, Artificial Sequence
  Synthetic sequence
gcaagtgccaaacagttctc

SEQ ID NO: 58 - DNA, 21 bp, Artificial Sequence
  Synthetic sequence
gattccagcacacttgagtct

SEQ ID NO: 59 - DNA, 20 bp, Artificial Sequence
  Synthetic sequence
ccgttgcaggtttaaatggc

SEQ ID NO: 60 - DNA, 20 bp, Artificial Sequence
  Synthetic sequence
caagagtgctttatgggcct

SEQ ID NO: 61 - DNA, 21 bp, Artificial Sequence
  Synthetic sequence
gcactcaaggagatcagactg

SEQ ID NO: 62 - DNA, 21 bp, Artificial Sequence
  Synthetic sequence
ggctatcgaactacaaccaca

SEQ ID NO: 63 - DNA, 21 bp, Artificial Sequence
  Synthetic sequence
gtagctgtctgtggtgtgatc

SEQ ID NO: 64 - DNA, 22 bp, Artificial Sequence
  Synthetic sequence
caagaaacttcgagccttagca

SEQ ID NO: 65 - DNA, 20 bp, Artificial Sequence
  Synthetic sequence
gtgaaccagtccgagtgaaa

SEQ ID NO: 66 - DNA, 21 bp, Artificial Sequence
  Synthetic sequence
gcaaatgatgttcagcaccac

SEQ ID NO: 67 - DNA, 20 bp, Artificial Sequence
  Synthetic sequence
ttcctccaccgaacgtgtct

SEQ ID NO: 68 - DNA, 20 bp, Artificial Sequence
  Synthetic sequence
gccctattgcaagccctctt

SEQ ID NO: 69 - DNA, 40 bp, Artificial Sequence
  Synthetic sequence
ttcctccaccgaacgtgtctagaccagcacaacttactcg

SEQ ID NO: 70 - DNA, 37 bp, Artificial Sequence
  Synthetic sequence
ttcctccaccgaacgtgtctccaaatgcacctgcctg

SEQ ID NO: 71 - DNA, 41 bp, Artificial Sequence
  Synthetic sequence
ttcctccaccgaacgtgtctagtttggacaaaggcaattcg

SEQ ID NO: 72 - DNA, 44 bp, Artificial Sequence
  Synthetic sequence
ttcctccaccgaacgtgtcttgagcttagccaatatcaagaagg

SEQ ID NO: 73 - DNA, 42 bp, Artificial Sequence
  Synthetic sequence
ttcctccaccgaacgtgtctacgtgaactttccttggtacac

SEQ ID NO: 74 - DNA, 44 bp, Artificial Sequence
  Synthetic sequence
ttcctccaccgaacgtgtcttgaagatgttctaataccttgccg

SEQ ID NO: 75 - DNA, 38 bp, Artificial Sequence
  Synthetic sequence
ttcctccaccgaacgtgtctcagtgtggagactgaacg

SEQ ID NO: 76 - DNA, 43 bp, Artificial Sequence
  Synthetic sequence
ttcctccaccgaacgtgtctaggcagggtaatgtcatgaaatg

SEQ ID NO: 77 - DNA, 37 bp, Artificial Sequence
  Synthetic sequence
ttcctccaccgaacgtgtctgattgtctggagcgctg

SEQ ID NO: 78 - DNA, 36 bp, Artificial Sequence
  Synthetic sequence
ttcctccaccgaacgtgtctagggagcaataggccg

SEQ ID NO: 79 - DNA, 37 bp, Artificial Sequence
  Synthetic sequence
ttcctccaccgaacgtgtctctgcagggtacaacacg

SEQ ID NO: 80 - DNA, 38 bp, Artificial Sequence
  Synthetic sequence
ttcctccaccgaacgtgtctcgtatctgggaagacggc

SEQ ID NO: 81 - DNA, 40 bp, Artificial Sequence
  Synthetic sequence
ttcctccaccgaacgtgtctcctgtaatcccttgcaatgc

SEQ ID NO: 82 - DNA, 38 bp, Artificial Sequence
  Synthetic sequence
ttcctccaccgaacgtgtctggtctcagcacggttctg

SEQ ID NO: 83 - DNA, 37 bp, Artificial Sequence
  Synthetic sequence
ttcctccaccgaacgtgtctgcacctccctaccacac

SEQ ID NO: 84 - DNA, 41 bp, Artificial Sequence
  Synthetic sequence
ttcctccaccgaacgtgtctgcctctagctagagagaagtc

SEQ ID NO: 85 - DNA, 39 bp, Artificial Sequence
  Synthetic sequence
ttcctccaccgaacgtgtctctggcagtctagccgttac

SEQ ID NO: 86 - DNA, 43 bp, Artificial Sequence
  Synthetic sequence
ttcctccaccgaacgtgtcttgtcttagaatttggcaactggc

SEQ ID NO: 87 - DNA, 39 bp, Artificial Sequence
  Synthetic sequence
ttcctccaccgaacgtgtctgcaggaaagcctactgaac

SEQ ID NO: 88 - DNA, 39 bp, Artificial Sequence
  Synthetic sequence
ttcctccaccgaacgtgtctgggagccagagaaatgtcc

SEQ ID NO: 89 - DNA, 44 bp, Artificial Sequence
  Synthetic sequence
ttcctccaccgaacgtgtcttgtctccagttccacttcatttag

SEQ ID NO: 90 - DNA, 39 bp, Artificial Sequence
  Synthetic sequence
ttcctccaccgaacgtgtctcccgttaattgcctactcg

SEQ ID NO: 91 - DNA, 38 bp, Artificial Sequence
  Synthetic sequence
ttcctccaccgaacgtgtctctcggtcccactggaaag

SEQ ID NO: 92 - DNA, 41 bp, Artificial Sequence
  Synthetic sequence
ttcctccaccgaacgtgtctacacccatgattcagttactg

SEQ ID NO: 93 - DNA, 41 bp, Artificial Sequence
  Synthetic sequence
ttcctccaccgaacgtgtctgctagtatgaacatcacaggc

SEQ ID NO: 94 - DNA, 43 bp, Artificial Sequence
  Synthetic sequence
ttcctccaccgaacgtgtctacaaatgagtaagaagcgagtcg

SEQ ID NO: 95 - DNA, 38 bp, Artificial Sequence
  Synthetic sequence
ttcctccaccgaacgtgtctgataagggttgctctgcg

SEQ ID NO: 96 - DNA, 37 bp, Artificial Sequence
  Synthetic sequence
ttcctccaccgaacgtgtctccatgcaccagctaccc

SEQ ID NO: 97 - DNA, 40 bp, Artificial Sequence
  Synthetic sequence
ttcctccaccgaacgtgtctaactgtaccctactcccagc

SEQ ID NO: 98 - DNA, 41 bp, Artificial Sequence
  Synthetic sequence
ttcctccaccgaacgtgtctaggaccaagggaccagtttag

SEQ ID NO: 99 - DNA, 42 bp, Artificial Sequence
  Synthetic sequence
ttcctccaccgaacgtgtctagagttcctccaagaaattgcg

SEQ ID NO: 100 - DNA, 44 bp, Artificial Sequence
  Synthetic sequence
ttcctccaccgaacgtgtctacattatacagcatgctggctatc

SEQ ID NO: 101 - DNA, 41 bp, Artificial Sequence
  Synthetic sequence
ttcctccaccgaacgtgtctgaggaagaaagtgaggtttgc

SEQ ID NO: 102 - DNA, 44 bp, Artificial Sequence
  Synthetic sequence
ttcctccaccgaacgtgtctctgaattatgtgcttaccaagagc

SEQ ID NO: 103 - DNA, 44 bp, Artificial Sequence
  Synthetic sequence
ttcctccaccgaacgtgtcttgggttctgataaccttatcaagc

SEQ ID NO: 104 - DNA, 39 bp, Artificial Sequence
  Synthetic sequence
ttcctccaccgaacgtgtctggttagtcaaacatgctgc

SEQ ID NO: 105 - DNA, 42 bp, Artificial Sequence
  Synthetic sequence
ttcctccaccgaacgtgtctgacactggcagaatcaaatcac

SEQ ID NO: 106 - DNA, 44 bp, Artificial Sequence
  Synthetic sequence
ttcctccaccgaacgtgtctagagttacacctttagctaaccac

SEQ ID NO: 107 - DNA, 38 bp, Artificial Sequence
  Synthetic sequence
ttcctccaccgaacgtgtctccaggagttcaagaagcg

SEQ ID NO: 108 - DNA, 41 bp, Artificial Sequence
  Synthetic sequence
ttcctccaccgaacgtgtctaccactcctttctcccatctc

SEQ ID NO: 109 - DNA, 44 bp, Artificial Sequence
  Synthetic sequence
ttcctccaccgaacgtgtctgtcttatgggacaatggttgatag

SEQ ID NO: 110 - DNA, 38 bp, Artificial Sequence
  Synthetic sequence
ttcctccaccgaacgtgtctctaccctcaaccctcgtc

SEQ ID NO: 111 - DNA, 38 bp, Artificial Sequence
  Synthetic sequence
ttcctccaccgaacgtgtctccaagactgatcatgccg

SEQ ID NO: 112 - DNA, 40 bp, Artificial Sequence
  Synthetic sequence
gccctattgcaagccctcttagaccagcacaacttactta

SEQ ID NO: 113 - DNA, 37 bp, Artificial Sequence
  Synthetic sequence
gccctattgcaagccctcttccaaattcacctgccca

SEQ ID NO: 114 - DNA, 41 bp, Artificial Sequence
  Synthetic sequence
gccctattgcaagccctcttagtttggacaaaggcgattta

SEQ ID NO: 115 - DNA, 44 bp, Artificial Sequence
  Synthetic sequence
gccctattgcaagccctctttgagcttagccaatatcaacaaga

SEQ ID NO: 116 - DNA, 42 bp, Artificial Sequence
  Synthetic sequence
gccctattgcaagccctcttacgtgaactttccttggtaaat

SEQ ID NO: 117 - DNA, 44 bp, Artificial Sequence
  Synthetic sequence
gccctattgcaagccctctttgaagatgttctaataccttgcta

SEQ ID NO: 118 - DNA, 38 bp, Artificial Sequence
  Synthetic sequence
gccctattgcaagccctcttcagtgtggagaccgaaca

SEQ ID NO: 119 - DNA, 43 bp, Artificial Sequence
  Synthetic sequence
gccctattgcaagccctcttaggcagggtaatgtcatgaagtt

SEQ ID NO: 120 - DNA, 37 bp, Artificial Sequence
  Synthetic sequence
gccctattgcaagccctcttgattgtctggagggctc

SEQ ID NO: 121 - DNA, 36 bp, Artificial Sequence
  Synthetic sequence
gccctattgcaagccctcttagggagcaataggcta

SEQ ID NO: 122 - DNA, 37 bp, Artificial Sequence
  Synthetic sequence
gccctattgcaagccctcttctgcagggtacaagaca

SEQ ID NO: 123 - DNA, 38 bp, Artificial Sequence
  Synthetic sequence
gccctattgcaagccctcttcgtatctgggaagatggg

SEQ ID NO: 124 - DNA, 40 bp, Artificial Sequence
  Synthetic sequence
gccctattgcaagccctcttcctgtaatcccttgcaataa

SEQ ID NO: 125 - DNA, 38 bp, Artificial Sequence
  Synthetic sequence
gccctattgcaagccctcttggtctcagcacggtcctt

SEQ ID NO: 126 - DNA, 37 bp, Artificial Sequence
  Synthetic sequence
gccctattgcaagccctcttgcacctccctatcacat

SEQ ID NO: 127 - DNA, 41 bp, Artificial Sequence
  Synthetic sequence
gccctattgcaagccctcttgcctctagctagagagaagcg

SEQ ID NO: 128 - DNA, 39 bp, Artificial Sequence
  Synthetic sequence
gccctattgcaagccctcttctggcagtctagccattat

SEQ ID NO: 129 - DNA, 43 bp, Artificial Sequence
  Synthetic sequence
gccctattgcaagccctctttgtcttagaatttggcaactagt

SEQ ID NO: 130 - DNA, 39 bp, Artificial Sequence
  Synthetic sequence
gccctattgcaagccctcttgcaggaaagcctattgaat

SEQ ID NO: 131 - DNA, 39 bp, Artificial Sequence
  Synthetic sequence
gccctattgcaagccctcttgggagccagagaaatttct

SEQ ID NO: 132 - DNA, 44 bp, Artificial Sequence
  Synthetic sequence
gccctattgcaagccctctttgtctccagttccacttcatgtaa

SEQ ID NO: 133 - DNA, 39 bp, Artificial Sequence
  Synthetic sequence
gccctattgcaagccctcttcccgttaattgcctattta

SEQ ID NO: 134 - DNA, 38 bp, Artificial Sequence
  Synthetic sequence
gccctattgcaagccctcttctcggtcccactgggaaa

SEQ ID NO: 135 - DNA, 41 bp, Artificial Sequence
  Synthetic sequence
gccctattgcaagccctcttacacccatgattcagttacca

SEQ ID NO: 136 - DNA, 41 bp, Artificial Sequence
  Synthetic sequence
gccctattgcaagccctcttgctagtatgaacatcacaagt

SEQ ID NO: 137 - DNA, 43 bp, Artificial Sequence
  Synthetic sequence
gccctattgcaagccctcttacaaatgagtaagaagcgagtta

SEQ ID NO: 138 - DNA, 38 bp, Artificial Sequence
  Synthetic sequence
gccctattgcaagccctcttgataagggttgctctaca

SEQ ID NO: 139 - DNA, 37 bp, Artificial Sequence
  Synthetic sequence
gccctattgcaagccctcttccatgcaccagctacta

SEQ ID NO: 140 - DNA, 40 bp, Artificial Sequence
  Synthetic sequence
gccctattgcaagccctcttaactgtaccctactcccaat

SEQ ID NO: 141 - DNA, 41 bp, Artificial Sequence
  Synthetic sequence
gccctattgcaagccctcttaggaccaagggaccagttcac

SEQ ID NO: 142 - DNA, 42 bp, Artificial Sequence
  Synthetic sequence
gccctattgcaagccctcttagagttcctccaagaaattgta

SEQ ID NO: 143 - DNA, 44 bp, Artificial Sequence
  Synthetic sequence
gccctattgcaagccctcttacattatacagcatgctggttaga

SEQ ID NO: 144 - DNA, 41 bp, Artificial Sequence
  Synthetic sequence
gccctattgcaagccctcttgaggaagaaagtgagatttgt

SEQ ID NO: 145 - DNA, 44 bp, Artificial Sequence
  Synthetic sequence
gccctattgcaagccctcttctgaattatgtgcttaccaggagt

SEQ ID NO: 146 - DNA, 44 bp, Artificial Sequence
  Synthetic sequence
gccctattgcaagccctctttgggttctgataaccttatcaact

SEQ ID NO: 147 - DNA, 39 bp, Artificial Sequence
  Synthetic sequence
gccctattgcaagccctcttggttagtcaaacatgttgt

SEQ ID NO: 148 - DNA, 42 bp, Artificial Sequence
  Synthetic sequence
gccctattgcaagccctcttgacactggcagaatcaaaccaa

SEQ ID NO: 149 - DNA, 44 bp, Artificial Sequence
  Synthetic sequence
gccctattgcaagccctcttagagttacacctttagctaactag

SEQ ID NO: 150 - DNA, 38 bp, Artificial Sequence
  Synthetic sequence
gccctattgcaagccctcttccaggagttcaaggagca

SEQ ID NO: 151 - DNA, 41 bp, Artificial Sequence
  Synthetic sequence
gccctattgcaagccctcttaccactcctttctcccgtctt

SEQ ID NO: 152 - DNA, 44 bp, Artificial Sequence
  Synthetic sequence
gccctattgcaagccctcttgtcttatgggacaatggtcgatat

SEQ ID NO: 153 - DNA, 38 bp, Artificial Sequence
  Synthetic sequence
gccctattgcaagccctcttctaccctcaaccctcatt

SEQ ID NO: 154 - DNA, 38 bp, Artificial Sequence
  Synthetic sequence
gccctattgcaagccctcttccaagactgatcatgcta

SEQ ID NO: 155 - DNA, 61 bp, Artificial Sequence
  Synthetic sequence
cacttgacaaagttctcacgcgccgaagttctccgaaggatgccctcatcttcttccctgc

SEQ ID NO: 156 - DNA, 61 bp, Artificial Sequence
  Synthetic sequence
cattagggattaacggcttgggacagactgacggagcttcagccctcatcttcttccctgc

SEQ ID NO: 157 - DNA, 63 bp, Artificial Sequence
  Synthetic sequence
cacacgttaagaagactttctgctgactctgccgcacatgatcgccctcatcttcttccctgc

SEQ ID NO: 158 - DNA, 62 bp, Artificial Sequence
  Synthetic sequence
ctaagtgccctccatgagaaaggatccgatagccctctgcaggccctcatcttcttccctgc

SEQ ID NO: 159 - DNA, 60 bp, Artificial Sequence
  Synthetic sequence
gcacagatttcccacactctcaacaggcctgctaaacaccgccctcatcttcttccctgc

SEQ ID NO: 160 - DNA, 61 bp, Artificial Sequence
  Synthetic sequence
cttacaggaggtctggcatcaggtcaacaaccgagggactcgccctcatcttcttccctgc

SEQ ID NO: 161 - DNA, 59 bp, Artificial Sequence
  Synthetic sequence
ccacaatgagaaggcagagttgtcattaatgctggcggcgccctcatcttcttccctgc

SEQ ID NO: 162 - DNA, 60 bp, Artificial Sequence
  Synthetic sequence
gctgtggcatagctacactccggtgacggtttgcaactttgccctcatcttcttccctgc

SEQ ID NO: 163 - DNA, 60 bp, Artificial Sequence
  Synthetic sequence
cagggtaatttgtgggtctggtccggcagttaagggtctcgccctcatcttcttccctgc

SEQ ID NO: 164 - DNA, 62 bp, Artificial Sequence
  Synthetic sequence
gggctatccagaaagataagaatactcacaaacgactgcgcagccctcatcttcttccctgc

SEQ ID NO: 165 - DNA, 63 bp, Artificial Sequence
  Synthetic sequence
cataactggtggagtatttcactcgtatatggccgactggagggccctcatcttcttccctgc

SEQ ID NO: 166 - DNA, 64 bp, Artificial Sequence
  Synthetic sequence
cttcaaggaagaaattcaacagggtagggtttgcggcgataagggccctcatcttcttccctgc

SEQ ID NO: 167 - DNA, 62 bp, Artificial Sequence
  Synthetic sequence
catggattcaacacagcaaacaccaagtcaaccacccgagacgccctcatcttcttccctgc

SEQ ID NO: 168 - DNA, 62 bp, Artificial Sequence
  Synthetic sequence
ctctgacctccttcactcttacacttccctggccttccttctgccctcatcttcttccctgc

SEQ ID NO: 169 - DNA, 62 bp, Artificial Sequence
  Synthetic sequence
gctttcatttgtgctaaacctcgcttgggtcctctcctgaacgccctcatcttcttccctgc

SEQ ID NO: 170 - DNA, 58 bp, Artificial Sequence
  Synthetic sequence
catcccagatgccctcataacgtccgaaccacaatgctgccctcatcttcttccctgc

SEQ ID NO: 171 - DNA, 62 bp, Artificial Sequence
  Synthetic sequence
gtagaaatcccaaggcaatcagctcctcgcatccaacagtcggccctcatcttcttccctgc

SEQ ID NO: 172 - DNA, 63 bp, Artificial Sequence
  Synthetic sequence
gaacaactaactccacagaacccccaccgtagcactccttcttgccctcatcttcttccctgc

SEQ ID NO: 173 - DNA, 59 bp, Artificial Sequence
  Synthetic sequence
gtgcagaggacaggaagaacggagcgtcggtagtgtaaagccctcatcttcttccctgc

SEQ ID NO: 174 - DNA, 63 bp, Artificial Sequence
  Synthetic sequence
ggtgcttcaagacatacaccttaacaactcgacgaacctaccggccctcatcttcttccctgc

SEQ ID NO: 175 - DNA, 60 bp, Artificial Sequence
  Synthetic sequence
ggaacctctgtgaccttggatggcccatccttatgtgctggccctcatcttcttccctgc

SEQ ID NO: 176 - DNA, 59 bp, Artificial Sequence
  Synthetic sequence
cccagtggtaccttctgaaggtcgttattgctcaagcccgccctcatcttcttccctgc

SEQ ID NO: 177 - DNA, 63 bp, Artificial Sequence
  Synthetic sequence
cttctgttgcttatttgggtaacttgattctggccctcccatcgccctcatcttcttccctgc

SEQ ID NO: 178 - DNA, 56 bp, Artificial Sequence
  Synthetic sequence
cccactggatgcctccctcacgccggctatttaggtgccctcatcttcttccctgc

SEQ ID NO: 179 - DNA, 58 bp, Artificial Sequence
  Synthetic sequence
cggagagacgcatctgaaagtctgggtaggtggaggacgccctcatcttcttccctgc

SEQ ID NO: 180 - DNA, 61 bp, Artificial Sequence
  Synthetic sequence
caggatttccagcttacagggcgactgagccacatccaactgccctcatcttcttccctgc

SEQ ID NO: 181 - DNA, 60 bp, Artificial Sequence
  Synthetic sequence
cttgcaagatgtgcctcttagagcctcagccggaattgaagccctcatcttcttccctgc

SEQ ID NO: 182 - DNA, 62 bp, Artificial Sequence
  Synthetic sequence
gggtggtttctctaaacacaaattgccattctgcaccaatgcgccctcatcttcttccctgc

SEQ ID NO: 183 - DNA, 62 bp, Artificial Sequence
  Synthetic sequence
gcagggtattgagagaaggatctattggtgttcgcggctgatgccctcatcttcttccctgc

SEQ ID NO: 184 - DNA, 62 bp, Artificial Sequence
  Synthetic sequence
gtgcacatttcttgatgaagggatgggcgtaacaggaggactgccctcatcttcttccctgc

SEQ ID NO: 185 - DNA, 62 bp, Artificial Sequence
  Synthetic sequence
gagcaatgcctgtttcatgagaggaatggcctacctgcatcagccctcatcttcttccctgc

SEQ ID NO: 186 - DNA, 64 bp, Artificial Sequence
  Synthetic sequence
gttaacattatacagcatggtggccccgttgttgtcatcgcatcgccctcatcttcttccctgc

SEQ ID NO: 187 - DNA, 59 bp, Artificial Sequence
  Synthetic sequence
gcagaacatgtcctgaagcgttcgatgcgtcccatgagtgccctcatcttcttccctgc

SEQ ID NO: 188 - DNA, 60 bp, Artificial Sequence
  Synthetic sequence
cagcttgttcccaaacccatcaacccgcgtagatgttcctgccctcatcttcttccctgc

SEQ ID NO: 189 - DNA, 61 bp, Artificial Sequence
  Synthetic sequence
caaagtgtggaagttgcttccgccagctcaagagtgtagccgccctcatcttcttccctgc

SEQ ID NO: 190 - DNA, 58 bp, Artificial Sequence
  Synthetic sequence
ggtcgactttgtccatccttcttgatcctgcgcgatgtgccctcatcttcttccctgc

SEQ ID NO: 191 - DNA, 59 bp, Artificial Sequence
  Synthetic sequence
ctctgttgcctgtggactcatcgcaggcgttccctatacgccctcatcttcttccctgc

SEQ ID NO: 192 - DNA, 64 bp, Artificial Sequence
  Synthetic sequence
ctaactagaattagtctgcctgcctattggacctccgaccacgagccctcatcttcttccctgc

SEQ ID NO: 193 - DNA, 59 bp, Artificial Sequence
  Synthetic sequence
gtgagccataatcgtgtcaagccaccatttagatccgcggccctcatcttcttccctgc

SEQ ID NO: 194 - DNA, 63 bp, Artificial Sequence
  Synthetic sequence
gagaattaatgctccctctcctggaccagtagaagtctgcccggccctcatcttcttccctgc

SEQ ID NO: 195 - DNA, 59 bp, Artificial Sequence
  Synthetic sequence
gtggtctgctgttgaccaatttcagaatggccgagctgtgccctcatcttcttccctgc

SEQ ID NO: 196 - DNA, 60 bp, Artificial Sequence
  Synthetic sequence
ggttgcaactgctgatctataggtgaccttcttgtacgccgccctcatcttcttccctgc

SEQ ID NO: 197 - DNA, 58 bp, Artificial Sequence
  Synthetic sequence
ctttcccagtcaaggcagggcgcgtccttatttccatcgccctcatcttcttccctgc

SEQ ID NO: 198 - DNA, 20 bp, Artificial Sequence
  Synthetic sequence
agaccagcacaacttactcg

SEQ ID NO: 199 - DNA, 17 bp, Artificial Sequence
  Synthetic sequence
ccaaatgcacctgcctg

SEQ ID NO: 200 - DNA, 21 bp, Artificial Sequence
  Synthetic sequence
agtttggacaaaggcaattcg

SEQ ID NO: 201 - DNA, 24 bp, Artificial Sequence
  Synthetic sequence
tgagcttagccaatatcaagaagg

SEQ ID NO: 202 - DNA, 22 bp, Artificial Sequence
  Synthetic sequence
acgtgaactttccttggtacac

SEQ ID NO: 203 - DNA, 24 bp, Artificial Sequence
  Synthetic sequence
tgaagatgttctaataccttgccg

SEQ ID NO: 204 - DNA, 18 bp, Artificial Sequence
  Synthetic sequence
cagtgtggagactgaacg

SEQ ID NO: 205 - DNA, 23 bp, Artificial Sequence
  Synthetic sequence
aggcagggtaatgtcatgaaatg

SEQ ID NO: 206 - DNA, 17 bp, Artificial Sequence
  Synthetic sequence
gattgtctggagcgctg

SEQ ID NO: 207 - DNA, 16 bp, Artificial Sequence
  Synthetic sequence
agggagcaataggccg

SEQ ID NO: 208 - DNA, 17 bp, Artificial Sequence
  Synthetic sequence
ctgcagggtacaacacg

SEQ ID NO: 209 - DNA, 18 bp, Artificial Sequence
  Synthetic sequence
cgtatctgggaagacggc

SEQ ID NO: 210 - DNA, 20 bp, Artificial Sequence
  Synthetic sequence
cctgtaatcccttgcaatgc

SEQ ID NO: 211 - DNA, 18 bp, Artificial Sequence
  Synthetic sequence
ggtctcagcacggttctg

SEQ ID NO: 212 - DNA, 17 bp, Artificial Sequence
  Synthetic sequence
gcacctccctaccacac

SEQ ID NO: 213 - DNA, 21 bp, Artificial Sequence
  Synthetic sequence
gcctctagctagagagaagtc

SEQ ID NO: 214 - DNA, 19 bp, Artificial Sequence
  Synthetic sequence
ctggcagtctagccgttac

SEQ ID NO: 215 - DNA, 23 bp, Artificial Sequence
  Synthetic sequence
tgtcttagaatttggcaactggc

SEQ ID NO: 216 - DNA, 19 bp, Artificial Sequence
  Synthetic sequence
gcaggaaagcctactgaac

SEQ ID NO: 217 - DNA, 19 bp, Artificial Sequence
  Synthetic sequence
gggagccagagaaatgtcc

SEQ ID NO: 218 - DNA, 24 bp, Artificial Sequence
  Synthetic sequence
tgtctccagttccacttcatttag

SEQ ID NO: 219 - DNA, 19 bp, Artificial Sequence
  Synthetic sequence
cccgttaattgcctactcg

SEQ ID NO: 220 - DNA, 18 bp, Artificial Sequence
  Synthetic sequence
ctcggtcccactggaaag

SEQ ID NO: 221 - DNA, 21 bp, Artificial Sequence
  Synthetic sequence
acacccatgattcagttactg

SEQ ID NO: 222 - DNA, 21 bp, Artificial Sequence
  Synthetic sequence
gctagtatgaacatcacaggc

SEQ ID NO: 223 - DNA, 23 bp, Artificial Sequence
  Synthetic sequence
acaaatgagtaagaagcgagtcg

SEQ ID NO: 224 - DNA, 18 bp, Artificial Sequence
  Synthetic sequence
gataagggttgctctgcg

SEQ ID NO: 225 - DNA, 17 bp, Artificial Sequence
  Synthetic sequence
ccatgcaccagctaccc

SEQ ID NO: 226 - DNA, 20 bp, Artificial Sequence
  Synthetic sequence
aactgtaccctactcccagc

SEQ ID NO: 227 - DNA, 21 bp, Artificial Sequence
  Synthetic sequence
aggaccaagggaccagtttag

SEQ ID NO: 228 - DNA, 22 bp, Artificial Sequence
  Synthetic sequence
agagttcctccaagaaattgcg

SEQ ID NO: 229 - DNA, 24 bp, Artificial Sequence
  Synthetic sequence
acattatacagcatgctggctatc

SEQ ID NO: 230 - DNA, 21 bp, Artificial Sequence
  Synthetic sequence
gaggaagaaagtgaggtttgc

SEQ ID NO: 231 - DNA, 24 bp, Artificial Sequence
  Synthetic sequence
ctgaattatgtgcttaccaagagc

SEQ ID NO: 232 - DNA, 24 bp, Artificial Sequence
  Synthetic sequence
tgggttctgataaccttatcaagc

SEQ ID NO: 233 - DNA, 19 bp, Artificial Sequence
  Synthetic sequence
ggttagtcaaacatgctgc

SEQ ID NO: 234 - DNA, 22 bp, Artificial Sequence
  Synthetic sequence
gacactggcagaatcaaatcac

SEQ ID NO: 235 - DNA, 24 bp, Artificial Sequence
  Synthetic sequence
agagttacacctttagctaaccac

SEQ ID NO: 236 - DNA, 18 bp, Artificial Sequence
  Synthetic sequence
ccaggagttcaagaagcg

SEQ ID NO: 237 - DNA, 21 bp, Artificial Sequence
  Synthetic sequence
accactcctttctcccatctc

SEQ ID NO: 238 - DNA, 24 bp, Artificial Sequence
  Synthetic sequence
gtcttatgggacaatggttgatag

SEQ ID NO: 239 - DNA, 18 bp, Artificial Sequence
  Synthetic sequence
ctaccctcaaccctcgtc

SEQ ID NO: 240 - DNA, 18 bp, Artificial Sequence
  Synthetic sequence
ccaagactgatcatgccg

SEQ ID NO: 241 - DNA, 20 bp, Artificial Sequence
  Synthetic sequence
agaccagcacaacttactta

SEQ ID NO: 242 - DNA, 17 bp, Artificial Sequence
  Synthetic sequence
ccaaattcacctgccca

SEQ ID NO: 243 - DNA, 21 bp, Artificial Sequence
  Synthetic sequence
agtttggacaaaggcgattta

SEQ ID NO: 244 - DNA, 24 bp, Artificial Sequence
  Synthetic sequence
tgagcttagccaatatcaacaaga

SEQ ID NO: 245 - DNA, 22 bp, Artificial Sequence
  Synthetic sequence
acgtgaactttccttggtaaat

SEQ ID NO: 246 - DNA, 24 bp, Artificial Sequence
  Synthetic sequence
tgaagatgttctaataccttgcta

SEQ ID NO: 247 - DNA, 18 bp, Artificial Sequence
  Synthetic sequence
cagtgtggagaccgaaca

SEQ ID NO: 248 - DNA, 23 bp, Artificial Sequence
  Synthetic sequence
aggcagggtaatgtcatgaagtt

SEQ ID NO: 249 - DNA, 17 bp, Artificial Sequence
  Synthetic sequence
gattgtctggagggctc

SEQ ID NO: 250 - DNA, 16 bp, Artificial Sequence
  Synthetic sequence
agggagcaataggcta

SEQ ID NO: 251 - DNA, 17 bp, Artificial Sequence
  Synthetic sequence
ctgcagggtacaagaca

SEQ ID NO: 252 - DNA, 18 bp, Artificial Sequence
  Synthetic sequence
cgtatctgggaagatggg

SEQ ID NO: 253 - DNA, 20 bp, Artificial Sequence
  Synthetic sequence
cctgtaatcccttgcaataa

SEQ ID NO: 254 - DNA, 18 bp, Artificial Sequence
  Synthetic sequence
ggtctcagcacggtcctt

SEQ ID NO: 255 - DNA, 17 bp, Artificial Sequence
  Synthetic sequence
gcacctccctatcacat

SEQ ID NO: 256 - DNA, 21 bp, Artificial Sequence
  Synthetic sequence
gcctctagctagagagaagcg

SEQ ID NO: 257 - DNA, 19 bp, Artificial Sequence
  Synthetic sequence
ctggcagtctagccattat

SEQ ID NO: 258 - DNA, 23 bp, Artificial Sequence
  Synthetic sequence
tgtcttagaatttggcaactagt

SEQ ID NO: 259 - DNA, 19 bp, Artificial Sequence
  Synthetic sequence
gcaggaaagcctattgaat

SEQ ID NO: 260 - DNA, 19 bp, Artificial Sequence
  Synthetic sequence
gggagccagagaaatttct

SEQ ID NO: 261 - DNA, 24 bp, Artificial Sequence
  Synthetic sequence
tgtctccagttccacttcatgtaa

SEQ ID NO: 262 - DNA, 19 bp, Artificial Sequence
  Synthetic sequence
cccgttaattgcctattta

SEQ ID NO: 263 - DNA, 18 bp, Artificial Sequence
  Synthetic sequence
ctcggtcccactgggaaa

SEQ ID NO: 264 - DNA, 21 bp, Artificial Sequence
  Synthetic sequence
acacccatgattcagttacca

SEQ ID NO: 265 - DNA, 21 bp, Artificial Sequence
  Synthetic sequence
gctagtatgaacatcacaagt

SEQ ID NO: 266 - DNA, 23 bp, Artificial Sequence
  Synthetic sequence
acaaatgagtaagaagcgagtta

SEQ ID NO: 267 - DNA, 18 bp, Artificial Sequence
  Synthetic sequence
gataagggttgctctaca

SEQ ID NO: 268 - DNA, 17 bp, Artificial Sequence
  Synthetic sequence
ccatgcaccagctacta

SEQ ID NO: 269 - DNA, 20 bp, Artificial Sequence
  Synthetic sequence
aactgtaccctactcccaat

SEQ ID NO: 270 - DNA, 21 bp, Artificial Sequence
  Synthetic sequence
aggaccaagggaccagttcac

SEQ ID NO: 271 - DNA, 22 bp, Artificial Sequence
  Synthetic sequence
agagttcctccaagaaattgta

SEQ ID NO: 272 - DNA, 24 bp, Artificial Sequence
  Synthetic sequence
acattatacagcatgctggttaga

SEQ ID NO: 273 - DNA, 21 bp, Artificial Sequence
  Synthetic sequence
gaggaagaaagtgagatttgt

SEQ ID NO: 274 - DNA, 24 bp, Artificial Sequence
  Synthetic sequence
ctgaattatgtgcttaccaggagt

SEQ ID NO: 275 - DNA, 24 bp, Artificial Sequence
  Synthetic sequence
tgggttctgataaccttatcaact

SEQ ID NO: 276 - DNA, 19 bp, Artificial Sequence
  Synthetic sequence
ggttagtcaaacatgttgt

SEQ ID NO: 277 - DNA, 22 bp, Artificial Sequence
  Synthetic sequence
gacactggcagaatcaaaccaa

SEQ ID NO: 278 - DNA, 24 bp, Artificial Sequence
  Synthetic sequence
agagttacacctttagctaactag

SEQ ID NO: 279 - DNA, 18 bp, Artificial Sequence
  Synthetic sequence
ccaggagttcaaggagca

SEQ ID NO: 280 - DNA, 21 bp, Artificial Sequence
  Synthetic sequence
accactcctttctcccgtctt

SEQ ID NO: 281 - DNA, 24 bp, Artificial Sequence
  Synthetic sequence
gtcttatgggacaatggtcgatat

SEQ ID NO: 282 - DNA, 18 bp, Artificial Sequence
  Synthetic sequence
ctaccctcaaccctcatt

SEQ ID NO: 283 - DNA, 18 bp, Artificial Sequence
  Synthetic sequence
ccaagactgatcatgcta

SEQ ID NO: 284 - DNA, 21 bp, Artificial Sequence
  Synthetic sequence
cacttgacaaagttctcacgc

SEQ ID NO: 285 - DNA, 21 bp, Artificial Sequence
  Synthetic sequence
cattagggattaacggcttgg

SEQ ID NO: 286 - DNA, 23 bp, Artificial Sequence
  Synthetic sequence
cacacgttaagaagactttctgc

SEQ ID NO: 287 - DNA, 22 bp, Artificial Sequence
  Synthetic sequence
ctaagtgccctccatgagaaag

SEQ ID NO: 288 - DNA, 20 bp, Artificial Sequence
  Synthetic sequence
gcacagatttcccacactct

SEQ ID NO: 289 - DNA, 21 bp, Artificial Sequence
  Synthetic sequence
cttacaggaggtctggcatca

SEQ ID NO: 290 - DNA, 19 bp, Artificial Sequence
  Synthetic sequence
ccacaatgagaaggcagag

SEQ ID NO: 291 - DNA, 20 bp, Artificial Sequence
  Synthetic sequence
gctgtggcatagctacactc

SEQ ID NO: 292 - DNA, 20 bp, Artificial Sequence
  Synthetic sequence
cagggtaatttgtgggtctg

SEQ ID NO: 293 - DNA, 22 bp, Artificial Sequence
  Synthetic sequence
gggctatccagaaagataagaa

SEQ ID NO: 294 - DNA, 23 bp, Artificial Sequence
  Synthetic sequence
cataactggtggagtatttcact

SEQ ID NO: 295 - DNA, 24 bp, Artificial Sequence
  Synthetic sequence
cttcaaggaagaaattcaacaggg

SEQ ID NO: 296 - DNA, 22 bp, Artificial Sequence
  Synthetic sequence
catggattcaacacagcaaaca

SEQ ID NO: 297 - DNA, 22 bp, Artificial Sequence
  Synthetic sequence
ctctgacctccttcactcttac

SEQ ID NO: 298 - DNA, 22 bp, Artificial Sequence
  Synthetic sequence
gctttcatttgtgctaaacctc

SEQ ID NO: 299 - DNA, 18 bp, Artificial Sequence
  Synthetic sequence
catcccagatgccctcat

SEQ ID NO: 300 - DNA, 22 bp, Artificial Sequence
  Synthetic sequence
gtagaaatcccaaggcaatcag

SEQ ID NO: 301 - DNA, 23 bp, Artificial Sequence
  Synthetic sequence
gaacaactaactccacagaaccc

SEQ ID NO: 302 - DNA, 19 bp, Artificial Sequence
  Synthetic sequence
gtgcagaggacaggaagaa

SEQ ID NO: 303 - DNA, 23 bp, Artificial Sequence
  Synthetic sequence
ggtgcttcaagacatacacctta

SEQ ID NO: 304 - DNA, 20 bp, Artificial Sequence
  Synthetic sequence
ggaacctctgtgaccttgga

SEQ ID NO: 305 - DNA, 19 bp, Artificial Sequence
  Synthetic sequence
cccagtggtaccttctgaa

SEQ ID NO: 306 - DNA, 23 bp, Artificial Sequence
  Synthetic sequence
cttctgttgcttatttgggtaac

SEQ ID NO: 307 - DNA, 16 bp, Artificial Sequence
  Synthetic sequence
cccactggatgcctcc

SEQ ID NO: 308 - DNA, 18 bp, Artificial Sequence
  Synthetic sequence
cggagagacgcatctgaa

SEQ ID NO: 309 - DNA, 21 bp, Artificial Sequence
  Synthetic sequence
caggatttccagcttacaggg

SEQ ID NO: 310 - DNA, 20 bp, Artificial Sequence
  Synthetic sequence
cttgcaagatgtgcctctta

SEQ ID NO: 311 - DNA, 22 bp, Artificial Sequence
  Synthetic sequence
gggtggtttctctaaacacaaa

SEQ ID NO: 312 - DNA, 22 bp, Artificial Sequence
  Synthetic sequence
gcagggtattgagagaaggatc

SEQ ID NO: 313 - DNA, 22 bp, Artificial Sequence
  Synthetic sequence
gtgcacatttcttgatgaaggg

SEQ ID NO: 314 - DNA, 22 bp, Artificial Sequence
  Synthetic sequence
gagcaatgcctgtttcatgaga

SEQ ID NO: 315 - DNA, 24 bp, Artificial Sequence
  Synthetic sequence
gttaacattatacagcatggtggc

SEQ ID NO: 316 - DNA, 19 bp, Artificial Sequence
  Synthetic sequence
gcagaacatgtcctgaagc

SEQ ID NO: 317 - DNA, 20 bp, Artificial Sequence
  Synthetic sequence
cagcttgttcccaaacccat

SEQ ID NO: 318 - DNA, 21 bp, Artificial Sequence
  Synthetic sequence
caaagtgtggaagttgcttcc

SEQ ID NO: 319 - DNA, 18 bp, Artificial Sequence
  Synthetic sequence
ggtcgactttgtccatcc

SEQ ID NO: 320 - DNA, 19 bp, Artificial Sequence
  Synthetic sequence
ctctgttgcctgtggactc

SEQ ID NO: 321 - DNA, 24 bp, Artificial Sequence
  Synthetic sequence
ctaactagaattagtctgcctgcc

SEQ ID NO: 322 - DNA, 19 bp, Artificial Sequence
  Synthetic sequence
gtgagccataatcgtgtca

SEQ ID NO: 323 - DNA, 23 bp, Artificial Sequence
  Synthetic sequence
gagaattaatgctccctctcctg

SEQ ID NO: 324 - DNA, 19 bp, Artificial Sequence
  Synthetic sequence
gtggtctgctgttgaccaa

SEQ ID NO: 325 - DNA, 20 bp, Artificial Sequence
  Synthetic sequence
ggttgcaactgctgatctat

SEQ ID NO: 326 - DNA, 18 bp, Artificial Sequence
  Synthetic sequence
ctttcccagtcaaggcag

SEQ ID NO: 327 - DNA, 20 bp, Artificial Sequence
  Synthetic sequence
gccgaagttctccgaaggat

SEQ ID NO: 328 - DNA, 20 bp, Artificial Sequence
  Synthetic sequence
gacagactgacggagcttca

SEQ ID NO: 329 - DNA, 20 bp, Artificial Sequence
  Synthetic sequence
tgactctgccgcacatgatc

SEQ ID NO: 330 - DNA, 20 bp, Artificial Sequence
  Synthetic sequence
gatccgatagccctctgcag

SEQ ID NO: 331 - DNA, 20 bp, Artificial Sequence
  Synthetic sequence
caacaggcctgctaaacacc

SEQ ID NO: 332 - DNA, 20 bp, Artificial Sequence
  Synthetic sequence
ggtcaacaaccgagggactc

SEQ ID NO: 333 - DNA, 20 bp, Artificial Sequence
  Synthetic sequence
ttgtcattaatgctggcggc

SEQ ID NO: 334 - DNA, 20 bp, Artificial Sequence
  Synthetic sequence
cggtgacggtttgcaacttt

SEQ ID NO: 335 - DNA, 20 bp, Artificial Sequence
  Synthetic sequence
gtccggcagttaagggtctc

SEQ ID NO: 336 - DNA, 20 bp, Artificial Sequence
  Synthetic sequence
tactcacaaacgactgcgca

SEQ ID NO: 337 - DNA, 20 bp, Artificial Sequence
  Synthetic sequence
cgtatatggccgactggagg

SEQ ID NO: 338 - DNA, 20 bp, Artificial Sequence
  Synthetic sequence
tagggtttgcggcgataagg

SEQ ID NO: 339 - DNA, 20 bp, Artificial Sequence
  Synthetic sequence
ccaagtcaaccacccgagac

SEQ ID NO: 340 - DNA, 20 bp, Artificial Sequence
  Synthetic sequence
acttccctggccttccttct

SEQ ID NO: 341 - DNA, 20 bp, Artificial Sequence
  Synthetic sequence
gcttgggtcctctcctgaac

SEQ ID NO: 342 - DNA, 20 bp, Artificial Sequence
  Synthetic sequence
aacgtccgaaccacaatgct

SEQ ID NO: 343 - DNA, 20 bp, Artificial Sequence
  Synthetic sequence
ctcctcgcatccaacagtcg

SEQ ID NO: 344 - DNA, 20 bp, Artificial Sequence
  Synthetic sequence
ccaccgtagcactccttctt

SEQ ID NO: 345 - DNA, 20 bp, Artificial Sequence
  Synthetic sequence
cggagcgtcggtagtgtaaa

SEQ ID NO: 346 - DNA, 20 bp, Artificial Sequence
  Synthetic sequence
acaactcgacgaacctaccg

SEQ ID NO: 347 - DNA, 20 bp, Artificial Sequence
  Synthetic sequence
tggcccatccttatgtgctg

SEQ ID NO: 348 - DNA, 20 bp, Artificial Sequence
  Synthetic sequence
ggtcgttattgctcaagccc

SEQ ID NO: 349 - DNA, 20 bp, Artificial Sequence
  Synthetic sequence
ttgattctggccctcccatc

SEQ ID NO: 350 - DNA, 20 bp, Artificial Sequence
  Synthetic sequence
ctcacgccggctatttaggt

SEQ ID NO: 351 - DNA, 20 bp, Artificial Sequence
  Synthetic sequence
agtctgggtaggtggaggac

SEQ ID NO: 352 - DNA, 20 bp, Artificial Sequence
  Synthetic sequence
cgactgagccacatccaact

SEQ ID NO: 353 - DNA, 20 bp, Artificial Sequence
  Synthetic sequence
gagcctcagccggaattgaa

SEQ ID NO: 354 - DNA, 20 bp, Artificial Sequence
  Synthetic sequence
ttgccattctgcaccaatgc

SEQ ID NO: 355 - DNA, 20 bp, Artificial Sequence
  Synthetic sequence
tattggtgttcgcggctgat

SEQ ID NO: 356 - DNA, 20 bp, Artificial Sequence
  Synthetic sequence
atgggcgtaacaggaggact

SEQ ID NO: 357 - DNA, 20 bp, Artificial Sequence
  Synthetic sequence
ggaatggcctacctgcatca

SEQ ID NO: 358 - DNA, 20 bp, Artificial Sequence
  Synthetic sequence
cccgttgttgtcatcgcatc

SEQ ID NO: 359 - DNA, 20 bp, Artificial Sequence
  Synthetic sequence
gttcgatgcgtcccatgagt

SEQ ID NO: 360 - DNA, 20 bp, Artificial Sequence
  Synthetic sequence
caacccgcgtagatgttcct

SEQ ID NO: 361 - DNA, 20 bp, Artificial Sequence
  Synthetic sequence
gccagctcaagagtgtagcc

SEQ ID NO: 362 - DNA, 20 bp, Artificial Sequence
  Synthetic sequence
ttcttgatcctgcgcgatgt

SEQ ID NO: 363 - DNA, 20 bp, Artificial Sequence
  Synthetic sequence
atcgcaggcgttccctatac

SEQ ID NO: 364 - DNA, 20 bp, Artificial Sequence
  Synthetic sequence
tattggacctccgaccacga

SEQ ID NO: 365 - DNA, 20 bp, Artificial Sequence
  Synthetic sequence
agccaccatttagatccgcg

SEQ ID NO: 366 - DNA, 20 bp, Artificial Sequence
  Synthetic sequence
gaccagtagaagtctgcccg

SEQ ID NO: 367 - DNA, 20 bp, Artificial Sequence
  Synthetic sequence
tttcagaatggccgagctgt

SEQ ID NO: 368 - DNA, 20 bp, Artificial Sequence
  Synthetic sequence
aggtgaccttcttgtacgcc

SEQ ID NO: 369 - DNA, 20 bp, Artificial Sequence
  Synthetic sequence
ggcgcgtccttatttccatc

(19) United States
(12) Patent Application Publication
(10) Pub. No.: US 2018/0023138 A1
(43) Pub. Date: Jan. 25, 2018

(54) Assays for Single Molecule Detection and Use Thereof

(71) Applicant: Singular Bio, Inc., San Francisco, CA (US)

(72) Inventors: Patrick James Collins, San Francisco, CA (US); Hywel Bowden Jones, San Francisco, CA (US); Alexandria Hui Wang, San Francisco, CA (US)

(73) Assignee: Singular Bio, Inc., San Francisco, CA (US)

(21) Appl. No.: 15/523,134

(22) PCT Filed: Oct. 31, 2015
(86) PCT No.: PCT/US15/58529

§ 371 Date: Apr. 28, 2017

Related U.S. Application Data
(60) Provisional application No. 62/073,982, filed on Nov. 1, 2014.
(60) Provisional application No. 62/073,979, filed on Nov. 1, 2014.
(60) Provisional application No. 62/135,421, filed on Mar. 19, 2015.

(51) Int. Cl. C12Q 1/68

(52) U.S. Cl. CPC C12Q 1/6876; C12Q 1/6827; C12Q 1/6837; C12Q 2600/158; C12Q 2600/156

(57) ABSTRACT

The invention relates to methods of detecting a genetic variation in a genetic sample from a subject. The invention further relates to methods of detecting a genetic variation in a genetic sample from a subject using labeled probes and counting the number of labels in the probes.

### BACKGROUND OF THE INVENTION

The invention relates to methods of detecting a genetic variation in a genetic sample from a subject. Detecting a genetic variation is important in many aspects of human biology.

### SUMMARY

The invention relates to methods of detecting a genetic variation in a genetic sample from a subject. The invention further relates to methods of detecting a genetic variation in a genetic sample from a subject using labeled probes and counting the number of labels in the probes.

### BRIEF DESCRIPTION OF THE DRAWINGS

FIG. 1 depicts exemplary array members comprising binding partners, tags, affinity tags, tagging probes, probe sets, and/or litigated probe sets described herein on a substrate.

FIG. 2 depicts a normalized histogram of signal intensity measured from both single label samples and multi-label antibodies.

FIG. 3 depicts average bleaching profiles from various labels.

FIGS. 4-13 show the integrated label intensity graphs over time for various Alexa 488 labels.

FIG. 14 depicts excitation spectrum and emission spectrum through a standard operation when excitation of a fluorophore is achieved by illuminating with a narrow spectral band aligned with the absorption maxima of that species.

FIG. 15 depicts excitation spectrum and emission spectrum through interrogation with various excitation colors and collected emission bands different from (or in addition to) the case for the standard operation.

FIG. 16 shows results when the light from these various imaging configurations, e.g., various emission filters, is collected and compared to calibration values for the fluorophores of interest.

FIG. 17 shows results collected with various references, including those with a flat emission profile (Contaminant 1; triangles), or a blue-weighted profile (Contaminant 2; stars).

FIG. 18 depicts significantly-different excitation bands of two fluorophores.

FIG. 19 depicts an exemplary system flow chart.

FIG. 20 depicts an exemplary system flow chart including various methods for analyzing data.

FIGS. 21-46 depict exemplary probe sets described herein.

FIGS. 47 and 48 show the resulting fluorescence patterns when products contain unique affinity tag sequences and the underlying substrate contains complements to each of the unique affinity tags within the same location (e.g., as the same member) on a substrate.

FIGS. 49 and 51 show the resulting fluorescence patterns when different products contain identical affinity tag sequences and the underlying substrate contains the complement to the affinity tag.

FIGS. 50 and 52 show zoomed-in locations of FIGS. 49 and 51, respectively.

FIGS. 53 and 54 show the resulting fluorescence patterns when products contain unique affinity tag sequences and the underlying substrate has one location (e.g., as one member) containing the complement to one affinity tag complement, and another separate location (e.g., as another member) containing the complement to the other affinity tag.

FIG. 55 depicts two probe sets; one probe set for Locus 1 and one probe set for Locus 2—although as aforementioned, multiple probes sets may be designed for each genomic locus.

FIGS. 56A and 56B depict the procedural workflow that would be applied to the collection of probe sets.

FIG. 57 depicts a modified version of the procedural workflow illustrated in FIGS. 56A and 56B.

FIGS. 58A-58C provide an example of how probe products for Locus 1 and Locus 2 may be labeled with different label molecules.

FIG. 59 provides evidence that probe products representing a multitude of genomic locations for one locus may be generated in a ligase enzyme specific manner using the hybridization-ligation process.

FIGS. 60A-60B provide data indicating that probe sets may be used to detect relative changes in copy number state.

FIGS. 61A-61C provide evidence that mixtures of probe products may be used to generate quantitative microarray data.

FIGS. 62-64 illustrate modifications of the general procedure described in FIGS. 55 to 58.

FIGS. 65A-65B depict a further embodiment of the modified procedure described in FIG. 62.

FIGS. 66A-66C depict yet another embodiment of the procedure depicted in FIG. 65.

FIGS. 67A-67C depict exemplary probe sets used in methods described herein.

FIGS. 68A-68C depict exemplary probe sets used in methods described herein when translocations that have known breakpoints are assayed.

FIGS. 69A-69B depict exemplary probe sets used in methods described herein when mutations at SNPs are targeted.

FIGS. 70A-70C depict an exemplary workflow for methods according to some embodiments of the present invention.

FIGS. 71A-71B depict an exemplary workflow for methods according to additional embodiments of the present invention.

FIG. 72 depicts an exemplary workflow for methods using a junction capture probe according to additional embodiments of the present invention.

FIG. 73 depicts another exemplary workflow for methods using a junction capture probe according to additional embodiments of the present invention.

FIG. 74 depicts another exemplary workflow for methods using a junction capture probe according to additional embodiments of the present invention.

FIG. 75 depicts an exemplary workflow for methods according to some embodiments of the present invention.

FIG. 76 depicts the results of an exemplary Exonuclease I treatment.

### DETAILED DESCRIPTION OF THE INVENTION

The methods described herein may employ, unless otherwise indicated, conventional techniques and descriptions of molecular biology (including recombinant techniques), cell biology, biochemistry, and microarray and sequencing technology, which are within the skill of those who practice in the art. Such conventional techniques include polymer array synthesis, hybridization and ligation of oligonucleotides, sequencing of oligonucleotides, and detection of hybridization using a label. Specific illustrations of suitable techniques can be had by reference to the examples herein. However, equivalent conventional procedures can, of course, also be used. Such conventional techniques and descriptions can be found, for example, in Kimmel and Oliver, DNA Microarrays (2006) Elsevier; Campbell, DNA Microarray, Synthesis and Synthetic DNA (2012) Nova Science; Bowtell and Sambrook, DNA Microarrays: Molecular Cloning Manual (2003) Cold Spring Harbor Laboratory Press. Before the present compositions, research tools and methods are described, it is to be understood that this invention is not limited to the specific methods, compositions, targets and uses described, as such may, of course, vary. It is also to be understood that the terminology used herein is for the purpose of describing particular aspects only and is not intended to limit the scope of the present invention, which will be limited only by appended claims.

The invention also relates to methods of detecting a genetic variation in a genetic sample from a subject, comprising contacting first and second probe sets to the genetic sample, wherein the first probe set comprises a first labeling probe and a first tagging probe, and the second probe set comprises a second labeling probe and a second tagging probe; hybridizing at least parts of the first and second probe sets to first and second nucleic acid regions of interest in nucleotide molecules of the genetic sample, respectively; ligating the first probe set at least by ligating the first labeling probe and the first tagging probe; ligating the second probe set at least by ligating the second labeling probe and the second tagging probe; optionally amplifying the ligated probe sets; immobilizing the tagging probes to a pre-determined location on a substrate, wherein the first and second labeling probes and/or the amplified labeling probes thereof ligated to the immobilized tagging probes comprise first and second labels, respectively, the first and second labels are different, the immobilized labels are optically resolvable, the immobilized first and second tagging probes and/or the amplified tagging probes thereof comprise first and second tags, respectively, and the immobilizing step is performed by immobilizing the tags to the predetermined location; counting (i) a first number of the first label immobilized to the substrate, and (ii) a second number of the second label immobilized to the substrate; and comparing the first and second numbers to determine the genetic variation in the genetic sample. The methods may further comprise labeling the first and second labeling probes with the first and second labels, respectively, prior to the contacting step. The methods may further comprise tagging the first and second tagging probes with first and second tags, respectively, prior to the contacting step. The methods may further comprise amplifying the ligated probe sets with or without labeling the probes during the amplification. In some embodiments, each of the first and second labeling probes comprises a forward or reverse priming sequence, and each of the first and second tagging probes comprises a corresponding reverse or forward priming sequence and a tagging nucleotide sequence as a tag; the methods comprise amplifying the ligated probe sets; the amplifying step comprises amplifying (i) the ligated first labeling and tagging probes with first forward and reverse primers hybridizing to the forward and reverse priming sequences, respectively, wherein the first forward or reverse primer hybridizing to the first labeling probe comprises the first label, and (ii) the ligated second labeling and tagging probes with second forward and reverse primers hybridizing to the forward and reverse priming sequences, respectively, wherein the second forward or reverse primer hybridizing to the second labeling probe comprises the second label; the amplified tagging nucleotide sequences of the tagging probes are immobilized to a pre-determined location on a substrate, wherein the amplified tagging nucleotide sequences of the first and second tagging probes are the first and second tags; the first number is the number of the first label in the amplified first probe set immobilized to the substrate, and the second number is the number of the second label in the amplified second probe set immobilized to the substrate. In additional embodiments, each of the first and second labeling probes comprises a reverse priming sequence, and each of the first and second tagging probes comprises a tagging nucleotide sequence as a tag; the method comprises amplifying the ligated probe sets; the amplifying step comprises amplifying (i) the ligated first labeling and tagging probes with a first reverse primer hybridizing to a first reverse priming sequence of the first labeling probe, wherein the first reverse primer comprises the first label, and (ii) the ligated second labeling and tagging probes with a second reverse primer hybridizing to a second reverse priming sequence of the second labeling probe, wherein the second reverse primer comprises the second label; the amplified tagging nucleotide sequences of the tagging probes are immobilized to a pre-determined location on a substrate, wherein the amplified tagging nucleotide sequences of the first and second tagging probes are the first and second tags; the first number is the number of the first label in the amplified first probe set immobilized to the substrate, and the second number is the number of the second label in the amplified second probe set immobilized to the substrate. In yet additional embodiments, the method may comprise producing separate amplification products by using the primers comprising a label in separate amplification reactions. For example, the ligated first labeling and tagging probes may be amplified in a separate PCR reaction without the presence of the ligated second labeling and tagging probes, and the ligated second labeling and tagging probes may be amplified in a separate PCR reaction without the presence of the ligated first labeling and tagging probes.

In further embodiments, the methods comprise contacting third and fourth probe sets to the genetic sample, wherein the third probe set comprises a third labeling probe and a third tagging probe, and the fourth probe set comprises a fourth labeling probe and a fourth tagging probe; hybridizing the at least parts of the first and second probe sets to first and second sense nucleic acid strands of interest in single stranded nucleotide molecules from the double stranded nucleotide molecules of the genetic sample, respectively; hybridizing at least parts of the third and fourth probe sets to anti-sense nucleic acid strands of the first and second sense nucleic acid strands of interest, respectively; producing first, second, third, and fourth ligated probe sets at least by ligating (i) the first labeling probe and the first tagging probe, (ii) the second labeling probe and the second tagging probe, (iii) the third labeling probe and the third tagging probe, and (iv) the fourth labeling probe and the fourth tagging probe; performing a ligase chain reaction comprising hybridizing at least parts of non-ligated first, second, third and fourth probe sets to the third, fourth, first, and second ligated probe sets, respectively, and ligating at least (i) the first labeling probe and the first tagging probe, (ii) the second labeling probe and the second tagging probe, (iii) the third labeling probe and the third tagging probe, and (iv) the fourth labeling probe and the fourth tagging probe of the non-ligated probe sets; immobilizing the tagging probes to the pre-determined location on a substrate, wherein the first, second, third and fourth labeling probes ligated to the immobilized tagging probes comprise first, second, third and fourth labels, respectively, the immobilized labels are optically resolvable, the immobilized first, second, third and fourth tagging probes comprise first, second, third and fourth tags, respectively, and the immobilizing step is performed by immobilizing the tags to the predetermined location; counting (i) the first sum of the first and third labels immobilized to the substrate, and (ii) the second sum of the second and fourth labels immobilized to the substrate; and comparing the first and second sums to determine the genetic variation in the genetic sample. The methods may further comprise labeling the first, second, third and fourth labeling probes with the first, second, third and fourth labels, respectively, prior to the contacting step. The first and third labels may be the same, and the second and fourth labels may be the same. In further embodiments, the methods may contact third and fourth probe sets to the genetic sample, wherein the third probe set comprises a third labeling probe and a third tagging probe, and the fourth probe set comprises a fourth labeling probe and a fourth tagging probe, the first and third labeling probes comprises a first reverse priming sequence, the second and fourth labeling probes comprises a second reverse priming sequence, and each of the tagging probes comprises a tagging nucleotide sequence as a tag; hybridizing the at least parts of the first and second probe sets to first and second sense nucleic acid strands of interest, respectively, in single stranded nucleotide molecules from double stranded nucleotide molecules of the genetic sample; hybridizing at least parts of the third and fourth probe sets to anti-sense nucleic acid strands of the first and second sense nucleic acid strands of interest, respectively; producing ligated first, second, third, and fourth probe sets by ligating (i) the first labeling probe and the first tagging probe, (ii) the second labeling probe and the second tagging probe, (iii) the third labeling probe and the third tagging probe, and (iv) the fourth labeling probe and the fourth tagging probe; performing a ligase chain reaction comprising hybridizing at least parts of the non-ligated first, second, third and fourth probe sets to the ligated third, fourth, first, and second probe sets, respectively, and ligating (i) the first labeling probe and the first tagging probe, (ii) the second labeling probe and the second tagging probe, (iii) the third labeling probe and the third tagging probe, and (iv) the fourth labeling probe and the fourth tagging probe of the non-ligated probe set; amplifying (i) the ligated first and third probe sets with a first reverse primer hybridizing to the first reverse priming sequence, wherein the first reverse primer comprises the first label, and (ii) the ligated second and fourth probe sets with a second reverse primer hybridizing to the second reverse priming sequence, wherein the second reverse primer comprises the second label, the amplified tagging nucleotide sequences of the tagging probes are immobilized to a pre-determined location on a substrate, wherein the amplified tagging nucleotide sequences of the first, second, third and fourth tagging probes are first, second, third and fourth tags, the first number is the number of the first label in the amplified first and third probe sets immobilized to the substrate, and the second number is the number of the second label in the amplified second and fourth probe sets immobilized to the substrate. The ligated first and second labeling probes may be at the 3′-end of the first and second ligated probe set and comprise first and second reverse priming sequences hybridizing to the first and second reverse primers, respectively; the first and second reverse primers may comprise the first and second labels; and the ligated first and second tagging probes may be at the 5′-end of the first and second probe set. The ligated first and second labeling probes may be at the 3′-end of the first and second ligated probe set and comprise first and second reverse priming sequences hybridizing to the first and second reverse primers, respectively; the first and second reverse primers may comprise the first and second labels; and the ligated first and second tagging probes may be at the 5′-end of the first and second ligated probe set and comprise first and second corresponding forward priming sequences hybridizing to the first and second forward primers, respectively. The amplifying step may comprise contacting an exonuclease to the amplified probe, digesting the 5′-end of the amplified probe set that does not have any label at the 5′-end. The 5′-end of the amplified probe set may comprise the label at the 5′-end is protected from exonuclease digestion. The determined genetic variation may indicate presence or absence of cancer, pharmacokinetic variability, drug toxicity, transplant rejection, or aneuploidy in the subject. The genetic variation may be aneuploidy. The subject may be a pregnant subject, and the genetic variation may be a genetic variation in the fetus of the pregnant subject. The genetic variation may be selected from the group consisting of trisomy 13, trisomy 18, trisomy 21, aneuploidy of X, and aneuploidy of Y in the fetus of the pregnant subject. The genetic variation may be a variation in the fetus of the pregnant subject selected from the group consisting of 22q11.2, 1q21.1, 9q34, 1p36, 15q, 11q, 8q, 5p, 4p and 22q13. The genetic variation may be a variation in the fetus of the pregnant subject that causes or increases the risk of specific disease, syndromes or conditions including Down syndrome, Edwards syndrome, Patau syndrome, DiGeorge syndrome, Angelman/Prader-Willi syndromes, Jacobsen syndrome, Langer-Giedion syndrome, Cri-du-chat syndrome, Wolf-Hirschhorn syndrome and 1p36 deletion syndrome. The different labels may have different optical properties. The method may detect first and second genetic variations, and the method may further comprise contacting a fifth probe set to the genetic sample, wherein the fifth probe set comprises a fifth labeling probe and a fifth tagging probe; hybridizing at least a part of the fifth probe set to the third nucleic acid region of interest in nucleotide molecules of the genetic sample, wherein the third nucleic acid region of interest is different from the first and second nucleic acid regions of interest; ligating the fifth probe set at least by ligating the fifth labeling probe and the fifth tagging probe; optionally amplifying the ligated probe sets; immobilizing each of the tagging probe to a pre-determined location on a substrate, wherein the fifth labeling probe and/or the amplified labeling probe thereof ligated to the immobilized tagging probe comprise a fifth label, the fifth label is different from the first and second labels, the immobilized labels are optically resolvable, the immobilized fifth tagging probe and/or the amplified tagging probe thereof comprise a fifth tag, and the immobilizing step is performed by immobilizing the tags to the predetermined location; counting a third number of the fifth label immobilized to the substrate; and comparing the third number to the first and/or second number(s) to determine the second genetic variation in the genetic sample. The subject is a pregnant subject; the first genetic variation may be trisomy 21 in the fetus of the pregnant subject, and the second genetic variation may be selected from the group consisting of trisomy 13, trisomy 18, aneuploidy of X, and aneuploidy of Y in the fetus of the pregnant subject. The method may further comprise contacting a sixth probe set to the genetic sample, wherein the sixth probe set comprises a sixth labeling probe and a sixth tagging probe; hybridizing at least a part of the sixth probe set to the fourth nucleic acid region of interest in nucleotide molecules of the genetic sample, wherein the fourth nucleic acid region of interest is different from the first, second, and third nucleic acid regions of interest; ligating the sixth probe set at least by ligating the sixth labeling probe and the sixth tagging probe; optionally amplifying the ligated probe sets; immobilizing each of the tagging probes to a pre-determined location on a substrate, wherein the sixth labeling probe and/or the amplified labeling probe thereof ligated to the immobilized tagging probe comprise a sixth label, the sixth label is different from the first and second labels, the immobilized labels are optically resolvable, the immobilized sixth tagging probe and/or the amplified tagging probe thereof comprise a sixth tag, and the immobilizing step is performed by immobilizing the tags to the predetermined location; counting a fourth number of the sixth label immobilized to the substrate; and comparing the fourth number to the first, second and/or third number to determine the third genetic variation in the genetic sample. The subject may be a pregnant subject; and the first, second, and third genetic variations may be trisomy 18, trisomy 21 and trisomy 13 in the fetus of the pregnant subject, respectively. The method may comprise contacting a seventh probe set to the genetic sample, wherein the seventh probe set comprises a seventh labeling probe and a seventh tagging probe; hybridizing at least a part of the seventh probe set to the fifth nucleic acid region of interest in nucleotide molecules of the genetic sample, wherein the fifth nucleic acid region of interest is different from the first, second, third and fourth nucleic acid regions of interest; ligating the seventh probe set at least by ligating the seventh labeling probe and the seventh tagging probe; optionally amplifying the ligated probe sets; immobilizing each of the tagging probes to a pre-determined location on a substrate, wherein the seventh labeling probe and/or the amplified labeling probe thereof ligated to the immobilized tagging probe comprise a seventh label, the seventh label is different from the first and second labels, the immobilized labels are optically resolvable, the immobilized seventh tagging probe and/or the amplified tagging probe thereof comprise a seventh tag, and the immobilizing step is performed by immobilizing the tags to the predetermined location; counting a fifth number of the seventh label immobilized to the substrate; and comparing the fifth number to the first, second, third and/or fourth number(s) to determine the fourth genetic variation in the genetic sample. The method may comprise contacting an eighth probe set to the genetic sample, wherein the eighth probe set comprises a eighth labeling probe and a eighth tagging probe; hybridizing at least a part of the eighth probe set to the sixth nucleic acid region of interest in nucleotide molecules of the genetic sample, wherein the sixth nucleic acid region of interest is different from the first, second, third, fourth, and fifth nucleic acid regions of interest; ligating the eighth probe set at least by ligating the eighth labeling probe and the eighth tagging probe; optionally amplifying the ligated probe sets; immobilizing each of the tagging probes to a pre-determined location on a substrate, wherein the eighth labeling probe and/or the amplified labeling probe thereof ligated to the immobilized tagging probe comprise a eighth label, the eighth label is different from the first and second labels, the immobilized labels are optically resolvable, the immobilized eighth tagging probe and/or the amplified tagging probe thereof comprise a eighth tag, and the immobilizing step is performed by immobilizing the tags to the predetermined location; counting a sixth number of the eighth label immobilized to the substrate; and comparing the sixth number to the first, second, third, fourth and/or fifth number(s) to determine the fifth genetic variation in the genetic sample. The first and second probe sets may further comprise third and fourth labeling probes, respectively; the immobilized first probe set and/or amplified first probe set may further comprise a ninth label in the third labeling probe and/or amplified product thereof; and the immobilized second probe set and/or amplified second probe set may further comprise a tenth label in the fourth labeling probe and/or amplified product thereof. The subject may be a pregnant subject; the genetic variation may be a genetic variation in the fetus of the pregnant subject; and the method may further comprise contacting maternal and paternal probe sets to the genetic sample, wherein the maternal probe set comprises a maternal labeling probe and a maternal tagging probe, and the paternal probe set comprises a paternal labeling probe and a paternal tagging probe; hybridizing at least a part of each of the maternal and paternal probe sets to a nucleic acid region of interest in nucleotide molecules of the genetic sample, the nucleic acid region of interest comprising a predetermined Single Nucleotide Polymorphism (SNP) site, wherein the at least a part of the maternal probe set hybridizes to a first allele At the SNP site, the at least a part of the paternal probe set hybridizes to a second allele at the SNP site, and the first and second alleles are different from each other, ligating the maternal and paternal probe sets at least by ligating (i) the maternal labeling and tagging probes, and (ii) the paternal labeling and tagging probes, optionally amplifying the ligated probe sets; immobilizing the tagging probes to a pre-determined location on a substrate, wherein the maternal and paternal labeling probes and/or the amplified labeling probes thereof ligated to the immobilized tagging probes comprise maternal and paternal labels, respectively, the maternal and paternal labels are different, and the immobilized labels are optically resolvable; counting the numbers of the maternal and paternal labels; and determining whether a proportion of a fetal material in the genetic sample is sufficient to detect the genetic variation in the fetus based on the numbers of the maternal and paternal labels. The subject may be a pregnant subject; the genetic variation is a genetic variation in the fetus of the pregnant subject; and the method may further comprise contacting allele A and B probe sets that are allele-specific to the genetic sample, wherein the allele A probe set comprises an allele A labeling probe and an allele A tagging probe, and the allele B probe set comprises an allele B labeling probe and an allele B tagging probe; hybridizing at least a part of each of the allele A and allele B probe sets to a nucleic acid region of interest in nucleotide molecules of the genetic sample, the nucleic acid region of interest comprising a predetermined single nucleotide polymorphism (SNP) site for which a maternal allelic profile differs from a fetal allelic profile at the SNP site, wherein the at least a part of the allele A probe set hybridizes to a first allele at the SNP site, the at least a part of the allele B probe set hybridizes to a second allele at the SNP site, and the first and second alleles are different from each other; ligating the allele A and B probe sets at least by ligating (i) the allele A labeling and tagging probes, and (ii) the allele B labeling and tagging probes; optionally amplifying the ligated probe sets; immobilizing the tagging probes to a pre-determined location on a substrate, wherein the allele A and allele B labeling probes and/or the amplified labeling probes thereof ligated to the immobilized tagging probes comprise allele A and allele B labels, respectively, the allele A and allele B labels are different, and the immobilized labels are optically resolvable; counting the numbers of the allele A and allele B labels; and determining whether a proportion of a fetal material in the genetic sample is sufficient to detect the genetic variation in the fetus based on the numbers of the allele A and allele B labels.

The invention relates to methods of detecting a genetic variation in a genetic sample from a subject. The genetic variation herein may include, but is not limited to, one or more substitution, inversion, insertion, deletion, or mutation in nucleotide sequences (e.g., DNA and RNA) and proteins (e.g., peptide and protein), one or more rare allele, polymorphism, single nucleotide polymorphism (SNP), large-scale genetic polymorphism, such as inversions and translocations, differences in the abundance and/or copy number (e.g., copy number variants, CNVs) of one or more nucleotide molecules (e.g., DNA), trisomy, monosomy, and genomic rearrangements. In some embodiments, the genetic variation may be related to metastasis, presence, absence, progression, stage and/or risk of a disease, such as cancer, pharmacokinetic variability, drug toxicity, adverse events, recurrence, and/or presence, absence, progression or risk of organ transplant rejection in the subject. For example, copy number changes in the HER2 gene affect whether a breast cancer patient will respond to Herceptin treatment or not. Similarly, detecting an increase in copy number of chromosome 21 (or 18, or 13, or sex chromosomes) in blood from a pregnant woman may be used to as a non-invasive diagnostic for Down's Syndrome in an unborn child. An additional example is the detection of alleles from a transplanted organ that are not present in the recipient genome—monitoring the frequency, or copy number, of these alleles may identify signs of potential organ rejection. Various methods may be used to detect such changes (e.g., rtPCR, sequencing and microarrays). One of the methods is to count individual, labeled molecules to either detect the presence of a mutation (e.g., EGFR mutation in cancer) or an excess of a specific genomic sequence or region (e.g., Chromosome 21 in Down's Syndrome). Counting single molecules may be done in a number of ways, with a common readout being to deposit the molecules on a surface and image.

Moreover, the genetic variation may be de novo genetic mutations, such as single- or multi-base mutations, translocations, subchromosomal amplifications and deletions, and aneuploidy. In some embodiments, the genetic variation may mean an alternative nucleotide sequence at a genetic locus that may be present in a population of individuals and that includes nucleotide substitutions, insertions, and deletions with respect to other members of the population. In additional embodiments, the genetic variation may be aneuploidy. In yet additional embodiments, the genetic variation may be trisomy 13, trisomy 18, trisomy 21, aneuploidy of X (e.g., trisomy XXX and trisomy XXY), or aneuploidy of Y (e.g., trisomy XYY). In further embodiments, the genetic variation may be in region 22q11.2, 1q21.1, 9q34, 1p36, 4p, 5p, 7q11.23, 11q24.1, 17p, 11p15, 18q, or 22q13. In further embodiments, the genetic variation may be a microdeletion or microamplification.

In some embodiments, detecting, discovering, determining, measuring, evaluating, counting, and assessing the genetic variation are used interchangeably and include quantitative and/or qualitative determinations, including, for example, identifying the genetic variation, determining presence and/or absence of the genetic variation, and quantifying the genetic variation. In further embodiments, the methods of the present disclosure may detect multiple genetic variations. The term “and/or” used herein is defined to indicate any combination of the components. Moreover, the singular forms “a,” “an,” and “the” may further include plural referents unless the context clearly dictates otherwise. Thus, for example, reference to “a nucleotide region” refers to one, more than one, or mixtures of such regions, and reference to “an assay” may include reference to equivalent steps and methods known to those skilled in the art, and so forth.

“Sample” means a quantity of material from a biological, environmental, medical, or patient source in which detection, measurement, or labeling of target nucleic acids, peptides, and/or proteins is sought. On the one hand it is meant to include a specimen or culture (e.g., microbiological cultures). On the other hand, it is meant to include both biological and environmental samples. A sample may include a specimen of synthetic origin. Environmental samples include environmental material, such as surface matter, soil, water and industrial samples, as well as samples obtained from food and dairy processing instruments, apparatus, equipment, utensils, disposable and non-disposable items. “Genetic sample” may be any liquid or solid sample with heritable and/or non-heritable biological information coded in the nucleotide sequences of nucleic acids. The sample may be obtained from a source, including, but not limited to, whole blood, serum, plasma, urine, saliva, sweat, fecal matter, tears, intestinal fluid, mucous membrane samples, lung tissue, tumors, transplanted organs, fetus, and/or other sources. Genetic samples may be from an animal, including human, fluid, solid (e.g., stool) or tissue. Genetic samples may include materials taken from a patient including, but not limited to cultures, blood, saliva, cerebral spinal fluid, pleural fluid, milk, lymph, sputum, semen, needle aspirates, and the like. Moreover, the genetic sample may be a fetal genetic material from a maternal blood sample. The fetal genetic material may be isolated and separated from the maternal blood sample. The genetic sample may be a mixture of fetal and maternal genetic material. In addition, the genetic sample may include aberrant genetic sequences arising from tumor formation or metastasis, and/or donor DNA signatures present in a transplant recipient. In additional embodiments, when the genetic sample is plasma, the method may comprise isolating the plasma from a blood sample of the subject. In further embodiments, when genetic sample is serum, the method may comprise isolating the serum from a blood sample of the subject. In yet additional embodiments, when the genetic sample is a cell free DNA (cfDNA) sample, the method further comprises isolating the cell free DNA sample from a sample obtained from the source described herein. The cell free DNA sample herein means a population of DNA molecules circulating freely in the bloodstream, outside of any cell or organelle. In the case of a pregnancy, cell free DNA from the mother carries a mixture of both maternal DNA as well as fetal DNA. These examples are not to be construed as limiting the sample types applicable to the present invention.

In some embodiments, the method of the present disclosure may comprise selecting and/or isolating genetic locus or loci of interest, and quantifying the amount of each locus present (for example for determining copy number) and/or the relative amounts of different locus variants (for example two alleles of a given DNA sequence). Region, region of interest, locus, or locus of interest in reference to a genome or target polynucleotide used herein means a contiguous sub-region or segment of the genome or target polynucleotide. As used herein, region, regions or interest, locus, locus, or locus of interest in a nucleotide molecule may refer to the position of a nucleotide, a gene or a portion of a gene in a genome, including mitochondrial DNA or other non-chromosomal DNA, or it may refer to any contiguous portion of genomic sequence whether or not it is within, or associated with, a gene. A region, region of interest, locus, locus, or locus of interest in a nucleotide molecule may be from a single nucleotide to a segment of a few hundred or a few thousand nucleotides in length or more. In some embodiments, a region or locus of interest may have a reference sequence associated with it. “Reference sequence” used herein denotes a sequence to which a locus of interest in a nucleic acid is being compared. In certain embodiments, a reference sequence is considered a “wild type” sequence for a locus of interest. A nucleic acid that contains a locus of interest having a sequence that varies from a reference sequence for the locus of interest is sometimes referred to as “polymorphic” or “mutant” or “genetic variation.” A nucleic acid that contains a locus of interest having a sequence that does not vary from a reference sequence for the locus of interest is sometimes referred to as “non-polymorphic” or “wild type” or “non-genetic variation.” In certain embodiments, a locus of interest may have more than one distinct reference sequence associated with it (e.g., where a locus of interest is known to have a polymorphism that is to be considered a normal or wild type). In some embodiments, the method of the present disclosure may also comprise electing and/or isolating peptide or peptides of interest, and qualifying the amount of each peptide present and/or relative amounts of different peptides.

In additional embodiments, the region of interest described herein may include “consensus genetic variant sequence” which refers to the nucleic acid or protein sequence, the nucleic or amino acids of which are known to occur with high frequency in a population of individuals who carry the gene which codes for a protein not functioning normally, or in which the nucleic acid itself does not function normally. Moreover, the region of interest described herein may include “consensus normal gene sequence” which refers to a nucleic acid sequence, the nucleic acid of which are known to occur at their respective positions with high frequency in a population of individuals who carry the gene which codes for a protein not functioning normally, or which itself does not function normally. In further embodiments, the control region that is not the region of interest or the reference sequence described herein may include “consensus normal sequence” which refers to the nucleic acid or protein sequence, the nucleic or amino acids of which are known to occur with high frequency in a population of individuals who carry the gene which codes for a normally functioning protein, or in which the nucleic acid itself has normal function.

The methods described herein may produce highly accurate measurements of genetic variation. One type of variation described herein includes the relative abundance of two or more distinct genomic loci. In this case, the loci may be small (e.g., as small as about 300, 250, 200, 150, 100, or 50 nucleotides or less), moderate in size (e.g., from 1,000, 10,000, 100,000 or one million nucleotides), and as large as a portion of a chromosome arm or the entire chromosome or sets of chromosomes. The results of this method may determine the abundance of one locus to another. The precision and accuracy of the methods of the present disclosure may enable the detection of very small changes in copy number (as low as about 25, 10, 5, 4, 3, 2, 1, 0.5, 0.1, 0.05, 0.02 or 0.01% or less), which enables identification of a very dilute signature of genetic variation. For Example, a signature of fetal aneuploidy may be found in a maternal blood sample where the fetal genetic aberration is diluted by the maternal blood, and an observable copy number of change of about 2% is indicative of fetal trisomy.

As used herein, the term “about” means modifying, for example, lengths of nucleotide sequences, degrees of errors, dimensions, the quantity of an ingredient in a composition, concentrations, volumes, process temperature, process time, yields, flow rates, pressures, and like values, and ranges thereof, refers to variation in the numerical quantity that may occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of, for example, a composition, formulation, or cell culture with a particular initial concentration or mixture, and amounts that differ due to mixing or processing a composition or formulation with a particular initial concentration or mixture. Whether modified by the term “about” the claims appended hereto include equivalents to these quantities. The term “about” further may refer to a range of values that are similar to the stated reference value. In certain embodiments, the term “about” refers to a range of values that fall within 50, 25, 10, 9, 8, 7, 6, 5, 4, 3, 2, 1 percent or less of the stated reference value.

In some embodiments, the subject may be a pregnant subject, human, a subject with a high risk of a genetic disease (e.g., cancer), all of the various families of domestic animals, as well as feral or wild animals. In some embodiments, the genetic variation may be a genetic variation in the fetus of the pregnant subject (e.g., copy number variants and aneuploidy in the fetus). In some embodiments, the subject is a pregnant subject, and the genetic variation is a variation in the fetus of the pregnant subject in a region selected from the group consisting of 22q11.2, 1q21.1, 9q34, 1p36, 4p, 5p, 7q11.23, 11q24.1, 17p, 11p15, 18q, and 22q13, (e.g., a mutation and/or copy number change in any of regions 22q11.2, 1q21.1, 9q34, 1p36, 4p, 5p, 7q11.23, 11q24.1, 17p, 11p15, 18q, and 22q13). Fetus described herein means an unborn offspring of a human or other animal. In some embodiments, the fetus may be the offspring more than 1, 2, 3, 4, 5, 6, 7, 8, 9, 10, 11, 12, 13, 14, 15, 16, 17, 18, 19, 20 weeks after conception. In additional embodiments, the fetus may be an offspring conceived by implants, in vitro fertilization, multiple pregnancies, or twinning. In additional embodiments, the fetus may be part of a pair of twins (identical or non-identical), or a trio of triplets (identical or non-identical) or other multiple fetus pregnancy.

The inventions according to some embodiments encompass at least two major components: an assay for the selective identification of genomic loci, and a technology for quantifying these loci with high accuracy. The assay may include methods of selectively labeling and/or isolating one or more nucleic acid sequences, in such a manner that the labeling step itself is sufficient to yield molecules (defined as “probe products,” “ligated probe set,” “conjugated probe set,” “ligated probes,” “conjugated probes,” or “labeled molecules” in this invention) containing all necessary information for identification of a particular sequence in the context of a particular assay. For example, the assay may comprise contacting, binding, and/or hybridizing probes to a sample, ligating and/or conjugating the probes, optionally amplifying the ligated/conjugated probes, and immobilizing the probes to a substrate. In some embodiments, the assays and methods described herein may be performed on a single input sample in parallel as a multiplex assay as described herein

The probe product, ligated probe set, conjugated probe set, ligated probes, conjugated probes, and labeled molecules may be single, contiguous molecule resulting from the performance of enzymatic action on a probe set, such as an assay. In a probe product or a labeled molecule, one or more individual probes from a probe set may be covalently modified such that they form a singular distinct molecular species as compared to either probes or probe sets. As a result, probe products or a labeled molecule may be chemically distinct and may therefore be identified, counted, isolated, or further manipulated apart from probes or probe sets.

For example, probe products may contain one or more identification labels, and one or more affinity tags for isolation and/or immobilization. In some embodiments, no additional modifications of probe products (e.g., DNA sequence determination) need to be performed. In some embodiments, no additional interrogations of the DNA sequence are required. The probe products containing the labels may be directly counted, typically after an immobilization step onto a solid substrate. For example, organic fluorophore labels are used to label probe products, and the probe products are directly counted by immobilizing the probe products to a glass substrate and subsequent imaging via a fluorescent microscope and a digital camera. In other embodiments, the label may be selectively quenched or removed depending on whether the labeled molecule has interacted with its complementary genomic locus. In additional embodiments, two labels on opposite portions of the probe product may work in concert to deliver a fluorescence resonance energy transfer (FRET) signal depending on whether the labeled molecule has interacted with its complementary genomic locus. For a given genomic locus, labeling probes containing the labels be designed for any sequence region within that locus. A set of multiple labeling probes with same or different labels may also be designed for a single genomic locus. In this case, a probe may selectively isolate and label a different region within a particular locus, or overlapping regions within a locus. In some embodiments, the probe products containing affinity tags are immobilized onto the substrate via the affinity tags. For example, affinity tags are used to immobilize probe products onto the substrate, and the probe products containing the affinity tags are directly counted. For a given genomic locus, tagging probes containing the affinity tags be designed for any sequence region within that locus. A set of multiple tagging probes with same or different affinity tags may also be designed for a single genomic locus. In this case, a probe may selectively isolate and tag a different region within a particular locus, or overlapping regions within a locus.

In one aspect, the methods of the present disclosure may comprise contacting probe sets described herein with the genetic sample described herein. In some embodiments, the methods of the present disclosure may comprise contacting multiple probe sets, such as first and second probe sets, to the genetic sample. In additional embodiments, each of the probe sets comprises a labeling probe and a tagging probe. For example, the first probe set comprises a first labeling probe and a first tagging probe, and the second probe set comprises a second labeling probe and a second tagging probe.

Contacting the probe sets to the genetic sample may be performed simultaneously or after hybridizing, ligating, amplifying and/or immobilizing the probes. Moreover, contacting the probe sets to the genetic sample may be performed simultaneously or before hybridizing, ligating, amplifying, and/or immobilizing the probes.

For a given genomic locus or region of a nucleotide molecule in the genetic sample, a single nucleic acid sequence within that locus, or multiple nucleic acid sequences within that locus may be interrogated and/or quantified via the creation of probe products. The interrogated sequences within a genomic locus may be distinct and/or overlapping, and may or may not contain genetic polymorphisms. A probe product is formed by the design of one or more oligonucleotides called a “probe set.” For example, the probe product may be formed by ligating the probe set by ligating the probes in the probe set. A probe set comprises at least one probe that hybridize, conjugate, bind, or immobilize to a target molecule, including nucleic acids (e.g., DNA and RNA), peptides, and proteins. In some embodiments, a probe may comprise an isolated, purified, naturally-occurring, non-naturally occurring, and/or artificial material, for example, including oligonucleotides of any length (e.g., 5, 10, 20, 30, 40, 50, 100, or 150 nucleotides or less), in which at least a portion(s) (e.g., 50, 60, 70, 80, 85, 90, 91, 92, 93, 94, 95, 96, 97, 98, 99, or 100%) of the oligonucleotide sequences is complementary to a sequence motif and/or hybridization domain present in one or more target molecules, such that the probe is configured to hybridize (or interact in a similar manner) in part or in total to one or more target molecules or nucleic acid region of interest. The part of the target molecule or the nucleic acid region of interest to which a probe hybridizes is called the probe's “hybridization domain,” which may be in part or in total of the target molecule or the nucleic acid region of interest as described herein.

A probe may be single-stranded or double-stranded. In some embodiments, the probe may be prepared from in a purified restriction digest or produced synthetically, recombinantly or by PCR amplification. In additional embodiments, the probe may comprise a material that binds to a particular peptide sequence. A probe set described herein may comprise a set of one or more probes designed to correspond to a single genomic location or a peptide in a protein sequence.

“Nucleotide” used herein means either a deoxyribonucleotide or a ribonucleotide or any nucleotide analogue (e.g., DNA and RNA). Nucleotide analogues include nucleotides having modifications in the chemical structure of the base, sugar and/or phosphate, including, but not limited to, 5′-position pyrimidine modifications, 8-position purine modifications, modifications at cytosine exocyclic amines, substitution of 5-bromo-uracil, and the like; and 2′-position sugar modifications, including but not limited to, sugar-modified ribonucleotides in which the 2′-OH is replaced by a group selected from H, OR, R, halo, SH, SR, NH₂, NHR, NR₂, or CN. shRNAs also may comprise non-natural elements such as non-natural nucleotides, e.g., ionosin and xanthine, non-natural sugars, e.g., 2′-methoxy ribose, or non-natural phosphodiester linkages, e.g., methylphosphonates, phosphorothioates and peptides. In one embodiment, the shRNA further comprises an element or a modification that renders the shRNA resistant to nuclease digestion. “Polynucleotide” or “oligonucleotide” is used interchangeably and each means a linear polymer of nucleotide monomers. Monomers making up polynucleotides and oligonucleotides are capable of specifically binding to a natural and/or artificial polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogues thereof, e.g., naturally occurring or non-naturally occurring analogues. Non-naturally occurring analogues may include PNAs, LNAs, phosphorothioate internucleosidic linkages, nucleotides containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogues of internucleosidic linkages, sugar moieties, or nucleotides at any or some positions. Polynucleotides typically range in size from a few monomeric units when they are referred to as “oligonucleotides” to several thousand monomeric units. Whenever a polynucleotide or oligonucleotide is represented by a sequence of letters (upper or lower case), such as “ATGCCTG,” it will be understood that the nucleotides are in 5′→3′ order from left to right. Usually polynucleotides comprise the four natural nucleosides (e.g., deoxyadenosine, deoxycytidine, deoxyguanosine, deoxythymidine for DNA or their ribose counterparts for RNA) linked by phosphodiester linkages; however, they may also comprise non-natural nucleotide analogues, e.g., including modified nucleotides, sugars, or internucleosidic linkages. It is clear to those skilled in the art that where an enzyme has specific oligonucleotide or polynucleotide substrate requirements for activity, e.g., single stranded DNA, RNA, RNA/DNA duplex, or the like, then selection of appropriate composition for the oligonucleotide or polynucleotide substrates is well within the knowledge of one of ordinary skill.

In another aspect, the methods of the present disclosure may comprise hybridizing at least parts of the first and second probe sets to first and second nucleic acid regions of interest in nucleotide molecules of the genetic sample, respectively. The hybridization of the probes to the nucleic acid of interest may be performed simultaneously or after contacting the probes to the genetic sample, ligating, amplifying and/or immobilizing the probes. Moreover, the hybridization of the probes to the nucleic acid of interest may be performed simultaneously or before ligating, amplifying, and/or immobilizing the probes. A part or full part of the probe may hybridize to a part or full part of the region of interest in single or double stranded nucleotide molecules, protein, or antibody in a sample. The region of interest hybridized to the probe may be from 1 to 50 nucleotides, 50 to 1000 nucleotides, 100 to 500 nucleotides, 5, 10, 50, 100, 200 nucleotides or less, or 2, 5, 10, 50, 100, 200, 500, 1000 nucleotides or more. Probes may be designed or configured to hybridize perfectly with a target region or molecule, or they may be designed such that a single-base mismatch (e.g., at a single nucleotide polymorphism, or SNP site), or a small number of such mismatches, fails to yield a hybrid of probe and target molecule.

In additional embodiments, the first labeling probe and/or the first tagging probe are hybridized to the first nucleic acid region of interest, and the second labeling probe and/or the second tagging probes are hybridized to the second nucleic acid region of interest. In additional embodiments, multiple or all probes and/or other components (e.g., labelling probes, tagging probes, and gap probes) of a probe set that are hybridized to a nucleic acid region of interest are adjacent to each other. When two of the probes and/or components hybridized to the nucleic acid region of interest are “adjacent” or “immediately adjacent,” there is no nucleotide between the hybridization domains of the two probes in the nucleic acid region of interest. In this embodiment, the different probes within a probe set may be covalently ligated together to form a larger oligonucleotide molecule. In another embodiment, a probe set may be designed to hybridize to a non-contiguous, but proximal, portion of the nucleic acid region of interest, such that there is a “gap” of one or more nucleotides on the nucleic acid region of interest, in between hybridized probes from a probe set, that is not occupied by a probe. In this embodiment, a DNA polymerase or another enzyme may be used to synthesize a new polynucleotide sequence, in some cases covalently joining two probes from a single probe set. Within a probe set, any probe may bear one or more labels, or affinity tags used for either locus identification or isolation. In one aspect, the first and second labeling probes are hybridized to the first and second nucleic acid regions of interest in nucleotide molecules of the genetic sample, respectively; the first and second tagging probes are hybridized to the first and second nucleic acid regions of interest in nucleotide molecules of the genetic sample, respectively; the first labeling probe is hybridized to a region adjacent to where the first tagging probe is hybridized; and the second labeling probe is hybridized to a region adjacent to where the second tagging probe is hybridized.

The hybridization occurs in such a manner that the probes within a probe set may be modified to form a new, larger molecular entity (e.g., a probe product). The probes herein may hybridize to the nucleic acid regions of interest under stringent conditions. As used herein the term “stringency” is used in reference to the conditions of temperature, ionic strength, and the presence of other compounds such as organic solvents, under which nucleic acid hybridizations are conducted. “Stringency” typically occurs in a range from about Tₘ° C. to about 20° C. to 25° C. below Tₘ. A stringent hybridization may be used to isolate and detect identical polynucleotide sequences or to isolate and detect similar or related polynucleotide sequences. Under “stringent conditions” the nucleotide sequence, in its entirety or portions thereof, will hybridize to its exact complement and closely related sequences. Low stringency conditions comprise conditions equivalent to binding or hybridization at 68° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5×Denhardt's reagent (50×Denhardt's contains per 500 ml: 5 g Ficoll (Type 400), 5 g BSA) and 100 μg/ml denatured salmon sperm DNA followed by washing in a solution comprising 2.0+SSPE, 0.1% SDS at room temperature when a probe of about 100 to about 1000 nucleotides in length is employed. It is well known in the art that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol), as well as components of the hybridization solution may be varied to generate conditions of low stringency hybridization different from, but equivalent to, the above listed conditions. In addition, conditions which promote hybridization under conditions of high stringency (e.g., increasing the temperature of the hybridization and/or wash steps, the use of formamide in the hybridization solution, etc.) are well known in the art. High stringency conditions, when used in reference to nucleic acid hybridization, comprise conditions equivalent to binding or hybridization at 68° C. in a solution consisting of 5+SSPE, 1% SDS, 5×Denhardt's reagent and 100 μg/ml denatured salmon sperm DNA followed by washing in a solution comprising 0.1+SSPE and 0.1% SDS at 68° C. when a probe of about 100 to about 1000 nucleotides in length is employed.

In some embodiments, the probe product may be formed only if the probes within a probe set are correctly hybridized. Therefore, the probe products may be formed with high stringency and high accuracy. Again, the probe products may contain sufficient information for identifying the genomic sequence for which the probe product was designed to interrogate. Therefore, generation and direct quantification of a particular probe product (in this case, by molecular counting) may reflect the abundance of a particular genetic sequence in the originating sample.

In additional embodiments, the nucleic acid regions of interest, to which the probes are configured to hybridize to, are located in different chromosomes. For example, the first nucleic acid region of interest is located in chromosome 21, and the second nucleic acid region of interest is not located in chromosome 21 (e.g., located in chromosome 18).

In yet additional embodiments, the specificity and accuracy of probe product formation may be enhanced by making one or more modifications to the process. For example, the temperature of the mixture of probes and the genetic sample may be increased prior to addition of a ligation agent such that only labeling probes and tagging probes that are an exact match to the genomic sequence they have been designed to interrogate can hybridize to it. In this embodiment, the ligation agent may be added to the mixture at this increased temperature so that ligation can only occur between a labeling probe and a tagging probe that map to genomic sequences that are immediately adjacent to one another to form the correct probe product. A further embodiment would include the addition of stabilizing agents to the mixture of probes and the genetic sample prior to addition of the ligation agent to stabilize the hybridized duplexes formed at higher temperatures. Such agents include, but are not limited to, polyamines, such as spermidine or spemine, or betaines, such as N,N,N-trimethylglycine.

In another aspect, the methods of the present disclosure may comprise ligating the first labeling probe and the first tagging probe, and ligating the second labeling probe and the second tagging probe. The ligation of the probes may be performed simultaneously or after contacting the probes to the genetic sample, amplifying and/or immobilizing the probes. Moreover, the ligation of the probes may be performed simultaneously or before contacting the probes to the genetic sample, amplifying, and/or immobilizing the probes. The ligation herein means the process of joining two probes (e.g., joining two nucleotide molecules) together. For example, ligation herein may involve the formation of a 3′,5′-phosphodiester bond that links two nucleotides, and a joining agent that is an agent capable of causing ligation may be an enzyme or a chemical.

In another aspect, the methods of the present disclosure may comprise amplifying the ligated probes and/or ligated probe sets. The amplification of the ligated probes may be performed simultaneously or after contacting the probes to the genetic sample, ligating, hybridizing and/or immobilizing the probes. Moreover, the amplification of the ligated probes may be performed simultaneously or before immobilizing the probes. Amplification herein is defined as the production of additional copies of the probe and/or probe product and may be carried out using polymerase chain reaction technologies well known in the art. As used herein, the term “polymerase chain reaction” (“PCR”) refers to a method for increasing the concentration of a segment of a target sequence (e.g., in a mixture of genomic DNA) without cloning or purification. The length of the amplified segment of the desired target sequence is determined by the relative positions of two oligonucleotide primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.” With PCR, it is possible to amplify a single copy of a specific target sequence in genomic DNA to a level detectable by several different methodologies (e.g., hybridization with a labeled probe). In addition to genomic DNA, any oligonucleotide sequence may be amplified with the appropriate set of primer molecules. In particular, the amplified segments created by the PCR process itself are, themselves, efficient templates for subsequent PCR amplifications. An amplification may be a “real-time” amplification if a detection chemistry is available that permits a reaction product to be measured as the amplification reaction progresses, e.g., “real-time PCR,” or “real-time NASBA” as described in Leone et al, Nucleic Acids Research, 26: 2150-2155 (1998).

As discussed above, the methods described herein may comprise amplification with or without labeling during the amplification. In some embodiments, as described herein, the methods may comprise amplifying with at least one primer comprising a label. In additional embodiments, the method may comprise producing multiple amplification products by using multiple labeling primers separately. For example, if the labels interact with one another, they may bias one or both of the PCR reactions when performed together. Instead, a first amplification reaction may be performed with a first forward or reverse primer comprising a first label and optionally a common reverse or forward corresponding primer to produce a first labeled amplification product. A second amplification reaction may be performed with a second forward or reverse primer comprising a second label and optionally the common reverse or forward corresponding primer to produce a second labeled amplification product. The first and second labeled amplification products may then be combined prior to immobilization. In some instances, normalization may be performed prior to combining the first and second labeled amplification products. Examples of such normalization include normalizing or adjusting the mass of the two first and second labeled amplification products to be equal in the combined product.

Primers are usually single-stranded for maximum efficiency in amplification, but may alternatively be double-stranded. If double-stranded, the primer is usually first treated to separate its strands before being used to prepare extension products. This denaturation step is typically influenced by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded nucleotides linked at its 3′ end complementary to the template in the process of DNA synthesis.

A “primer pair” as used herein refers to a forward primer and a corresponding reverse primer, having nucleic acid sequences suitable for nucleic acid-based amplification of a target nucleic acid. Such primer pairs generally include a first primer having a sequence that is the same or similar to that of a first portion of a target nucleic acid, and a second primer having a sequence that is complementary to a second portion of a target nucleic acid to provide for amplification of the target nucleic acid or a fragment thereof. Reference to “first” and “second” primers herein is arbitrary, unless specifically indicated otherwise. For example, the first primer may be designed as a “forward primer” (which initiates nucleic acid synthesis from a 5′-end of the target nucleic acid) or as a “reverse primer” (which initiates nucleic acid synthesis from a 5′-end of the extension product produced from synthesis initiated from the forward primer). Likewise, the second primer may be designed as a forward primer or a reverse primer.

In some embodiments, the nucleic acid region of interest in the nucleotide molecule herein may be amplified by the amplification methods described herein. The nucleic acids in a sample may or may not be amplified prior to analysis, using a universal amplification method (e.g., whole genome amplification and whole genome PCR). The amplification of the nucleic acid region of interest may be performed simultaneously or after contacting the probes to the genetic sample, ligating, amplifying and/or immobilizing the probes. Moreover, the amplification of the ligated probes may be performed simultaneously or before contacting the probes to the genetic sample, ligating the probes, immobilizing the probes, and/or counting the labels.

In additional embodiments, the method excludes amplification of the nucleotide molecules of the genetic sample after the hybridization or the ligation. In further embodiments, the method excludes amplification of the nucleotide molecules of the genetic sample after the hybridization and the ligation.

In another aspect, the methods of the present disclosure may comprise immobilizing the tagging probes to a predetermined location on a substrate. The immobilization of the probe to a substrate may be performed simultaneously or after contacting the probes to the genetic sample, hybridizing the probes to the nucleic acid region of interest, ligating and/or amplifying the probes. Moreover, the immobilization of the probe to a substrate may be performed simultaneously or before contacting the probes to the genetic sample, hybridizing the probes to the nucleic acid region of interest, ligating, amplifying and/or counting the probes. Immobilization herein means directly or indirectly binding the tagging probes to the pre-determined location on the substrate by a physical or chemical bond. In some embodiments, the substrate herein may comprise a binding partner that is configured to contact and bind to a part or full tag in the tagging probe described herein and immobilize the tag and thus the tagging probe comprising the tag. The tag of the tagging probe may comprise a corresponding binding partner of the binding partner on the substrate as described herein.

Immobilization may be performed by hybridizing a part or full tagging probe to a part or full binding partner on the substrate. For example, the immobilizing step comprises hybridizing at least a part of the tag or tagging nucleotide sequence to a corresponding nucleotide molecule immobilized on the substrate. Here, the corresponding nucleotide molecule is a binding partner of the tag or tagging nucleotide sequence that is configured to hybridize partially or fully to the tag or tagging nucleotide sequence. In some embodiments, the oligonucleotide or polynucleotide binding partners may be single stranded and may be covalently attached to the substrate, for example, by 5′-end or a 3′-end. Immobilization may also be performed by the following exemplary binding partners and binding means: Biotin-oligonucleotide complexed with Avidin, Strepatavidin or Neutravidin; SH-oligonucleotide covalently linked via a disulphide bond to a SH-surface; Amine-oligonucleotide covalently linked to an activated carboxylate or an aldehyde group; Phenylboronic acid (PBA)-oligonucleotide complexed with salicylhydroxamic acid (SHA); Acrydite-oligonucleotide reacted with thiol or silane surface or co-polymerized with acrylamide monomer to form polyacrylamide, or by other methods known in the art. For some applications where it is preferable to have a charged surface, surface layers may be composed of a polyelectrolyte multilayer (PEM) structure as shown in U.S. Patent Application Publication No. 2002/025529. In some embodiments, the immobilization may be performed by well-known procedures, for example, comprising contacting the probes with the support having binding partners attached for a certain period of time, and after the probes are depleted for the extension, the support with the immobilized extension products is optionally rinsed using a suitable liquid. In additional embodiments, immobilizing probe products onto a substrate may allow for rigorous washing for removing components from the biological sample and the assay, thus reducing background noise and improving accuracy.

In another aspect, the tag may be at the end of the molecule to aid binding to the substrate. For example, if the tag is a nucleotide sequence and it is at the terminus of the ligation product, it may be more available for hybridization to its complementary molecule on the surface. Ligation products with the tag at the end may be produced in a number of different ways. For example, if the tagging probe has an internal tag (that is, the tag is at neither end of the molecule), then the tag can be made to be at the end by cleaving off part of the molecule. In some embodiments, a cleavage site may be placed next to the tag and then a part of the ligation product may be enzymatically cleaved off, producing a truncated ligation product with the tag at the end.

In other embodiments, the tag could be placed on one of the primers (if amplification is to take place). In this case, the tag is at the end of the primer sequence and thus is incorporated at the end of the molecule during amplification. Amplification may be either linear amplification or non-linear amplification (e.g. PCR) or some combination of the two.

In additional embodiments, the tag may be added either after the hybridization, the ligation and/or the amplification. For example, the tag may be ligated onto one or both ends of a probe at any stage during the assay to produce a ligation product with the tag at one or both ends.

“Solid support,” “support,” “substrate,” and “solid phase support” are used interchangeably and refer to a material or group of materials having a rigid or semi-rigid surface or surfaces. In some embodiments, at least one surface of the substrate will be substantially flat, although in some embodiments it may be desirable to physically separate synthesis regions for different compounds with, for example, wells, raised regions, pins, etched trenches, or the like. In additional embodiments, the substrate may comprise at least one planar solid phase support (e.g., a glass microscope slide). According to other embodiments, the substrate(s) will take the form of beads, resins, gels, microspheres, or other geometric configurations. In one aspect, the substrate according to some embodiments of the present disclosure excludes beads, resins, gels, and/or microspheres.

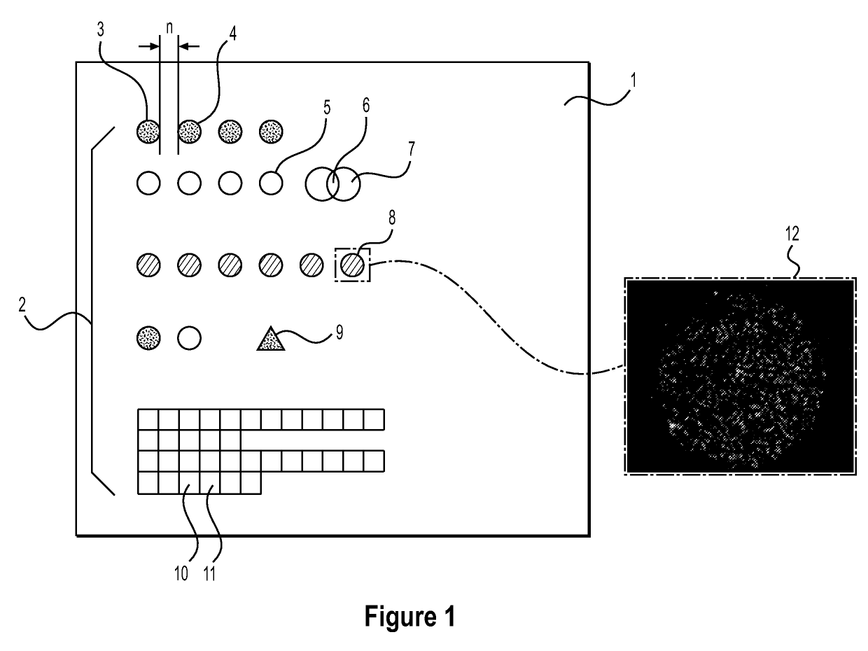

In some embodiments, as shown in FIG. 1, the binding partners, the tags, the affinity tags, labels, the probes (e.g., tagging probes and labeling probes), and/or the probe sets described herein may be immobilized on a substrate (1) as an array (2). The array herein has multiple members (3-10) that may or may not have an overlap (6) between the members. Each member may have at least an area with no overlap with another member (3-5 and 7-10). In additional embodiments, each member may have different shapes (e.g., circular spots (3-8), triangles (9), and squares (10)) and dimensions. A member of an array may have an area about from 1 to 10⁷ micron², from 100 to 10⁷ micron², from 10³ to 10⁸ micron², from 10⁴ to 10⁷ micron²; from 10⁵ to 10⁷ micron²; about 0.0001, 0.001, 0.01, 0.1, 1, 10, 100, 10³, 10⁴, 10⁵, 10⁶, 10⁷, 10⁸ or more micron²; and/or about 0.001, 0.01, 0.1, 1, 10, 100, 10³, 10⁴, 10⁵, 10⁶, 10⁷, 10⁸ or less micron². An image of an exemplary member (8) according to some embodiments of the present invention is shown as item 12. Moreover, two or more members comprising the binding partners, the tags, the affinity tags, labels, the probes (e.g., tagging probes and labeling probes), and/or the probe sets of the same type may have the same shape and dimension. Specifically, the members of an array comprising the binding partners, tags, affinity tags, labels, tagging probes and/or probe sets configured or used to detect the same genetic variation or a control according to the methods described herein may have the same shapes and dimensions. Further, each and every member of the arrays on the substrate may have the same shapes and dimensions. In other embodiments, the members of an array comprising the binding partners, tags, affinity tags, labels, probes and/or probe sets configured or used to detect different genetic variations and/or controls according to the methods described herein may have the same shapes and dimensions. In addition, each member of the array may comprise different binding partners, the tags, the affinity tags, labels, the probes, and/or the probe sets.

In some embodiments, two members of the array may be separated by (i) a distance, in which there may be no or only very few binding partners, the tags, the affinity tags, labels, the probes (e.g., tagging probes and labeling probes), and/or the probe sets immobilized, and/or (ii) any separator distinguishing one member from the other (e.g., heightened substrate, any material preventing binding of the binding partners, the tags, the affinity tags, the probes (e.g., tagging probes), and/or the probe sets to the substrate, and any non-probe material between the members). In additional embodiments, the members of the array may be distinguished from each other at least by their locations alone. The members of the array may be separated by a distance about from 0 to 10⁴ microns, from 0 to 10³ microns, from 10² to 10⁴ microns, or from 10² to 10³ microns; about 0, 0.001, 0.1, 1, 2, 3, 4, 5, 10, 50, 100, 10³, 10⁴, 10⁵, 10⁶, 10⁷, or 10⁸ microns or more; and/or about 0, 0.001, 0.1, 1, 2, 3, 4, 5, 10, 50, 100, 10³, 10⁴, 10⁵, 10⁶, 10⁷, or 10⁸ microns or less. Here, the distance by which two members of the array are separated may be determined by the shortest distance between the edges of the members. For example, in FIG. 1, the distance by which two members, items 3 and 4, of an array (2) are separated is the distance indicated by item n. Moreover, for example, the shortest distance by which the members of the array (2) on a substrate (1) are separated is 0, as the distance by which two members, items 10 and 11, of the array are separated. In other embodiments, two members of the array may not be separated and may be overlapped (6). In such embodiments, each member may have at least an area with no overlap with another member (7).

In further embodiments, an array and the members of the array of the binding partners, the tags, the affinity tags, labels, the probes, and/or the probe sets described herein may be located on predetermined locations on the substrate, and the shapes and dimensions of each member of the array and the distance between the members may be predetermined prior to the immobilization. The predetermined location herein means a location that is determined or identified prior to the immobilization. For example, the shape and dimension of each member of an array is determined or identified prior to the immobilization.

In additional embodiments, the substrate may comprise an array of binding partners, each member of the array comprising the binding partners, such as oligonucleotides or polynucleotides, that are immobilized (e.g., by a chemical bond that would be not broken during the hybridization of probes to the binding partners of the substrate described herein) to a spatially defined region or location; that is, the regions or locations are spatially discrete or separated by a defined region or location on the substrate. In further embodiments, the substrate may comprise an array, each member of which comprises binding partners binding to a spatially defined region or location. Each of the spatially defined locations configured to comprise the binding partners may additionally be “addressable” in that its location and the identity of its immobilized binding partners are known or predetermined, for example, prior to its use, analysis, or attaching to their binding partners in tagging probes and/or probe sets. The term “addressable” with respect to the probe sets immobilized to the substrate means that the nucleotide sequence or other physical and/or chemical characteristics of an end-attached part (e.g., a binding partner of the binding partner of the substrate, tag, affinity tag, and tagging probe) of a probe set described herein may be determined from its address, i.e., a one-to-one correspondence between the sequence or other property of the end-attached part of the probe set and a spatial location on, or characteristic of, the substrate to which the probe set is immobilized. For example, an address of an end-attached part of a probe set is a spatial location, e.g., the planar coordinates of a particular region immobilizing copies of the end-attached part of the probe set. However, end-attached parts of probe sets may be addressed in other ways too, e.g., by color, frequency of micro-transponder, or the like, e.g., Chandler et al, PCT publication WO 97/14028, which is herein incorporated by reference in their entirety for all purposes. In further embodiments, the methods described herein exclude “random microarray,” which refers to a microarray whose spatially discrete regions of binding partners (e.g., oligonucleotides or polynucleotides) of the substrate and/or the end-attached parts of probe sets are not spatially addressed. That is, the identity of the attached binding partners, tag, affinity tag, tagging probe, and/or probe sets is not discernible, at least initially, from its location. In one aspect, the methods described herein exclude random microarrays that are planar arrays of microbeads.

An array of nucleic acid according to some embodiments of the present disclosure may be produced by any method well known in the art, including but not limited to those described in U.S. Patent Application Publication No. 2013/0172216, which is incorporated by reference in its entirety for all purpose; Schena, Microarrays: A Practical Approach (IRL Press, Oxford, 2000). For example, a DNA capture array may be used. The DNA capture array is a solid substrate (e.g., a glass slide) with localized oligonucleotides covalently attached to the surface. These oligonucleotides may have one or more types on the surface, and may further be segregated geographically across the substrate. Under hybridization conditions, DNA capture arrays will preferentially bind complementary targets compared to other non-specific moieties, thereby acting to both localize targets to the surface and separate them from un-desired species.

In some embodiments, the first and second labeling probes and/or the amplified labeling probes thereof ligated to the immobilized tagging probes comprise first and second labels, respectively.

The labeling probe herein means a probe that comprises or is configured to bind to a label. The labeling probe itself may comprise a label or may be modified to comprise or bind to a label. The amplified probe herein is defined to be the additional copies of an initial probe produced after amplification of the initial probe as described herein. Accordingly, the amplified probes may have a sequence that is the nucleotide sequences of the initial probes and/or complementary sequence of the nucleotide sequences of the initial probes. The amplified probes may contain a sequence that is partial or complete match to the nucleotide sequences of the initial probes. The terms “complementary” or “complementarity” are used in reference to a sequence of nucleotides related by the base-pairing rules. For example, the sequence “5′-CAGT-3′,” is complementary to the sequence “5′-ACTG-3′.” Complementarity may be “partial” or “total.” “Partial” complementarity is where one or more nucleic acid nucleotides in a probe is not matched according to the base pairing rules while others are matched. “Total” or “complete” complementarity between nucleic acids is where each and every nucleic acid base in the probe is matched with another base under the base pairing rules.

Immobilized probe herein is defined to be a probe that is directly or indirectly binding to the substrate by a physical or chemical bond. In some embodiments, a labeling probe may be immobilized to a substrate indirectly via ligation to a tagging probe immobilized to the substrate described herein.

A label herein means an organic, naturally occurring, synthetic, artificial, or non-naturally occurring molecule, dye, or moiety having a property or characteristic that is capable of detection and, optionally, of quantitation. A label may be directly detectable (e.g., radioisotopes, fluorophores, chemiluminophores, enzymes, colloidal particles, fluorescent substances, Quantum dots or other nanoparticles, nanostructures, metal compounds, organometallic labels, and peptide aptamers); or a label may be indirectly detectable using specific binding partners. Examples of the fluorescent substances include fluorescent dyes such as fluorescein, phosphor, rhodamine, polymethine dye derivatives, and the like. Examples of a commercially available fluorescent substance include fluorescent dyes, such as BODYPY FL (trademark, produced by Molecular Probes, Inc.), FluorePrime (product name, produced by Amersham Pharmacia Biotech, Inc.), Fluoredite (product name, produced by Millipore Corporation), FAM (produced by ABI Inc.), Cy 3 and Cy 5 (produced by Amersham pharmacia), TAMRA (produced by Molecular Probes, Inc.), Pacific Blue, TAMRA, Alexa 488, Alexa 546, Alexa 555, Alexa 594, Alexa 647, Alexa 680, Atto 488, Atto 590, Atto 647N and the like. “Quantum dot” (QD) means a nano-scale semiconductor crystalline structure, usually made from cadmium selenide, and absorbs light and then re-emits it a couple of nanoseconds later in a specific color. QDs with a variety of conjugated or reactive surfaces, e.g., amino, carboxyl, streptavidin, protein A, biotin, and immunoglobulins, are also encompassed in the present disclosure.

In additional embodiments, the first and second labels are different so that the labels may be distinguished from each other. In further embodiments, the first and second labels are different in their physical, optical, and/or chemical properties.

In some embodiments, the immobilized labels are optically resolvable. The term “optically resolvable label” or “optically individually resolvable label” herein means a group of labels that may be distinguished from each other by their photonic emission, or other optical properties, for example, after immobilization as described herein. In additional embodiments, even though the labels may have the same optical and/or spectral emission properties, the immobilized labels may be distinguished from each other spatially. In some embodiments, the labels of the same type, which is defined to be labels having the same optical properties, are immobilized on the substrate, for example as a member of an array described herein, at a density and/or spacing such that the individual probe products are resolvable as shown in item 12 of FIG. 1. In this disclosure, the “same labels” are defined to be labels having identical chemical and physical compositions. The “different labels” herein mean labels having different chemical and/or physical compositions, including “labels of different types” having different optical properties. The “different labels of the same type” herein means labels having different chemical and/or physical compositions, but the same optical properties.

Item 12 of FIG. 1 depicts an image of an exemplary member of an array comprising immobilized labels. In these embodiments, the labels are spatially addressable as the location of a molecule specifies its identity (and in spatial combinatorial synthesis, the identity is a consequence of location). In additional embodiments, one member of the array on the substrate may have one or multiple labeled probes immobilized to the member. When multiple labeled probes are immobilized to one member of the array, the labels of the same type in the labeled probes immobilized to the one member of an array on the substrate may be distinguished from each other spatially as shown in item 12 of FIG. 1. In some embodiments, the immobilized labels of the same type are separated by a distance about from 1 to 1000 nm, from 5 to 100 nm, or from 10 to 100 nm; about 100, 150, 200, 250, 300, 350, or 400 nm or more; and/or about 50, 100, 150, 200, 250, 300, 350, or 400 nm or less in all dimensions. The density of the probe products and their labels on the substrates may be up to many millions (and up to one billion or more) probe products to be counted per substrate. The ability to count large numbers of probe products containing the labels allows for accurate quantification of nucleic acid sequences.

In some embodiments, the immobilized first and second tagging probes and/or the amplified tagging probes thereof comprise first and second tags, respectively. The tagging probe herein means a probe that is configured to directly or indirectly bind to the substrate. The tagging probe itself may bind to the substrate or may be modified to bind to the substrate. A tag or affinity tag herein means a motif for specific isolation, enrichment or immobilization of probe products. Examples of the tag or affinity tag include a binding partner described herein, unique DNA sequences allowing for sequence-specific capture including natural genomic and/or artificial non-genomic sequence, biotin-streptavidin, His-tags, FLAG octapeptide, click chemistry (e.g., pairs of functional groups that rapidly and selectively react with each other under mild, aqueous conditions), and antibodies (e.g., azide-cycline). For example, the immobilizing step comprises hybridizing at least a part of the tag, affinity tag, or tagging nucleotide sequence to a corresponding nucleotide molecule immobilized on the substrate. The tag or affinity tag is configured to bind to entities including, but not limited to a bead, a magnetic bead, a microscope slide, a coverslip, a microarray or a molecule. In some embodiments, the immobilizing step is performed by immobilizing the tags to the predetermined location of the substrate.

In another aspect, the numbers of different labels immobilized on the substrate and thus the numbers of different immobilized probe products comprising the labels are counted. For example, the probe products from each genetic locus are grouped together, and the labels in the immobilized probe products are counted. In some embodiments, multiple sequences within a genomic locus may be interrogated via the creation of multiple probe product types. For this example, different probe products for the same genomic locus may be combined (possibly via immobilization to a common location of a substrate, e.g., as a member of an array described herein), and the labels in these probe products may be directly counted. Different probe products for the same genomic locus may be also separated (possibly via immobilization to different locations of a substrate, e.g., as different members of an array described herein), and the labels in these probe products may be directly counted. In additional embodiments, the substrate may have one or more specific affinity tag in each location on a substrate, e.g., as a member of an array on the substrate. Therefore, another method for quantifying nucleic acid sequences occurs via immobilization of probe products for a single genomic locus (this may be one probe product type, or may be a set of more than one probe product for a particular genomic locus) to the same location of a substrate (e.g., as the same member of an array described herein) as probe products corresponding to a second genomic locus, which may or may not serve as a reference or control locus. In this case, the probe products from the first genomic locus will be distinguishable from the probe products from the second genomic locus, based on the presence of different labels used in generating the probe products.

In one example, for detecting trisomy 21 (aneuploidy) of a fetus through examination of a maternal blood sample, a set of probe products corresponding to chromosome 21 would be generated, for example with a red fluorophore label, and counted. A second set of probe products would also be generated from a reference, or control locus, for example chromosome 18, and counted. This second set of probe products may be generated, for example, with a green fluorophore label.

In some embodiments, these probe products may be prepared such that they are grouped together by locus (in this case chromosome 21 or chromosome 18) and counted separately on a substrate. That is, the probe products corresponding to chromosome 21 may be isolated and counted separately, and the probe products corresponding to chromosome 18 may be isolated and counted separately. In additional embodiments, these probe products may be also prepared in such a way that they are grouped together in the same location of a substrate (e.g., as the same member of an array described herein. In this case, on the same region of a substrate, the probe products bearing a red fluorophore will correspond to chromosome 21, and the probe products with a green fluorophore will correspond to chromosome 18. For example, since all of these probe products are individually resolvable and may therefore be counted very accurately, an increased frequency of chromosome 21 probe products relative to chromosome 18 probe products (even as small as 0.01, 0.1, one or more percent or less) will signify the presence of trisomy 21 in a fetus. In this case, the probe products for chromosome 18 may serve as a control.

In another aspect, the methods of the present disclosure may comprise counting the labels of the probe sets immobilized to the substrate. In some embodiments, the methods may comprise counting (i) a first number of the first label immobilized to the substrate, and (ii) a second number of the second label immobilized to the substrate. The counting step may be performed after immobilizing the ligated probe set to a substrate, and the substrate with immobilized ligated probe sets may be stored in a condition to prevent degradation of the ligated probe sets (e.g., at room temperature or a temperature below the room temperature) before the counting step is performed.

In order to accurately quantify the relative abundance of different genomic sequences, for example, for quantification of DNA copy number or for quantification of allele frequency, a large number of probe products may be counted. For example, a label may be detected and counted based on measuring, for example, physicochemical, electromagnetic, electrical, optoelectronic or electrochemical properties, or characteristics of the immobilized label.

In some embodiments, the label may be detected by scanning probe microscopy (SPM), scanning tunneling microscopy (STM) and atomic force microscopy (AFM), electron microscopy, optical interrogation/detection techniques including, but not limited to, near-field scanning optical microscopy (NSOM), confocal microscopy and evanescent wave excitation. More specific versions of these techniques include far-field confocal microscopy, two-photon microscopy, wide-field epi-illumination, and total internal reflection (TIR) microscopy. Many of the above techniques may also be used in a spectroscopic mode. The actual detection is by charge coupled device (CCD) cameras and intensified CCDs, photodiodes and/or photomultiplier tubes. In some embodiments, the counting step comprises an optical analysis, detecting an optical property of a label. In additional embodiments, the optical analysis comprises an image analysis as described herein.

In another aspect, the counting step comprises reading the substrate in first and second imaging channels that correspond to the first and second labels, respectively, and producing one or more images of the substrate, wherein the first and second labeling probes are resolvable in the one or more images. In some embodiments, the counting step comprises spatial filtering for image segmentation. In additional embodiments, the counting step comprises water shedding analysis, or a hybrid method for image segmentation.

The methods described herein may also look at the frequency of different alleles at the same genetic locus (e.g., two alleles of a given single nucleotide polymorphisms). The accuracy of these methods may detect very small changes in frequency (e.g., as low as about 10, 5, 4, 3, 2, 1, 0.5, 0.1 or 0.01% or less). As an example, in the case of organ transplantation, a blood sample will contain a very dilute genetic signature from the donated organ. This signature may be the presence of an allele that is not in the recipient of the donated organ's genome. The methods described herein may detect very small deviations in allele frequency (e.g., as low as about 10, 5, 4, 3, 2, 1, 0.5, 0.1 or 0.01% or less) and may identify the presence of donor DNA in a host sample (e.g., blood sample). An unhealthy transplanted organ may result in elevated levels of donor DNA in the host blood—a rise of only a few percent (e.g., as low as about 10, 5, 4, 3, 2, 1, 0.5, 0.1 or 0.01% or less). The methods described herein may be sensitive enough to identify changes in allele frequency with the necessary sensitivity, and therefore may accurately determine the presence and changing amounts of donor DNA in host blood.

In another aspect, the counting of the method described herein may comprise determining the presence of a label immobilized on a substrate by fitting a Gaussian model, for example, to an image of the labels. The intensity or other metric related of the label may be expected to decay with increasing distance from the label, and a Gaussian distribution can be used to model this. For example, a two dimensional Gaussian would be fit (representing the x and y coordinates on the substrate). More complex distributions may also be fit to the data to determine the presence of a label. Further, fitting Gaussian or other models may be used to distinguish one label from an aggregation of two or more labels as the presence of multiple labels would be expected to change the observed distribution (for example, of intensity or signal-to-noise). The method of fitting a distribution that models aspects of the expected size, shape, symmetry and magnitude of features of the label may increase the accuracy of detection over simpler methods. Further, algorithms may be optimized to allow rapid detection using model fitting. Various combinations of image analysis techniques may be used together. For example, water shedding analysis can be used to determine potential locations of labels, and then Gaussian fitting may be used to determine whether a single label or multiple labels are present in a given location. Given image analysis methods may also be used repeatedly, for example, watershedding using a first threshold may be used to identify potential locations of labels and then a second round of watershedding with a different threshold may be used to determine the number of labels at the said locations.

In another aspect, instead of counting integer numbers of the labels, probes, or probe sets, real values for the numbers of the labels, probes, or probe sets may be measured. When there is a high frequency of overlapping labels, this method may be especially advantageous. That is, when two or more labels are coincident but optically resolvable from other labels, they may appear to have many of the same characteristics as a single label. For example, the two or more labels may have similar symmetry and point-spread-function (PSF) as the single label. If all the co-localized labels are emitting, however, the intensity, signal to noise and other characteristics may be different. On average, two coincident labels should be brighter than a single label. Because there is variance in the labels (e.g. the intensity of labels may vary), it may not be possible to determine exactly how many labels are at a given location. In such a case, weighting each location that contains one or more labels by some measure of intensity, SNR or other property may capture information on the number of labels present at that location. In this way, instead counting integer numbers for the locations counting labels, counts are weighted by the appropriate metric (e.g. intensity), and these potentially non-integer, real numbers are summed. This is different from regular microarrays in that the array of this embodiment is still optically resolvable in that the locations with labels are optically resolvable from each other, with each location containing one or more labels. That is, there is not a continuous surface of labels, and the summation is not across the entire surface. Instead, the summation is specifically in the locations that contain one or more labels.

In another aspect, the methods of the present disclosure may comprise comparing the first and second numbers to determine the genetic variation in the genetic sample. In some embodiments, the comparing step comprises obtaining an estimate of a relative number of the nucleotide molecules having the first and second nucleic acid regions of interest.

In another aspect, the methods of the present disclosure may comprise labeling the first and second labeling probes with the first and second labels, respectively, prior to the contacting step (e.g., during manufacturing the probes). Labeling the probe may be performed simultaneously or after contacting the probes to the genetic sample, hybridizing, ligating, amplifying and/or immobilizing the probes. Moreover, labeling the probe may be performed simultaneously or before contacting the probes to the genetic sample, hybridizing, ligating, amplifying, and/or immobilizing the probes. Labeling a probe may comprise adding, immobilizing, or binding a label to the probe by a physical or chemical bond. Labels may be placed anywhere within the sequence of a probe, including at the 5′ or 3′-end.

In another aspect, the methods of the present disclosure may comprise tagging the first and second tagging probes with first and second tags, respectively, prior to the contacting step. (e.g., during the manufacturing the probes). Tagging the probe may be performed simultaneously or after contacting the probes to the genetic sample, hybridizing, ligating, amplifying and/or labeling the probes. Moreover, tagging the probe may be performed simultaneously or before contacting the probes to the genetic sample, hybridizing, ligating, amplifying, immobilizing and/or labeling the probes. Tagging a probe may comprise adding, immobilizing, or binding a tag to the probe by a physical or chemical bond. Tags may be placed anywhere within the sequence of a probe, including at the 5′ or 3′-end.

In another aspect, the probe sets herein may be designed to have tags according to the predetermined locations to which the tags are to be immobilized. In some embodiments, the tags in all probe sets configured to detect a genetic variation are the same and are configured to be immobilized to same locations on the substrate directly or indirectly. In additional embodiments, the first and second tags are the same, and each of the rest of the tags is different from the first or second tag. In further embodiments, each or a group of members of the array of multiple predetermined locations on a substrate may have a unique tag to be immobilized.

In another aspect, the probe sets according to some embodiments may be amplified, and labeled probe sets may be produced during the process of amplification. In another aspect, each of the labeling probes may comprise a forward or reverse priming sequence, and each of the tagging probes may comprise a corresponding reverse or forward priming sequence and a tagging nucleotide sequence as a tag. The forward and reverse priming sequences are the sequences that are configured to hybridize to the corresponding forward and reverse primers, respectively. In some embodiments, the amplifying step comprises amplifying (i) the ligated first labeling and tagging probes with first forward and reverse primers hybridizing to the forward and reverse priming sequences, respectively, wherein the first forward or reverse primer hybridizing to the first labeling probe comprises the first label, and (ii) the ligated second labeling and tagging probes with second forward and reverse primers hybridizing to the forward and reverse priming sequences, respectively, wherein the second forward or reverse primer hybridizing to the second labeling probe comprises the second label. In additional embodiments, the amplified tagging nucleotide sequences of the tagging probes are immobilized to a pre-determined location on a substrate, wherein the amplified tagging nucleotide sequences of the first and second tagging probes are the first and second tags. In some embodiments, the first and second tags are the same and/or are configured to bind to the same location on the substrate. In another embodiment, the first and second tags are different and/or are configured to bind to different locations on the substrate. In further embodiments, when the probes are amplified, the method comprises counting numbers of the labels in the amplified probes and/or probe sets immobilized on the substrate. For example, the first number is the number of the first label in the amplified first probe set immobilized to the substrate, and the second number is the number of the second label in the amplified second probe set immobilized to the substrate.

In another aspect, the probe sets according to some embodiments may be amplified, and labeled probe sets may be produced using labeled reverse primers without using a forward primer. In another aspect, each of the labeling probes may comprise a reverse priming sequence, and each of the tagging probes may comprise a tagging nucleotide sequence as a tag. In some embodiments, the amplifying step may comprise amplifying (i) the ligated first labeling and tagging probes with a first reverse primer hybridizing to a first reverse priming sequence of the first labeling probe, wherein the first reverse primer comprises the first label, and (ii) the ligated second labeling and tagging probes with a second reverse primer hybridizing to a second reverse priming sequence of the second labeling probe, wherein the second reverse primer comprises the second label. In additional embodiments, the amplified tagging nucleotide sequences of the tagging probes are immobilized to a pre-determined location on a substrate, wherein the amplified tagging nucleotide sequences of the first and second tagging probes are the first and second tags. In further embodiments, the first number is the number of the first label in the amplified first probe set immobilized to the substrate, and the second number is the number of the second label in the amplified second probe set immobilized to the substrate.

In another aspect, the ligated probe sets according to some embodiments may be produced using a ligase chain reaction. In another aspect, the method described herein comprises contacting third and fourth probe sets to the genetic sample, wherein the third probe set comprises a third labeling probe and a third tagging probe, and the fourth probe set comprises a fourth labeling probe and a fourth tagging probe. The method may further comprise hybridizing the first and second probe sets to first and second sense nucleic acid strands of interest in single stranded nucleotide molecules from the double stranded nucleotide molecules of the genetic sample, respectively; and hybridizing the third and fourth probe sets to anti-sense nucleic acid strands of the first and second sense nucleic acid strands of interest, respectively. The method may further comprise producing ligated first, second, third, and fourth probe sets at least by ligating (i) the first labeling probe and the first tagging probe, (ii) the second labeling probe and the second tagging probe, (iii) the third labeling probe and the third tagging probe, and (iv) the fourth labeling probe and the fourth tagging probe. The method may further comprise performing a ligase chain reaction known in the art to amplify the ligated probe and/or ligated probe sets. In some embodiments, the ligase chain reaction may comprise hybridizing non-ligated first, second, third and fourth probe sets to the ligated third, fourth, first, and second probe sets, respectively, and ligating at least (i) the first labeling probe and the first tagging probe, (ii) the second labeling probe and the second tagging probe, (iii) the third labeling probe and the third tagging probe, and (iv) the fourth labeling probe and the fourth tagging probe of the non-ligated probe sets. The method may further comprise immobilizing the tagging probes to the pre-determined location on a substrate, wherein the first, second, third and fourth labeling probes ligated to the immobilized first, second, third and fourth tagging probes, respectively, comprise first, second, third and fourth labels, respectively; the immobilized labels are optically resolvable; the immobilized first, second, third and fourth tagging probes comprise first, second, third and fourth tags, respectively, and the immobilizing step is performed by immobilizing the tags to the predetermined location. The method may further comprise counting (i) the first sum of the first and third labels immobilized to the substrate, and (ii) the second sum of the second and fourth labels immobilized to the substrate, and comparing the first and second sums to determine the genetic variation in the genetic sample. In yet additional embodiments, the method further comprises labeling the first, second, third and fourth labeling probes with the first, second, third and fourth labels, respectively, prior to the contacting step. In yet further embodiments, the first and third labels are the same, and the second and fourth labels are the same.

In another aspect, the method described herein comprises contacting third and fourth probe sets to the genetic sample, wherein the third probe set comprises a third labeling probe and a third tagging probe, and the fourth probe set comprises a fourth labeling probe and a fourth tagging probe, the first and third labeling probes comprises a first reverse priming sequence, the second and fourth labeling probes comprises a second reverse priming sequence, and each of the tagging probes comprises a tagging nucleotide sequence as a tag. The method may further comprise hybridizing the first and second probe sets to first and second sense nucleic acid strands of interest, respectively, in single stranded nucleotide molecules from double stranded nucleotide molecules of the genetic sample; and hybridizing at least parts of the third and fourth probe sets to anti-sense nucleic acid strands of the first and second sense nucleic acid strands of interest, respectively; producing ligated first, second, third, and fourth probe sets by ligating (i) the first labeling probe and the first tagging probe, (ii) the second labeling probe and the second tagging probe, (iii) the third labeling probe and the third tagging probe, and (iv) the fourth labeling probe and the fourth tagging probe. The method may further comprise performing a ligase chain reaction. In some embodiments, the ligase chain reaction comprises hybridizing at least parts of the non-ligated first, second, third and fourth probe sets to the ligated third, fourth, first, and second probe sets, respectively, and ligating (i) the first labeling probe and the first tagging probe, (ii) the second labeling probe and the second tagging probe, (iii) the third labeling probe and the third tagging probe, and (iv) the fourth labeling probe and the fourth tagging probe of the non-ligated probe set. The method may further comprise amplifying (i) the ligated first and third probe sets with a first reverse primer hybridizing to the first reverse priming sequence, wherein the first reverse primer comprises the first label, and (ii) the ligated second and fourth probe sets with a second reverse primer hybridizing to the second reverse priming sequence, wherein the second reverse primer comprises the second label, the amplified tagging nucleotide sequences of the tagging probes are immobilized to a pre-determined location on a substrate, wherein the amplified tagging nucleotide sequences of the first, second, third and fourth tagging probes are first, second, third and fourth tags, the first number is the number of the first label in the amplified first and third probe sets immobilized to the substrate, and the second number is the number of the second label in the amplified second and fourth probe sets immobilized to the substrate.

In another aspect, the ligated first and second labeling probes are at the 3′-end of the first and second ligated probe set and comprise first and second reverse priming sequences hybridizing to the first and second reverse primers, respectively. In some embodiments, the first and second reverse primers comprise the first and second labels. In additional embodiments, the ligated first and second tagging probes are at the 5′-end of the first and second ligated probe set. In further embodiments, the ligated first and second tagging probes are at the 5′-end of the first and second ligated probe set and comprise first and second corresponding forward priming sequences hybridizing to the first and second forward primers, respectively.

In another aspect, the method herein comprises digesting double stranded molecules in the sample to produce single stranded molecules. In some embodiments, the amplifying step comprises contacting an exonuclease to the amplified probe and/or probe set, and digesting the amplified probe and/or probe set from the 5′-end of one strand of the double stranded amplified probe and/or probe set. For example, the amplifying step comprises contacting an exonuclease to the amplified probe in a probe set, and digesting the amplified probe set from the 5′-end of one strand of the double stranded amplified probe set. In additional embodiments, the one strand of the amplified probe and probe set contacting the exonuclease does not have any label at the 5′-end. The contacting of the exonuclease to the unlabeled double stranded probes may digest the unlabeled strand from the 5′-end producing single stranded probes. In another aspect, the 5′-end of the amplified probe set comprising the label at the 5′-end may be protected from exonuclease digestion.

In another aspect, the present invention is also related to a method of isolating a ligated probe set hybridized to a genetic sample, comprising contacting probe sets to a genetic sample under a condition effective to hybridize the probe sets to a nucleic acid region of interest in nucleotide molecules of the genetic sample, wherein each of the probe sets comprises a first oligonucleotide probe at the 5′ end of the probe set and a second oligonucleotide probe at the 3′ end of the probe set, each of the oligonucleotide probes is configured to hybridize to a part of the nucleic acid region of interest, the first oligonucleotide probe comprises phosphorothioate bonds at three, four, five, six, seven, eight or more nucleotide bonds from the 5′ end, and/or the second oligonucleotide probe comprises phosphorothioate bonds at three, four, five, six, seven, eight or more nucleotide bonds from the 3′ end. In some embodiments, the first and second oligonucleotide probes may comprise a cap structure at the 5′ and 3′ ends, respectively. The cap structure described herein is a structure that resists digestion by one or more exonucleases. In additional embodiments, the cap structure may be any variety of 3′ to 3′ or 5′ to 5′ linkages, including but not limited to, a biological triphosphate version. The modification of the 5′ and 3′ ends may also be any combination of the cap structure and the phosphorylation. The method may also comprise ligating the first and second oligonucleotide probes that are hybridized to the nucleotide molecules, and digesting terminal phosphodiester bonds in non-hybridized oligonucleotide probes and/or partially hybridized nucleic acid molecules with one, two, three, four or more exonucleases. The method may further comprise isolating ligated oligonucleotide probes hybridized to digested nucleic acid molecules from the genetic sample.

In some embodiments, the one, two, three, four or more exonucleases comprises a mixture of exonuclease enzymes that digest both double and single-stranded oligonucleotide molecules from 5′ and 3′-end directions. In additional embodiments, the one, two, three, four or more exonuclease comprises one or more of Exonuclease I, Exonuclease III, Exonuclease VII, Lambda Exonuclease or T7 Gene 6 Exonuclease. In further embodiments, the first and/or second oligonucleotide probe comprises a label and/or a tag. The first and/or second oligonucleotide probe may also comprise biotin. In other embodiments, the probe set may further comprise a third oligonucleotide probe that is configured to hybridize to a nucleic acid region in the nucleotide molecules between the nucleic acid regions to which the first and second oligonucleotide probes hybridize, and the ligating step may comprise ligating the first, second and third oligonucleotide probes that are hybridized to the nucleotide molecules.

In another aspect, the method described herein may further comprise purifying the ligated probe sets or amplification products, after the exonuclease treatments described above, to remove salts, proteins and other material that may be present after the amplification and/or exonuclease treatment. Such purification may also remove digestion products from the exonuclease treatment. The purification also enables the concentration of the reaction product material if necessary, for example if a higher density of labels immobilized on a substrate is required. The purification could comprise one or more of the following methods: use of a DNA binding column, use of a size exclusion column, resolution on and extraction from an agarose or polyacrylamide gel, use of DNA binding magnetic beads, ethanol precipitation or other methods.

In another aspect, the present invention is related to a method of detecting a genetic variation in a genetic sample from a subject, comprising contacting probe sets with a genetic sample under a condition effective to hybridize the probe sets to a nucleic acid region of interest in nucleotide molecules of the genetic sample, wherein each of the probe sets comprises a first oligonucleotide probe at the 5′ end of the probe set and a second oligonucleotide probe at the 3′ end of the probe set, each of the oligonucleotide probes is configured to hybridize to a part of the nucleic acid region of interest, the first oligonucleotide probe comprises phosphorothioate bonds at three, four, five, six, seven, eight or more nucleotide bonds from the 5′ end, and the second oligonucleotide probe comprises phosphorothioate bonds at three, four, five, six, seven, eight or more nucleotide bonds from the 3′ end. The method may also comprise ligating the first and second oligonucleotide probes that are hybridized to the nucleotide molecules and digesting terminal phosphodiester bonds in non-hybridized oligonucleotide probes and/or partially hybridized nucleic acid molecules with one, two, three, four or more exonucleases. The method may further comprise detecting non-digested and ligated probe sets to determine the presence or absence of the genetic variation in a genetic sample from a subject. In some embodiments, the method may comprise amplifying the ligated probe sets before the digestion. In additional embodiments, the probes and/or probe sets digested by the exonuclease described herein may be single or double-stranded after the amplification.

In another aspect, the present invention is related to a method of detecting a genetic variation in a genetic sample from a subject, comprising (i) contacting first and second probe sets to the genetic sample under a condition effective to hybridize the probe sets to a nucleic acid region of interest in nucleotide molecules of the genetic sample, wherein the first probe set comprises a first labeling probe and a first tagging probe, and the second probe set comprises a second labeling probe and a second tagging probe, and each of the probes is configured to hybridize to a part of the nucleic acid region of interest in the nucleic acid molecules and comprises phosphorothioate bonds at three, four, five, six, seven, eight or more nucleotide bonds from the 5′ or 3′end; and (ii) hybridizing at least parts of the first and second probe sets to first and second nucleic acid regions of interest in nucleotide molecules of the genetic sample, respectively. The method may also comprise (i) ligating the first probe set by ligating the first labeling probe and the first tagging probe to produce a first ligated probe set comprising the phosphorothioate bonds at the 5′ and 3′ends; (ii) ligating the second probe set by ligating the second labeling probe and the second tagging probe to produce a second ligated probe set comprising the phosphorothioate bonds at the 5′ and 3′ends; and (iii) digesting terminal phosphodiester bonds in non-hybridized probe sets and/or partially hybridized nucleic acid molecules with one, two, three, four or more exonucleases. The method may further comprise immobilizing the tagging probes to a pre-determined location on a substrate, wherein the first and second labeling probes and/or the amplified labeling probes thereof ligated to the immobilized tagging probes comprise first and second labels, respectively, the first and second labels are different, the immobilized labels are optically resolvable, and the immobilized first and second tagging probes and/or the amplified tagging probes thereof comprise first and second tags, respectively. The method may additionally comprise counting (i) a first number of the first label immobilized to the substrate, and (ii) a second number of the second label immobilized to the substrate, and comparing the first and second numbers to determine the genetic variation in the genetic sample.

In another aspect, the method described herein may detect from 1 to 100, from 1 to 50, from 2 to 40, or from 5 to 10 genetic variations; 2, 3, 4, 5, 6, 7, 8, 9, 10 or more genetic variations; and 100, 50, 30, 20, 10 or less genetic variations. In some embodiments, the method described herein may detect x number of genetic variations using at least (x+1) number of different probe sets. In these embodiments, a number of labels from one type of probe sets may be compared with one or more numbers of labels from the rest of the different types of probe sets. In some embodiments, the method described herein may detect genetic variation in a continuous manner across the entire genome at various resolutions, for example, at 300,000 base resolution such that 100 distributed variations across all chromosomes are separately interrogated and quantified. In additional embodiments, the base resolution is in the range of one or ten to 100 thousand nucleotides up to one million, ten million, or 100 million or more nucleotides.

In another aspect, the method according to some embodiments may detect at least two genetic variations. In some embodiments, the method described herein may further comprise contacting a fifth probe set to the genetic sample, wherein the fifth probe set comprises a fifth labeling probe and a fifth tagging probe. The method may further comprise hybridizing at least a part of the fifth probe set to the third nucleic acid region of interest in nucleotide molecules of the genetic sample, wherein the third nucleic acid region of interest is different from the first and second nucleic acid regions of interest. The method may further comprise ligating the fifth probe set at least by ligating the fifth labeling probe and the fifth tagging probe. The method may further comprise amplifying the ligated probe sets. The method may further comprise immobilizing each of the tagging probe to a pre-determined location on a substrate, wherein the fifth labeling probe and/or the amplified labeling probe thereof ligated to the immobilized tagging probe comprise a fifth label, the fifth label is different from the first and second labels, the immobilized labels are optically resolvable, the immobilized fifth tagging probe and/or the amplified tagging probe thereof comprise a fifth tag, and the immobilizing step is performed by immobilizing the tags to the predetermined location. The method may comprise counting a third number of the fifth label immobilized to the substrate, and comparing the third number to the first and/or second number(s) to determine the second genetic variation in the genetic sample. In some embodiments, the subject may be a pregnant subject, the first genetic variation is trisomy 21 in the fetus of the pregnant subject, and the second genetic variation is selected from the group consisting of trisomy 13, trisomy 18, aneuploidy of X, and aneuploidy of Y in the fetus of the pregnant subject.

In another aspect, the method according to some embodiments may detect at least three genetic variations. In some embodiments, the method described herein further comprises contacting a sixth probe set to the genetic sample, wherein the sixth probe set comprises a sixth labeling probe and a sixth tagging probe. The method may further comprise hybridizing at least a part of the sixth probe set to the fourth nucleic acid region of interest in nucleotide molecules of the genetic sample, wherein the fourth nucleic acid region of interest is different from the first, second, and third nucleic acid regions of interest. The method may further comprise ligating the sixth probe set at least by ligating the sixth labeling probe and the sixth tagging probe. The method may further comprise amplifying the ligated probe sets. The method may further comprise immobilizing each of the tagging probes to a pre-determined location on a substrate, wherein the sixth labeling probe and/or the amplified labeling probe thereof ligated to the immobilized tagging probe comprise a sixth label, the sixth label is different from the first and second labels, the immobilized labels are optically resolvable, the immobilized sixth tagging probe and/or the amplified tagging probe thereof comprise a sixth tag, and the immobilizing step is performed by immobilizing the tags to the predetermined location. The method may further comprise counting a fourth number of the sixth label immobilized to the substrate, and comparing the fourth number to the first, second and/or third number to determine the third genetic variation in the genetic sample.

In another aspect, the method may according to some embodiments detect at least four genetic variations. In some embodiments, the method described herein further comprises contacting a seventh probe set to the genetic sample, wherein the seventh probe set comprises a seventh labeling probe and a seventh tagging probe. The method may further comprise hybridizing at least a part of the seventh probe set to the fifth nucleic acid region of interest in nucleotide molecules of the genetic sample, wherein the fifth nucleic acid region of interest is different from the first, second, third and fourth nucleic acid regions of interest. The method may further comprise ligating the seventh probe set at least by ligating the seventh labeling probe and the seventh tagging probe. The method may further comprise optionally amplifying the ligated probe sets. The method may further comprise immobilizing each of the tagging probes to a pre-determined location on a substrate, wherein the seventh labeling probe and/or the amplified labeling probe thereof ligated to the immobilized tagging probe comprise a seventh label, the seventh label is different from the first and second labels, the immobilized labels are optically resolvable, the immobilized seventh tagging probe and/or the amplified tagging probe thereof comprise a seventh tag, and the immobilizing step is performed by immobilizing the tags to the predetermined location. The method may further comprise counting a fifth number of the seventh label immobilized to the substrate, and comparing the fifth number to the first, second, third and/or fourth number(s) to determine the fourth genetic variation in the genetic sample.

In another aspect, the method according to some embodiments may detect at least five genetic variations. In some embodiments, the method described herein further comprises contacting an eighth probe set to the genetic sample, wherein the eighth probe set comprises a eighth labeling probe and a eighth tagging probe. The method may further comprise hybridizing at least a part of the eighth probe set to the sixth nucleic acid region of interest in nucleotide molecules of the genetic sample, wherein the sixth nucleic acid region of interest is different from the first, second, third, fourth, and fifth nucleic acid regions of interest. The method may further comprise ligating the eighth probe set at least by ligating the eighth labeling probe and the eighth tagging probe. The method may further comprise amplifying the ligated probe sets. The method may further comprise immobilizing each of the tagging probes to a pre-determined location on a substrate, wherein the eighth labeling probe and/or the amplified labeling probe thereof ligated to the immobilized tagging probe comprise a eighth label, the eighth label is different from the first and second labels, the immobilized labels are optically resolvable, the immobilized eighth tagging probe and/or the amplified tagging probe thereof comprise a eighth tag, and the immobilizing step is performed by immobilizing the tags to the predetermined location. The method may further comprise counting a sixth number of the eighth label immobilized to the substrate, and comparing the sixth number to the first, second, third, fourth and/or fifth number(s) to determine the fifth genetic variation in the genetic sample. In some embodiments, the subject is a pregnant subject, and the first, second, third, fourth, and fifth genetic variations are trisomy 13, trisomy 18, trisomy 21, aneuploidy X, and aneuploidy Y in the fetus of the pregnant subject.

In another aspect, the subject is a pregnant subject, the genetic variation is trisomy 21 in the fetus of the pregnant subject, the first nucleic acid region of interest is located in chromosome 21, and the second nucleic acid region of interest is not located in the chromosome 21.

In another aspect, the subject is a pregnant subject, the genetic variation is trisomy 21 in the fetus of the pregnant subject, the first nucleic acid region of interest is located in chromosome 21, and the second nucleic acid region of interest is located in chromosome 18.

In one aspect, the probe set herein may comprise two, three, four, five or more labeling probes, and/or two, three, four, five or more labels. In some embodiments, the method described herein may further comprise the first and second probe sets further comprise third and fourth labeling probes, respectively; the immobilized first probe set and/or amplified first probe set further comprise a ninth label in the third labeling probe and/or amplified product thereof; and the immobilized second probe set and/or amplified second probe set further comprise a tenth label in the fourth labeling probe and/or amplified product thereof. In these embodiments, if the ninth and tenth labels are different from the first and second labels, this method may be used to confirm the number counted for the first and second labels. If the ninth and tenth labels are the same from the first and second labels, respectively, this method may be used to improve the accuracy of detection labels immobilized to each of the nucleic acid regions of interest. For example, using multiple labels would be brighter than using one label, and therefore multiple labels may be more easily detected than one label.

In additional embodiments, (i) the immobilized first probe set and/or amplified first probe set further comprise an eleventh label in the labeling probe, and (ii) the immobilized second probe set and/or amplified second probe set further comprises a twelfth label that is different from the eleventh label in the labeling probe. In further embodiments, wherein the first, second, eleventh and twelfth labels are different from one another, and the counting step further comprises counting numbers of the eleventh and twelfth labels immobilized on the substrate.

In another aspect, the method described herein may be performed with a control sample. In some embodiments, the method may further comprise repeating the steps with a control sample different from the genetic sample from the subject. The method may further comprise counting control numbers of the labels immobilized to the substrate, and comparing the control numbers to the first, second, third, fourth, fifth and/or sixth number to confirm the genetic variation in the genetic sample.

In another aspect, the subject may be a pregnant subject, and the genetic variation is a genetic variation in the fetus of the pregnant subject. In such embodiments, the method may use a Single Nucleotide Polymorphism (SNP) site to determine whether the proportion (e.g., concentration, and number percentage based on the number of nucleotide molecules in the sample) of fetal material (e.g., the fetal fraction) is sufficient so that the genetic variation of the fetus may be detected from a sample from the pregnant subject with a reasonable statistical significance. In additional embodiments, the method may further comprise contacting maternal and paternal probe sets to the genetic sample, wherein the maternal probe set comprises a maternal labeling probe and a maternal tagging probe, and the paternal probe set comprises a paternal labeling probe and a paternal tagging probe. The method may further comprise hybridizing at least a part of each of the maternal and paternal probe sets to a nucleic acid region of interest in nucleotide molecules of the genetic sample, the nucleic acid region of interest comprising a predetermined SNP site, wherein the at least a part of the maternal probe set hybridizes to a first allele at the SNP site, the at least a part of the paternal probe set hybridizes to a second allele at the SNP site, and the first and second alleles are different from each other. The method may further comprise ligating the material and paternal probe sets at least by ligating (i) the maternal labeling and tagging probes, and (ii) the paternal labeling and tagging probes. The method may further comprise amplifying the ligated probes. The method may further comprise immobilizing the tagging probes to a pre-determined location on a substrate, wherein the maternal and paternal labeling probes and/or the amplified labeling probes thereof ligated to the immobilized tagging probes comprise maternal and paternal labels, respectively; the maternal and paternal labels are different, and the immobilized labels are optically resolvable. The method may further comprise counting the numbers of the maternal and paternal labels, and determining whether a proportion of a fetal material in the genetic sample is sufficient to detect the genetic variation in the fetus based on the numbers of the maternal and paternal labels. The method may further comprise determining the proportion of the fetal material in the genetic sample.

In some embodiments, when the subject is a pregnant subject, and the genetic variation is a genetic variation in the fetus of the pregnant subject, the method may further comprise contacting allele A and allele B probe sets that are allele-specific to the genetic sample, wherein the allele A probe set comprises an allele A labeling probe and an allele A tagging probe, and the allele B probe set comprises an allele B labeling probe and an allele B tagging probe. The method may further comprise hybridizing at least a part of each of the allele A and allele B probe sets to a nucleic acid region of interest in nucleotide molecules of the genetic sample, the nucleic acid region of interest comprising a predetermined single nucleotide polymorphism (SNP) site for which a maternal allelic profile (i.e., genotype) differs from a fetal allelic profile at the SNP site (For example, maternal allelic composition may be AA and fetal allelic composition may be AB, or BB. In another example, maternal allelic composition may be AB and fetal allelic composition may be AA, or BB.), wherein the at least a part of the allele A probe set hybridizes to a first allele at the SNP site, the at least a part of the allele B probe set hybridizes to a second allele at the SNP site, and the first and second alleles are different from each other. The method may further comprise ligating the allele A and allele B probe sets at least by ligating (i) the allele A labeling and tagging probes, and (ii) the allele B labeling and tagging probes. The method may further comprise amplifying the ligated probe sets. The method may further comprise immobilizing the tagging probes to a pre-determined location on a substrate, wherein the allele A and allele B labeling probes and/or the amplified labeling probes thereof ligated to the immobilized tagging probes comprise allele A and allele B labels, respectively, the allele A and allele B labels are different, and the immobilized labels are optically resolvable. The method may further comprise counting the numbers of the allele A and allele B labels, and determining whether a proportion of a fetal material in the genetic sample is sufficient to detect the genetic variation in the fetus based on the numbers of the allele A and allele B labels. The method may further comprise determining the proportion of the fetal material in the genetic sample.

In some embodiments, when the subject is a pregnant subject, the genetic variation is a genetic variation in the fetus of the pregnant subject, and the genetic sample comprises a Y chromosome, the method may further comprise contacting maternal and paternal probe sets to the genetic sample, wherein the maternal probe set comprises a maternal labeling probe and a maternal tagging probe, and the paternal probe set comprises a paternal labeling probe and a paternal tagging probe. The method may further comprise hybridizing at least parts of the maternal and paternal probe sets to maternal and paternal nucleic acid regions of interest in nucleotide molecules of the genetic sample, respectively, wherein the paternal nucleic acid region of interest is located in the Y chromosome, and the maternal nucleic acid region of interest is not located in the Y chromosome. The method may further comprise ligating the maternal and paternal probe sets at least by ligating (i) the maternal labeling and tagging probes, and (ii) the paternal labeling and tagging probes. The method may further comprise amplifying the ligated probes. The method may further comprise nucleic acid region of interest comprising a predetermined single nucleotide polymorphism (SNP) site containing more than one SNP, for example two or three SNPs. Further, the SNP site may contain SNPs with high linkage disequilibrium such that labeling and tagging probes are configured to take advantage of the improved energetics of multiple SNP matches or mismatches versus only one. The method may further comprise immobilizing the tagging probes to a pre-determined location on a substrate, wherein the maternal and paternal labeling probes and/or the amplified labeling probes thereof ligated to the immobilized tagging probes comprise maternal and paternal labels, respectively, the maternal and paternal labels are different, and the immobilized labels are optically resolvable. The method may further comprise counting the numbers of the maternal and paternal labels, and determining whether a proportion of a fetal material in the genetic sample is sufficient to detect the genetic variation in the fetus based on the numbers of the maternal and paternal labels. The method may further comprise determining the proportion of the fetal material in the genetic sample.

In additional embodiments, other genetic variations (e.g., single base deletion, microsatellite, and small insertions) may be used in place of the genetic variation at the SNP site described herein.

In one aspect, the probe set described herein may comprise three or more probes, including at least one probe between the labeling and tagging probes. In some embodiments, the first and second probe sets further comprises first and second gap probes, respectively; the first gap probe hybridizes to a region between the regions where the first labeling probe and the first tagging probe hybridize; the second gap probe hybridizes to a region between the regions where the second labeling probe and the second tagging probe hybridize. The method may further comprise the ligating step comprises ligating at least (i) the first labeling probe, the first tagging probe, and the first gap probe, and (ii) the second labeling probe, the second tagging probe, and the second gap probe. In additional embodiments, the gap probe may comprise a label. For example, the first and second gap probes and/or amplified products thereof are labeled with labels (e.g., thirteenth and fourteenth labels, respectively), and each of the labels may be different from the rest of the labels (e.g., the first and second labels). The labels in the gap probes (e.g., thirteenth and fourteenth labels) may be the same or different from each other. In another aspect, the first and second labeling probes are hybridized to the first and second nucleic acid regions of interest in nucleotide molecules of the genetic sample, respectively; the first and second tagging probes are hybridized to the first and second nucleic acid regions of interest in nucleotide molecules of the genetic sample, respectively; the first and second gap probes are hybridized to the first and second nucleic acid regions of interest in nucleotide molecules of the genetic sample, respectively. In some embodiments, there are from 0 to 100 nucleotides, 1 to 100 nucleotides, 2 to 50 nucleotides; 3 to 30 nucleotides, 0, 1, 2, 3, 4, 5, 6, 7, 8, 9, 10, 20, 30, 40, 50, 100, 150, or 200 or more; or 1, 2, 3, 4, 5, 6, 7, 8, 9, 10, 15, 25, 35, 45, 55, 110, 160, or 300 or less between the regions where the first labeling probe and tagging probes are hybridized; and there are from 0 to 100 nucleotides, 1 to 100 nucleotides, 2 to 50 nucleotides; 3 to 30 nucleotides, 0, 1, 2, 3, 4, 5, 6, 7, 8, 9, 10, 20, 30, 40, 50, 100, 150, or 200 nucleotides or more; or 1, 2, 3, 4, 5, 6, 7, 8, 9, 10, 15, 25, 35, 45, 55, 110, 160, or 300 nucleotides or less between the regions where the second labeling probe and tagging probes are hybridized. In additional embodiments, the gap probe between a labeling probe and a tagging probe may have a length from 0 to 100 nucleotides, 1 to 100 nucleotides, 2 to 50 nucleotides; 3 to 30 nucleotides, 0, 1, 2, 3, 4, 5, 6, 7, 8, 9, 10, 20, 30, 40, 50, 100, 150, or 200 or more; or 1, 2, 3, 4, 5, 6, 7, 8, 9, 10, 15, 25, 35, 45, 55, 110, 160, or 300 or less.

In another aspect, the probe set described herein may comprise a spacer ligated and/or conjugated to the labeling probe and the tagging probe. The spacer may or may not comprise oligonucleotides. The spacer may comprise an isolated, purified, naturally-occurring, or non-naturally occurring material, including oligonucleotide of any length (e.g., 5, 10, 20, 30, 40, 50, 100, or 150 nucleotides or less). In some embodiments, the probe may be in a purified restriction digest or produced synthetically, recombinantly or by PCR amplification. For example, the first labeling and tagging probes are conjugated by a first spacer, the second labeling and tagging probes are conjugated by a second spacer, and the first and second spacers are not hybridized to the nucleotide molecules of the genetic sample. In some embodiments, the method further comprises digesting the hybridized genetic sample with an enzyme, and breaking a bond in the first and second spacers after the digestion.

In another aspect, the method described herein excludes identifying a sequence in the nucleotide molecules of the genetic sample, and/or sequencing of the nucleic acid region(s) of interest and/or the probes. In some embodiments, the method excluding sequencing of the probes includes excluding sequencing a barcode and/or affinity tag in a tagging probe. In additional embodiments, the immobilized probe sets to detect different genetic variations, nucleotide regions of interest, and/or peptides of interest need not be detected or scanned separately because sequencing is not required in the methods described herein. In additional embodiments, the numbers of different labels immobilized to the substrate were counted simultaneously (e.g., by a single scanning and/or imaging), and thus the numbers of different labels were not separately counted. In another aspect, the method described herein excludes bulk array readout or analog quantification. The bulk array readout herein means a single measurement that measures the cumulative, combined signal from multiple labels of a single type, optionally combined with a second measurement of the cumulative, combined signal from numerous labels of a second type, without resolving a signal from each label. A result is drawn from the combination of the one or more such measurements in which the individual labels are not resolved. In another aspect, the method described herein may include a single measurement that measures the same labels, different labels of the same type, and/or labels of the same type in which the individual labels are resolved. The method described herein may exclude analog quantification and may employ digital quantification, in which only the number of labels is determined (ascertained through measurements of individual label intensity and shape), and not the cumulative or combined optical intensity of the labels.

In another aspect, the probe set described herein may comprise a binder. A binder is the same material as the tag or affinity tag describe herein. In some embodiments, the method further comprises immobilizing the binder to a solid phase after the ligating steps. The method may further comprise isolating the ligated probe sets from non-ligated probes. In additional embodiments, the binder comprises biotin, and the solid phase comprises a magnetic bead.

In another aspect, the counting step described herein may further comprise calibrating, verifying, and/or confirming the counted numbers. Calibrating herein means checking and/or adjusting the accuracy of the counted number. Verifying and confirming herein mean determining whether the counted number is accurate or not, and/or how much the error is, if exists.

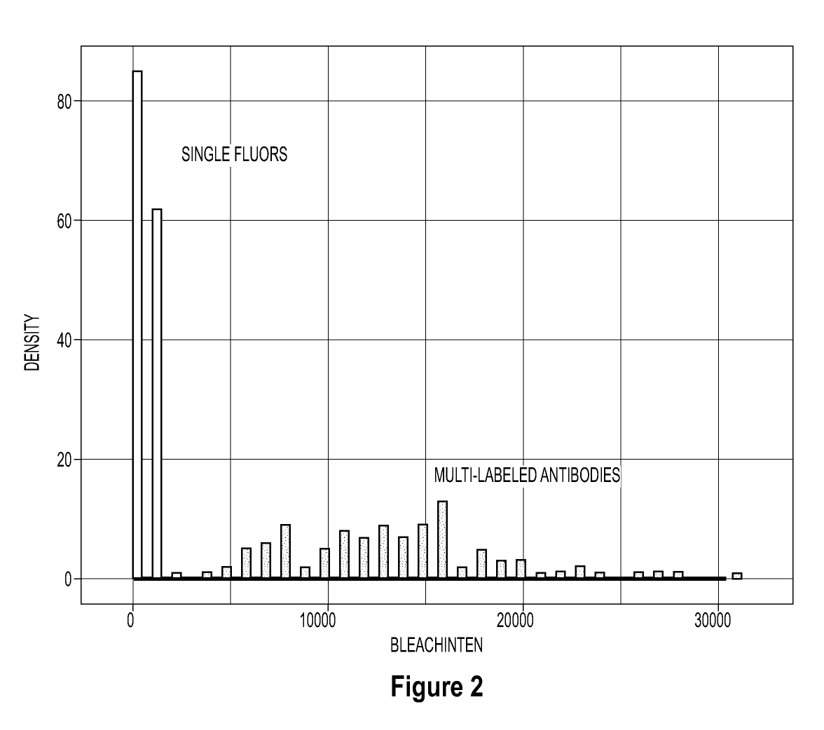

In another aspect, intensity and/or single-to-noise is used as a method of identifying single labels. When dye molecules or other optical labels are in close proximity, they are often impossible to discriminate with fluorescence-based imaging due to the intrinsic limit of the diffraction of light. That is, two labels that are close together will be indistinguishable with no visible gap between them. One exemplary method for determining the number of labels at a given location is to examine the relative signal and/or signal-to-noise compared to locations known to have a single fluor. Two or more labels will usually emit a brighter signal (and one that can more clearly be differentiated from the background) than will a single fluor. FIG. 2 shows the normalized histogram of signal intensity measured from both single label samples and multi-label antibodies (both Alexa 546; verified through bleach profiles). The two populations were clearly separable, and multiple labels may be clearly distinguished from single labels.

In some embodiments, the counting step may comprise measuring optical signals from the immobilized labels, and calibrating the counted numbers by distinguishing an optical signal from a single label from the rest of the optical signals from background and/or multiple labels. In some embodiments, the distinguishing comprises calculating a relative signal and/or single-to-noise intensity of the optical signal compared to an intensity of an optical signal from a single label. The distinguishing may further comprise determining whether the optical signal is from a single label. In additional embodiments, the optical signal is from a single label if the relative signal and/or single-to-noise intensity of an optical signal differs from an intensity of an optical signal from a single label by a predetermined amount or less. In further embodiments, the predetermined amount is from 0% to 100%, from 0% to 150%, 10% to 200%, 0, 1, 2, 3, 4, 5, 10, 20, 30, or 40% or more, and/or 300, 200, 100, 50, 30, 10, or 5% or less of the intensity of the optical signal from a single label.

In another aspect, different labels may have different blinking and bleaching properties. They may also have different excitation properties. In order to compare the number of dye molecules for two different labels, it is necessary to ensure that the two dyes are behaving in a similar manner and have similar emission characteristics. For example, if one dye is much dimmer than another, the number of molecules may be under-counted in this channel. Several factors may be titrated to give the optimal equivalence between the dyes. For example, the counting step and/or calibrating step may comprise optimizing (i) powers of light sources to excite the labels, (ii) types of the light sources, (ii) exposure times for the labels, and/or (iv) filter sets for the labels to match the optical signals from the labels, and measuring optical signals from the labels. These factors may be varied singly or in combination. Further, the metric being optimized may vary. For example, it may be overall intensity, signal-to-noise, least background, lowest variance in intensity or any other characteristic.

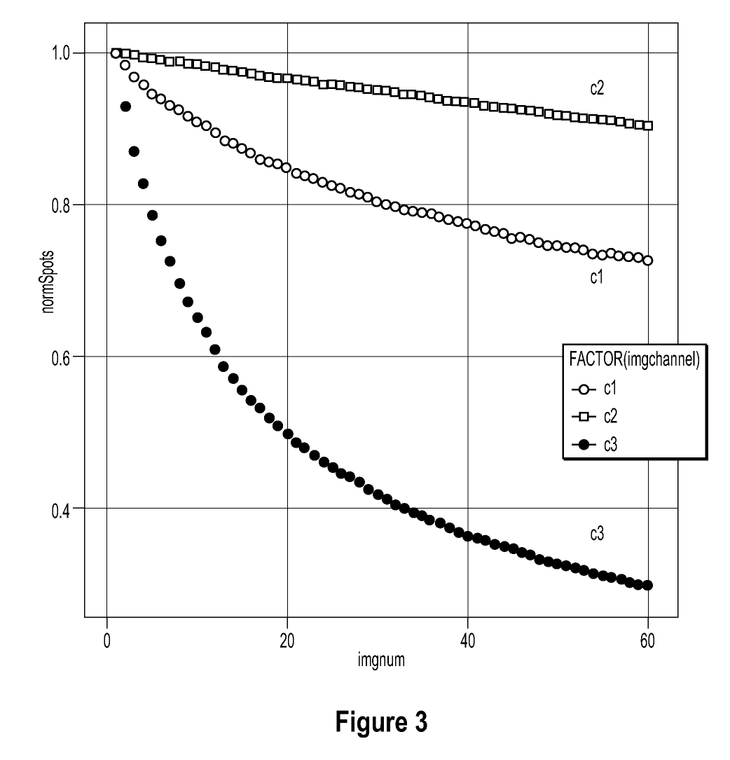

Bleaching profiles are label specific and may be used to add information for distinguishing label types. FIG. 3 shows average bleaching profiles from various labels. The plot shows the normalized counts per label type as a function of successive images that were collected over a 60 second interval. Item c1 is Cy3 fluor, item c2 is Atto647 fluor, and item c3 is Alexa488 fluor.

In another aspect, blinking behavior may be used as a method of identifying single labels. Many dye molecules are known to temporarily go into a dark state (e.g., Burnette et al., Proc. Natl. Acad. Sci. USA (2011) 108: 21081-21086). This produces a blinking effect, where a label will go through one or more steps of bright-dark-bright. The length and number of these dark periods may vary. The current invention uses this blinking behavior to discriminate one label from two or more labels that may appear similar in diffraction limited imaging. If there are multiple labels present, it is unlikely the signal will completely disappear during the blinking. More likely is that the intensity will fall as one of the labels goes dark, but the others do not. The probability of all the labels blinking simultaneously (and so looking like a single fluor) may be calculated based on the specific blinking characteristics of a dye.

In some embodiments, the optical signals from the labels are measured for at least two time points, and an optical signal is from a single label if the intensity of the optical signal is reduced by a single step function. In some embodiments, the two time points may be separated by from 0.1 to 30 minutes, from 1 second to 20 minutes, from 10 seconds to 10 minutes; 0.01, 0.1, 1, 2, 3, 4, 5, 10, 20, 30, 40, 50, 60 seconds or more; and/or 1, 2, 3, 4, 5, 10, 20, 30, 40, 50, 60 seconds or less. In additional embodiments, an intensity of the optical signal from a single label has a single step decrease over time, and an intensity of the optical signal from two or more labels has multiple step decreases over time. In further embodiments, the optical signals from the labels are measured for at least two time points and are normalized to bleaching profiles of the labels. In another aspect, the method described herein and/or the counting step may further comprises measuring an optical signal from a control label for at least two time points, and comparing the optical signal from the control label with the optical signals from the labels to determine an increase or decrease of the optical signal from the labels.

In another aspect, the counting step further comprises confirming the counting by using a control molecule. A control molecule may be used to determine the change in frequency of a molecule type. Often, the experimental goal is to determine the abundance of two or more types of molecules either in the absolute or in relation to one another. Consider the example of two molecules labeled with two different dyes. If the null hypothesis is that they are at equal frequency, they may be enumerated on a single-molecule array and the ratio of the counts compared to the null hypothesis. The “single-molecule array” herein is defined as an array configured to detect a single molecule, including, for example, the arrays described in U.S. Patent Application Publication No. 2013/0172216. If the ratio varies from 1:1, this implies they two molecules are at different frequencies. However, it may not be clear a priori whether one has increased abundance or the other has decreased abundance. If a third dye is used as a control molecule that should also be at equal frequency, this should have a 1:1 ratio with both the other dyes. Consider the example of two molecules labeled with dyes A and B, the goal being to see if the molecule labeled with dye B is at increased or decreased frequency compared to the molecule labeled with dye A. A third molecule labeled with dye C is included in the experiment in a way that it should be at the same abundance as the other two molecules. If the ratio of molecules labeled A and B respectively is 1:2, then either the first molecule has decreased frequency or the second has increased frequency. If the ratio of the molecules labeled A and C is 1:1 and the ratio of molecules labeled B and C is 1:2, then it is likely that the molecule labeled with dye B has increased with frequency with respect to the molecule labeled with dye A. An example of this would be in determining DNA copy number changes in a diploid genome. It is important to know if one sequence is amplified or the other deleted and using a control molecule allows for this determination. Note the control may be another region of the genome or an artificial control sequence.

In another aspect, estimation of the ratio in detecting trisomy may be performed without knowledge of the fetal fraction. For example, diploid chromosomes would expect to yield a ratio of 1, in which the ratio is chromosome1/chromosome2 and chromosome 1 represents the copy number of the first chromosome of interest and chromosome2 represents the copy number of the second chromosome of interest. In the case of trisomy for the first chromosome of interest, however, the ratio would be greater than 1. If f is the fetal fraction, the ratio would be given by ((1−f)×chromosome1+f×chromsome1)/((1−f)×chromosome2+f×chromsome2). In the normal diploid case, both chromosome1 and chromosome2 are equal to 2, and the ratio is 1. For the trisomy of the first chromosome, the results is ((1−f)×2+f×3)/((1−f)×2+f×2)=((1−f)×2+f×3)/2. Thus, as the fetal fraction increases, the ratio increases. In the extreme case of a pure fetal sample, the fetal fraction is 1 and the ratio is 3/2=1.5. If the fetal fraction is zero, the trisomy is undetectable and the ratio is 1.

In another aspect, the counting step further comprises confirming the counting and/or assay results by using measured fetal fraction. For example, in the case where the observed ratio between the numbers of molecules (i.e. labels, ligated probe sets, amplified products thereof, and/or copies of the chromosomes of interest) is 1.25, the expected fetal fraction can be calculated to be 0.5. The fetal fraction, however, may be calculated or measured independently from the observed ratio between the numbers of labels, ligated probe sets, amplified products thereof, or copies of the chromosomes of interest, and may further be compared to the expected fetal fraction. For example, if the calculated or measured fetal fraction is 0.05, the expected ratio is 1.025, which is lower than the observed ratio of 1.25. This discrepancy between the expected and observed ratios may be due to chance since there will sample sampling variance, or may indicate an error in the process, experiment or calculation of the ratio. The discrepancy may also represent a sample switch (e.g. different samples were used in the measurement of fetal fraction and the ratio). Such a discrepancy between the expected and observed ratios may warrant additional counting of molecules on the substrate, additional analysis and/or a repeat of some or all of the DNA extraction, assay, immobilization on the substrate, imaging or calibration.

In some embodiments, the results of the method described herein (e.g., counted numbers of labels) may be confirmed by using different labels but the same tags used in the initial method. Such confirming may be performed simultaneously with the initial method or after performing the initial method. In additional embodiments, the confirming described herein comprises contacting first and second control probe sets to the genetic sample, wherein the first control probe set comprises a first control labeling probe and the first tagging probe, which is the same tag of the first probe set described herein, and the second control probe set comprises a second control labeling probe and the second tagging probe, which is the same tag of the second probe set described herein. The confirmation may further comprise hybridizing at least a part of the first and second control probe sets to the first and second nucleic acid regions of interest in nucleotide molecules of the genetic sample, respectively. The confirmation may further comprise ligating the first control probe set at least by ligating the first control labeling probe and the first tagging probe. The confirmation may further comprise ligating the second control probe set at least by ligating the second control labeling probe and the second tagging probe. The confirmation may further comprise amplifying the ligated probe sets. The confirmation may further comprise immobilizing each of the tagging probes to a pre-determined location on a substrate, wherein the first and second control labeling probes and/or the amplified labeling probes thereof ligated to the immobilized tagging probes comprise first and second control labels, respectively, the first and second control labels are different, and the immobilized labels are optically resolvable. The confirmation may further comprise measuring the optical signals from the control labels immobilized to the substrate. The confirmation may further comprise comparing the optical signals from the immobilized first and second control labels to the optical signals from the immobilized first and second labels to determine whether an error based on the labels exists. The “error based on a label” used herein means any error caused by the label that may not have occurred if a different label is used in the method. In some embodiments, the first label and the second control label are the same, and the second label and the first control label are the same.

Bleaching may be used as a method of identifying single labels. A key element of the readout is that individual labels be “resolvable,” i.e., distinct. This is trivial at low densities on a surface when the likelihood of labels in close proximity is very low. For higher densities, assuming the labels are at random locations (i.e., Poissonian), the chances of close neighbors increases to the point where significant numbers of labels have neighbors whose fluorescent emission partially (or fully) overlaps with their own emission. At this point, the labels are no longer “resolvable,” and in a transition regime exists between single-label detection (i.e., digital detection) and classic multi-label array-type detection (e.g., analogue detection) where the average signal from many molecules is measured. Put differently, a digital counting regime of individual molecules is switched to an analog regime of average-fluorescent-intensity from many molecules.

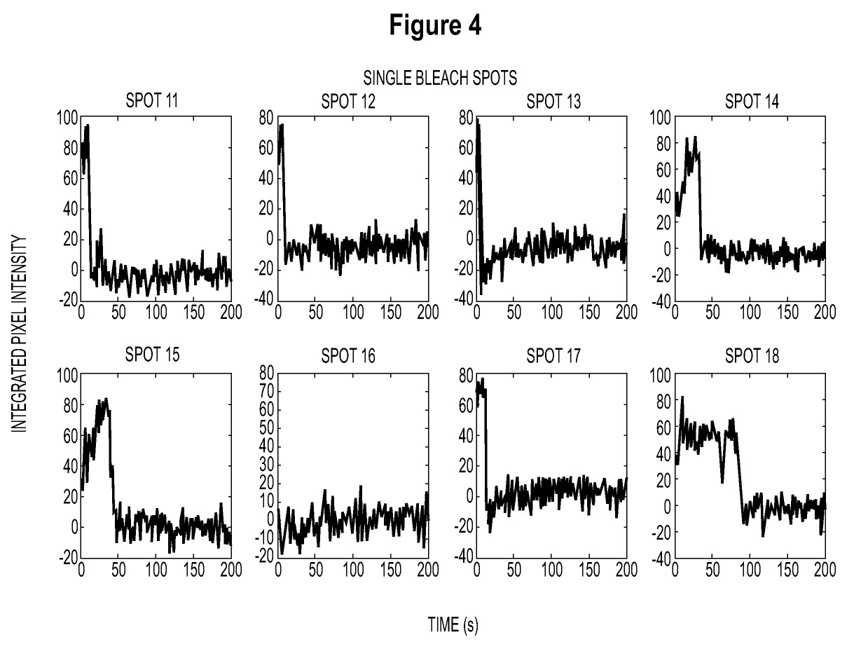

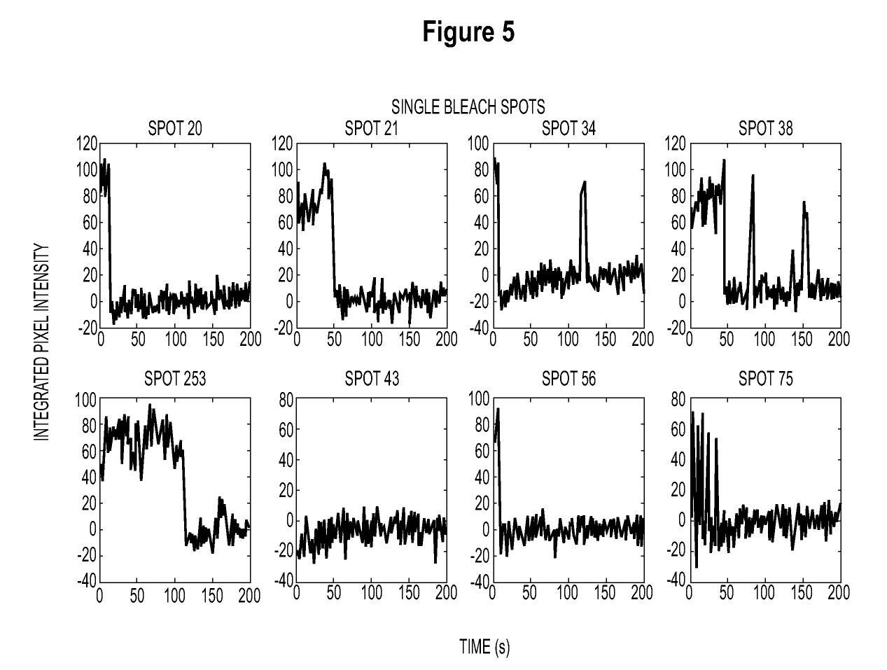

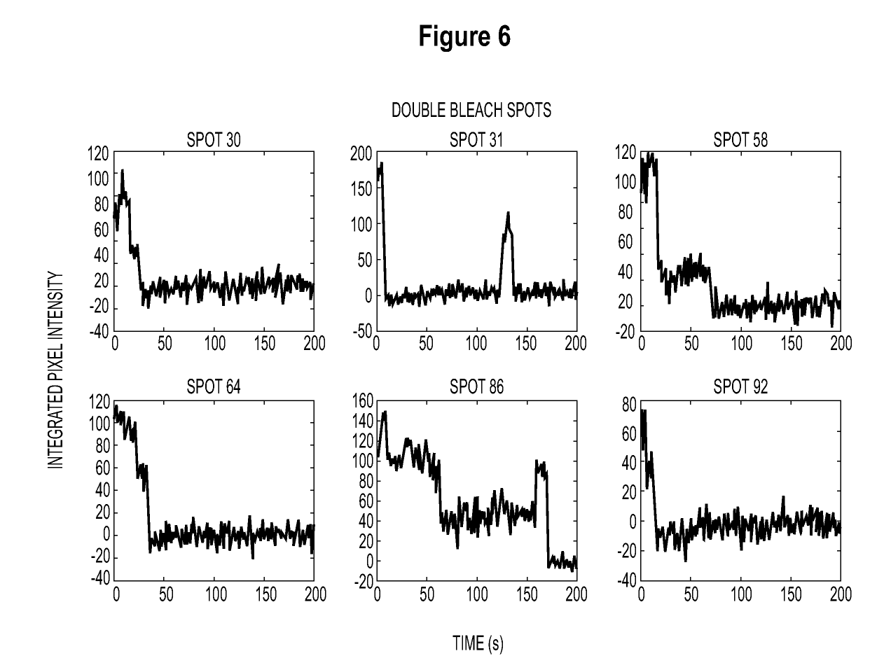

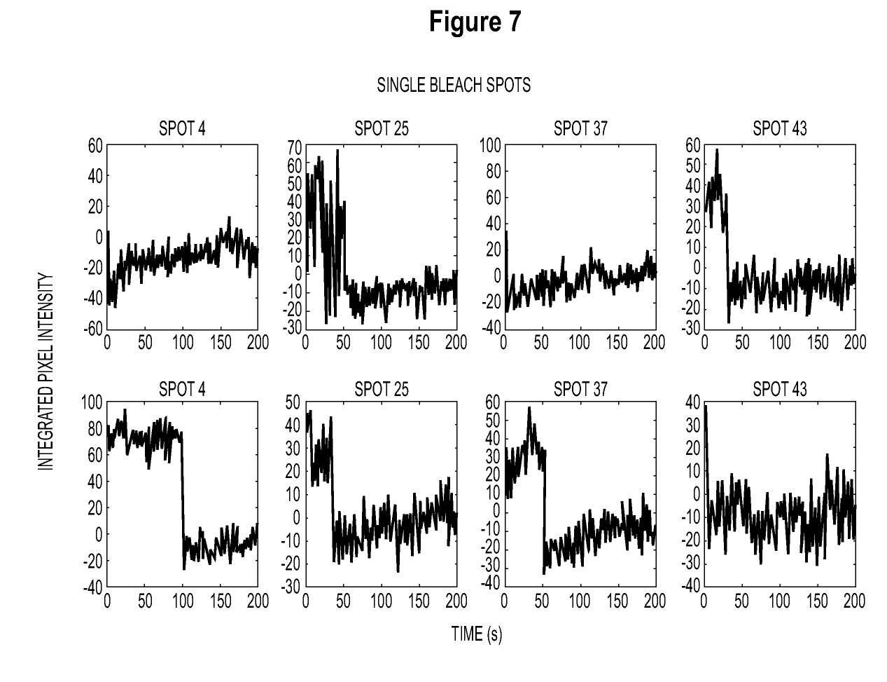

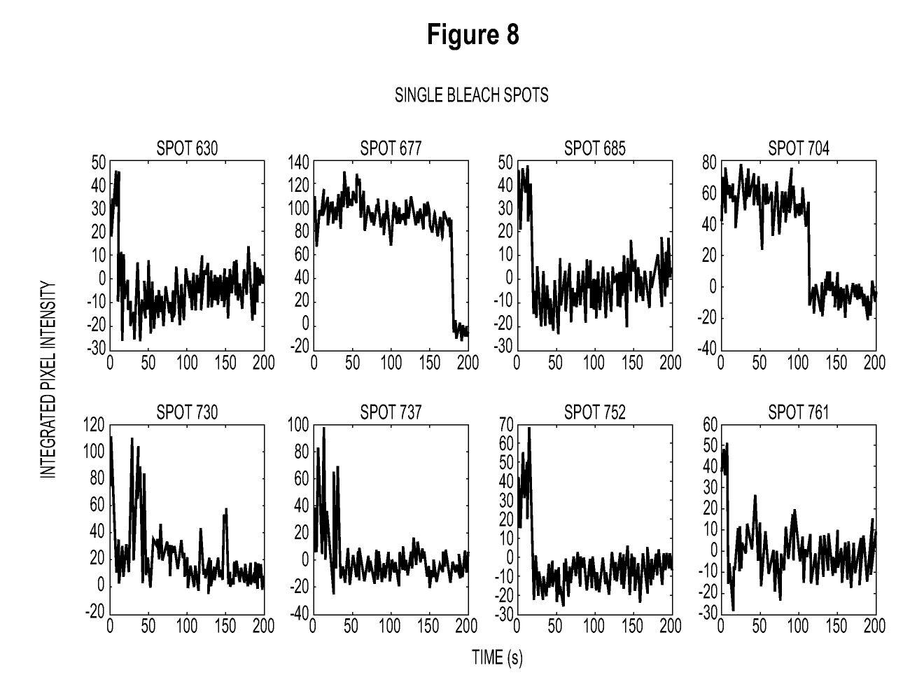

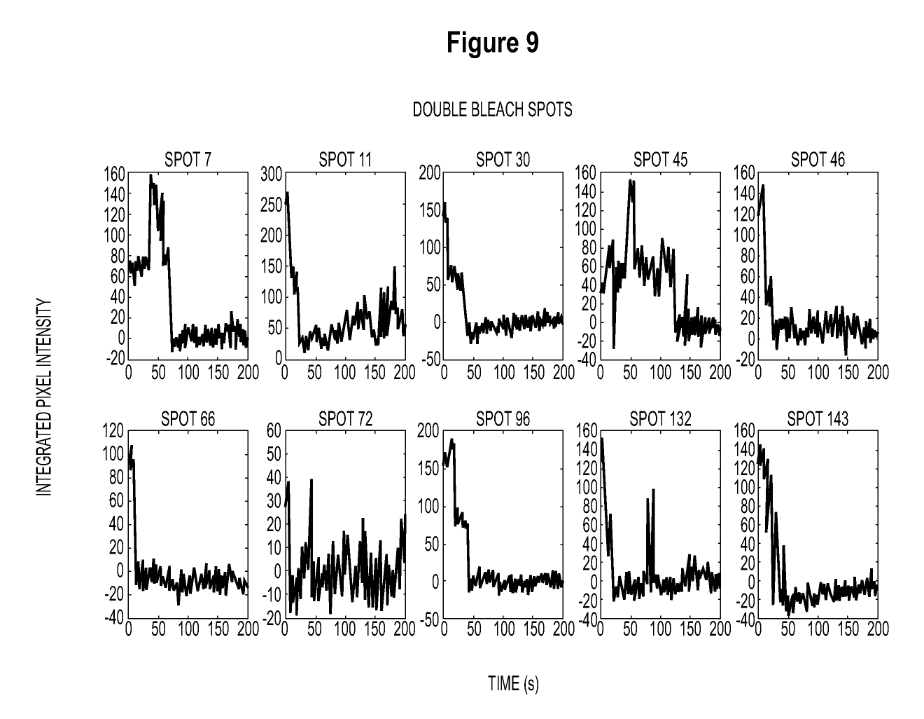

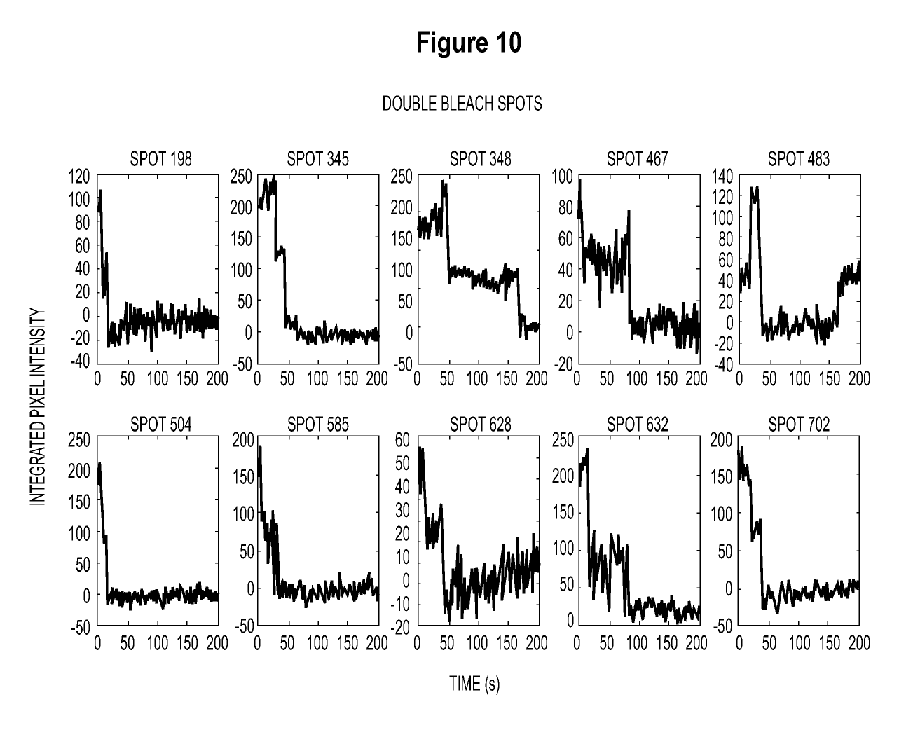

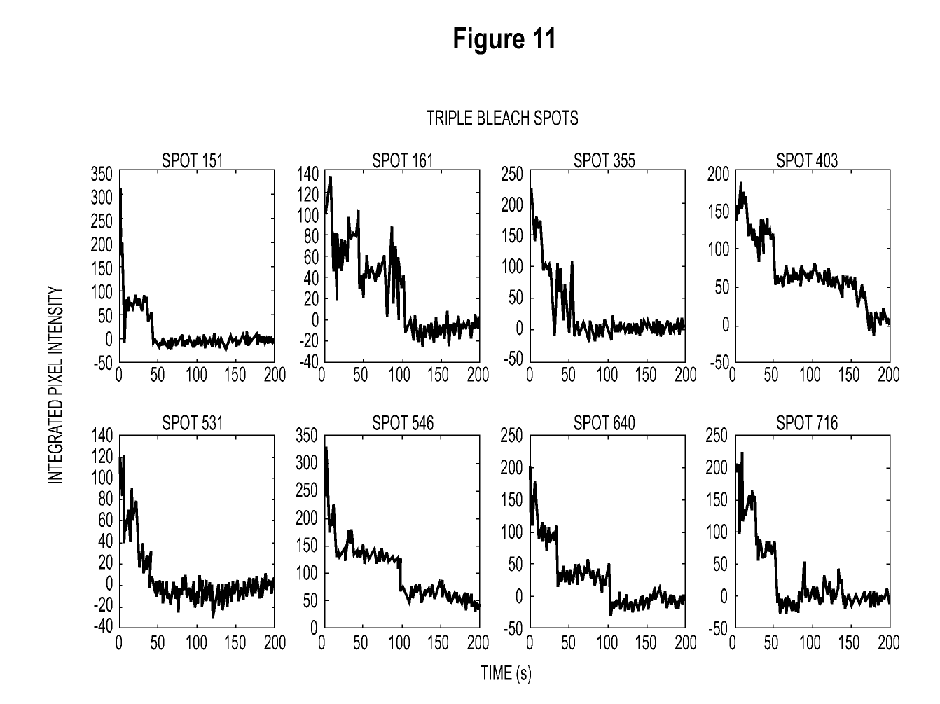

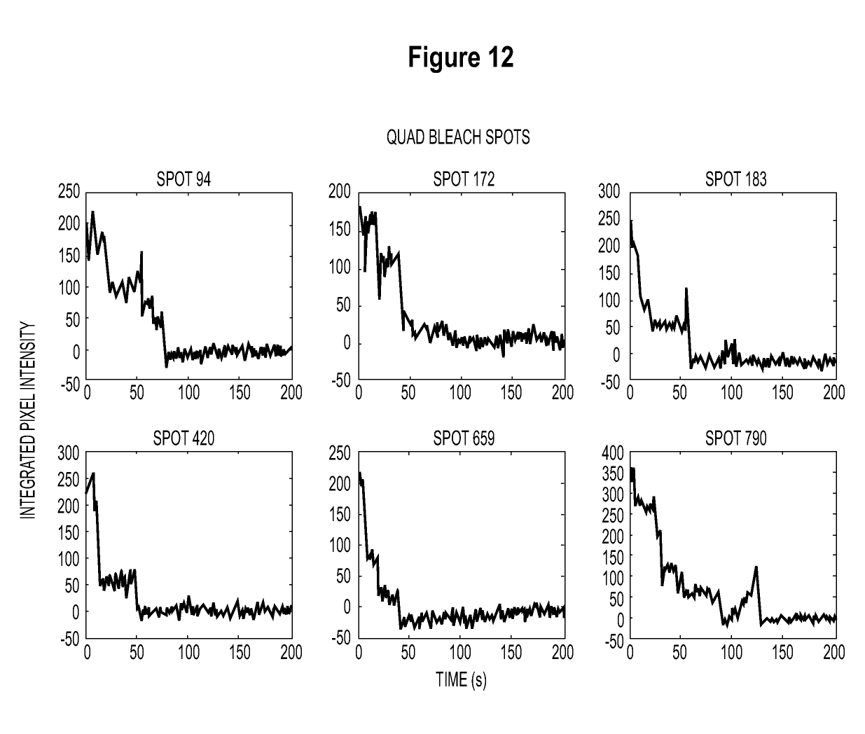

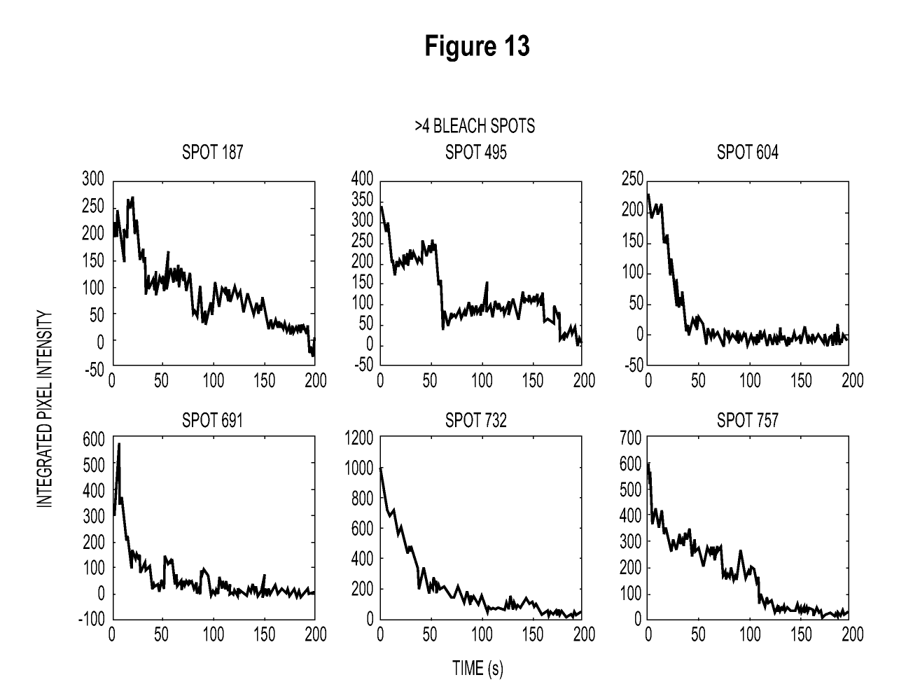

One solution to increase the loading range while maintaining individual resolvability is to take advantage of fluorophore bleaching. Extended exposure to light may cause labels to bleach, that is, lose their property of fluorescence. That is, over time, a label may be extinguished. This usually occurs as a step function, with the label appearing to “switch off.” The current invention may use this bleaching behavior to discriminate one label from two or more labels that may appear similar in diffraction limited imaging. For multiple labels, extinction would be expected to occur via a series of step-wise decreases in the signal intensity. For example, FIGS. 4-13 show the integrated label intensity vs. time (showing bleaching events as changes in intensity) graphs that were obtained for various Alexa 488 labels. Single versus multiple label species may be easily differentiated (e.g. depending on whether the intensity of the optical signal is reduced by single versus multiple step(s) as shown in the graphs).

In another aspect, the method herein may comprise calibrating and/or confirming the counted numbers by label swapping or dye swapping. In some embodiments where probe product 1 and 2 are labeled with labels 1 and 2, respectively, various modes of error may mimic the differential frequency of the probe products. For example, if a ratio of 1:2 is observed between label 1 and label 2, this may be due to genuine differences in frequency (probe product 2 is twice as common as probe product 1), differences in hybridization efficiency (the probe products are at equal abundance, but probe product 2 hybridizes more efficiently than probe product 1) or differences in the properties of the labels (for example, if the labels are fluorescent dyes, label 1 may bleach faster, blink more frequently, give lower signal or lower signal-to-noise than label 2). If the same experiment is repeated with the labels switched, the ratio should be reversed, if it is a genuine observation of different frequencies of the molecules, with label 1 now twice as common as label 2. However, if it is due to differential hybridization efficiency the ratio will be ≦2:1. If the 1:2 ratio was due to the properties of the labels, the ratio will switch to 2:1 of label 1 to label 2 if they are actually at equal frequency. This approach can be extended to any number of labeled probe sets.

In some embodiments, the first nucleic acid region of interest is located in a first chromosome, and the second nucleic acid region of interest is located in a second chromosome, different from the first chromosome. The counting step may further comprise confirming the counting, wherein the confirming step comprises contacting first and second control probe sets to the genetic sample, wherein the first control probe set comprises a first control labeling probe and a first control tagging probe, and the second control probe set comprises a second control labeling probe and the second control tagging probe. The confirming step may further comprise hybridizing at least a part of the first and second control probe sets to first and second control regions located in the first and second chromosomes, respectively, wherein the first and second control regions are different from the first and second nucleic acid regions of interest. The confirming step may further comprise ligating the first and second control probe sets at least by ligating (i) the first control labeling and tagging probes, and (ii) the second control labeling and tagging probes. The confirming step may further comprise amplifying the ligated probe sets. The confirming step may further comprise immobilizing (i) the first probe set and the second control probe set to a first pre-determined location, and (ii) the second probe set and the first control probe set to a second pre-determined location. In some embodiments, the first and second control labeling probes and/or the amplified labeling probes thereof ligated to the immobilized tagging probes comprise a first and second control labels, respectively, the first label and the second control label are different, the second label and the first control labels are different, the immobilized labels are optically resolvable, the immobilized first and second control tagging probes and/or the amplified tagging probes thereof comprise first and second control tags, respectively, and the immobilizing step is performed by immobilizing the tags to the predetermined locations. The confirming step may further comprise measuring the optical signals from the control labels immobilized to the substrate. The confirming step may further comprise comparing the optical signals from the immobilized control labels to the optical signals from the immobilized first and second labels to determine whether an error based on the nucleic acid region of interest exists. In further embodiments, the first tag and the second control tag are the same, and the second tag and the first control tag are the same.

In another aspect, the counting step of the method described herein may further comprise calibrating and/or confirming the counted numbers by (i) repeating some or all the steps of the methods (e.g., steps including the contacting, binding, hybridizing, ligating, amplifying, and/or immobilizing) described herein with a different probe set(s) configured to bind and/or hybridize to the same nucleotide and/or peptide region(s) of interest or a different region(s) in the same chromosome of interest, and (ii) averaging the counted numbers of labels in the probe sets bound and/or hybridized to the same a nucleotide and/or peptide region of interest or to the same chromosome of interest. In some embodiments, the averaging step may be performed before the comparing step so that the averaged counted numbers of labels in a group of different probe sets that bind and/or hybridize to the same nucleotide and/or peptide region of interest are compared, instead of the counted numbers of the labels in the individual probe sets. In another aspect, the method described herein may further comprise calibrating and/or confirming the detection of the genetic variation by (i) repeating some or all the steps of the methods (e.g., steps including the contacting, binding, hybridizing, ligating, amplifying, immobilizing, and/or counting) described herein with different probe sets configured to bind and/or hybridize to control regions that does not have any known genetic variation, and (ii) averaging the counted numbers of labels in the probe sets bound and/or hybridized to the control regions. In some embodiments, the averaged numbers of the labels in the probe sets that bind and/or hybridize to control regions are compared to the numbers of the labels in the probe sets that bind and/or hybridized to the regions of interest described herein to confirm the genetic variation in the genetic sample. In another aspect, the steps of the calibrating and/or confirming may be repeated simultaneously with the initial steps, or after performing the initial steps.

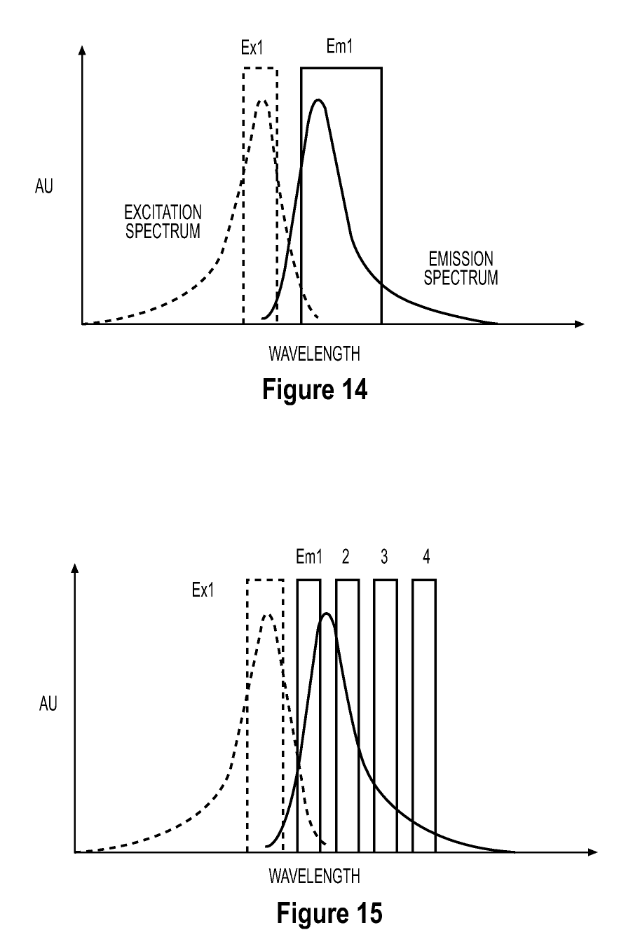

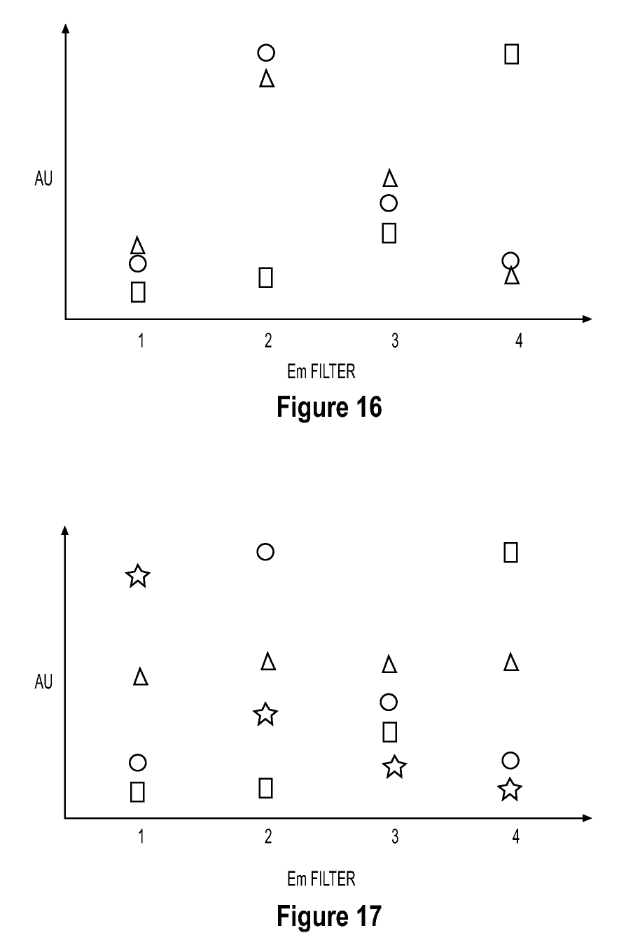

In another aspect, labels (e.g., fluorescent dyes) from one or more populations may be measured and/or identified based on their underlying spectral characteristics. Most fluorescent imaging systems include the option of collecting images in multiple spectral channels, controlled by the combination of light source and spectral excitation/emission/dichroic filters. This enables the same fluorescent species on a given sample to be interrogated with multiple different input light color bands as well as capturing desired output light color bands. Under normal operation, excitation of a fluorophore is achieved by illuminating with a narrow spectral band aligned with the absorption maxima of that species (e.g., with a broadband LED or arclamp and excitation filter to spectrally shape the output, or a spectrally homogenous laser), and the majority of the emission from the fluorophore is collected with a matched emission filter and a long-pass dichroic to differentiate excitation and emission (FIG. 14). In alternate operations, the unique identity of a fluorescent moiety may be confirmed through interrogation with various excitation colors and collected emission bands different from (or in addition to) the case for standard operation (FIG. 15). The light from these various imaging configurations, e.g., various emission filters, is collected and compared to calibration values for the fluorophores of interest (FIG. 16). In the example case, the experimental measurement (dots) matches the expected calibration/reference data for that fluorophore (triangles) but does not agree well with an alternate hypothesis (squares). Given test and calibration data for one or more channels, a goodness-of-fit or chi-squared may be calculated for each hypothesis calibration spectrum, and the best fit selected, in an automated and robust fashion. Various references may be of interest, including fluorophores used in the system, as well as common fluorescent contaminants, e.g., those with a flat emission profile (Contaminant 1; triangle), or a blue-weighted profile (Contaminant 2; stars) (FIG. 17).

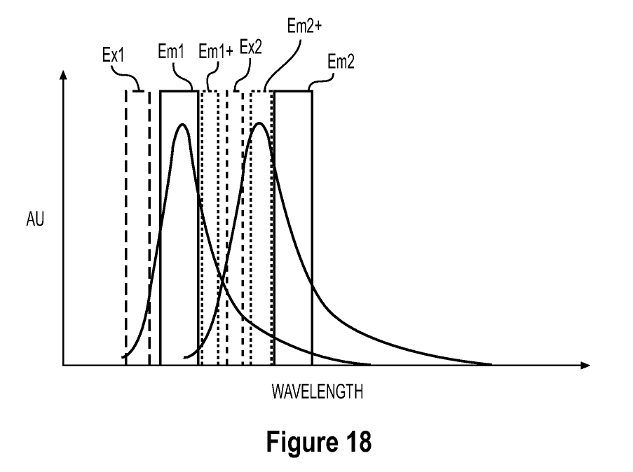

The design constraints for filter selection may be different from standard designs for which the goal is simply to maximize collected light in a single channel while avoiding significant contributions from other channels. In our invention the goal is spectral selectivity rather than solely light collection. For example, consider two fluorophores with significantly-different excitation bands, shown in FIG. 18 (note, only the excitation regions are shown and no excitation spectra). A standard design would maximize the capture of Fluor 1 emission (with Em1 filter, solid line) and minimize catching the leading edge from Fluor 2, and Fluor 2 would be optimally captured by Em2 (which is slightly red-shifted to avoid significant collection of Fluor 1 light). In our design, verifying the presence of Fluor 2 with the Em1 filter is desired leading to widening of the band to be captured (“Em1+”, fine dashed line). This creates additional information to verify the identity of Fluor 2. Similarly, Em2 may be widened or shifted towards Fluor 1 to capture more of that fluor's light (Em2+, fine dashed line). This increase in spectral information must also be balanced with the total available light from a given fluorophore to maintain detectability. Put differently, the contribution from a given fluorophore in a given channel is only significant if the corresponding signal is above the background noise, and therefore informative, unless a negative control is intended. In this way, the spectral signature of a fluorescent entity may be used for robust identification and capturing more light may be a second priority if species-unique features may be more effectively quantitated.

Given probe products may be labeled with more than one type of fluorophore such that the spectral signature is more complex. For example, probe products may always carry a universal fluor, e.g., Alexa647, and a locus-specific fluorophore, e.g., Alexa 555 for locus 1 and Alexa 594 for locus 2. Since contaminants will rarely carry yield the signature of two fluors, this may further increase the confidence of contamination rejection. Implementation would involve imaging in three or more channels in this example such that the presence or absence of each fluor may be ascertained, by the aforementioned goodness-of-fit method comparing test to reference, yielding calls of locus 1, locus 2 or not a locus product. Adding extra fluors aids fluor identification since more light is available for collection, but at the expense of yield of properly formed assay products and total imaging time (extra channels may be required). Other spectral modifiers may also be used to increase spectral information and uniqueness, including FRET pairs that shift the color when in close proximity or other moieties.

In another aspect, as described herein, the method of the present disclosure may be used to detect a genetic variation in peptide or proteins. In such as case, the methods may comprise contacting first and second probe sets to the genetic sample, wherein the first probe set comprises a first labeling probe and a first tagging probe, and the second probe set comprises a second labeling probe and a second tagging probe. The methods may further comprise binding the probe sets to peptide regions of interest by a physical or chemical bond, in place of the hybridizing step described herein in the case of detecting the genetic variation in nucleic acid molecules. Specifically, the methods may further comprise binding at least parts of the first and second probe sets to first and second peptide regions of interest in a peptide of protein of the genetic sample, respectively. For example, the binding may be performed by having a binder in at least one probe in the probe set that specifically binds to the peptide region of interest.

In some embodiments, the methods to detect a genetic variation in peptide or proteins may further comprise conjugating the first probe set by a chemical bond at least by conjugating the first labeling probe and the first tagging probe, and conjugating the second probe set at least by conjugating the second labeling probe and the second tagging probe, in place of the ligating step described herein in the case of detecting the genetic variation in nucleic acid molecules. The method may further comprise immobilizing the tagging probes to a pre-determined location on a substrate as described herein. In additional embodiments, the first and second labeling probes conjugated to the immobilized tagging probes comprise first and second labels, respectively; the first and second labels are different; the immobilized labels are optically resolvable; the immobilized first and second tagging probes and/or the amplified tagging probes thereof comprise first and second tags, respectively; and the immobilizing step is performed by immobilizing the tags to the predetermined location. The methods may further comprise, as described herein, counting (i) a first number of the first label immobilized to the substrate, and (ii) a second number of the second label immobilized to the substrate; and comparing the first and second numbers to determine the genetic variation in the genetic sample.

In one aspect, the present invention relates to methods of detecting a genetic variation in a genetic sample from a subject, comprising (i) contacting probes or probe sets to the genetic sample; (ii) hybridizing the probes or probe sets to a nucleic acid region of interest in nucleotide molecules of the genetic sample; (iii) amplifying the probes or probe sets with forward and reverse primers, wherein the forward or reverse primers comprise one or more label; (iv) digesting terminal phosphodiester bonds in single-stranded oligonucleotides of the probes or probe sets with a first exonuclease after the amplifying. After the digesting with the first exonuclease, the first exonuclease may be inactivated. After the inactivating, a 5′ and/or 3′ end of the amplified double-stranded probes or probe sets may be digested with a second exonuclease to produce an amplified single-stranded probe or probe sets. After the digesting with the second exonuclease, single-stranded oligonucleotides of the probes or probe sets may be detected and/or measured to determine the presence or absence of the genetic variation in a genetic sample from a subject. In some embodiments, the first exonuclease is Exonuclease I, and the second exonuclease is lambda Exonuclease. In additional embodiments, the first exonuclease is inactivated by heat. In further embodiments, the probe set is contacted to the genetic sample; the probe set comprises a labeling probe and a tagging probe; and the method further comprises ligating the labeling probe and the tagging probe prior to the amplifying. The ligated probe set may comprise the labeling probe at the 3′-end and the tagging probe at the 5′-end; the labeling probe hybridizes to the reverse primer; the tagging probe comprises the isolating tag; the tagging probe hybridizes to the forward primer; and the reverse primer comprises the label.

In another aspect, the present invention also relates to methods of isolating amplified products of a probe and/or a ligated probe set comprising immobilizing a composition comprising single-stranded ligated probe sets and second probes on a substrate, wherein each of the single-stranded ligated probe set comprises a labeling probe and a tagging probe ligated to each other; each of the second probes comprises the labeling probe or the tagging probe; and the labeling probe or the tagging probe comprises an isolating tag configured to bind to the substrate. In some embodiments, the methods may further comprise removing non-immobilized probes. In another aspect, the present invention also relates to methods of isolating amplified products of a probe and/or a ligated probe set comprising (i) amplifying one or more of the ligated probe sets with forward and reverse primers after the immobilizing to form one or more double-stranded ligated probe set, wherein the forward or reverse primer hybridizing to the labeling probe of the one or more of the ligated probe sets comprises a label, (ii) digesting terminal phosphodiester bonds in the second probes and/or the single-stranded ligated probe sets with one or more exonuclease after the amplifying, and (iii) isolating the non-digested and ligated probe sets after the digesting. In some embodiments, the isolating tag is biotin, and the substrate comprises streptavidin. In additional embodiments, the substrate comprises a streptavidin magnetic bead. The ligated probe set may comprise the labeling probe at the 3′-end and the tagging probe at the 5′-end; the second probes comprise the tagging probe; the tagging probe comprises the isolating tag; the labeling probe hybridizes to the reverse primer; the tagging probe hybridizes to the forward primer; and the reverse primer comprises the label.

In another aspect, the present invention also relates to methods of detecting a genetic variation in a genetic sample from a subject, comprising (i) contacting single-stranded probe sets to the genetic sample, wherein each of the single-stranded probe sets comprises a labeling probe and a tagging probe, and the labeling probe or the tagging probe comprises an isolating tag configured to bind to the substrate; (ii) hybridizing the single-stranded probe sets to a nucleic acid region of interest in nucleotide molecules of the genetic sample; (iii) ligating the single-stranded probe sets at least by ligating the labeling probe and the tagging probe to produce first single-stranded ligated probe sets; and (iv) immobilizing the first single-stranded ligated probe sets on a substrate. The methods described herein may also comprise (i) amplifying the first single-stranded ligated probe sets with forward and reverse primers after the immobilizing to a form double-stranded ligated probe set, wherein the forward or reverse primer hybridizing to the labeling probe of the first single-stranded ligated probe set comprises one or more labels; (ii) digesting terminal phosphodiester bonds in the single-stranded probe sets with a first exonuclease after the amplifying; (iii) after the digesting with the first exonuclease, inactivating the first exonuclease; (iv) after the inactivating, digesting an end of the amplified double-stranded ligated probe sets with a second exonuclease to produce second single-stranded ligated probe set; and (v) after the digesting with the second exonuclease, detecting the second single-stranded ligated probe sets to determine the presence or absence of the genetic variation in a genetic sample from a subject.

In another aspect, the present invention also relates to methods of detecting a genetic variation in a genetic sample from a subject, comprising (i) contacting non-ligated first and second probe sets to the genetic sample, wherein each of the first probe sets comprises a first labeling probe and a first tagging probe, and each of the second probe sets comprises a second labeling probe and a second tagging probe; (ii) hybridizing one or more of the first probe set and one or more of the second probe set to first and second nucleic acid regions of interest in nucleotide molecules of the genetic sample, respectively, to produce hybridized first and second probe sets; (iii) ligating the hybridized first probe set at least by ligating the first labeling probe and the first tagging probe to produce a ligated first probe set; (iv) ligating the hybridized second probe set at least by ligating the second labeling probe and the second tagging probe to produce a ligated second probe set; (v) immobilizing one or more of the tagging probes or the labeling probes of each of the non-ligated and ligated probe sets on one or more beads; (vi) amplifying the ligated probe set with forward and reverse primers after the immobilizing, wherein the forward or reverse primer hybridizing to the labeling probe of the ligated probe set comprises a label; (vii) digesting terminal phosphodiester bonds in non-ligated probes with one or more exonucleases after amplifying; (v) immobilizing the tagging probes to a pre-determined location on a substrate, wherein the first and second labeling probes and/or the amplified labeling probes thereof ligated to the immobilized tagging probes comprise first and second labels, respectively, the first and second labels are different, the immobilized labels are optically resolvable, and the immobilized first and second tagging probes and/or the amplified tagging probes thereof comprise first and second tags, respectively; (vi) counting (a) a first number of the first label immobilized to the substrate, and (b) a second number of the second label immobilized to the substrate; and (vii) comparing the first and second numbers to determine the genetic variation in the genetic sample.

In another aspect, certain molecular assays involve the joining or connecting of assay components. For example, two or more probes may be ligated together to form a ligated probe set described herein. In some instances (e.g. when the probes are present at high concentration), however, chimeras may be formed. Chimeras are structures that are analogous to ligated probe sets but formed by incorrect probe combinations. For example, if a probe set consists of two probes designed to hybridize to the genome such that they are close enough to ligate together, the goal of the assay may be to create a ligated probe set of these two probes. Multiple probes sets can be used together in an analogous manner. In some cases, probes from two or more different probe sets may be ligated together to form a chimera or chimeric ligated probe set. For example, the probes may have similar sequences, and one may bind to the wrong location in the genome (i.e. cross-hybridization) or to another probe that can act as a template for ligation events; some ligases may allow in-solution ligation and probes from different probe sets may be ligated before they hybridize to the genome (or if they become detached from the genome during ligation); other ligases may allow ligation to occur based on a template and one or more probes may act as a template for probe hybridization, and two probes may be partially hybridized to another probe allow ligation to occur. Even if the hybridization is temporary, the ligation may occur. Accordingly, some embodiments of the present disclosure provide a way to separate the ligated probe set comprising the correct probes from the chimeras.

For example, the present invention also relates to methods of isolating the ligated probe set comprising at least two probes hybridized to a genetic sample described herein. The probe set may comprise first and second oligonucleotide probes. The methods of isolating the ligated probe set hybridized to a genetic sample may comprise contacting a probe set to a genetic sample, for example, under a condition effective to hybridize the probe set to a nucleic acid region of interest in nucleotide molecules of the genetic sample, and hybridizing the probe set to the genetic sample. The methods of isolating the ligated probe set hybridized to a genetic sample may also comprise ligating the first and second oligonucleotide probes to form a ligated probe set. The methods of isolating the ligated probe set hybridized to a genetic sample may also comprise denaturing the ligated probe set from the genetic sample. Denaturing herein refers to the separation of at least a portion of hybridized base pairs. For example, the ligated probe set hybridized to a genetic sample may be placed under suitable denaturing condition by heat above its melting temperature, whereby one strand of the double-stranded structure to release the ligated probe set from the genetic sample. In some embodiments, the nucleic acid may be exposed to a temperature of at least 90° C. and the amount of time (e.g., at least 30 seconds up to 30 minutes) to allow nucleic acid denaturation. In some embodiments, the conditions may be used to cause complete or partial denaturation of the double-stranded structure so that the double-stranded structure is completely or partially separate. In other embodiments, the portions may be caused by the use of denaturing conditions (e.g., lower than the temperature caused by completely denaturing conditions) to separate a specific part of the double-stranded structure. In other embodiments, nucleic acids may also be chemically modified (e.g., using urea or NaOH).

The methods of isolating the ligated probe set hybridized to a genetic sample may further comprise hybridizing at least a part of a junction capture probe to the ligated probe set, wherein the junction capture probe is hybridized to (i) at least a part of the first oligonucleotide probe and (ii) at least a part of the second oligonucleotide probe. In some embodiments, the junction capture probe may comprise a sequence hybridizing to a larger number of nucleotides in one oligonucleotide probe compared to the nucleotides in the other oligonucleotide probe. The junction capture probe herein refers to any probe that is configured to hybridize, conjugate, bind, or immobilize to at least a portion of each of at least two probes described herein. For example, the junction capture probe may comprise an oligonucleotide configured to hybridize to at least a portion of each of at least two oligonucleotide probes in a ligated probe set described herein. In some embodiments, the junction capture probe may comprise an oligonucleotide having at least two sequences that are complementary to at least a portion of at least two oligonucleotide probes in a ligated probe set described herein. For example, when the junction probe is 20 base long and hybridizes to a ligated probe set comprising first and second probes, all or most of 20 bases of the junction probe may be hybridized. If there is a chimeric ligated product not comprising both of the first and second probes, however, the junction probe may hybridize only to one or none of the probes and whichever bases happen to match on the other side of the chimeric ligated product by chance. In this example, the hybridization will be more frequent and stronger for correctly formed ligated probe set comprising the first and second probes compared to the chimeric ligated product.

In some embodiments, (i) said at least a part of the first oligonucleotide probe hybridized to the junction capture probe and (ii) said at least a part of the second oligonucleotide probe hybridized to the junction capture probe are adjacent to each other.

The methods of isolating the ligated probe set hybridized to a genetic sample may further comprise isolating the ligated probe set hybridized to the junction capture probe. In some embodiments, the isolating comprises isolating the ligated probe set hybridized to the junction capture probe from the genetic sample. In additional embodiments, the isolating comprises isolating the ligated probe set hybridized to the junction capture probe from the chimeras and other products that are not the ligated probe set comprising at least two probes, each of which is hybridized to the junction capture probe. In further embodiments the isolating may or may not comprise isolating the ligated probe set hybridized to the junction capture probe from the substrate.

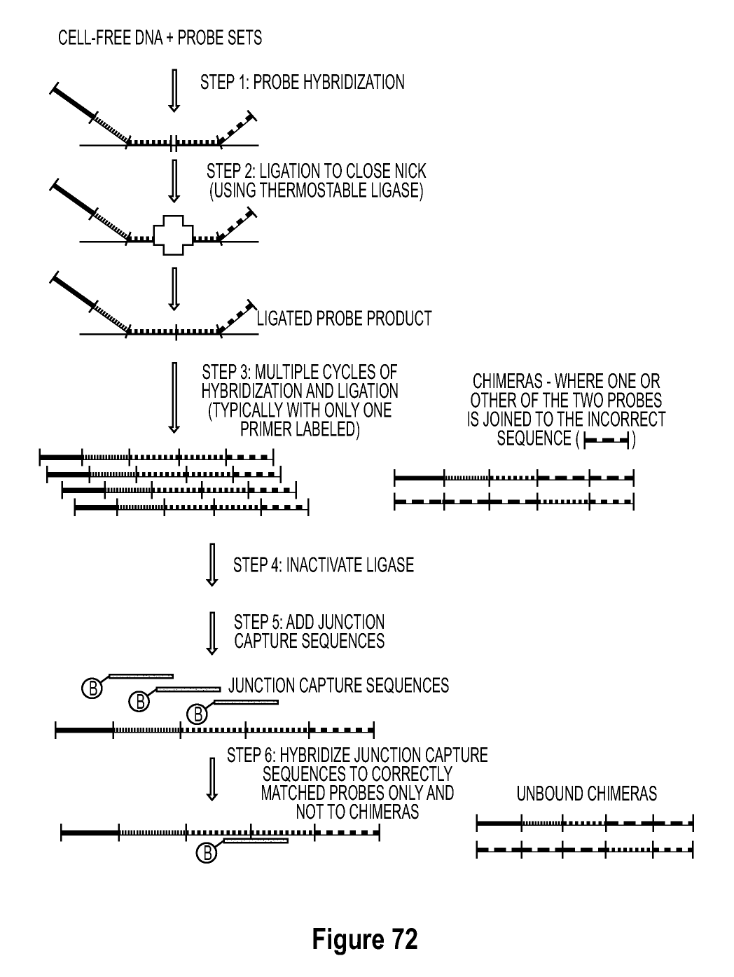

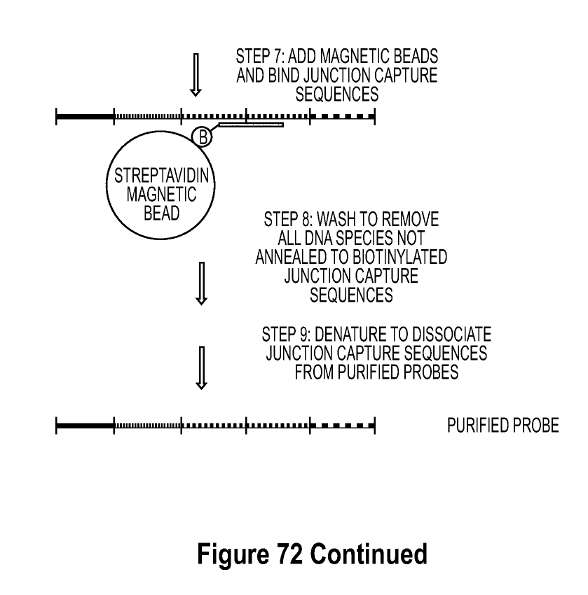

In some embodiments, the junction capture probe comprises a tag or affinity tag described herein (e.g. biotin), which may immobilize the junction capture probe on a substrate, and the isolating described above comprises immobilizing the tag on a substrate and washing the substrate. In additional embodiments, the substrate may comprise streptavidin or a streptavidin magnetic bead. In yet additional embodiments, the isolating comprises separating the ligated probe set from the substrate. In further embodiments, the junction capture probe is immobilized on the substrate after hybridizing at least a part of a junction capture probe to the ligated probe set. The exemplary embodiment is shown in FIG. 72.

After washing and removal of some or all of the chimeras, genomic DNA or other oligonucleotides, and the junction probes may be removed from the ligated probe set making the ligated probe set available for further analysis.

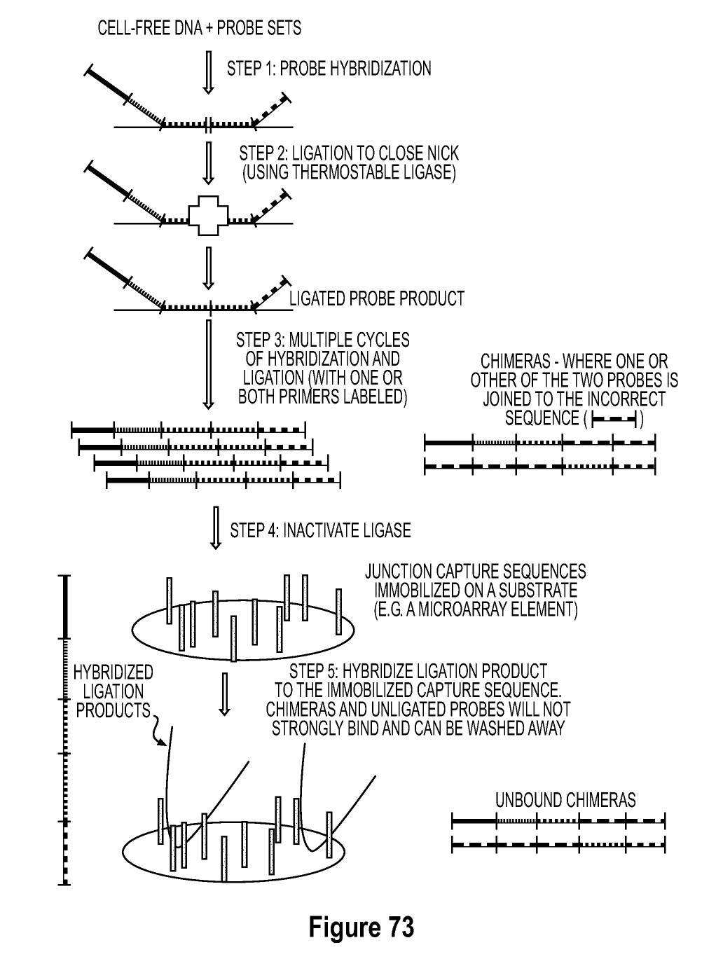

In additional embodiments, the immobilizing the tag of the junction capture probe is performed prior to the hybridizing the at least a part of a junction capture probe to the ligated probe set, and the washing is performed after the hybridizing. Thus, the junction capture probe may pull-down and immobilize the ligated probe set comprising probes each of which is configured to hybridize to the junction capture probe on the substrate. Chimeric ligated probe sets may either not hybridize or may hybridize less strongly compared to the ligated probe set comprising correct probes, and thus may be washed away from the substrate. The exemplary embodiment is shown in FIG. 73. In further embodiments, the ligated probe set may comprise at least two probes each of which hybridizes to the junction capture probe and each probe comprises a label described herein. After washing the substrate, the labels of the probes may be detected to confirm that the ligated probe sets comprising correct probes are immobilized and are to be counted according to the methods described herein. For example, if the ligated probe set comprises probes with same fluorescent labels, the fluorescent signal is boosted, allowing the ligated probe set to be more easily detected. If the ligated probe set comprises probes with different fluorescent labels (e.g. labels emitting fluorescence in different wavelengths), the coincidence of the fluorescent signal on the substrate can be used to differentiate a ligated probe set comprising correct probes from single probes (which may contain a single fluor type), contamination or chimeras. That is, the probability of observing two fluorescent labels of particular characteristics at the same location may be low. Such chance events will be rare, and thus the co-location of the signal from the two fluorescent labels can be used to confirm the identity of the ligated probe set that is being observed. In some embodiments, the junction capture probe may comprise a genomic sequence and thus may reduce the fidelity of the hybridization to the ligated probe set compared to other DNA tags that are orthogonal to the genome. In further embodiments, the ligated probe set may be amplified by methods described herein. If the ligated probe sets are amplified prior to the hybridization, however, the complexity is greatly reduced as the genome will be at much lower frequency than the amplified ligated probe set. In such a case, the junction probe may hybridize to the ligated probe set under the same conditions so that the probe sets can be multiplexed (i.e. run in the same reaction volume).

In some embodiments, a junction capture probes described herein may comprise a label. This label on the junction capture probe may act to boost signal (if the oligonucleotide probes are labeled with the same wavelength dye) or provide a combination of labels that is unique to correct ligation products. In these embodiments, the junction capture probe may comprise an oligonucleotide and may not overlap with the sequence of oligonucleotide tag or affinity tag on the substrate immobilizing the ligated probe set. In such embodiments, the junction capture probe may both confirm the ligated probe set comprising correct probes and provides a secondary optical signal to increase the brightness and/or intensity or allow two labels of different wavelengths to be present at the same location. The existence of co-located labels of different wavelengths may help differentiate the ligated probe set comprising correct probes from chimeras and/or from labeled primers. In additional embodiments, the junction capture probes described herein may hybridize to the ligated probe sets after the ligated probe sets are immobilized on a substrate, and the number of the junction capture probes hybridized to the ligated probe sets may be counted to determine the number of the ligated probe sets in the methods of detecting a genetic variation in a genetic sample from a subject as described herein.

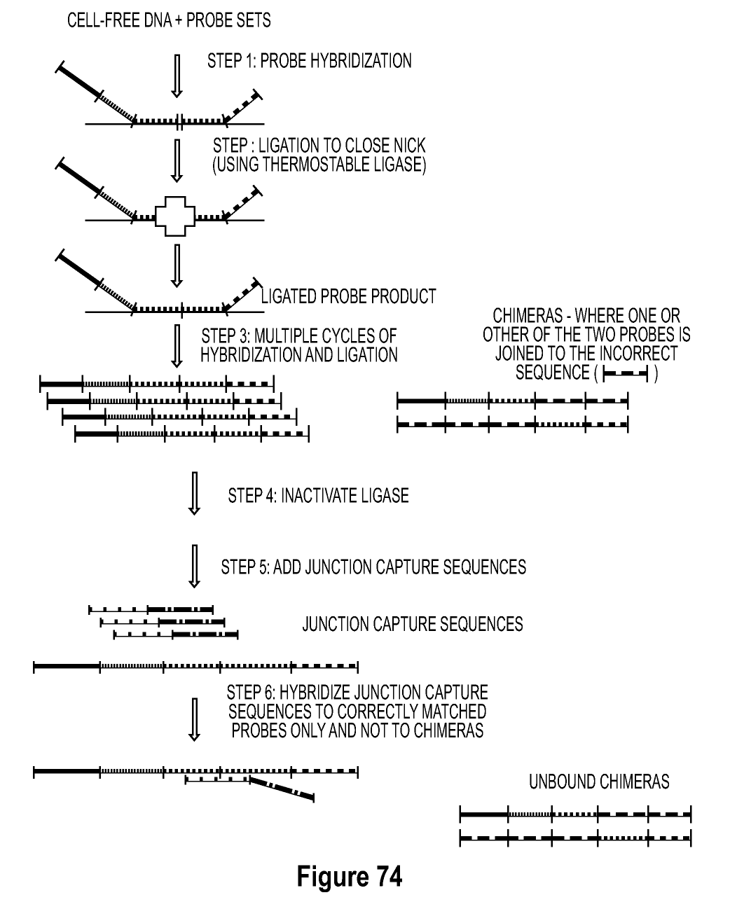

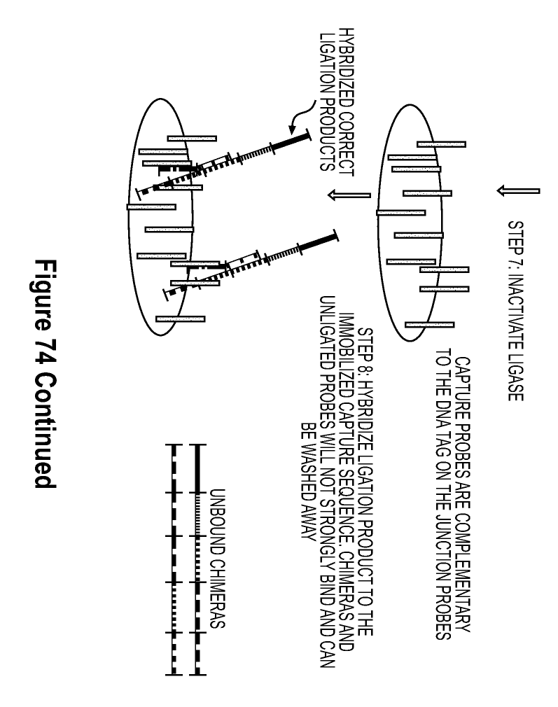

In some embodiments, the substrate comprises an anchor tag, and the immobilizing comprises immobilizing the tag to the anchor tag of the substrate. The anchor tag may be made of any tag or affinity tag described herein. For example, the tag of the junction capture probe and the anchor tag of the substrate comprise complementary oligonucleotide sequences, and the immobilizing comprises hybridizing the tag to the anchor tag of the substrate. The exemplary embodiment is shown in FIG. 74. As shown in this figure, in some embodiments, each of at least two probes in the ligated probe set may comprise a fluorescent label, and the junction probe would select the ligated probe set comprising correct probes (and exclude chimeras) and act as a means of pulling and immobilizing the ligated probe set to a substrate (e.g. an array). In additional embodiments, the anchor tag may comprise a sequence that does not exist in the genome may be used.

In additional embodiments, the present invention also relates to methods of detecting a genetic variation in a genetic sample from a subject, comprising (i) contacting a probe set to a genetic sample under a condition effective to hybridize the probe set to a nucleic acid region of interest in nucleotide molecules of the genetic sample, wherein the probe set comprises first and second oligonucleotide probes, and each of the oligonucleotide probes is configured to hybridize to a part of the nucleic acid region of interest; (ii) ligating the first and second oligonucleotide probes that are hybridized to the nucleotide molecules to form a ligated probe set; (iii) denaturing the ligated probe set from the genetic sample; (iv) hybridizing at least a part of a junction capture probe to the ligated probe set, wherein the junction capture probe is hybridized to at least a part of the first oligonucleotide and at least a part of the second oligonucleotide; (v) isolating the ligated probe set hybridized to the junction capture probe, (vi) amplifying the ligated probe set to form amplified ligated probe sets, and (vii) detecting the amplified ligated probe sets to determine the presence or absence of the genetic variation in a genetic sample from a subject.

In another aspect, the present invention also relates to methods of detecting a genetic variation in a genetic sample from a subject, comprising (i) contacting first and second probe sets to the genetic sample under a condition effective to hybridize the probe sets to a nucleic acid region of interest in nucleotide molecules of the genetic sample, wherein the first probe set comprises a first labeling probe and a first tagging probe, and the second probe set comprises a second labeling probe and a second tagging probe; (ii) denaturing the ligated probe set from the genetic sample; (iii) ligating at least parts of the first probe set at least by ligating the first labeling probe and the first tagging probe to form a first ligated probe set; (iv) ligating at least parts of the second probe set at least by ligating the second labeling probe and the second tagging probe to form a second ligated probe set; (v) hybridizing at least a part of each of first and second junction capture probes to the first and second ligated probe sets, respectively, wherein the first junction capture probe is hybridized to at least a part of each of the first labeling probe and the first tagging probe, and the second junction capture probe is hybridized to at least a part of each of the second labeling probe and the second tagging probe; (vi) isolating at least a part of the first and second ligated probe sets that are hybridized to the first and second junction capture probes, respectively, to form first and second isolated ligated probe sets; (vii) amplifying (a) the first isolated ligated probe set with first forward and reverse primers, wherein at least one of the first forward and reverse primers comprises a first label, and (b) the second isolated ligated probe set with second forward and reverse primers, wherein at least one of the second forward and reverse primers comprises a second label, to form amplified first and second ligated probe sets comprising the first and second labels, respectively, wherein the first and second labels are different; (viii) immobilizing at least parts of the amplified first and second ligated probe sets on a substrate, wherein the first and second labels of the amplified first and second ligated probe sets are optically resolvable after immobilization; (ix) counting (a) a first number of the first label in the amplified first probe set immobilized to the substrate, and (b) a second number of the second label in the amplified second probe set immobilized to the substrate, and (x) comparing the first and second numbers to determine the presence or absence of the genetic variation in a genetic sample from a subject.

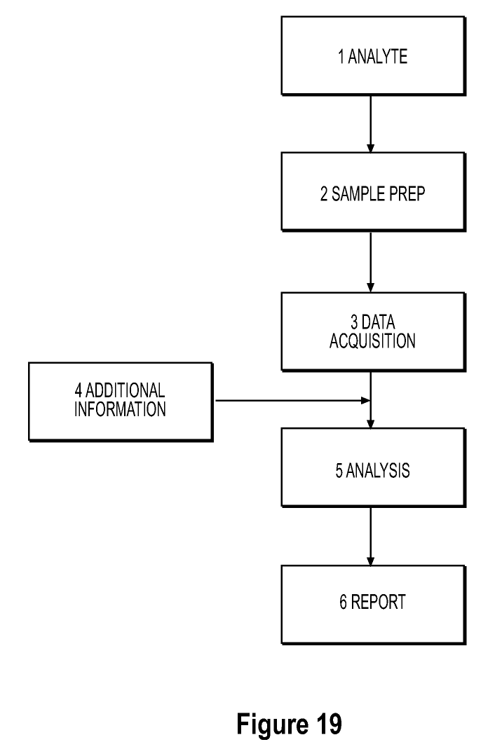

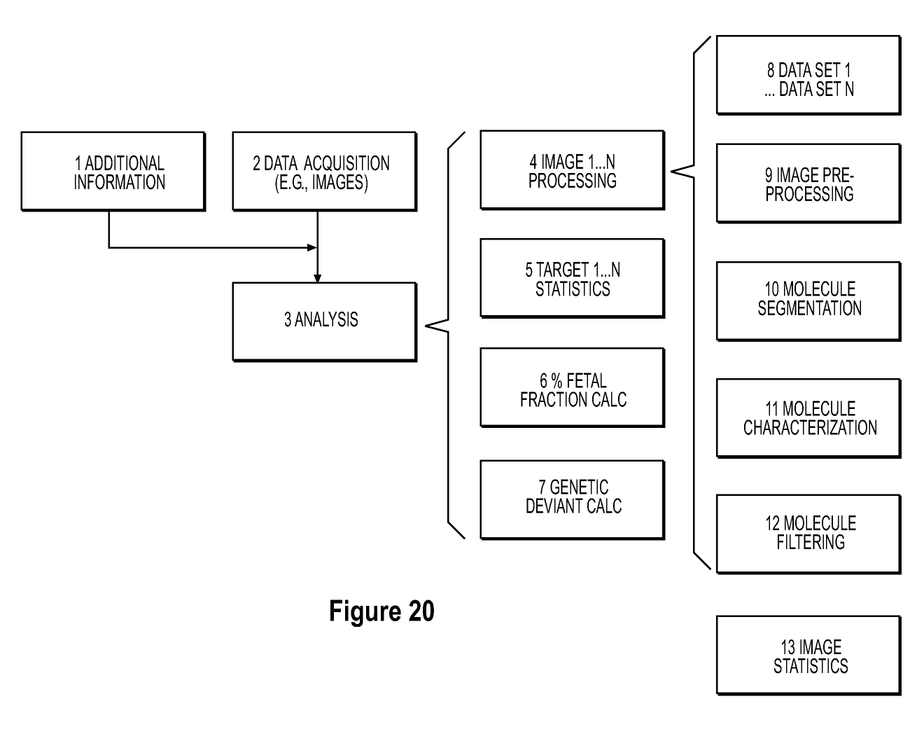

A system to detect a genetic variation according to the methods described herein includes various elements. Some elements include transforming a raw biological sample into a useful analyte. This analyte is then detected, generating data that are then processed into a report. Various modules that may be included in the system are shown in FIG. 19. More details of various methods for analyzing data, including e.g., image processing, are shown in FIG. 20. Analysis may be performed on a computer, and involve both a network connected to the device generating the data and a data server for storage of data and report. Optionally, additional information beyond the analyte data may be incorporated into the final report, e.g., maternal age or prior known risks. In some embodiments, the test system includes a series of modules, some of which are optional or may be repeated depending on the results of earlier modules. The test may comprise: (1) receiving a requisition, e.g., from an ordering clinician or physician, (2) receiving a patient sample, (3) performing an assay including quality controls on that sample resulting in a assay-product on an appropriate imaging substrate (e.g., contacting, binding, and/or hybridizing probes to a sample, ligating the probes, optionally amplifying the ligated probes, and immobilizing the probes to a substrate as described herein), (4) imaging the substrate in one or more spectral channels, (5) analyzing image data, (6) performing statistical calculations (e.g., comparing the first and second numbers to determine the genetic variation in the genetic sample), (7) creating and approving the clinical report, and (8) returning the report to the ordering clinician or physician. The test system may comprise a module configured to receive a requisition, e.g., from an ordering clinician or physician, a module configured to receive a patient sample, (3) a module configured to perform an assay including quality controls on that sample resulting in a assay-product on an appropriate imaging substrate, (4) a module configured to image the substrate in one or more spectral channels, (5) a module configured to analyze the image data, (6) a module configured to perform statistical calculations, (7) a module configured to create and confirm the clinical report, and and/or (8) a module configured to return the report to the ordering clinician or physician.

In one aspect, the assays and methods described herein may be performed on a single input sample simultaneously. For example, the method may comprise verifying the presence of fetal genomic molecules at or above a minimum threshold as described herein, followed by a step of estimating the target copy number state if and only if that minimum threshold is met. Therefore, one may separately run an allele-specific assay on the input sample for performing fetal fraction calculation, and a genomic target assay for computing the copy number state. In other embodiments, both assays and methods described herein may be carried out in parallel on the same sample at the same time in the same fluidic volume. Further quality control assays may also be carried out in parallel with the same universal assay processing steps. Since tags, affinity tags, and/or tagging probes in the probe products, ligated probe set, or labeled molecule to be immobilized to the substrate may be uniquely designed for every assay and every assay product, all of the parallel assay products may be localized, imaged and quantitated at different physical locations on the imaging substrate. In another aspect, the same assay or method (or some of their steps) described herein using the same probes and/or detecting the same genetic variation or control may be performed on multiple samples simultaneously either in the same or different modules (e.g., testing tube) described herein. In another aspect, assays and methods (or some of their steps) described herein using different probes and/or detecting different genetic variations or controls may be performed on single or multiple sample(s) simultaneously either in the same or different modules (e.g., testing tube).

In another aspect, image analysis may include image preprocessing, image segmentation to identify the labels, characterization of the label quality, filtering the population of detected labels based on quality, and performing statistical calculations depending on the nature of the image data. In some instances, such as when an allele-specific assay is performed and imaged, the fetal fraction may be computed. In others, such as the genomic target assay and imaging, the relative copy number state between two target genomic regions is computed. Analysis of the image data may occur in real-time on the same computer that is controlling the image acquisition, or on a networked computer, such that results from the analysis may be incorporated into the test workflow decision tree in near real-time.

In another aspect, steps (4) and (5) of the test above may be repeated multiple times for different portions of the imaging substrate such that the results dictate next steps. For example, the tests and methods described herein comprise confirming the presence and precise level of a fetal sample in a genetic sample obtained from a subject before testing for the relative copy number state of genomic targets. As described herein, an allele sensitive assay may be used to quantify the levels of fetal DNA relative to maternal DNA. The resulting probe products may be pulled down to a fetal fraction region 1 on the substrate, and imaged. In some embodiments, if and only if the calculated fetal fraction is above the minimum system requirement, the test may proceed and yield a valid result. In this way, testing of samples that fail to confirm at least the minimum input fetal fraction may be terminated before additional imaging and analysis takes place. Conversely, if the fetal fraction is above the minimum threshold, further imaging (step 4 of the test) of the genomic targets (e.g., chromosome 21, 18 or 13) may proceed followed by additional analysis (step 5 of the test). Other criteria may also be used and tested.

In another aspect, not every SNP probed in the allele-specific assay may result in useful information. For example, the maternal genomic material may have heterozygous alleles for a given SNP (e.g., allele pair AB), and the fetal material may also be heterozygous at that site (e.g., AB), hence the fetal material is indistinguishable and calculation of the fetal fraction fails. Another SNP site for the same input sample, however, may again show the maternal material to be heterozygous (e.g., AB) while the fetal material is homozygous (e.g., AA). In this example, the allele-specific assay may yield slightly more A counts than B counts due to the presence of the fetal DNA, from which the fetal fraction may be calculated. Since the SNP profile (i.e., genotype) cannot be known a priori for a given sample, multiple or numerous SNP sites should be designed such that nearly every possible sample will yield an informative SNP site. Each SNP site may be localized to a different physical location on the imaging substrate, for example by using a different tag for each SNP. However, for a given test, the fetal fraction may only be calculated successfully once. Therefore, a single or multiple locations on the substrate used to interrogate SNPs may be imaged and analyzed (e.g., in groups of one, two, three, four, five, ten, twenty, fifty or less and/or one, two, three, four, five, ten, twenty, fifty or more) until an informative SNP is detected. By alternating imaging and analysis, one may bypass imaging all possible SNP spots and significantly reduce average test duration while maintaining accuracy and robustness.

In another aspect, determining the fetal fraction of a sample may aide other aspects of the system beyond terminating tests for which the portion of fetal fraction in a sample is inadequate. For example, if the fetal fraction is high (e.g., 20%) then for a given statistical power, the number of counts required per genetic target (e.g., chr21) will be lower; if the fetal fraction is low (e.g., 1%) then for the same statistical power, a very high number of counts is required per genomic target to reach the same statistical significance. Therefore, following (4-1) imaging of the fetal fraction region 1, (5-1) analysis of those data resulting in a required counting throughput per genomic target, (4-2) imaging of genomic target region 2 commences at the required throughput, followed by (5-2) analysis of those image data and the test result for genomic variation of the input targets.

In another aspect, steps (4) and (5) of the test above may be repeated further for quality control purposes, including assessment of background levels of fluors on the imaging substrate, contaminating moieties, positive controls, or other causes of copy number variation beyond the immediate test (e.g., cancer in the mother or fetus, fetal chimeraism, twinning). Because image analysis may be real-time, and does not require completion of the entire imaging run before generating results (unlike DNA sequencing methods), intermediate results may dictate next steps from a decision tree, and tailor the test for ideal performance on an individual sample. Quality control may also encompass verification that the sample is of acceptable quality and present, the imaging substrate is properly configured, that the assay product is present and/or at the correct concentration or density, that there is acceptable levels of contamination, that the imaging instrument is functional and that analysis is yielding proper results, all feeding in to a final test report for review by the clinical team.

In another aspect, the test above comprises one or more of the following steps: (1) receiving a requisition (from, for example, an ordering clinician or physician), (2) receiving a patient sample, (3) performing an assay (including a allele-specific portion, genomic target portion and quality controls) on that sample resulting in a assay-product-containing imaging substrate, (4-1) imaging the allele-specific region of the substrate in one or more spectral channels, (5-1) analyzing allele-specific image data to compute the fetal fraction, (pending sufficient fetal fraction) (4-2) imaging the genomic target region of the substrate in one or more spectral channels, (5-2) analyzing genomic target region image data to compute the copy number state of the genomic targets, (4-3) imaging the quality control region of the substrate in one or more spectral channels, (5-3) analyzing quality control image data to compute validate and verify the test, (6) performing statistical calculations, (7) creating and approving the clinical report, and (8) sending the report back to the ordering clinician or physician.

In the following description, various exemplary embodiments are set forth in view of the Figures.

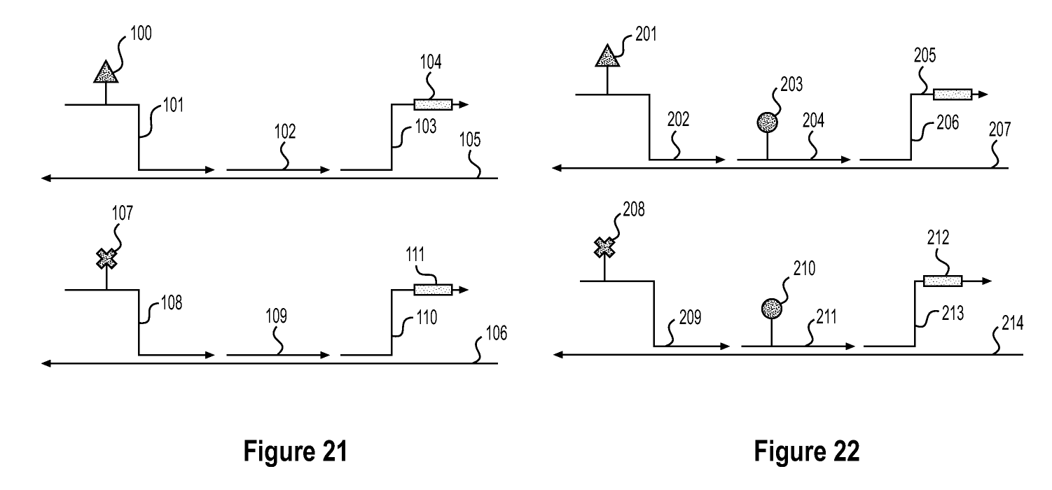

FIG. 21 is an implementation of an assay for quantifying genomic copy number at two genomic loci. In this embodiment of the assay, 105 and 106 are target molecules. 105 contains sequence corresponding to the first genomic locus “Locus 1” interrogated for copy number (example, chromosome 21), and 106 contains sequence corresponding the second genomic locus “Locus 2” interrogated for copy number (example, chromosome 18). FIG. 21 contains an example of one probe set per genomic locus, but in some embodiments of this assay, multiple probe sets will be designed to interrogate multiple regions within a genomic locus. For example, more than 10, or more than 100, or more than 500 probe sets may be designed that correspond to chromosome 21. FIG. 21 illustrates only a single probe set for each genomic locus, but importantly the scope of this invention allows for multiple probe sets for each genomic locus. FIG. 21 also illustrates a single hybridization event between a target molecule and a probe set. In practice, there will be multiple target molecules present in an assay sample. Many target molecules will contain the necessary sequences for hybridization to a probe set, and formation of a probe product. Different target molecules may hybridize to probe sets, as certain target molecules will bear genetic polymorphisms. In addition, target molecules that arise from genomic DNA may have a random assortment of molecule sizes, as well various beginning and ending sequences. In essence, there are multiple target molecules that may hybridize to a given probe set. In a single assay, multiple copies of a given probe set are added. Therefore, in a single assay up to thousands, or hundreds of thousands, or millions of specific probe products may be formed.

FIG. 21 depicts two probe sets, one probe set for Locus 1 and one probe set for Locus 2, although as aforementioned, multiple probes sets may be designed for each genomic locus. A first probe sets contains member probes 101, 102, 103. Item 101 contains label (100) type “A.” Item 103 contains an affinity tag (104) which may be used for isolation and identification of the probe product. 102 may contain no modifications, such as a label or barcode. A second probe set with member probes 108, 109, 110 carries respective features as in the first probe set. However, 108 contains a label (107) of type “B,” distinguishable from type “A.” Item 110 contains an affinity tag (111) which may be identical to or unique from 104. Many probe sets may designed that target “Locus 1,” containing unique probe sequences but the same label type “A.” Similarly, many probe sets may be designed that target “Locus 2,” containing unique probe sequences but the same label type “B.” In this embodiment, the affinity tags for the many probe sets for Locus 1 may be identical or unique, and the affinity tags for the many probe sets for Locus 2 may be identical or unique.

One or more probe sets are added to target molecules in a single vessel and exposed to sequence-specific hybridization conditions.

For each probe set, the three probes (e.g., 101, 102, 103) are hybridized (or attached via a similar probe-target interaction) to the target molecule (105) such there are no gaps in between the probes on the target molecule. That is, the probes from the probe set are adjacent to one another and ligation competent.

Ligase is added to the hybridized probes and exposed to standard ligase conditions. The ligated probes form a probe product. All (or a majority of) probe products from Locus 1 have label type “A.” All probe products from Locus 2 have label type “B.” Quantification of the probe products corresponding to the genomic loci 1 & 2 occurs using labels “A” and “B.”

In some embodiments, the probe products are immobilized onto a substrate using their affinity tags. For example, if the affinity tag is a DNA sequence, the probe products may be hybridized to regions of a DNA capture array at appropriate density for subsequent imaging.

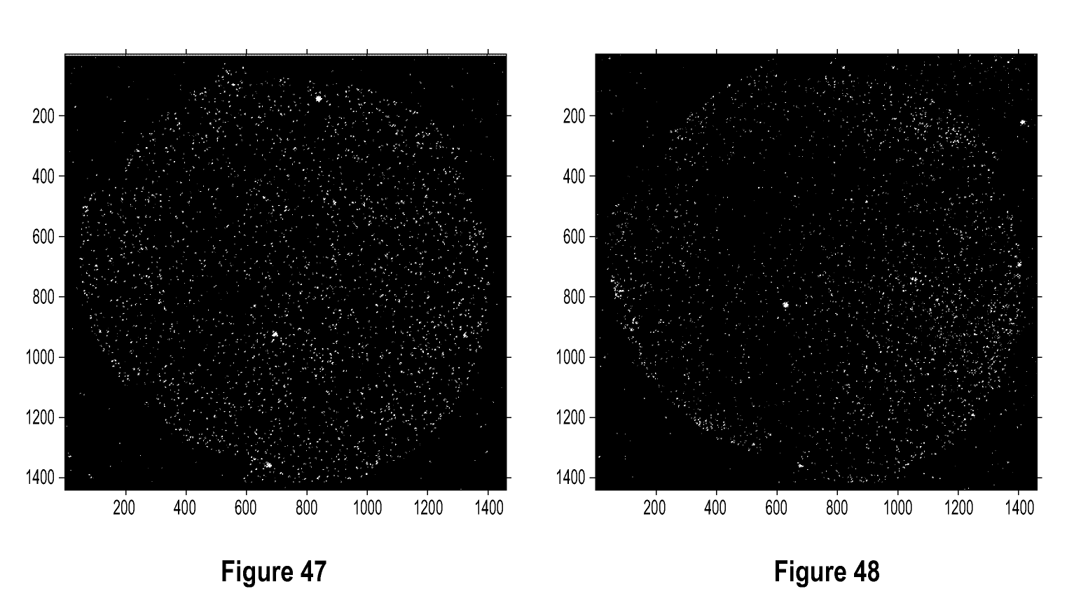

In some embodiments, affinity tags 104 and 111 contain unique and orthogonal sequences that allow surface-based positioning to one or more locations, which may be shared between hybridization products or not. FIGS. 47 and 48 show the resulting fluorescence patterns when products contain unique affinity tag sequences and the underlying substrate contains complements to each of the unique affinity tags within the same region (e.g., as the same member of an array) on a substrate. The images are of the same region of a substrate, but FIG. 47 shows Cy3 labels (covalently bound to chromosome 18 product), and FIG. 48 shows Alexa Fluor 647 labels (covalently bound to chromosome 21 product). Similar patterns may be generated for other assay embodiments that follow.

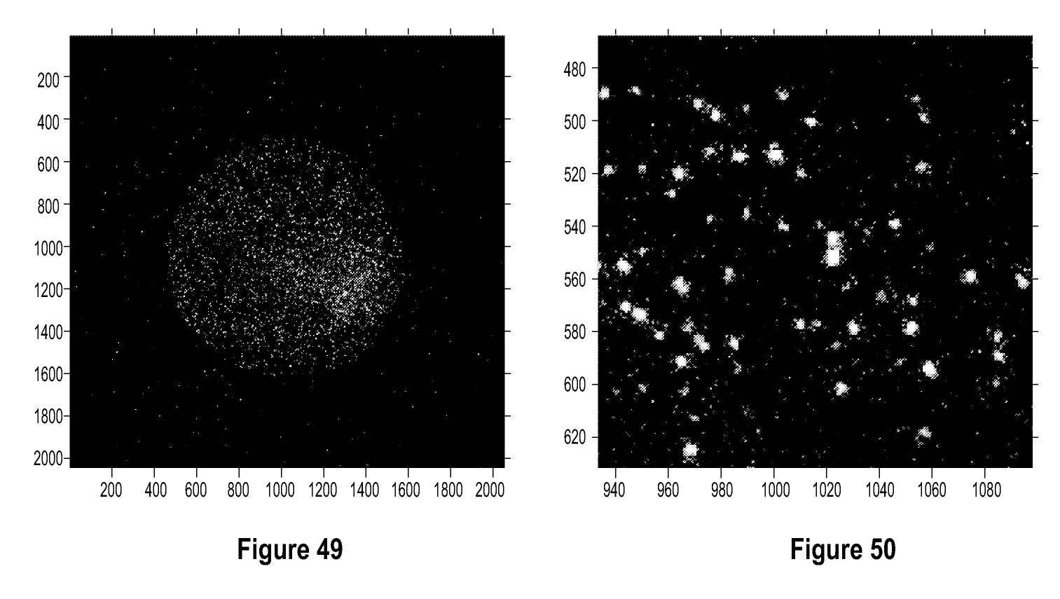

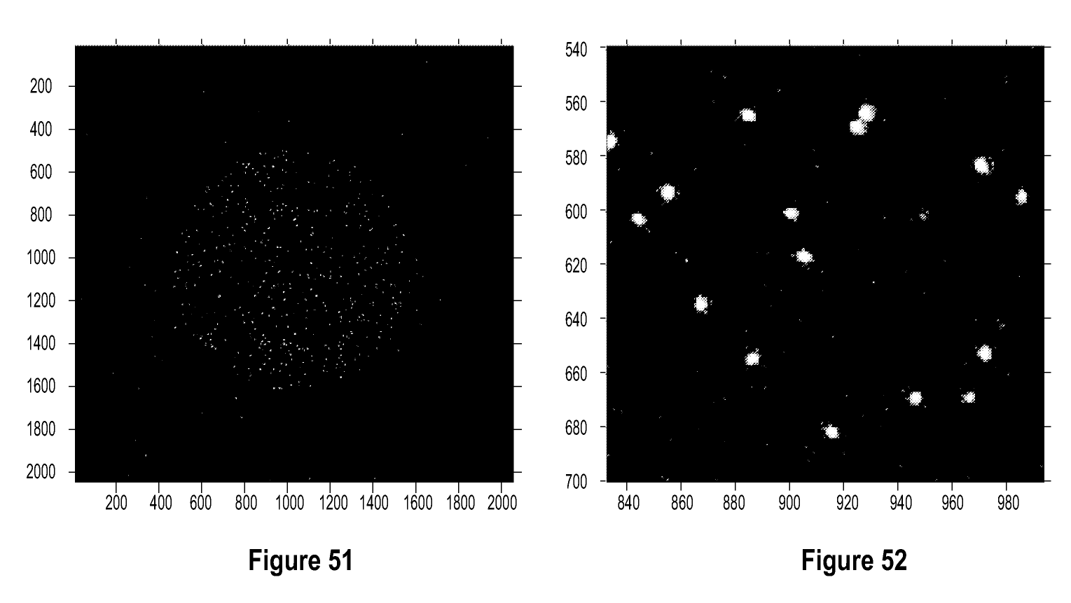

In another embodiment, affinity tags 104 and 111 contain identical sequences that allow surface-based positioning to the same region (e.g., as the same member of an array) on a substrate. That is, different products compete for the same binding sites. FIGS. 49 and 51 show the resulting fluorescence patterns when different products contain identical affinity tag sequences and the underlying substrate contains the complement to the affinity tag. The images are of the same location on a substrate, but FIG. 49 shows Cy3 labels (covalently bound to chromosome 18 product) and FIG. 51 shows Alexa Fluor 647 labels (covalently bound to chromosome 21 product). FIGS. 50 and 52 show zoomed-in regions of FIGS. 49 and 51, respectively, clearly demonstrating single-molecule resolution and individually-distinguishable labels. Similar patterns may be generated for other assay embodiments that follow.

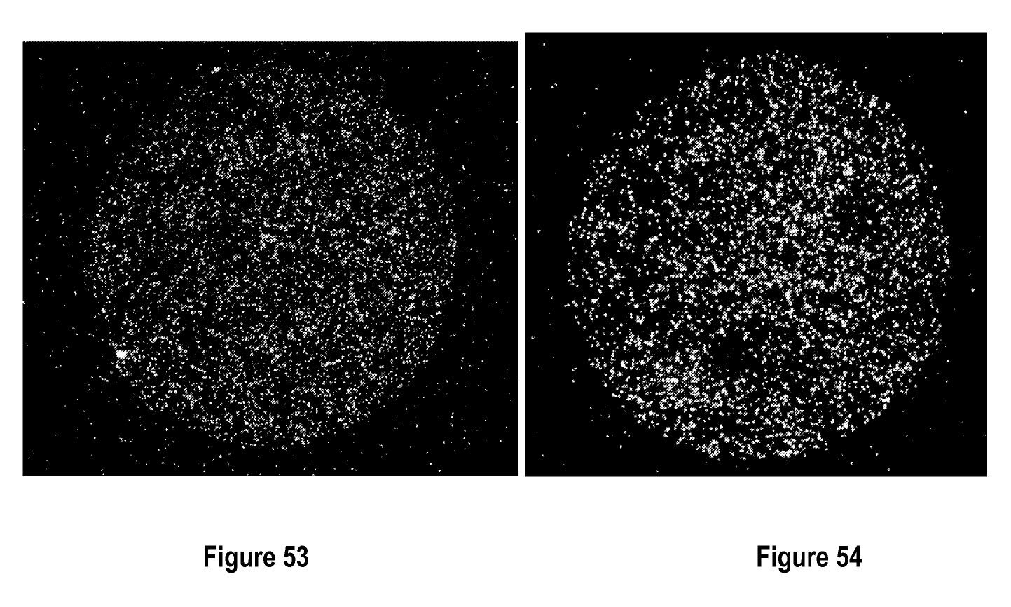

In another embodiment, affinity tags 104 and 111 contain unique and orthogonal sequences that allow surface-based positioning to more than one location on a substrate. FIGS. 53 and 54 show the resulting fluorescence patterns when products contain unique affinity tag sequences and the underlying substrate has one region containing the complement to one affinity tag complement, and another separate region containing the complement to the other affinity tag. The images are of two separate regions of a substrate, with each region containing a single affinity tag complement as previously described. FIG. 53 shows Cy3 labels (covalently bound to chromosome 21 product), and FIG. 54 shows Alexa Fluor 647 labels (covalently bound to chromosome 18 product). Similar patterns may be generated for other assay embodiments that follow.

One feature of this invention according to some embodiments is that specificity is achieved through the combination of multiple adjacent probes that must be successfully ligated together in order for the probe product to be successfully formed, captured and detected. If a probe product is not successfully formed for any reason, then it cannot be isolated, or enriched for using an affinity tag and detected. For example, if probe 101 is not successfully ligated to probe 102, then the resulting product cannot be detected. Similarly, if probe 103 is not successfully ligated to probe 102, then the resulting product cannot be isolated or enriched using an affinity tag.

Requiring all probes from the probe set to successfully hybridize to the target molecule and successfully ligate together provides high specificity and greatly reduces issues of cross-hybridization and therefore false positive signals.

In this assay, specificity is achieved through sequence-specific hybridization and ligation. In a preferred embodiment, the specificity of forming probe products occurs in the reaction vessel, prior to isolating or enriching for probe products, for example immobilization onto a surface or other solid substrate. This side-steps the challenge of standard surface based hybridization (e.g., genomic microarray) in which specificity must be entirely achieved through hybridization only with long (>40 bp) oligonucleotide sequences (e.g., Agilent and Affymetrix arrays).

The use of affinity tags allows the probe products to be immobilized on a substrate and therefore excess unbound probes to be washed away using standard methods or removed using standard methods. Therefore all or most of the labels on the surface are a part of a specifically formed probe product that is immobilized to the surface.

One feature of this invention according to some embodiments is that the surface capture does not affect the accuracy. That is, it does not introduce any bias. In one example, if the same affinity tag is used for probe sets from different genomic loci, with probe sets targeting each locus having a different label. Probe products from both genomic loci may be immobilized to the same location on the substrate using the same affinity tag. That is probe products from Locus 1 and Locus 2 will be captured with the same efficiency, so not introducing any locus specific bias.

In some embodiments, some or all of the unbound probes and/or target molecules are removed prior to surface capture using standard methods. This decreases interference between unbound probes and/or target molecules and the probe products during surface capture.

One feature of this invention according to some embodiments is that multiple affinity tag types may be placed in the same region of the substrate (for example, the same array spot or member of the array). This has many advantages, including placement of control or calibration markers. FIGS. 22-46 describe additional exemplary embodiments of this invention. These Figures do not represent all possible embodiments, and all other variations of this assay are included as a part of this invention. Additionally, all features of the embodiment described in FIG. 21 are applicable to all additional other embodiments of the assay described in this application.

FIG. 22 depicts a modification of the general procedure described in FIG. 21. FIG. 22 depicts two probe sets, one probe set for Locus 1 and one probe set for Locus 2, although as aforementioned, multiple probes sets may be designed for each genomic locus. 207 and 214 are target molecules corresponding to Locus 1 and Locus 2, respectively. A first probe sets contains member probes 202, 204, 206. 202 contains a label (201) of type “A.” 206 contains an affinity tag (205) which may be used for isolation and identification of the probe product. A second probe set with member probes 209, 211, 231 carries respective features as in the first probe set. However, 209 contains a label (208) of type “B,” distinguishable from type “A.” 213 contains an affinity tag (212) which may be identical to or unique from 205. Many probe sets may be designed such that target “Locus 1,” containing unique probe sequences but the same label type “A.” Similarly, many probe sets may be designed that target “Locus 2,” containing unique probe sequences but the same label type “B.” In this embodiment, the affinity tags for the many probe sets for Locus 1 may be identical or unique or a mixture of identical and unique, and the affinity tags for the many probe sets for Locus 2 may be identical or unique or a mixture of identical and unique. In this embodiment, the probes 204 and 211 may contain one or more labels (203, 210) of type “C.” Therefore, probe products will contain a combination of labels. For Locus 1, probe products will contains labels of type “A” and type “C,” whereas probe products from Locus 2 will contain labels of type “B” and type “C.”

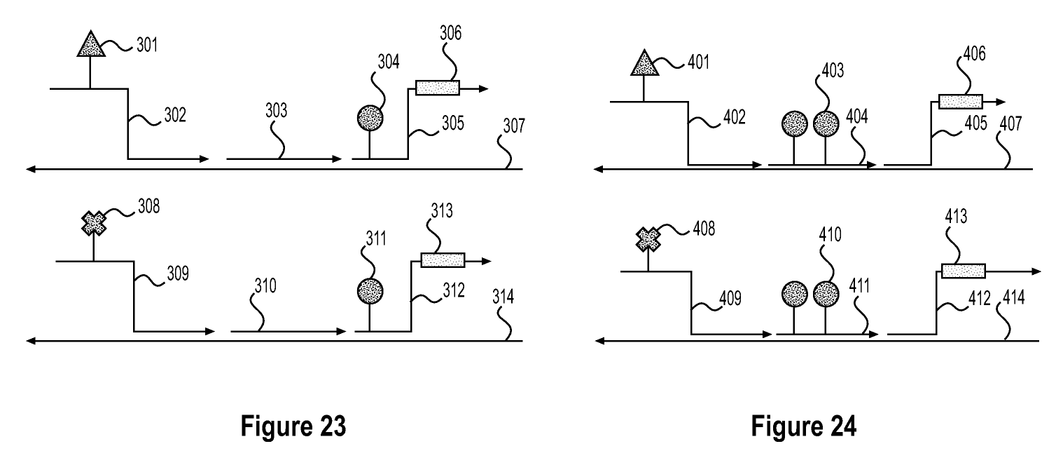

FIG. 23 depicts a modification of the general procedure described in FIG. 21. FIG. 23 depicts two probe sets, one probe set for Locus 1 and one probe set for Locus 2, although as aforementioned, multiple probes sets may be designed for each genomic locus. 307 and 314 are target molecules corresponding to Locus 1 and Locus 2, respectively. A first probe set contains member probes 302, 303, 305. 302 contains a label (301) of type “A.” 305 contains an affinity tag (306) which may be used for isolation and identification of the probe product. A second probe set with member probes 309, 310, 312 carries respective features as in the first probe set. However, 309 contains a label (308) of type “B,” distinguishable from type “A.” 312 contains an affinity tag (313) which may be identical to or unique from 306. Many probe sets may designed that target “Locus 1,” containing unique probe sequences but the same label type “A.” Similarly, many probe sets may be designed that target “Locus 2,” containing unique probe sequences but the same label type “B.” In this embodiment, the affinity tags for the many probe sets for Locus 1 may be identical or unique, and the affinity tags for the many probe sets for Locus 2 may be identical or unique. In this embodiment, the probes 305 and 312 contain one or more labels (304, 311) of type “C.” Therefore, probe products will contain a combination of labels. For Locus 1, probe products will contains labels of type “A” and type “C,” whereas probe products from Locus 2 will contain labels of type “B” and type “C.”

FIG. 24 depicts a modification of the general procedure described in FIG. 21. FIG. 24 depicts two probe sets, one probe set for Locus 1 and one probe set for Locus 2, although as aforementioned, multiple probes sets may be designed for each genomic locus. 407 and 414 are target molecules corresponding to Locus 1 and Locus 2, respectively.

A first probe sets contains member probes 402, 405. 402 contains a label (401) of type “A.” 405 contains an affinity tag (406) which may be used for isolation and identification of the probe product.

A second probe set with member probes 409, 412 carries respective features as in the first probe set. However, 409 contains a label (408) of type “B,” distinguishable from type “A.” 412 contains an affinity tag (413) which may be identical to or unique from 406. Many probe sets may designed that target “Locus 1,” containing unique probe sequences but the same label type “A.” Similarly, many probe sets may be designed that target “Locus 2,” containing unique probe sequences but the same label type “B.” In this embodiment, the affinity tags for the many probe sets for Locus 1 may be identical or unique, and the affinity tags for the many probe sets for Locus 2 may be identical or unique.

In this embodiment, probes 402 and 405 hybridize to sequences corresponding to Locus 1, but there is a “gap” on the target molecule consisting of one or more nucleotides between hybridized probes 402 and 405. In this embodiment, a DNA polymerase or other enzyme may be used to synthesize a new polynucleotide species (404) that covalently joins 402 and 405. That is, the probe product formed in this example is a single contiguous nucleic acid molecule with a sequence corresponding to Locus 1, and bearing the labels and/or affinity tags above. Additionally, 404 may contain one or more labels of type “C,” possibly as a result of incorporation of a one of more nucleotides bearing a label of type “C.” This example also conveys to the probe product formed for Locus 2, containing probes 409 and 412. Therefore, probe products will contain a combination of labels. For Locus 1, probe products will contains labels of type “A” and type “C,” whereas probe products from Locus 2 will contain labels of type “B” and type “C.”

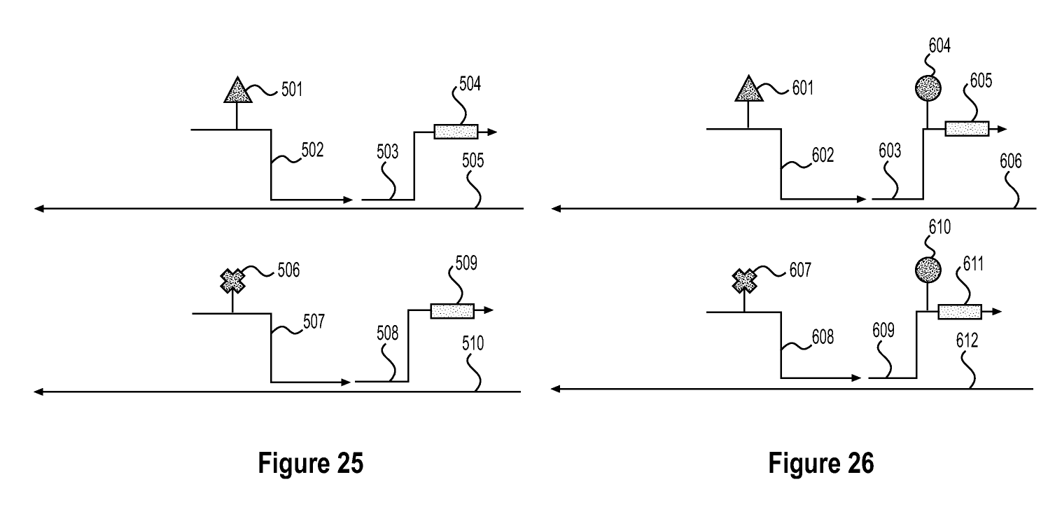

FIG. 25 depicts a modification of the general procedure described in FIG. 21. FIG. 25 depicts two probe sets, one probe set for Locus 1 and one probe set for Locus 2, although as aforementioned, multiple probes sets may be designed for each genomic locus. 505 and 510 are target molecules corresponding to Locus 1 and Locus 2, respectively. A first probe sets contains member probes 502, 503. 502 contains a label (501) of type “A.” 503 contains an affinity tag (504) which may be used for isolation and identification of the probe product. A second probe set with member probes 507, 508 carries respective features as in the first probe set. However, 507 contains a label (506) of type “B,” distinguishable from type “A.” 508 contains an affinity tag (509) which may be identical to or unique from 504. Many probe sets may designed that target “Locus 1,” containing unique probe sequences but the same label type “A.” Similarly, many probe sets may be designed that target “Locus 2,” containing unique probe sequences but the same label type “B.” In this embodiment, the affinity tags for the many probe sets for Locus 1 may be identical or unique, and the affinity tags for the many probe sets for Locus 2 may be identical or unique.

FIG. 26 depicts a modification of the general procedure described in FIG. 21. FIG. 26 depicts two probe sets, one probe set for Locus 1 and one probe set for Locus 2, although as aforementioned, multiple probes sets may be designed for each genomic locus. 606 and 612 are target molecules corresponding to Locus 1 and Locus 2, respectively. A first probe sets contains member probes 602, 603. 602 contains a label (601) of type “A.” 603 contains an affinity tag (605) which may be used for isolation and identification of the probe product. A second probe set with member probes 608, 609 carries respective features as in the first probe set. However, 608 contains a label (607) of type “B,” distinguishable from type “A.” 609 contains an affinity tag (611) which may be identical to or unique from 605. Many probe sets may designed that target “Locus 1,” containing unique probe sequences but the same label type “A.” Similarly, many probe sets may be designed that target “Locus 2,” containing unique probe sequences but the same label type “B.” In this embodiment, the affinity tags for the many probe sets for Locus 1 may be identical or unique, and the affinity tags for the many probe sets for Locus 2 may be identical or unique.

In this embodiment, the probes 603 and 609 contain one or more labels (604, 610) of type “C.” Therefore, probe products will contain a combination of labels. For Locus 1, probe products will contains labels of type “A” and type “C,” whereas probe products from Locus 2 will contain labels of type “B” and type “C.”

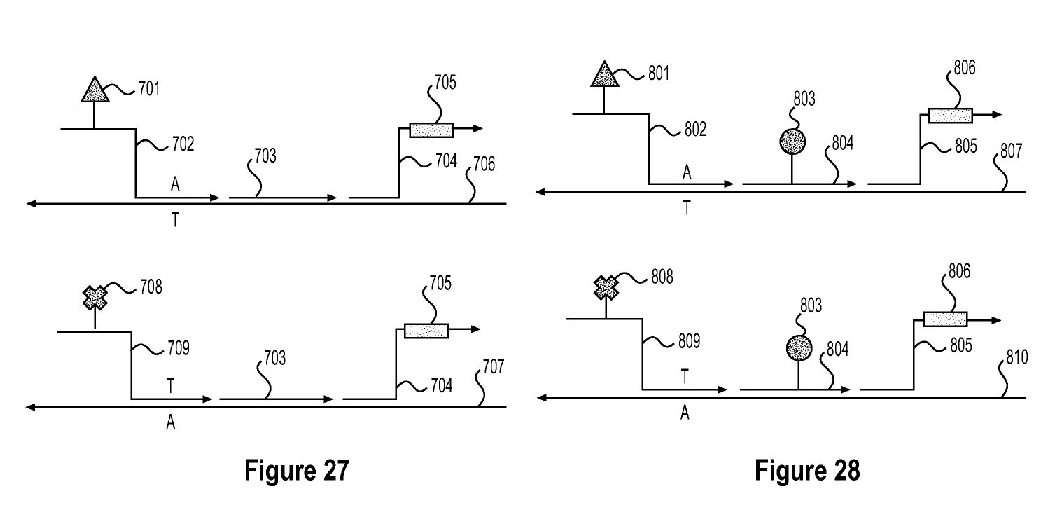

FIG. 27 depicts a modification of the general procedure described in FIG. 21. FIG. 27 depicts two probe sets for identifying various alleles of the same genomic locus. For example, for distinguishing maternal and fetal alleles, in the case of cell free DNA isolated from a pregnant woman, or for distinguishing host and donor alleles, in the case of cell free DNA from a recipient of an organ transplant. FIG. 27 depicts two probe sets—one probe set for Allele 1 and one probe set for Allele 2. 706 and 707 are target molecules corresponding to Allele 1 and Allele 2, respectively. A first probe set contains member probes 702, 703, 704. 702 contains a label (701) of type “A.” 704 contains an affinity tag (705) which may be used for isolation and identification of the probe product. A second probe set with member probes 709, 703, 704 carries respective features as in the first probe set. In this embodiment, 703 and 704 are identical for both probe sets. However, 709 contains a label (708) of type “B,” distinguishable from type “A.” In this embodiment, 702 and 709 contain sequences that are nearly identical, and differ by only one nucleotide in the sequence. Therefore, hybridization sequences of these two probes, which are configured to hybridize to the regions for Allele 1 and Allele 2, contains complementary regions for Allele 1 (702), and Allele 2 (709). Further, the length of each hybridization domain on 702 and 709, as well as experimental hybridization conditions are designed such that probe 702 will only hybridize to Allele 1 and probe 709 will only hybridize to Allele 2. The purpose of this assay type is to accurately quantify the frequency of Allele 1 and Allele 2 in a sample.

FIG. 28 depicts a modification of the general procedure described in FIG. 21. FIG. 28 depicts two probe sets for identifying various alleles of the same genomic locus. For example, for distinguishing maternal and fetal alleles, in the case of cell free DNA isolated from a pregnant woman, or for distinguishing host and donor alleles, in the case of cell free DNA from a recipient of an organ transplant. FIG. 28 depicts two probe sets—one probe set for Allele 1 and one probe set for Allele 2. 807 and 810 are target molecules corresponding to Allele 1 and Allele 2, respectively. A first probe set contains member probes 802, 804, 805. 802 contains a label (801) of type “A.” 805 contains an affinity tag (806) which may be used for isolation and identification of the probe product. A second probe set with member probes 809, 804, 805 carries respective features as in the first probe set. In this embodiment, 804 and 805 are identical for both probe sets. However, 809 contains a label (808) of type “B,” distinguishable from type “A.” In this embodiment, 802 and 809 contain sequences that are nearly identical, and differ by only one nucleotide in the sequence. Therefore, hybridization sequences of these two probes contain complementary regions for Allele 1 (802), and Allele 2 (809). Further, the length of each hybridization domain on 802 and 809, as well as experimental hybridization conditions are designed such that probe 802 will only hybridize to Allele 1 and probe 809 will only hybridize to Allele 2. The purpose of this assay type is to be able to accurately quantify the frequency of Allele 1 and Allele 2 in a sample. In this embodiment, the probe 804 contains one or more labels (803) of type “C.” Therefore, probe products will contain a combination of labels. For Allele 1, probe products will contain labels of type “A” and type “C,” whereas probe products from Allele 2 will contain labels of type “B” and type “C.”

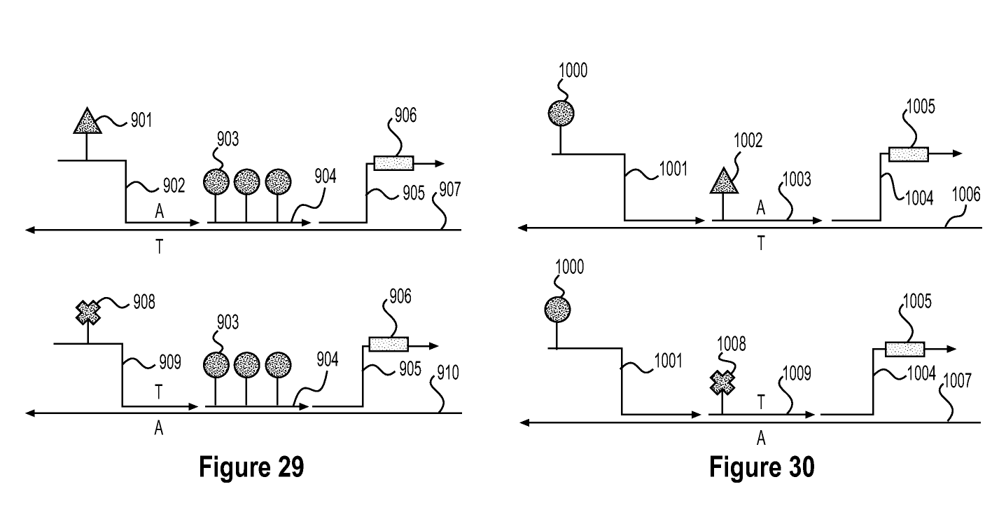

FIG. 29 depicts a modification of the general procedure described in FIG. 21. FIG. 29 depicts two probe sets for identifying various alleles of the same genomic locus. For example, for distinguishing maternal and fetal alleles, in the case of cell free DNA isolated from a pregnant woman, or for distinguishing host and donor alleles, in the case of cell free DNA from a recipient of an organ transplant. FIG. 29 depicts two probe sets, one probe set for Allele 1 and one probe set for Allele 2.

907 and 910 are target molecules corresponding to Allele 1 and Allele 2, respectively. A first probe set contains member probes 902, 905. 902 contains a label (901) of type “A.” Item 905 contains an affinity tag (906) which may be used for isolation and identification of the probe product. A second probe set with member probes 909, 905 carries respective features as in the first probe set. In this embodiment, 905 is identical for both probe sets. However, 909 contains a label (908) of type “B,” distinguishable from type “A.” In this embodiment, 902 and 909 contain sequences that are nearly identical, and differ by only one nucleotide in the sequence. Therefore, hybridization sequences of these two probes contain complementary regions for Allele 1 (902), and Allele 2 (909). Further, the length of each hybridization domain on 902 and 909, as well as experimental hybridization conditions are designed such that probe 902 will only hybridize to Allele 1 and probe 909 will only hybridize to Allele 2. The purpose of this assay type is to be able to accurately quantify the frequency of Allele 1 and Allele 2 in a sample.

In this embodiment, probes 902 and 905 hybridize to sequences corresponding to Allele 1, such that there is a “gap” on the target molecule consisting of one or more nucleotides between hybridized probes 902 and 905. In this embodiment, a DNA polymerase or other enzyme may be used to synthesize a new polynucleotide species (904) that covalently joins 902 and 905. That is, the probe product formed in this example is a single contiguous nucleic acid molecule with a sequence corresponding to Allele 1, and bearing the labels and/or affinity tags above. Additionally, 904 may contain one or more labels of type “C,” possibly as a result of incorporation of a nucleotide bearing a label of type “C.” This example also conveys to the probe product formed for Allele 2, containing probes 909 and 905.

FIG. 30 depicts a modification of the general procedure described in FIG. 21. FIG. 30 depicts two probe sets for identifying various alleles of the same genomic locus. For example, for distinguishing maternal and fetal alleles, in the case of cell free DNA isolated from a pregnant woman, or for distinguishing host and donor alleles, in the case of cell free DNA from a recipient of an organ transplant. FIG. 30 depicts two probe sets, one probe set for Allele 1 and one probe set for Allele 2.

1006 and 1007 are target molecules corresponding to Allele 1 and Allele 2, respectively. A first probe set contains member probes 1001, 1003, 1004. 1003 contains a label (1002) of type “A.” 1004 contains an affinity tag (1005) which may be used for isolation and identification of the probe product.

A second probe set with member probes 1001, 1009, 1004 carries respective features as in the first probe set. In this embodiment, 1001 is identical for both probe sets and 1004 is identical for both probe sets. However, 1009 contains a label (1008) of type “B,” distinguishable from type “A.”

In this embodiment, 1003 and 1009 contain sequences that are nearly identical, and differ by only one nucleotide in the sequence. Therefore, hybridization sequences of these two probes contains complementary regions for Allele 1 (1003), and Allele 2 (1009), respectively. Further, the length of each hybridization domain on 1003 and 1009, as well as experimental hybridization conditions are designed such that probe 1003 will only hybridize to Allele 1 and probe 1009 will only hybridize to Allele 2. The purpose of this assay type is to be able to accurately quantify the frequency of Allele 1 and Allele 2 in a sample. In this embodiment, the probe 1001 contains one or more labels (1000) of type “C.” Therefore, probe products will contain a combination of labels. For Allele 1, probe products will contains labels of type “A” and type “C,” whereas probe products from Allele 2 will contain labels of type “B” and type “C.”

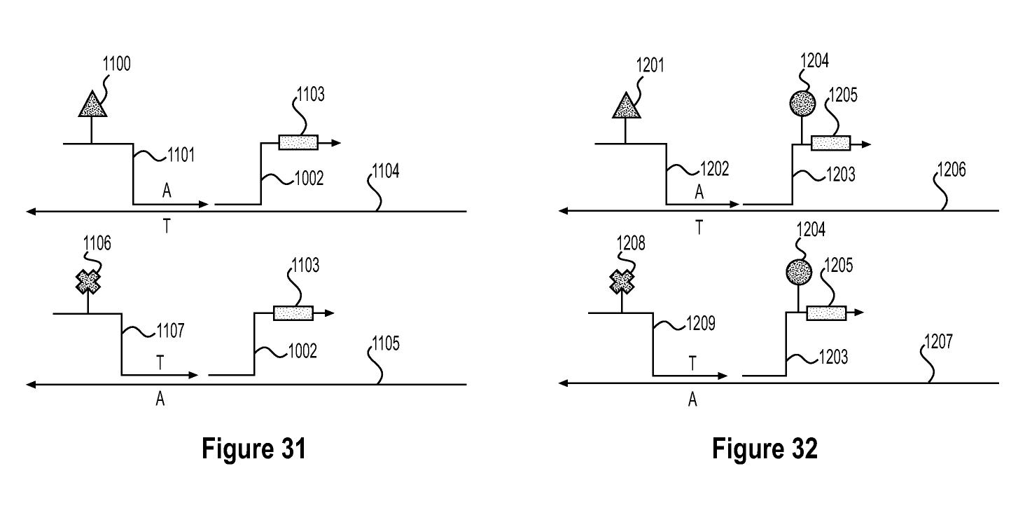

FIG. 31 depicts a modification of the general procedure described in FIG. 21. FIG. 31 depicts two probe sets for identifying various alleles of the same genomic locus. For example, for distinguishing maternal and fetal alleles, in the case of cell free DNA isolated from a pregnant woman, or for distinguishing host and donor alleles, in the case of cell free DNA from a recipient of an organ transplant. FIG. 31 depicts two probe sets—one probe set for Allele 1 and one probe set for Allele 2. 1104 and 1105 are target molecules corresponding to Allele 1 and Allele 2, respectively. A first probe set contains member probes 1101, 1102. 1101 contains a label (1100) of type “A.” 1102 contains an affinity tag (1103) which may be used for isolation and identification of the probe product. A second probe set with member probes 1107, 1102 carries respective features as in the first probe set. In this embodiment, 1102 is identical for both probe sets. However, 1107 contains a label (1106) of type “B,” distinguishable from type “A.” In this embodiment, 1101 and 1107 contain sequences that are nearly identical, and differ by only one nucleotide in the sequence. Therefore, hybridization sequences of these two probes contains complementary regions for Allele 1 (1101), and Allele 2 (1107). Further, the length of each hybridization domain on 1101 and 1107, as well as experimental hybridization conditions are designed such that probe 1101 will only hybridize to Allele 1 and probe 1107 will only hybridize to Allele 2. The purpose of this assay type is to be able to accurately quantify the frequency of Allele 1 and Allele 2 in a sample.

FIG. 32 depicts a modification of the general procedure described in FIG. 21. FIG. 32 depicts two probe sets for identifying various alleles of the same genomic locus. For example, for distinguishing maternal and fetal alleles, in the case of cell free DNA isolated from a pregnant woman, or for distinguishing host and donor alleles, in the case of cell free DNA from a recipient of an organ transplant. FIG. 32 depicts two probe sets—one probe set for Allele 1 and one probe set for Allele 2. 1206 and 1207 are target molecules corresponding to Allele 1 and Allele 2, respectively. A first probe set contains member probes 1202, 1203. 1202 contains a label (1201) of type “A.” 1203 contains an affinity tag (1205) which may be used for isolation and identification of the probe product. A second probe set with member probes 1209, 1203 carries respective features as in the first probe set. In this embodiment, 1203 is identical for both probe sets. However, 1209 contains a label (1208) of type “B,” distinguishable from type “A.” In this embodiment, 1202 and 1209 contain sequences that are nearly identical, and differ by only one nucleotide in the sequence. Therefore, hybridization sequences of these two probes contains complementary regions for Allele 1 (1202), and Allele 2 (1209). Further, the length of each hybridization domain on 1202 and 1209, as well as experimental hybridization conditions are designed such that probe 1202 will only hybridize to Allele 1 and probe 1209 will only hybridize to Allele 2. The purpose of this assay type is to be able to accurately quantify the frequency of Allele 1 and Allele 2 in a sample. In this embodiment, the probe 1203 contains one or more labels (1204) of type “C.” Therefore, probe product will contain a combination of labels. For Allele 1, probe products will contains labels of type “A” and type “C,” whereas probe products from Allele 2 will contain labels of type “B” and type “C.”

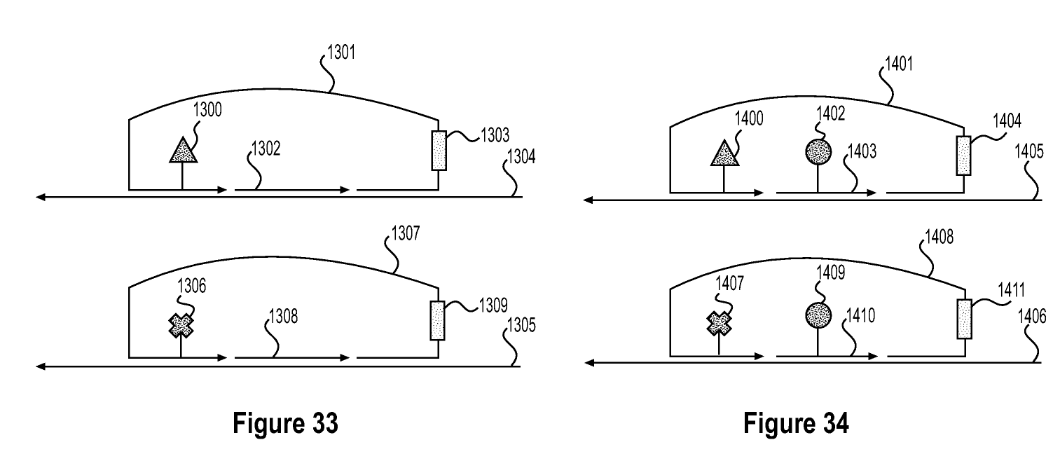

FIG. 33 depicts a modification of the general procedure described in FIG. 21. FIG. 33 depicts two probe sets, one probe set for Locus 1 and one probe set for Locus 2, although as aforementioned, multiple probes sets may be designed for each genomic locus. 1304 and 1305 are target molecules corresponding to Locus 1 and Locus 2, respectively. A first probe sets contains member probes 1301, 1302. 1301 contains a label (1300) of type “A.” 1301 contains an affinity tag (1303) which may be used for isolation and identification of the probe product. A second probe set with member probes 1307, 1308 carries respective features as in the first probe set. However, 1307 contains a label (1306) of type “B,” distinguishable from type “A.” 1307 contains an affinity tag (1309) which may be identical to or unique from 1303. Many probe sets may designed that target “Locus 1,” containing unique probe sequences but the same label type “A.” Similarly, many probe sets may be designed that target “Locus 2,” containing unique probe sequences but the same label type “B.” In this embodiment, the affinity tags for the many probe sets for Locus 1 may be identical or unique, and the affinity tags for the many probe sets for Locus 2 may be identical or unique. In this embodiment, the probes 1301 and 1307 have similar structures. For example, on probe 1301 there are two distinct hybridization domains, such that probe 1302 may be ligated to each end of 1301, forming a probe product consisting of a contiguous, topologically closed molecule of DNA (e.g., a circular molecule). The non-hybridizing sequence on probe 1301 may contain additional features, possibly restriction enzyme sites, or primer binding sites for universal amplification.

One feature of this embodiment is that all probe products are contiguous circular molecules. In this manner, probe products may be isolated from all other nucleic acids via enzymatic degradation of all linear nucleic acid molecules, for example, using an exonuclease.

FIG. 34 depicts a modification of the general procedure described in FIG. 21. FIG. 34 depicts two probe sets, one probe set for Locus 1 and one probe set for Locus 2, although as aforementioned, multiple probes sets may be designed for each genomic locus. 1405 and 1406 are target molecules corresponding to Locus 1 and Locus 2, respectively. A first probe sets contains member probes 1401, 1403. 1401 contains a label (1400) of type “A.” 1401 contains an affinity tag (1404) which may be used for isolation and identification of the probe product. A second probe set with member probes 1408, 1410 carries respective features as in the first probe set. However, 1408 contains a label (1407) of type “B,” distinguishable from type “A.” 1408 contains an affinity tag (1411) which may be identical to or unique from 1404. Many probe sets may designed that target “Locus 1,” containing unique probe sequences but the same label type “A.” Similarly, many probe sets may be designed that target “Locus 2,” containing unique probe sequences but the same label type “B.” In this embodiment, the affinity tags for the many probe sets for Locus 1 may be identical or unique, and the affinity tags for the many probe sets for Locus 2 may be identical or unique. In this embodiment, the probes 1401 and 1408 have similar structures. For example, on probe 1401 there are two distinct hybridization domains, such that probe 1403 may be ligated to each end of 1401, forming a probe product consisting of a contiguous, topologically closed molecule of DNA (e.g., a circular molecule). The non-hybridizing sequence on probe 1401 may contain additional features, possibly restriction enzyme sites, or primer binding sites for universal amplification.

One feature of this embodiment is that all probe products are contiguous circular molecules. In this manner, probe products may be isolated from all other nucleic acids via enzymatic degradation of all linear nucleic acid molecules, for example, using an exonuclease. In this embodiment, the probes 1403 and 1410 contain one or more labels (1402, 1409) of type “C.” Therefore, probe products will contain a combination of labels. For Locus 1, probe products will contains labels of type “A” and type “C,” whereas probe products from Locus 2 will contain labels of type “B” and type “C.”

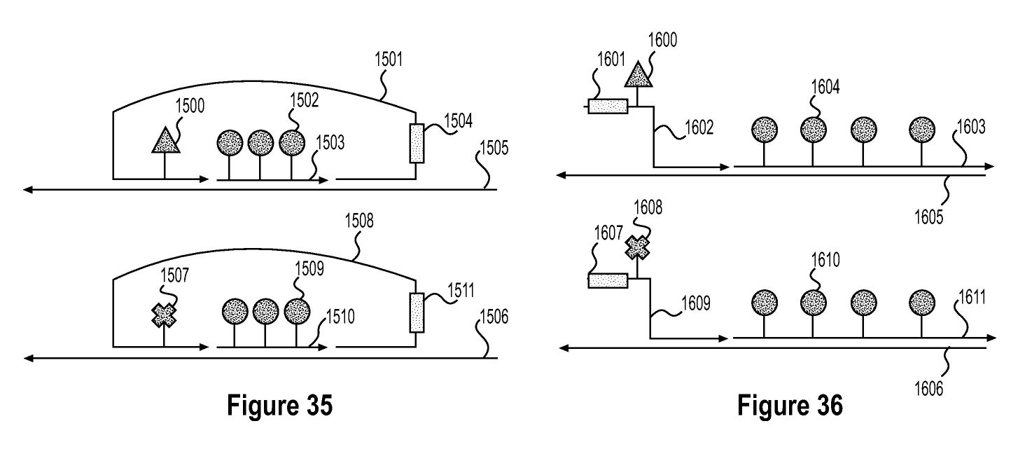

FIG. 35 depicts a modification of the general procedure described in FIG. 21. FIG. 35 depicts two probe sets, one probe set for Locus 1 and one probe set for Locus 2, although as aforementioned, multiple probes sets may be designed for each genomic locus. 1505 and 1506 are target molecules corresponding to Locus 1 and Locus 2, respectively. A first probe sets contains member probe 1501. 1501 contains a label (1500) of type “A.” 1501 contains an affinity tag (1504) which may be used for isolation and identification of the probe product. A second probe set with member probe 1508 carries respective features as in the first probe set. However, 1508 contains a label (1507) of type “B,” distinguishable from type “A.” 1508 contains an affinity tag (1511) which may be identical to or unique from 1504. Many probe sets may designed that target “Locus 1,” containing unique probe sequences but the same label type “A.” Similarly, many probe sets may be designed that target “Locus 2,” containing unique probe sequences but the same label type “B.” In this embodiment, the affinity tags for the many probe sets for Locus 1 may be identical or unique, and the affinity tags for the many probe sets for Locus 2 may be identical or unique. In this embodiment, the probes 1501 and 1508 have similar structures.

For example, on probe 1501 there are two distinct hybridization domains, such that when hybridized against a target molecule, there is a gap between the two hybridization domains. In this embodiment, a DNA polymerase or other enzyme may be used to synthesize a new polynucleotide species (1503) that covalently fills the gap between the hybridization domains of 1501. That is, the probe product formed in this example is a single, contiguous, topologically closed molecule of DNA (e.g., a circular molecule) with a sequence corresponding to Locus 1, and bearing the labels and/or affinity tags above. Additionally, 1503 may contain one or more labels of type “C,” possibly as a result of incorporation of a nucleotide bearing a label of type “C.” This example also conveys to the probe product formed for Locus 2, containing probe 1508. The non-hybridizing sequence on probe 1501 and probe 1508 may contain additional features, possibly restriction enzyme sites. One feature of this embodiment is that all probe products are contiguous circular molecules. In this manner, probe products may be isolated from all other nucleic acids via enzymatic degradation of all linear nucleic acid molecules, for example, using an exonuclease. Probe products will contain a combination of labels. For Locus 1, probe products will contains labels of type “A” and type “C,” whereas probe products from Locus 2 will contain labels of type “B” and type “C.”

FIG. 36 depicts a modification of the general procedure described in FIG. 21. FIG. 36 depicts two probe sets, one probe set for Locus 1 and one probe set for Locus 2, although as aforementioned, multiple probes sets may be designed for each genomic locus. 1605 and 1606 are target molecules corresponding to Locus 1 and Locus 2, respectively.

A first probe sets contains member probe 1602. 1602 contains a label (1600) of type “A.” 1602 contains an affinity tag (1601) which may be used for isolation and identification of the probe product.

A second probe set with member probe 1609 carries respective features as in the first probe set. However, 1609 contains a label (1608) of type “B,” distinguishable from type “A.” 1609 contains an affinity tag (1607) which may be identical to or unique from 1601. Many probe sets may designed that target “Locus 1,” containing unique probe sequences but the same label type “A.” Similarly, many probe sets may be designed that target “Locus 2,” containing unique probe sequences but the same label type “B.” In this embodiment, the affinity tags for the many probe sets for Locus 1 may be identical or unique, and the affinity tags for the many probe sets for Locus 2 may be identical or unique.

In this embodiment, probes 1602 and 1609 hybridize to sequences corresponding to Locus 1 or Locus 2 respectively, and a DNA polymerase or other enzyme may be used to synthesize a new polynucleotide sequence, for example 1603 in the case of Locus 1 or 1611 in the case of Locus 2. In this embodiment, 1603 and 1611 may contain one or more labels (1604) of type “C,” possibly as a result of incorporation of one of more nucleotides bearing a label of type “C.” This example also conveys to the probe product formed for Locus 2. Therefore, probe products will contain a combination of labels. For Locus 1, probe products will contains labels of type “A” and type “C,” whereas probe products from Locus 2 will contain labels of type “B” and type “C.” This embodiment results in probe products with high specificity for sequences in Locus 1 or Locus 2 respectively.

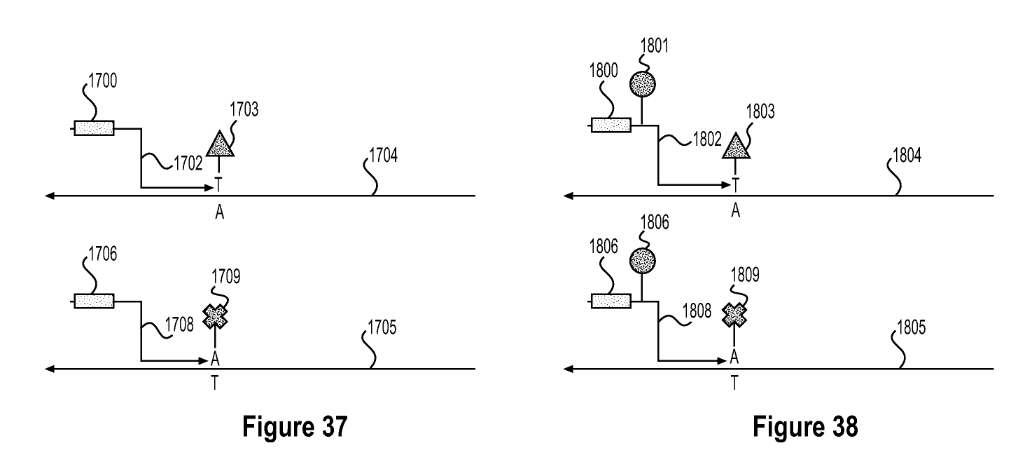

FIG. 37 depicts a modification of the general procedure described in FIG. 21. FIG. 37 depicts two probe sets, one probe set for Locus 1 and one probe set for Locus 2, although as aforementioned, multiple probes sets may be designed for each genomic locus. 1704 and 1705 are target molecules corresponding to Locus 1 and Locus 2, respectively.

A first probe sets contains member probe 1702. 1702 contains an affinity tag (1700) which may be used for isolation and identification of the probe product.

A second probe set with member probe 1708 carries respective features as in the first probe set. 1708 contains an affinity tag (1706) which may be identical to or unique from 1700. Many probe sets may designed that target “Locus 1,” containing unique probe sequences. Similarly, many probe sets may be designed that target “Locus 2,” containing unique probe sequences. In this embodiment, the affinity tags for the many probe sets for Locus 1 may be identical or unique, and the affinity tags for the many probe sets for Locus 2 may be identical or unique.

In this embodiment, probes 1702 and 1708 hybridize to sequences corresponding to Locus 1 and Locus 2 respectively. The designs of each probe for Locus 1 and Locus 2 are such that the first adjacent nucleotide next to the hybridization domains contains a different nucleotide for Locus 1 than for Locus 2. In this example, the first adjacent nucleotide next to the hybridization domain of 1702 is an “A,” whereas the first adjacent nucleotide next to the hybridization domain of 1708 is a “T.” In this embodiment, all probes for Locus 1 shall be designed such that the first nucleotide immediately adjacent to the hybridization domain shall consist of different nucleotide(s) than the first nucleotide immediately adjacent to the hybridization domain of the probes for Locus 2. That is, by design, probe sets from Locus 1 and Locus 2 may be distinguished from one another based on the identity of the first nucleotide immediately adjacent to the hybridization domain.

In this embodiment, a DNA polymerase or other enzyme will be used to add at least one additional nucleotide to each of the probe sequences. In this example, the nucleotide substrates for the DNA polymerase are competent for a single addition, for example, the nucleotides may be dideoxy chain terminators. That is, only one new nucleotide shall be added to each probe sequence. In this example, the nucleotide added to probe 1702 will contain one or more labels (1703) of type “A.” The nucleotide added to probe 1708 will contain one or more labels (1709) of type “B,” such that the probe products for Locus 1 may be distinguished from the probe products from Locus 2.

FIG. 38 depicts a modification of the general procedure described in FIG. 21. FIG. 38 depicts two probe sets, one probe set for Locus 1 and one probe set for Locus 2, although as aforementioned, multiple probes sets may be designed for each genomic locus. 1804 and 1805 are target molecules corresponding to Locus 1 and Locus 2, respectively.

A first probe sets contains member probe 1802. 1802 contains an affinity tag (1800) which may be used for isolation and identification of the probe product.

A second probe set with member probe 1808 carries respective features as in the first probe set. 1808 contains an affinity tag (1806) which may be identical to or unique from 1800. Many probe sets may be designed that target “Locus 1,” containing unique probe sequences. Similarly, many probe sets may be designed that target “Locus 2,” containing unique probe sequences. In this embodiment, the affinity tags for the many probe sets for Locus 1 may be identical or unique, and the affinity tags for the many probe sets for Locus 2 may be identical or unique.

In this embodiment, probes 1802 and 1808 hybridize to sequences corresponding to Locus 1 and Locus 2 respectively. The designs of each probe for Locus 1 and Locus 2 are such that the first adjacent nucleotide next to the hybridization domains contains a different nucleotide for Locus 1 than for Locus 2. In this example, the first adjacent nucleotide next to the hybridization domain of 1802 is an “A,” whereas the first adjacent nucleotide next to the hybridization domain of 1808 is a “T.” In this embodiment, all probes for Locus 1 shall be designed such that the first nucleotide immediately adjacent to the hybridization domain shall consist of different nucleotide(s) than the first nucleotide immediately adjacent to the hybridization domain of the probes for Locus 2. That is, by design, probe sets from Locus 1 and Locus 2 may be distinguished from one another based on the identity of the first nucleotide immediately adjacent to the hybridization domain.

In this embodiment, a DNA polymerase or other enzyme will be used to add at least one additional nucleotide to each of the probe sequences. In this example, the nucleotide substrates for the DNA polymerase are competent for a single addition, perhaps because the nucleotides added to the reaction mixture are dideoxy nucleotides. That is, only one new nucleotide shall be added to each probe sequence. In this example, the nucleotide added to probe 1802 will contain one or more labels (1803) of type “A.” The nucleotide added to probe 1808 will contain one or more labels (1809) of type “B,” such that the probe products for Locus 1 may be distinguished from the probe products from Locus 2.

In this embodiment, the probes 1802 and 1808 contain one or more labels (1801, 1806) of type “C.” Therefore, probe products will contain a combination of labels. For Locus 1, probe products will contains labels of type “A” and type “C,” whereas probe products from Locus 2 will contain labels of type “B” and type “C.”

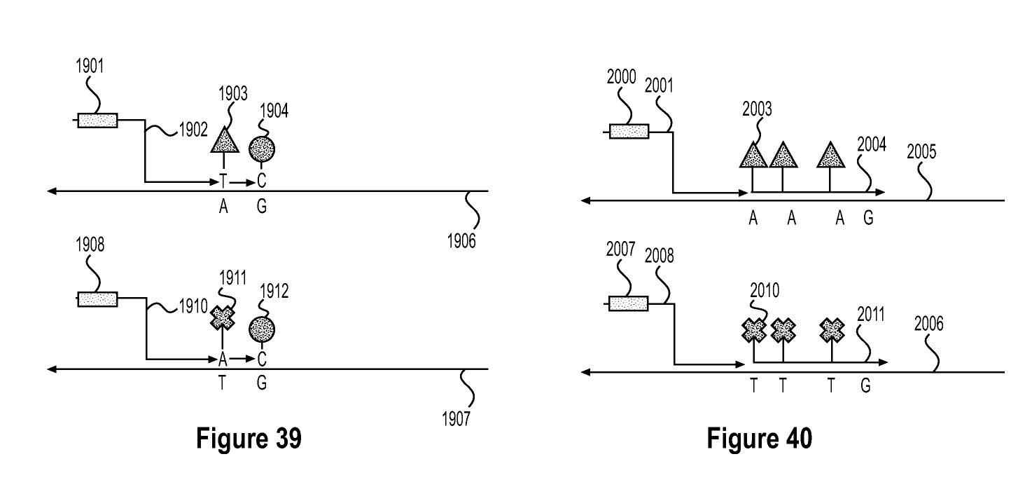

FIG. 39 depicts a modification of the general procedure described in FIG. 21. FIG. 39 depicts two probe sets, one probe set for Locus 1 and one probe set for Locus 2, although as aforementioned, multiple probes sets may be designed for each genomic locus. 1906 and 1907 are target molecules corresponding to Locus 1 and Locus 2, respectively.

A first probe set contains member probe 1902. 1902 contains an affinity tag (1901) which may be used for isolation and identification of the probe product.

A second probe set with member probe 1910 carries respective features as in the first probe set. 1910 contains an affinity tag (1908) which may be identical to or unique from 1901. Many probe sets may be designed that target “Locus 1,” containing unique probe sequences. Similarly, many probe sets may be designed that target “Locus 2,” containing unique probe sequences. In this embodiment, the affinity tags for the many probe sets for Locus 1 may be identical or unique, and the affinity tags for the many probe sets for Locus 2 may be identical or unique.

In this embodiment, probes 1902 and 1910 hybridize to sequences corresponding to Locus 1 and Locus 2 respectively. The designs of each probe for Locus 1 and Locus 2 are such that the first adjacent nucleotide next to the hybridization domains contains a different nucleotide for Locus 1 than Locus 2. In this example, the first adjacent nucleotide next to the hybridization domain of 1902 is an “A,” whereas the first adjacent nucleotide next to the hybridization domain of 1910 is a “T.” In this embodiment, all probes for Locus 1 shall be designed such that the first nucleotide immediately adjacent to the hybridization domain shall consist of different nucleotide(s) than the first nucleotide immediately adjacent to the hybridization domain of the probes for Locus 2. That is, by design, probe sets from Locus 1 and Locus 2 may be distinguished from one another nucleotide on the identity of the first nucleotide immediately adjacent to the hybridization domain. A different nucleotide, not one used to distinguish probes from Locus 1 or Locus 2 shall serve as a chain terminator. In this particular example, an “A” nucleotide on a target molecule is used do distinguish probes for Locus 1 and a “T” nucleotide is used to distinguish probes for Locus 2. In this example, a “C” nucleotide may serve as a chain terminator. In this case, a “C” nucleotide will be added to the assay not is not capable of chain elongation (for example, a dideoxy C). One additional constraint is that the probe sequences are designed such that there are no instances of an identifying nucleotide for Locus 2 present on 1906 in between the distinguishing nucleotide for Locus 1 and the chain terminating nucleotide. In this example, there will be no “T” nucleotides present on 1906 after the hybridization domain of 1902 and before the G, which will pair with the chain terminator C.

In this embodiment, DNA polymerase or a similar enzyme will be used to synthesize new nucleotide sequences, and the nucleotide added at the distinguishing nucleotide location for Locus 1 will contain one or more labels (1903) of type “A.” The nucleotide added at the distinguishing nucleotide location for Locus 2 will contain 1 or more labels (1911) of type “B,” such that the probe products for Locus 1 may be distinguished from the probe products from Locus 2. In this embodiment, the nucleotide added at the chain terminating position will contain one or more labels (1912) of type “C.” Therefore, probe products will contain a combination of labels. For Locus 1, probe products will contains labels of type “A” and type “C,” whereas probe products from Locus 2 will contain labels of type “B” and type “C.”

In another embodiment, the chain terminator may contain no label. In this embodiment, a fourth nucleotide may be added to the assay that contains one or more labels of type “C.” This fourth nucleotide does not pair with the identifying nucleotide for Allele 1 (in this example, A), does not pair with the identifying nucleotide for Allele 2 (in this example, T), does not pair with the chain terminating nucleotide (in this example G). In this example, the fourth nucleotide that would bear one or more labels of type “C” is G, and will pair with C locations on 1906 and 1907. Therefore, probe products will contain a combination of labels. For Locus 1, probe products will contains labels of type “A” and type “C,” whereas probe products from Locus 2 will contain labels of type “B” and type “C.”

FIG. 40 depicts a modification of the general procedure described in FIG. 21. FIG. 40 depicts two probe sets, one probe set for Locus 1 and one probe set for Locus 2, although as aforementioned, multiple probes sets may be designed for each genomic locus. 2005 and 2006 are target molecules corresponding to Locus 1 and Locus 2, respectively.

A first probe sets contains member probe 2001. 2001 contains an affinity tag (2000) which may be used for isolation and identification of the probe product.

A second probe set with member probe 2008 carries respective features as in the first probe set. 2008 contains an affinity tag (2007) which may be identical to or unique from 2000. Many probe sets may be designed that target “Locus 1,” containing unique probe sequences. Similarly, many probe sets may be designed that target “Locus 2,” containing unique probe sequences. In this embodiment, the affinity tags for the many probe sets for Locus 1 may be identical or unique, and the affinity tags for the many probe sets for Locus 2 may be identical or unique.

In this embodiment, probes 2001 and 2008 hybridize to sequences corresponding to Locus 1 and Locus 2 respectively. The designs of each probe for Locus 1 and Locus 2 are such that there are one or more instances of a distinguishing nucleotide (in this example, “A” is a distinguishing nucleotide for Locus 1 and “T” is a distinguishing nucleotide for Locus 2) followed by a chain terminating nucleotide (in this example “G”) adjacent to the hybridization domain of the probes. Importantly there will be no instances of the distinguishing nucleotide for Locus 2 (in this example, “T”) present in between the hybridization domain of 2001 on 2005 and the chain terminating nucleotide on 2005. Similarly, there will be no instance of the distinguishing nucleotide for Locus 1 (in this example, “A”) present in between the hybridization domain of 2008 on 2006 and the chain terminating nucleotide on 2006.

In this embodiment, DNA polymerase or a similar enzyme will be used to synthesize new nucleotide sequences (2004, 2011) until the addition of a chain terminating nucleotide, one possible example would be a dideoxy C. In this embodiment, the nucleotides added at the distinguishing nucleotide locations for Locus 1 will contain one or more labels (2003) of type “A.” The nucleotides added at the distinguishing nucleotide locations for Locus 2 will contain 1 or more labels (2010) of type “B,” such that the probe products for Locus 1 may be clearly distinguished from the probe products from Locus 2.

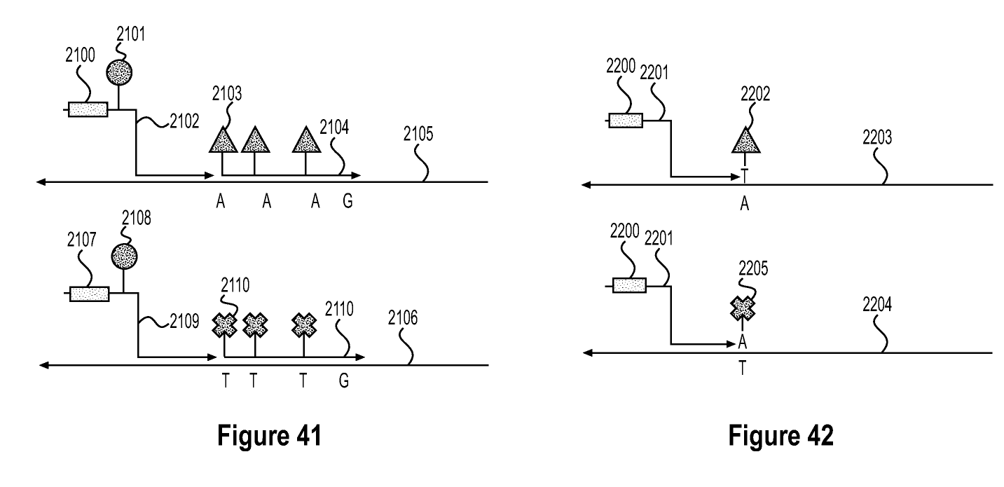

FIG. 41 depicts a modification of the general procedure described in FIG. 21. FIG. 41 depicts two probe sets, one probe set for Locus 1 and one probe set for Locus 2, although as aforementioned, multiple probes sets may be designed for each genomic locus. 2105 and 2106 are target molecules corresponding to Locus 1 and Locus 2, respectively.

A first probe sets contains member probe 2102. 2102 contains an affinity tag (2100) which may be used for isolation and identification of the probe product.

A second probe set with member probe 2109 carries respective features as in the first probe set. 2109 contains an affinity tag (2107) which may be identical to or unique from 2100. Many probe sets may be designed that target “Locus 1,” containing unique probe sequences. Similarly, many probe sets may be designed that target “Locus 2,” containing unique probe sequences. In this embodiment, the affinity tags for the many probe sets for Locus 1 may be identical or unique, and the affinity tags for the many probe sets for Locus 2 may be identical or unique.

In this embodiment, probes 2102 and 2109 hybridize to sequences corresponding to Locus 1 and Locus 2 respectively. The designs of each probe for Locus 1 and Locus 2 are such that there are one or more instances of a distinguishing nucleotide (in this example, “A” is a distinguishing nucleotide for Locus 1 and “T” is a distinguishing nucleotide for Locus 2) followed by a chain terminating nucleotide (in this example “G”) adjacent to the hybridization domain of the probes. Importantly there will be no instances of the distinguishing nucleotide for Locus 2 (in this example, “T”) present in between the hybridization domain of 2102 on 2105 and the chain terminating nucleotide on 2105. Similarly, there will be no instance of the distinguishing nucleotide for Locus 1 (in this example, “A”) present in between the hybridization domain of 2109 on 2106 and the chain terminating nucleotide on 2106.

In this embodiment, DNA polymerase or a similar enzyme will be used to synthesize new nucleotide sequences (2104, 2110) until the addition of a chain terminating nucleotide, one possible example would be a dideoxy C. In this embodiment, the nucleotides added at the distinguishing nucleotide locations for Locus 1 will contain one or more labels (2103) of type “A.” The nucleotides added at the distinguishing nucleotide locations for Locus 2 will contain 1 or more labels (2110) of type “B,” such that the probe products for Locus 1 may be clearly distinguished from the probe products from Locus 2.

In this embodiment, the probes 2102 and 2109 contain one or more labels (2101, 2108) of type “C.” Therefore, probe products will contain a combination of labels. For Locus 1, probe products will contains labels of type “A” and type “C,” whereas probe products from Locus 2 will contain labels of type “B” and type “C.”

FIG. 42 depicts a modification of the general procedure described in FIG. 21. FIG. 42 depicts two probe sets for identifying various alleles of the same genomic locus. For example, for distinguishing maternal and fetal alleles, in the case of cell free DNA isolated from a pregnant woman, or for distinguishing host and donor alleles, in the case of cell free DNA from a recipient of an organ transplant. FIG. 42 depicts two probe sets—one probe set for Allele 1 and one probe set for Allele 2. 2203 and 2204 are target molecules corresponding to Allele 1 and Allele 2, respectively.

A first probe sets contains member probe 2201. 2201 contains an affinity tag (2200) which may be used for isolation and identification of the probe product. In this embodiment, the probe sets used for identification of the two different alleles are the same. That is, the probe set for Allele 2 consists of member probe 2201. In this embodiment, probe 2201 hybridizes to a sequence corresponding to Allele 1 and Allele 2 respectively in FIG. 42. The design of probe 2201 is such that the first adjacent nucleotide next to the hybridization domain contains a different nucleotide for Allele 1 than Allele 2. In other words, the first nucleotide adjacent to the hybridization domain may be a single nucleotide polymorphism, or SNP. In this example, the first adjacent nucleotide on 2203 next to the hybridization domain of 2201 is an “A,” whereas the first adjacent nucleotide on 2204 next to the hybridization domain of 2201 is a “T.” That is, probe products from Allele 1 and Allele 2 may be distinguished from one another based on the identity of the first nucleotide immediately adjacent to the hybridization domain.

In this embodiment, a DNA polymerase or other enzyme will be used to add at least one additional nucleotide to each of the probe sequences. In this example, the nucleotide substrates for the DNA polymerase are competent for a single addition, perhaps because the nucleotides added to the reaction mixture are dideoxy nucleotides. That is, only one new nucleotide shall be added to each probe sequence. In this example, the nucleotide added to probe 2201 for Allele 1 will contain one or more labels (2202) of type “A.” The nucleotide added to probe 2201 for Allele 2 will contain one or more labels (2205) of type “B,” such that the probe products for Allele 1 may be clearly distinguished from the probe products from Allele 2. That is, the probe product for Allele 1 consists of probe 2201 plus one additional nucleotide bearing one or more labels of type “A,” and the probe products for Allele 2 consists of probe 2201 plus one additional nucleotide bearing one or more labels of type “B.”

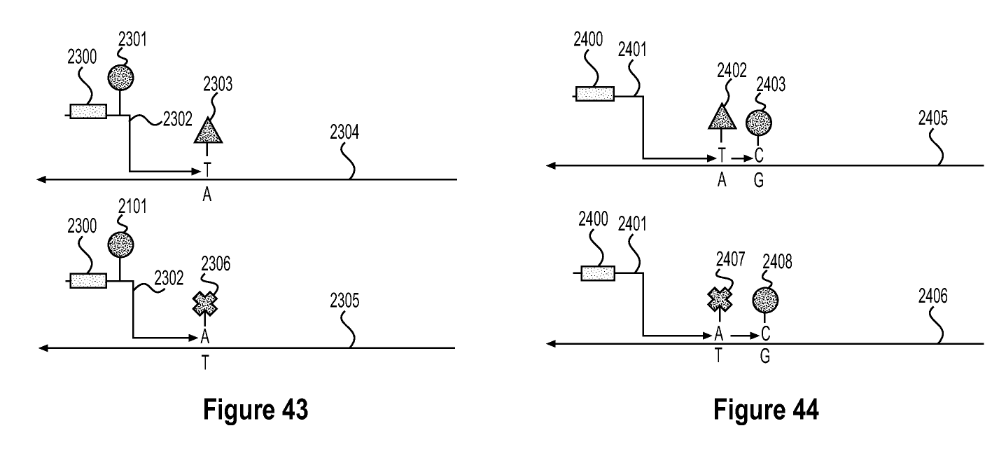

FIG. 43 depicts a modification of the general procedure described in FIG. 21. FIG. 43 depicts two probe sets for identifying various alleles of the same genomic locus. For example, for distinguishing maternal and fetal alleles, in the case of cell free DNA isolated from a pregnant woman, or for distinguishing host and donor alleles, in the case of cell free DNA from a recipient of an organ transplant. FIG. 43 depicts two probe sets—one probe set for Allele 1 and one probe set for Allele 2. 2304 and 2305 are target molecules corresponding to Allele 1 and Allele 2, respectively.

A first probe sets contains member probe 2302. 2302 contains an affinity tag (2300) which may be used for isolation and identification of the probe product. In this embodiment, the probe sets used for identification of the two different alleles are the same. That is, the probe set for Allele 2 consists of member probe 2302. In this embodiment, probe 2302 hybridizes to a sequence corresponding to Allele 1 and Allele 2 respectively in FIG. 43. The design of probe 2302 is such that the first adjacent nucleotide next to the hybridization domains contains a different nucleotide for Allele 1 than Allele 2. In other words, the first nucleotide adjacent to the hybridization domain may be a single nucleotide polymorphism, or SNP. In this example, the first adjacent nucleotide on 2304 next to the hybridization domain of 2302 is an “A,” whereas the first adjacent nucleotide on 2305 next to the hybridization domain of 2302 is a “T.” That is, probe products from Allele 1 and Allele 2 may be distinguished from one another based on the identity of the first nucleotide immediately adjacent to the hybridization domain.

In this embodiment, a DNA polymerase or other enzyme will be used to add at least one additional nucleotide to each of the probe sequences. In this example, the nucleotide substrates for the DNA polymerase are competent for a single addition, perhaps because the nucleotides added to the reaction mixture are dideoxy nucleotides. That is, only one new nucleotide shall be added to each probe sequence. In this example, the nucleotide added to probe 2302 for Allele 1 will contain one or more labels (2303) of type “A.” The nucleotide added to probe 2302 for Allele 2 will contain one or more labels (2306) of type “B,” such that the probe products for Allele 1 may be clearly distinguished from the probe products from Allele 2. That is, the probe product for Allele 1 consists of probe 2302 plus one additional nucleotide bearing one or more labels of type “A,” and the probe products for Allele 2 consists of probe 2302 plus one additional nucleotide bearing one or more labels of type “B.”

In this embodiment, the probes 2302 contain one or more labels (2301) of type “C.” Therefore, probe products will contain a combination of labels. For Allele 1, probe products will contains labels of type “A” and type “C,” whereas probe products from Allele 2 will contain labels of type “B” and type “C.”

FIG. 44 depicts a modification of the general procedure described in FIG. 21. FIG. 44 depicts two probe sets for identifying various alleles of the same genomic locus. For example, for distinguishing maternal and fetal alleles, in the case of cell free DNA isolated from a pregnant woman, or for distinguishing host and donor alleles, in the case of cell free DNA from a recipient of an organ transplant. FIG. 44 depicts two probe sets—one probe set for Allele 1 and one probe set for Allele 2. 2405 and 2406 are target molecules corresponding to Allele 1 and Allele 2, respectively.

A first probe sets contains member probe 2401. 2401 contains an affinity tag (2400) which may be used for isolation and identification of the probe product. In this embodiment, the probe sets used for identification of two different alleles are the same. That is, the probe set for Allele 2 consists of member probe 2401. In this embodiment, probe 2401 hybridizes to a sequence corresponding to Allele 1 and Allele 2 respectively in FIG. 44. The design of probe for 2401 is such that the first adjacent nucleotide next to the hybridization domains contains a different nucleotide for Allele 1 than Allele 2. In other words, the first nucleotide adjacent to the hybridization domain may be a single nucleotide polymorphism, or SNP. In this example, the first adjacent nucleotide on 2405 next to the hybridization domain of 2401 is an “A,” whereas the first adjacent nucleotide on 2406 next to the hybridization domain of 2401 is a “T.” That is, probe products from Allele 1 and Allele 2 may be distinguished from one another based on the identity of the first nucleotide immediately adjacent to the hybridization domain.

In this embodiment, a DNA polymerase or other enzyme will be used to add at least one additional nucleotide to each of the probe sequences. In this example, the nucleotide added to probe 2401 for Allele 1 will contain one or more labels (2402) of type “A.” The nucleotide added to probe 2401 for Allele 2 will contain one or more labels (2407) of type “B,” such that the probe products for Locus 1 may be clearly distinguished from the probe products from Locus 2. That is, the probe product for Allele 1 contains probe 2401 plus an additional nucleotide bearing one or more labels of type “A,” and the probe product for Allele 2 contains probe 2401 plus an additional nucleotide bearing one or more labels of type “B.” A different nucleotide, not one used to distinguish Allele 1 from Allele 2 shall serve as a chain terminator. In this particular example, an “A” nucleotide on a target molecule is used to identify Allele 1 and a “T” nucleotide is used to identify Allele 2. In this example, a “C” nucleotide may serve as a chain terminator. In this case, a “C” nucleotide will be added to the assay that is not is not capable of chain elongation (for example, a dideoxy C). One additional constraint is that the probe sequences are designed such that there are no instances of an identifying nucleotide for Allele 2 is present on 2405 in between the distinguishing nucleotide for Allele 1 and the chain terminating nucleotide. In this example, there will be no “T” nucleotides present on 2405 after the hybridization domain of 2401 and before a G, which will pair with the chain terminator C.

In this embodiment, DNA polymerase or a similar enzyme will be used to synthesize new nucleotide sequences, and the nucleotide added at the distinguishing nucleotide location for Allele 1 will contain one or more labels (2402) of type “A.” The nucleotide added at the distinguishing nucleotide location for Allele 2 will contain 1 or more labels (2407) of type “B,” such that the probe products for Allele 1 may be clearly distinguished from the probe products from Allele 2. In this embodiment, the nucleotide added at the chain terminating position will contain one or more labels (2403) of type “C.” Therefore, probe products will contain a combination of labels. For Allele 1, probe products will contains labels of type “A” and type “C,” whereas probe products from Allele 2 will contain labels of type “B” and type “C.”

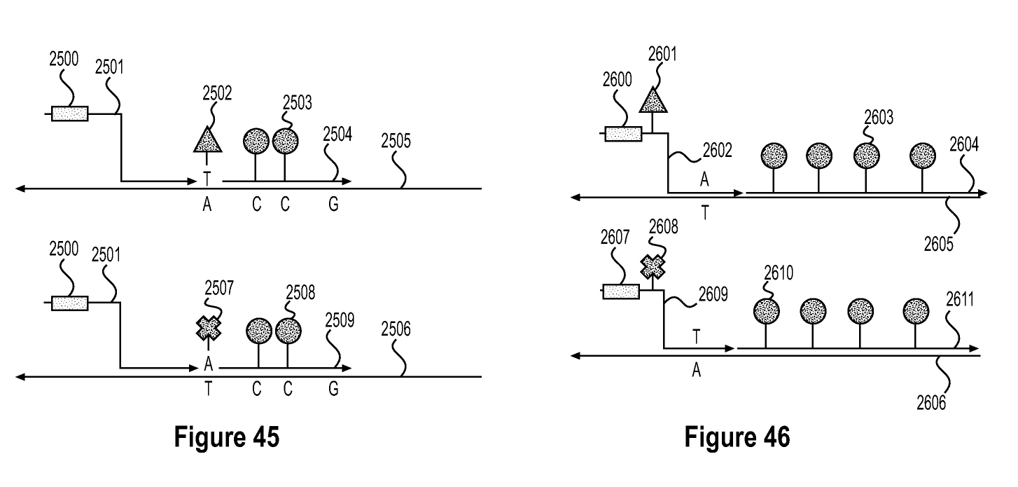

FIG. 45 depicts a modification of the general procedure described in FIG. 21. FIG. 45 depicts two probe sets for identifying various alleles of the same genomic locus. For example, for distinguishing maternal and fetal alleles, in the case of cell free DNA isolated from a pregnant woman, or for distinguishing host and donor alleles, in the case of cell free DNA from a recipient of an organ transplant. FIG. 45 depicts two probe sets—one probe set for Allele 1 and one probe set for Allele 2. 2505 and 2506 are target molecules corresponding to Allele 1 and Allele 2, respectively.

A first probe sets contains member probe 2501. 2501 contains an affinity tag (2500) which may be used for isolation and identification of the probe product. In this embodiment, the probe sets used for identification of two different alleles are the same. That is, the probe set for Allele 2 consists of member probe 2501. In this embodiment, probe 2501 hybridizes to a sequence corresponding to Allele 1 and Allele 2 respectively in FIG. 45. The design of probe for 2501 is such that the first adjacent nucleotide next to the hybridization domains contains a different nucleotide for Allele 1 than Allele 2. In other words, the first nucleotide adjacent to the hybridization domain may be a single nucleotide polymorphism, or SNP. In this example, the first adjacent nucleotide on 2505 next to the hybridization domain of 2501 is an “A,” whereas the first adjacent nucleotide on 2506 next to the hybridization domain of 2501 is a “T.” That is, probe products from Allele 1 and Allele 2 may be distinguished from one another based on the identity of the first base immediately adjacent to the hybridization domain.

In this embodiment, a DNA polymerase or other enzyme will be used to add at least one additional nucleotide to each of the probe sequences. In this example, the nucleotide added to probe 2501 for Allele 1 will contain one or more labels (2502) of type “A.” The nucleotide added to probe 2501 for Allele 2 will contain one or more labels (2507) of type “B,” such that the probe products for Locus 1 may be clearly distinguished from the probe products from Locus 2. That is, the probe product for Allele 1 contains probe 2501 plus an additional nucleotide bearing one or more labels of type “A,” and the probe product for Allele 2 contains probe 2501 plus an additional nucleotide bearing one or more labels of type “B.” A different nucleotide, not one used to distinguish Allele 1 from Allele 2 shall serve as a chain terminator. In this particular example, an “A” nucleotide on a target molecule is used to identify Allele 1 and a “T” nucleotide is used to identify Allele 2. In this example, a “C” nucleotide may serve as a chain terminator. In this case, a “C” nucleotide will be added to the assay that is not is not capable of chain elongation (for example, a dideoxy C). One additional constraint is that the probe sequences are designed such that no instances of an identifying nucleotide for Allele 2 are present on 2505 in between the distinguishing nucleotide for Allele 1 and the chain terminating nucleotide. In this example, there will be no “T” nucleotides present on 2505 after the hybridization domain of 2501 and before a G, which will pair with the chain terminator C.

In this embodiment, DNA polymerase or a similar enzyme will be used to synthesize new nucleotide sequences, and the nucleotide added at the distinguishing nucleotide location for Allele 1 will contain one or more labels (2502) of type “A.” The nucleotide added at the distinguishing nucleotide location for Allele 2 will contain 1 or more labels (2507) of type “B,” such that the probe products for Allele 1 may be clearly distinguished from the probe products from Allele 2. In this embodiment, a fourth nucleotide may be added to the assay that contains one or more labels (2508, 2503) of type “C.” This fourth nucleotide does not pair with the identifying nucleotide for Allele 1 (in this example, A), does not pair with the identifying nucleotide for Allele 2 (in this example, T), does not pair with the chain terminating nucleotide (in this example G). In this example, the fourth nucleotide that would bear one or more labels of type “C” is G, and will pair with C locations on 2505 and 2506. Therefore, probe products will contain a combination of labels. For Allele 1, probe products will contains labels of type “A” and type “C,” whereas probe products from Allele 2 will contain labels of type “B” and type “C.”

FIG. 46 depicts a modification of the general procedure described in FIG. 21. FIG. 46 depicts two probe sets for identifying various alleles of the same genomic locus. For example, for distinguishing maternal and fetal alleles, in the case of cell free DNA isolated from a pregnant woman, or for distinguishing host and donor alleles, in the case of cell free DNA from a recipient of an organ transplant. FIG. 46 depicts two probe sets—one probe set for Allele 1 and one probe set for Allele 2. 2605 and 2606 are target molecules corresponding to Allele 1 and Allele 2, respectively. A first probe set contains member probe 2602. 2602 contains a label (2601) of type “A.” 2602 contains an affinity tag (2600) which may be used for isolation and identification of the probe product.

A second probe set with member probe 2609 carries respective features as in the first probe set. However, 2609 contains a label (2608) of type “B,” distinguishable from type “A.” 2609 contains an affinity tag (2607) which may be identical to or unique from 2600.

In this embodiment, 2602 and 2609 contain sequences that are nearly identical, and differ by only one nucleotide in the sequence. Therefore, hybridization sequences of these two probes are complementary to Allele 1 (2605), or Allele 2 (2606). Further, the length of each hybridization domain on 2602 and 2609, as well as experimental hybridization conditions are designed such that probe 2602 will only hybridize to Allele 1 and probe 2609 will only hybridize to Allele 2. The purpose of this assay type is to be able to accurately quantify the frequency of Allele 1 and Allele 2 in a sample.

In this embodiment, DNA polymerase or other enzyme may be used to synthesize a new polynucleotide sequence, for example 2604 in the case of Allele 1 or 2611 in the case of Allele 2. In this embodiment, 2604 and 2611 may contain one or more labels (2603, 2610) of type “C,” possibly as a result of incorporation of a one of more nucleotides bearing a label of type “C.” Therefore, probe products will contain a combination of labels. For Allele 1, probe products will contains labels of type “A” and type “C,” whereas probe products from Allele 2 will contain labels of type “B” and type “C.” This embodiment results in probe products with high specificity for sequences in Allele 1 or Allele 2 respectively.

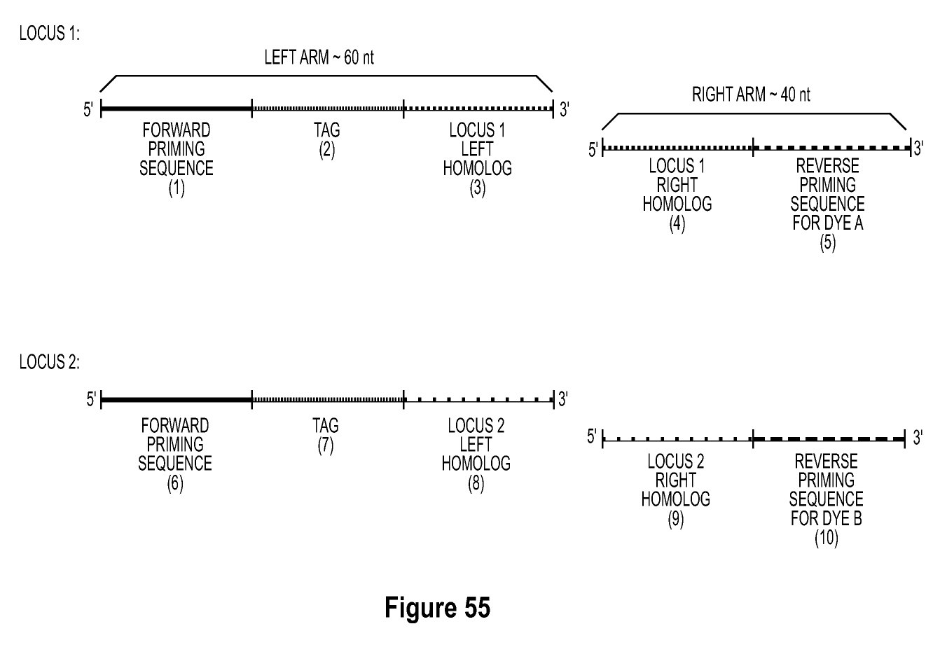

FIGS. 55-58 illustrate a modification of the general procedure described with respect to FIGS. 21-46. FIG. 55 depicts two probe sets; one probe set for Locus 1 and one probe set for Locus 2—although as aforementioned, multiple probes sets may be designed for each genomic locus. The left arm of the Locus 1 probe set consists of a forward priming sequence, an affinity tag sequence and a homolog to Locus 1 sequence. The right arm of the Locus 1 probe set consists of a homolog to Locus 1 sequence and a reverse priming sequence for labeling the Locus 1 probe set with label A. The left arm of the Locus 2 probe set consists of a forward priming sequence, an affinity tag sequence and a homolog to Locus 2 sequence. The right arm of the Locus 2 probe set consists of a homolog to Locus 2 sequence and a reverse priming sequence for labeling the Locus 2 probe set with label B. The forward priming sequence and the affinity tag sequence are identical for the probe sets for both Locus 1 and Locus 2. The homologous sequences are specific to a single genomic locus. Locus homologous sequences for each probe set are immediately adjacent to one another such that when they hybridize to their target loci, they immediately abut one another and thus may be ligated to form one continuous molecule. The reverse priming sequence is specific to the label (e.g., label A or label B) to be used in labeling probe products for a particular locus for a particular affinity tag sequence.

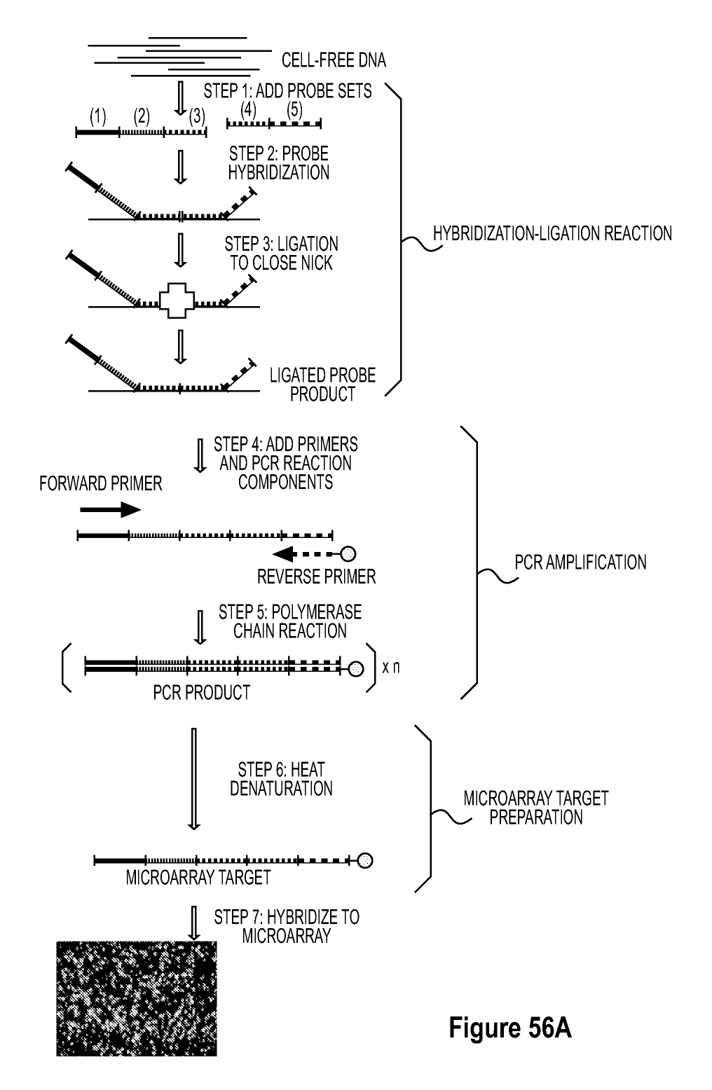

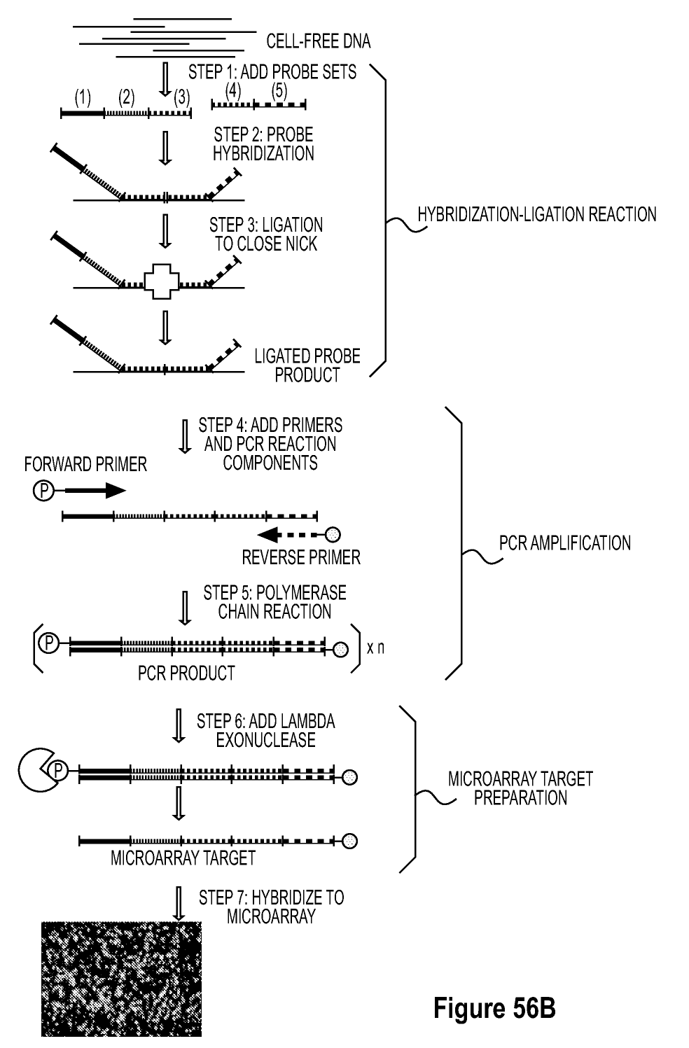

FIGS. 56A and 56B depict the procedural workflow that would be applied to the collection of probe sets, such as those probe sets illustrated in FIG. 55. This depiction is based on one probe set for one genomic locus (e.g., the probe set for Locus 1 shown in FIG. 55). In Step 1, the collection of probe sets is mixed with purified cell-free DNA. In Step 2, the locus specific sequences in each probe set hybridize to their corresponding homologous sequences in the cell-free DNA sample. In Step 3, a ligase enzyme is added to catalyze the formation of a phosphodiester bond between the 3′ base on the left arm homolog and the 5′ arm of the right homolog, closing the nick between the two arms and thus forming one continuous molecule which is the probe product.

As shown in FIG. 56A, primers and PCR reaction components (Taq polymerase, dNTPs, and reaction buffer) may be added to amplify the ligated probe product. A primer may be modified in that it contains the label that is specific to probe products for a particular locus for a particular affinity tag. In Step 5, the probe product is PCR amplified to yield a double-stranded PCR product in which one of the strands contains a 5′ label. In Step 6, heat denaturation can be used to separate the two strands of DNA, turning the double-stranded PCR product into a pair of single-stranded molecules. This allows the tag to be available for immobilization to the substrate. For example, if the tag is a nucleotide sequence, it is available to hybridize to the complementary sequence on the substrate in Step 7 of FIG. 56A.

As shown in FIG. 56B, in Step 4, modified primers and PCR reaction components (Taq polymerase, dNTPs, and reaction buffer) may be added to amplify the ligated probe product. The Forward Primer may be modified in that it has a 5′ phosphate group that makes it a preferred template for the Lambda exonuclease used in Step 6, and the Reverse Primer may be modified in that it contains the label that is specific to probe products for a particular locus for a particular affinity tag. In Step 5, the probe product is PCR amplified to yield a double-stranded PCR product in which the forward strand contains a 5′ phosphate group and the reverse strand contains a 5′ label. In Step 6, Lambda exonuclease is added to digest the forward strand in a 5′ to 3′ direction—the 5′ phosphate group on the forward strand makes it a preferred template for Lambda exonuclease digestion. The resulting material is single-stranded (reverse strand only) with a 5′ label. This represents the labeled target material for immobilization (e.g. by hybridization to a microarray, single molecule array or monolayer).

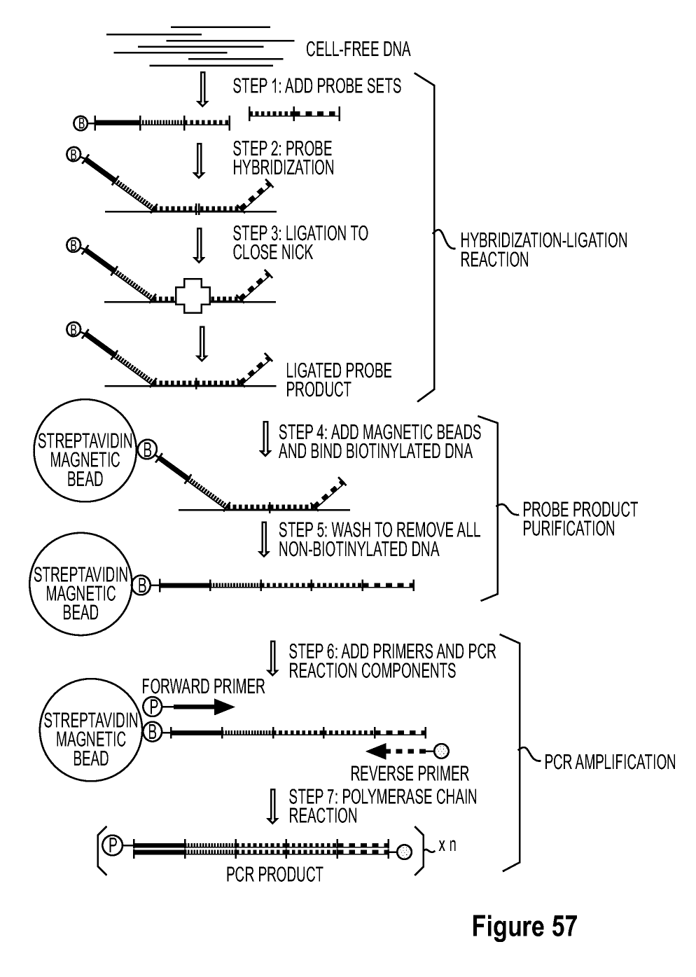

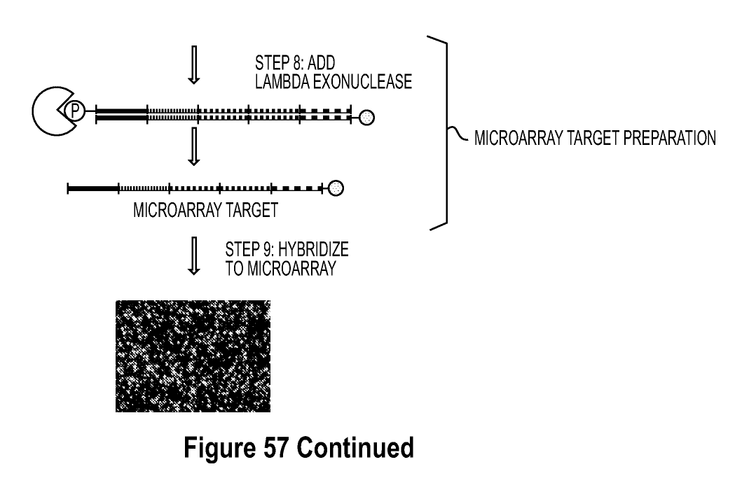

FIG. 57 depicts a modified version of the procedural workflow illustrated in FIG. 56. In this embodiment the left arm of each probe set contains a terminal biotin molecule as indicated by a “B” in Steps 1 to 6 of the Figure. This biotinylation enables the purification of the collection of probe products after completion of the hybridization-ligation reaction and prior to the PCR amplification. The workflow for this embodiment is identical to that described in FIG. 57 for Steps 1 to 3. In Step 4, streptavidin-coated magnetic beads are added to the hybridization-ligation reaction. The biotin molecule contained in the probe products will bind the products to the streptavidin. In Step 5, the magnetic beads are washed to remove the non-biotinylated DNA (cell-free genomic DNA and right arm oligonucleotides), resulting in a purified probe product. Steps 6 to 9 are performed in the same manner as described for Steps 4 to 7 in FIG. 56.

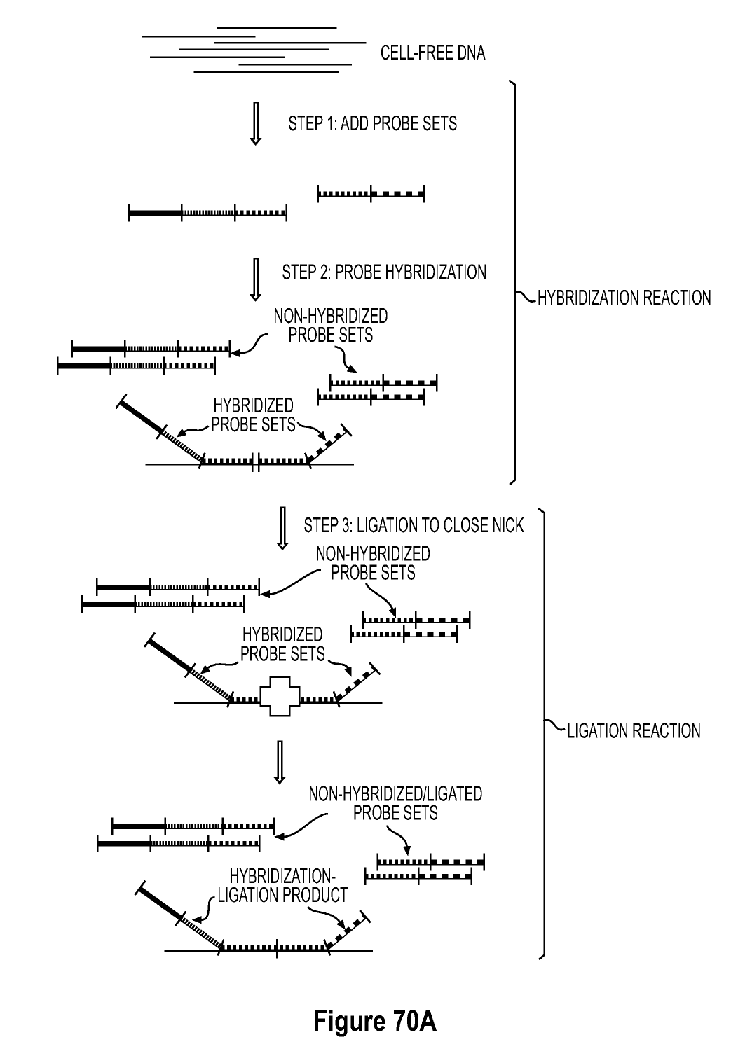

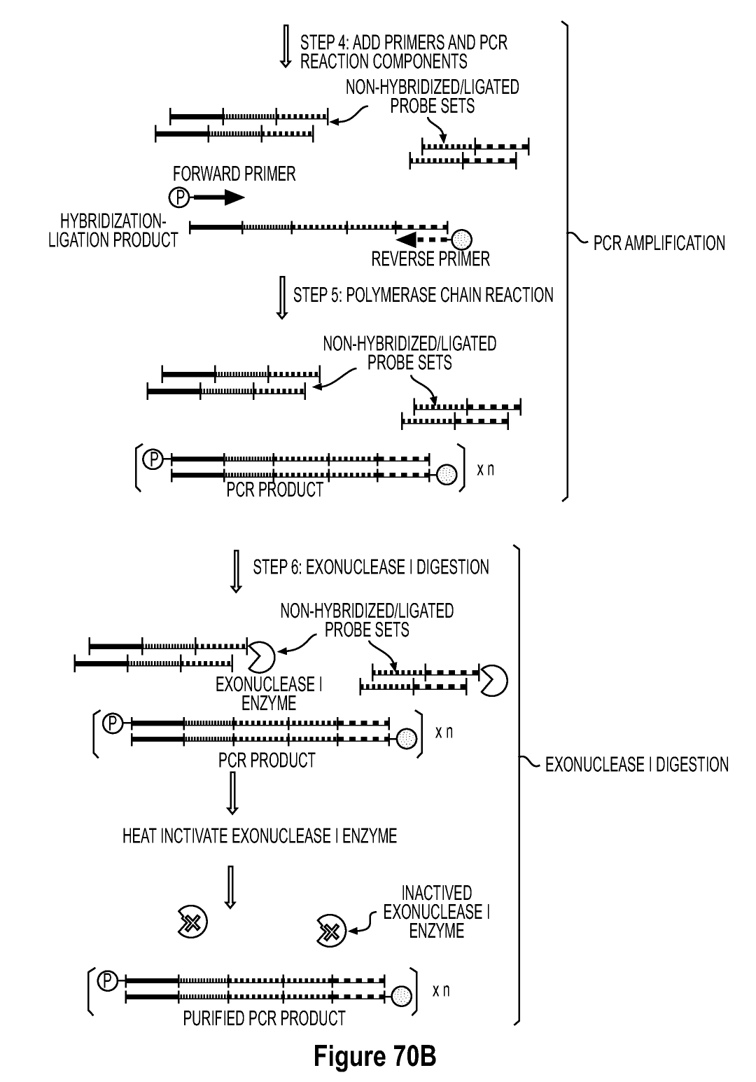

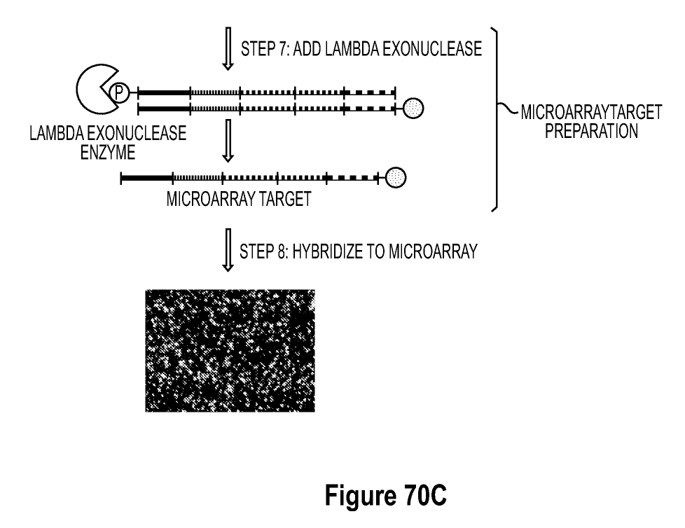

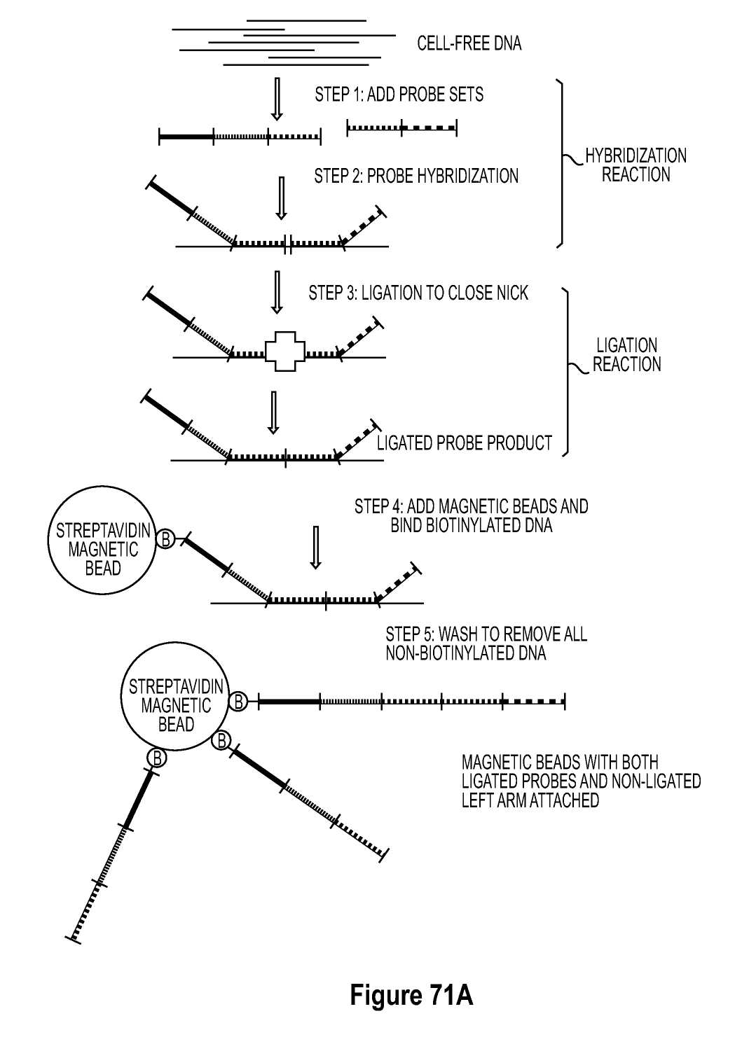

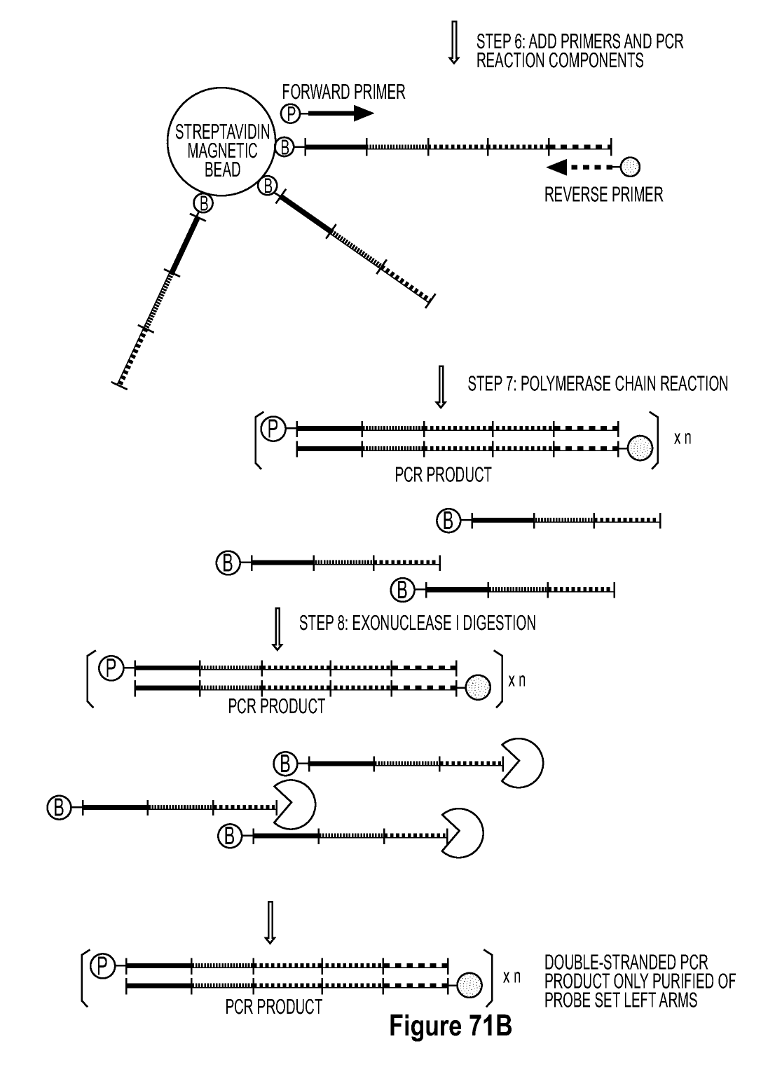

FIGS. 70A-70C and 71A-71B illustrate additional procedural workflow for the methods described herein. In some embodiments, as shown in FIGS. 70A-70C, Exonuclease I is used to digest residual non-ligated left arm probe molecules that may be present after completion of the PCR amplification. These molecules may contain sequences that are complementary to the tag sequences in the microarray target molecules and must be removed as they will otherwise compete with the microarray pull-down probes in hybridizing to the microarray targets. In some embodiments, the Exonuclease I digests residual single-stranded primers or other single-stranded material that may have not been extended to form double-stranded PCR products after completion of the PCR amplification. If not removed, the presence of dye-labeled primers during the process of microarray hybridization could result in an increased level of fluorescent background. In other embodiments, alternative exonuclease enzymes that are specific to single-stranded DNA templates could be used instead of, or in combination with Exonuclease I.

FIGS. 71A-71B depicts a magnetic bead after the purification step to remove all non-biotinylated molecules, and as shown in this figure, biotinylated probe set left arm molecules attached in addition to the attached ligated probe sets. A portion of these non-ligated left arm molecules may be released from the beads as a result of exposure to high temperatures during the PCR amplification process (FIG. 71A) so are present in the PCR reaction product. These non-ligated left arm molecules may be removed from the reaction by addition of an Exonuclease I digestion step (FIG. 71B) subsequent to the PCR amplification. This may eliminate the single-stranded non-ligated left arm molecules while the double-stranded PCR product molecules will be retained. Again, in other embodiments, alternative exonuclease enzymes that are specific to single-stranded DNA templates could be used instead of, or in combination with Exonuclease I

An alternative method to eliminate the single-stranded non-ligated left arm molecules from the PCR reaction product may be to use size exclusion gel filtration columns. This method would differentially eliminate the smaller (˜60 nucleotides) single-stranded while retaining the larger (˜100 base pairs) double stranded PCR product, based on the molecular weight difference between them.

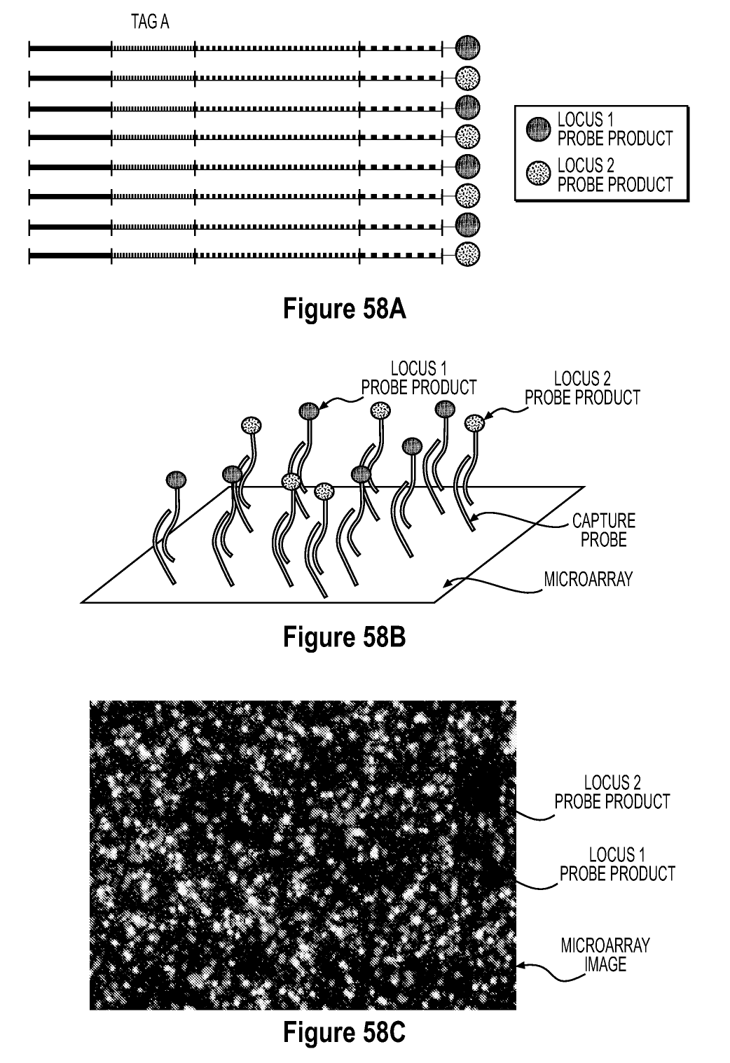

FIG. 58 provides an example of how probe products for Locus 1 and Locus 2 may be labeled with different label molecules. In FIG. 58A, Locus 1 probe products are labeled with label A (green) and Locus 2 probe products are labeled with label B (red) in one PCR amplification reaction. Probe products for both loci contain affinity tag sequence A. In FIG. 58B, the mixture of differentially labeled probe products is hybridized to a microarray location in which the capture probe sequence is complementary to the affinity tag A sequence. In FIG. 58C, the microarray location is imaged and the number of molecules of label A and label B counted to provide a relative measure of the levels of Locus 1 and Locus 2 present in the sample.

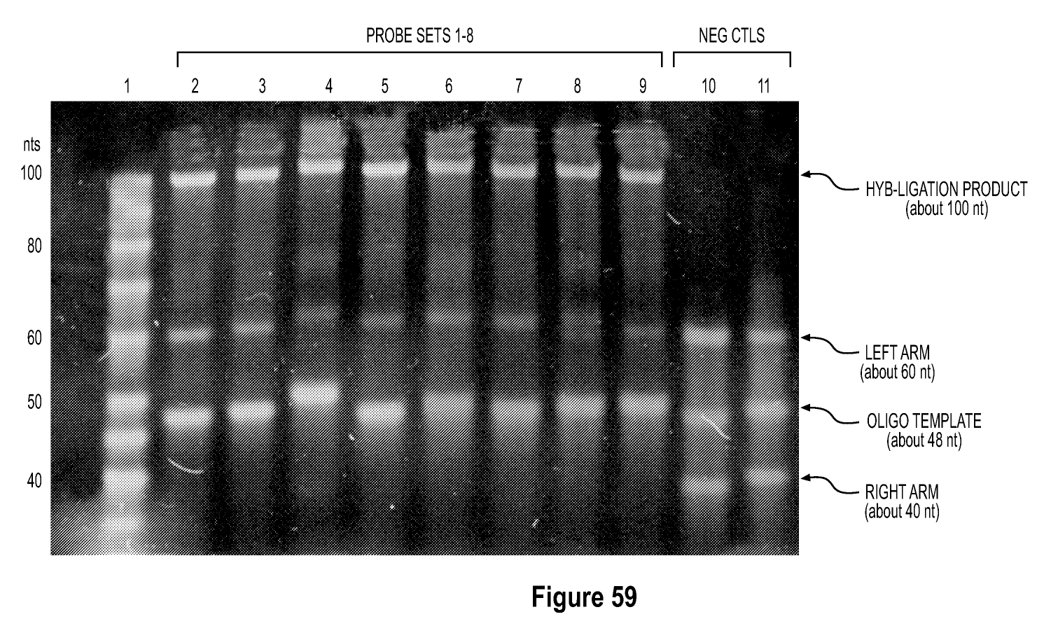

FIG. 59 provides evidence that probe products representing a multitude of genomic locations for one locus may be generated in a ligase enzyme specific manner using the hybridization-ligation process. Eight probe sets, each consisting of a left arm and right arm component as described in FIG. 55 and, containing homologs to eight chromosome 18 locations were hybridized to synthetic oligonucleotide templates (about 48 nucleotides) and ligated using a ligase enzyme to join the left and right arms for each. Reaction products were analyzed using denaturing polyacrylamide gel electrophoresis. Gel lane 1 contains a molecular weight ladder to indicate DNA band sizes. Lanes 2 to 9 contain hybridization-ligation reaction products for the eight chromosome 18 probe sets. A DNA band of about 100 nucleotides, representing the probe product of the about 60 nucleotide left arm and the about 40 nucleotide right arm, is present in each of lanes 2 to 9. Lanes 10 and 11 contain negative control reactions to which no ligase enzyme was added. No DNA band of about 100 nucleotides is present in lanes 10 and 11.

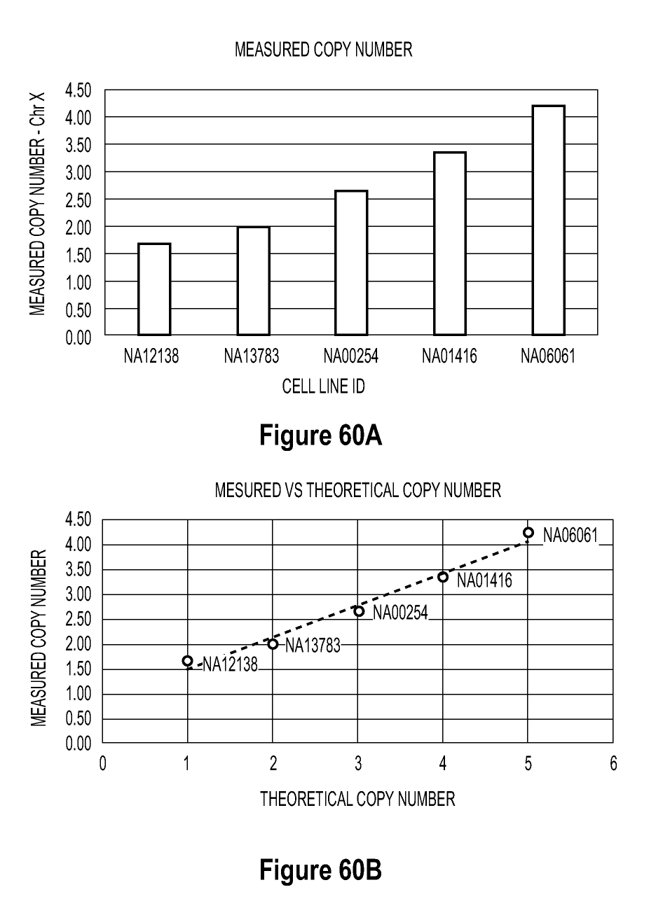

FIG. 60 provides data indicating that probe sets may be used to detect relative changes in copy number state. A mixture of eight probe sets containing homologs to eight distinct chromosome X locations was used to assay the cell lines containing different numbers of chromosome X indicated in Table 1.

TABLE 1
Cell lines containing different copy numbers of chromosome X

| Coriell Cell Line ID | Number of copies of chromosome X |
|---|---|
| NA12138 | 1 |
| NA13783 | 2 |
| NA00254 | 3 |
| NA01416 | 4 |
| NA06061 | 5 |

Quantitative PCR was used to determine the amount of probe product present for each cell line following the hybridization-ligation and purification processes described in FIG. 57 (Steps 1 to 5). As illustrated by FIG. 60A, the copy number state measured for the various cell lines followed the expected trend indicated in Table 1. For example, qPCR indicated a copy number state of less than two for NA12138, which has one copy of chromosome X. The measured copy number state for NA00254 (three copies of X) was greater than two, for NA01416 (four copies of X) was greater than three, and for NA06061 (five copies of X) was greater than four. The responsiveness of the process in detecting differences in copy number state is further illustrated by FIG. 60B in which the measured copy number state is plotted against the theoretical copy number state.

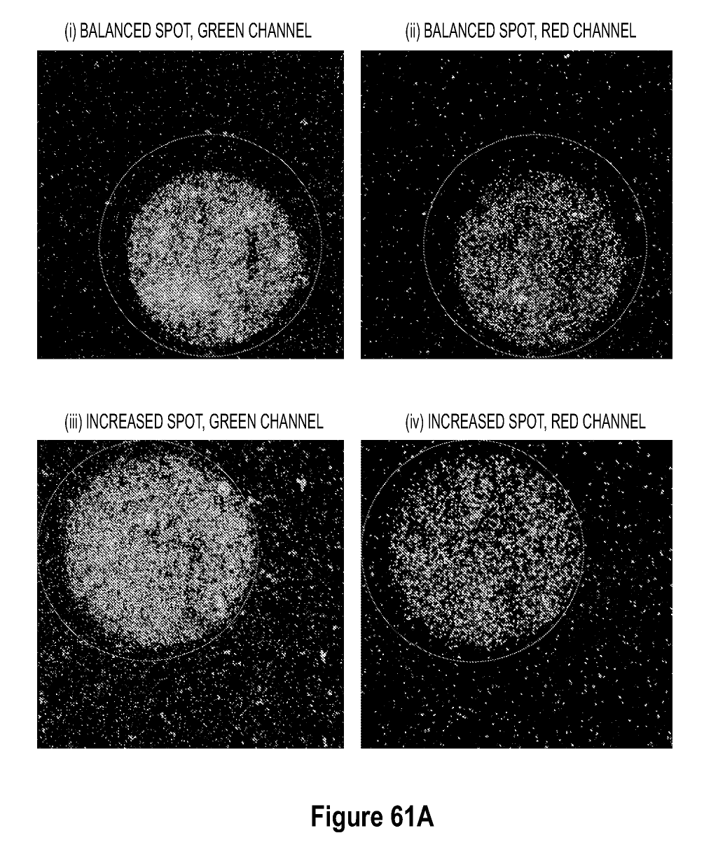

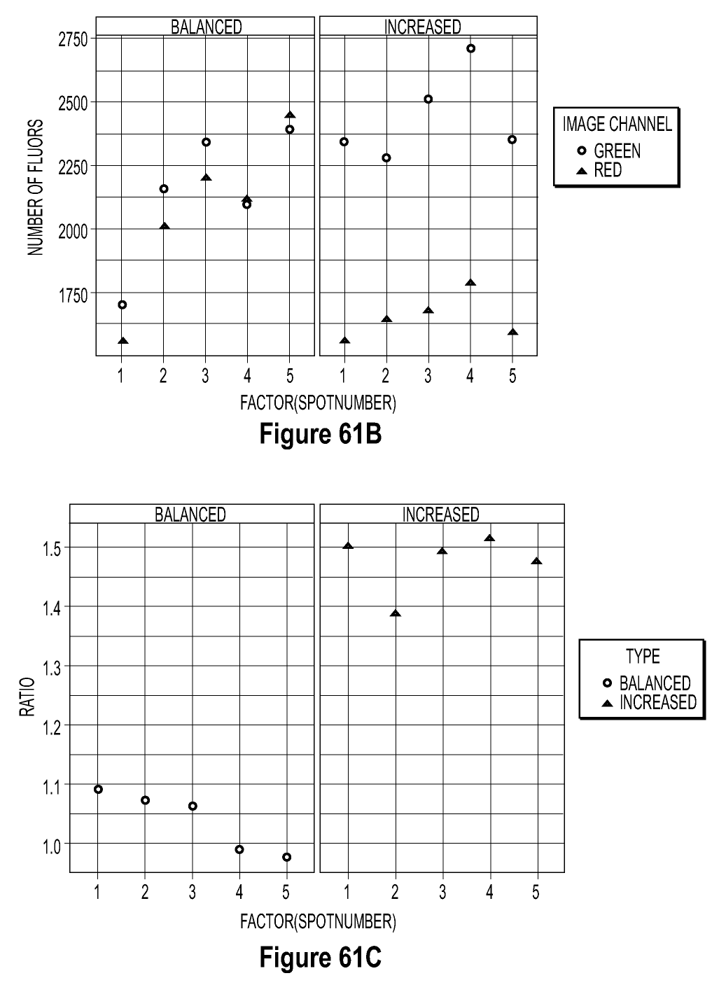

FIG. 61 provides evidence that mixtures of probe products may be used to generate quantitative microarray data as described in FIGS. 56 and 57.

FIG. 61A depicts representative fluorescence images of two array spots in two orthogonal imaging channels (Alexa 488: green, Alexa 594; red). A region of interest (ROI) is automatically selected (large circle), with any undesired bright contaminants being masked from the image (smaller outlined regions within the ROI). Single fluorophores on single hybridized assay products are visualized as small punctate features within the array spot. (i) A “Balanced” spot (representing genomic targets input at a 1:1 concentration ratio to the assay) imaged in the green channel and (ii) the same spot imaged in the red channel. (iii) An “Increased” spot (representing genomic targets input at a >1:1 concentration ratio to the assay) imaged in the green channel and (iv) the same spot imaged in the red channel.

FIG. 61B presents raw counts of the detected fluorophores in two channels for five spots each of the “Balanced” and “Increased” conditions. Despite some variation in the absolute number of fluors, the numbers in the two channels track closely for the “Balanced” case, but demonstrate clear separation in the “Increased” case.

FIG. 61C presents calculated ratio values for number of fluors in the green channel divided by the number of fluors in the red channel, for the five spots from each of the “Balanced” and “Increased” conditions. The “Balanced” case centers about a ratio of 1.0 and the “Increased” case is at an elevated ratio. Considering the “Balanced” case as comparing two balanced genomic loci and the “Increased” case as one where one locus is increased relative to the other, we may calculate the confidence of separation of the two conditions using an independent, 2-group T-test, yielding a p-value of 8×10⁻¹⁴.

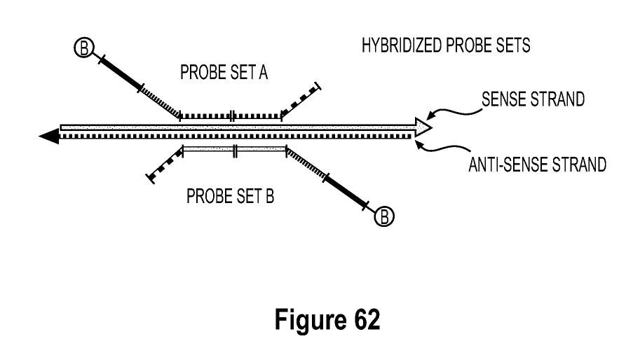

FIG. 62 illustrates a modification of the general procedure described in FIGS. 55 to 58. In this embodiment, a second probe set, Probe Set B is designed for each genomic location such that the genome homolog sequences in Probe Set B are a reverse complement of the genome homolog sequences in Probe Set A. Probe Set A will hybridize to the reverse strand of the genomic DNA and Probe Set B will hybridize to the forward strand of the genomic DNA. This embodiment will provide increased sensitivity relative to the embodiment described in FIGS. 55 to 58 as it will yield approximately double the number of probe products per locus.

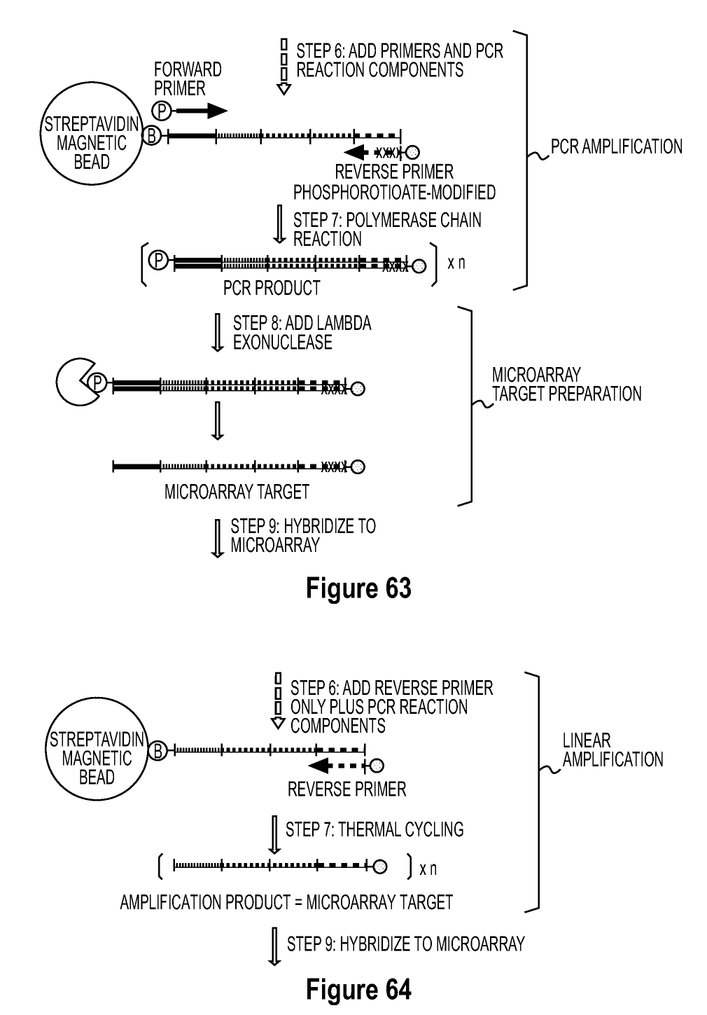

FIG. 63 illustrates a modification to the general procedure described in FIG. 57. In this embodiment, the Reverse Primer used in Step 6 is additionally modified in that the four bonds linking the first five nucleotides in the oligonucleotide sequence are phosphorothioate bonds. This modification will result in all PCR products generated during PCR amplification (Step 7) having a phosphorothioate modification on the 5′ end. This modification will protect the reverse strand from any digestion that might occur during the treatment with Lambda exonuclease in Step 8.

Although the 5′ phosphate group on the forward strand makes it a preferred template for Lambda exonuclease digestion, the reverse strand may still have some vulnerability to digestion. Phosphorothioate modification of the 5′ end of the reverse strand will reduce its vulnerability to Lambda exonuclease digestion.

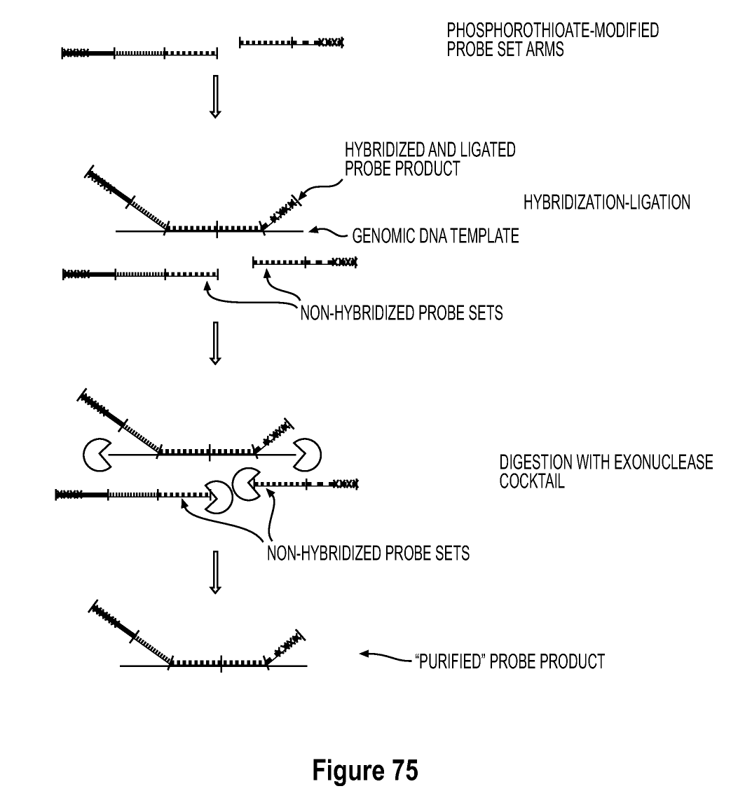

FIG. 75 illustrates another exemplary procedure for phosphorothioate modification of ligated probe sets as a means to differentially protect the ligated probes in an exonuclease-based purification process. In this example, the left and right arms of each probe set are modified by introduction of phosphorothioate bonds that will protect the ligated probe product from exonuclease digestion. As shown in FIG. 75, the left arm of the probe set would be modified by the substitution of the oxygen for sulphur in at least the four bonds linking the first five bases in the oligonucleotide sequence, and the right arm of the probe set would be modified by the substitution of the oxygen for sulphur in the four bonds linking the last five bases in the oligonucleotide sequence. Accordingly, the probe sets that successfully hybridize and ligate to form probe products are phosphorothioate-modified on both ends and thus would be prevented from being digested by exonuclease enzymes. Following the hybridization-ligation reaction, the product is subjected to exonuclease treatment by a cocktail of exonuclease enzymes that will digest both double and single-stranded DNA molecules and in both a 5′ to 3′ direction and a 3′ to 5′ direction. This will result in the digestion of genomic template DNA as well as any non-ligated probe set oligonucleotides, leaving only ligated probe sets in an intact state as shown in FIG. 75. This process constitutes a “purification” of the ligated probe products.

In this embodiment, the probe product is purified prior to PCR amplification as an alternative to magnetic bead purification. As such, neither biotinylation of the left arm of each probe set nor the use of streptavidin-coated magnetic beads is required to generate purified probe product material.

FIG. 64 illustrates a modification of the general procedure described in FIGS. 55 to 58. In this embodiment, PCR amplification of the probe product is replaced with linear amplification by adding the Reverse Primer but no Forward Primer to the amplification reaction in Step 6. If only the Reverse Primer is present the amplification product will be single stranded—the reverse strand with a label of the 5′ end. As the amplification product is already single-stranded, it does not require further processing before hybridization to a microarray, i.e., Lambda exonuclease digestion may be omitted. As a forward primer is not used in this embodiment, it is unnecessary for the left arm of the probe set to contain a forward priming sequence. The left arm would consist of an affinity tag sequence and a locus homolog sequence only as illustrated in FIG. 64.

A further embodiment of the general procedure described in FIGS. 55 to 58 is one in which the single ligation reaction process in Step 3 is replaced with a cycled ligation reaction process. This is accomplished by replacing the thermolabile ligase enzyme (e.g., T4 ligase) used to catalyze the ligation reaction with a thermostable ligase (e.g., Taq ligase). When a thermostable ligase is used, the hybridization-ligation reaction may be heated to a temperature that will melt all DNA duplexes (e.g., 95° C.) after the initial cycle of hybridization and ligation has occurred. This will make the genomic template DNA fully available for another probe set hybridization and ligation. Subsequent reduction of the temperature (e.g., to 45° C.) will enable this next hybridization and ligation event to occur. Each thermocycling of the hybridization and ligation reaction between a temperature that will melt DNA duplexes and one that will allow hybridization and ligation to occur will linearly increase the amount of probe product yielded from the reaction. If the reaction is exposed to 30 such cycles, up to 30 times the amount of probe product will be yielded than from a process in which a single ligation reaction is used.

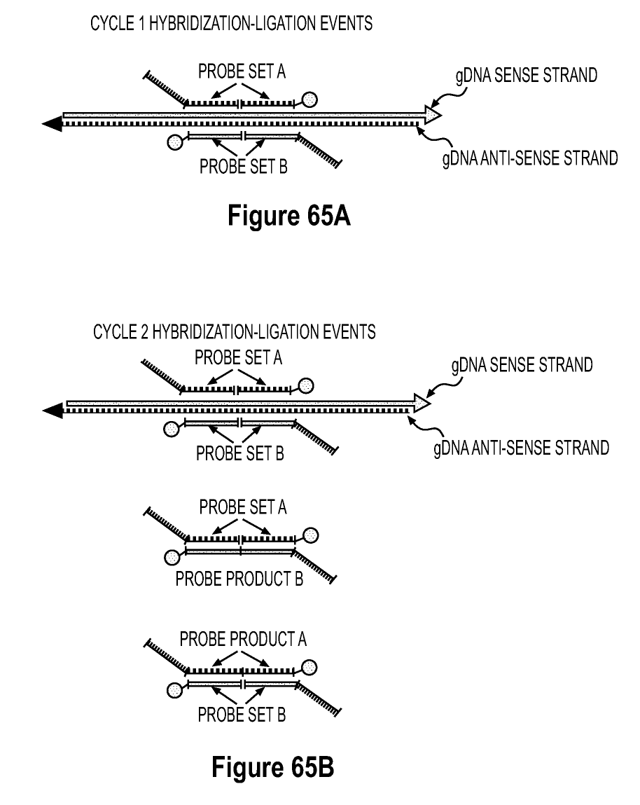

FIG. 65 depicts a further embodiment of the modified procedure described in FIG. 62. This embodiment takes advantage of the ligase chain reaction (LCR) in combining the presence of the reverse complement for each probe set with the use of a thermostable ligase to enable a cycled ligation reaction in which the product is exponentially amplified. FIG. 65 depicts two probe sets, Probe Set A and Probe Set B for one locus; where the genome homolog sequences in Probe Set B are the reverse complement of the genome homolog sequences in Probe Set A. The 5′ arm of each Probe Set consists of an affinity tag sequence and a homolog while the 3′ arm of each Probe Set consists of a homolog sequence with a label attached. In the first cycle of a thermocycled reaction, genomic DNA will be the only template available to enable hybridization and ligation to occur to generate a probe product as illustrated in FIG. 65A. However in the second cycle, Probe Product B generated in the first cycle will act as an additional template for Probe Set A and likewise Probe Product A generated in the first cycle will act as an additional template for Probe Set B as illustrated in FIG. 65B. In this same manner, the probe products from each successive cycle will act as template for probe set hybridization and ligation in the next cycle. This process would eliminate the need for PCR amplification of the probe product which may be directly used as microarray target.

Another embodiment of the procedure depicted in FIG. 65 is one which employs LCR but uses probe sets that have the structure described in FIG. 55, i.e., both left and right arms are flanked by priming sequences, the left arm contains a biotin molecule and the right arm does not contain a label. After completion of LCR, the probe products are purified using magnetic beads (optional) and then PCR amplified and microarray target prepared as illustrated in FIGS. 56 and 57.

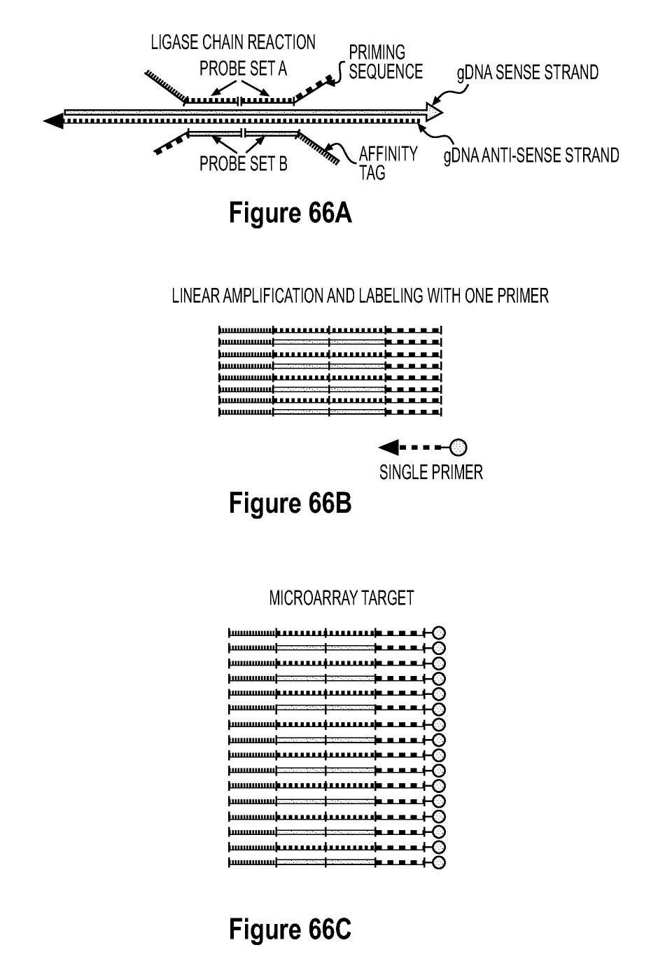

FIG. 66 depicts yet another embodiment of the procedure depicted in FIG. 65. The 5′ arm of each Probe Set consists of an affinity tag sequence and a homolog while the 3′ arm of each Probe Set consists of a homolog sequence and a priming sequence without a label attached as illustrated in FIG. 66A. After completion of the LCR, the probe product may be purified. The LCR product would then be amplified in a linear manner by the addition of a single primer that has a label attached, along with reaction components (Taq polymerase, dNTPs, and reaction buffer) as illustrated in FIG. 66B. The product of this amplification would be single-stranded (reverse strand only) with a 5′ label as illustrated in FIG. 66C. Consequently it would not be necessary to treat it with Lambda exonuclease but rather it could instead be directly used as microarray target.

In another aspect, the genetic variation determined by the methods described herein indicates presence or absence of cancer, pharmacokinetic variability, drug toxicity, transplant rejection, or aneuploidy in the subject. In another aspect, the determined genetic variation indicates presence or absence of cancer. Accordingly, the methods described herein may be performed to diagnose cancer.

A significant challenge in oncology is the early detection of cancer. This is particularly true in cancers that are hard to image or biopsy (e.g., pancreatic cancer, lung cancer). Cell free tumor DNA (tumor cfDNA) in a patient's blood offers a method to non-invasively detect a tumor. These may be solid tumors, benign tumors, micro tumors, liquid tumors, metastasis or other somatic growths. Detection may be at any stage in the tumor development, though ideally early (Stage I or Stage II). Early detection allows intervention (e.g., surgery, chemotherapy, pharmaceutical treatment) that may extend life or lead to remission. Further problems in oncology include the monitoring of the efficacy of treatment, the titration of the dose of a therapeutic agent, the recurrence of a tumor either in the same organ as the primary tumor or at distal locations and the detection of metastasis. The current invention may be used for all these applications. Another problem in oncology is the determination of whether a detected mass (e.g. a lung nodule) is cancerous or benign. For example, in lung cancer, nodules are often observed by CT scan or x-ray procedures. In many cases, these nodules are benign, but an invasive biopsy is typically performed to determine this. The current invention allows a blood based determination based on examination of the cfDNA from the patient. For example, a test could detect copy number change in some or all of the genome. Since a copy number change is a signature of cancer, the observation would indicate that the nodule may not be benign. Such a test could be used to determine which patients may need invasive procedures such as biopsies or be used in conjunction with imaging or other diagnostic procedures.

In some embodiments, the probe sets of the present disclosure may be configured to target known genetic variations associated with tumors. These may include mutations, SNPs, copy number variants (e.g., amplifications, deletions), copy neutral variants (e.g., inversions, translocations), and/or complex combinations of these variants. For example, the known genetic variations associated with tumors include those listed in cancer.sanger.ac.uk/cancergenome/projects/cosmic; nature.com/ng/journal/v45/n10/full/ng.2760.html#supplementary-information; and Tables 2 and 3 below: ^{B}GENE=p-value from corrected to FDR within peak; ^{K}Known frequently amplified oncogene or deleted TSG; ^{P}Putative cancer gene; ^{E}Epigenetic regulator; ^{M}Mitochondria-associated gene; **Immediately adjacent to peak region; ^{T}Adjacent to telomere or centromere of acrocentric chromosome.

TABLE 2
Exemplary genetic variations associated with tumors (Amplification of the gene)

| | | Genomic | | GISTIC | Gene | | Frequently |
|---|---|---|---|---|---|---|---|
| Peak Name | Rank | location | Peak region | q-value | count | Target(s) | mutated genes^{B} |
| CCND1 | 1 | 11q13.3 | chr11: 69464719-69502928 | 2.05E−278 | 2 | CCND1^{K} | CCND1 = 6.6e−08 |
| EGFR | 2 | 7p11.2 | chr7: 55075808-55093954 | 2.30E−240 | 1 | EGFR^{K} | EGFR = 2.2e−15 |
| MYC | 3 | 8q24.21 | chr8: 128739772-128762863 | 6.50E−180 | 1 | MYC^{K} | |
| TERC | 4 | 3q26.2 | chr3: 169389459-169490555 | 5.40E−117 | 2 | TERC^{P} | |
| ERBB2 | 5 | 17q12 | chr17: 37848534-37877201 | 1.59E−107 | 1 | ERBB2^{K} | ERBB2 = 13e−06 |
| CCNE1 | 6 | 19q12 | chr19: 30306758-30316875 | 4.77E−90 | 1 | CCNE1^{K} | |
| MCL1 | 7 | 1q21.3 | chr1: 150496857-150678056 | 1.25E−80 | 6 | MCL1^{K} | |
| MDM2 | 8 | 12q15 | chr12: 69183279-69260755 | 2.59E−62 | 2 | MDM2^{K} | |
| INTS4 | 9 | 11q14.1 | chr11: 77610143-77641464 | 1.01E−54 | 1 | INTS4 | |
| WHSC1L1 | 10 | 8p11.23 | chr8: 38191804-38260814 | 3.43E−46 | 2 | WHSC1L1^{E}, | |
| | | | | | | LETM2^{M} | |
| CDK4 | 11 | 12q14.1 | chr12: 58135797-58156509 | 5.14E−41 | 5 | CDK4^{K} | CDK4 = 0.0048 |
| KAT6A | 12 | 8p11.21 | chr8: 41751300-41897859 | 2.97E−39 | 2 | KAT6A^{P, E}, | |
| | | | | | | IKBKB** | |
| SOX2 | 13 | 3q26.33 | chr3: 181151312-181928394 | 1.21E−38 | 2 | SOX2^{K} | |
| PDGFRA | 14 | 4q12 | chr4: 54924794-55218386 | 1.08E−37 | 3 | PDGFRA^{K} | |
| BDH1 | 15 | 3q29 | chr3: 197212101-197335320 | 1.21E−31 | 1 | BDH1^{M} | |
| 1q44 | 16 | 1q44^{T} | chr1: 242979907-249250621 | 4.48E−31 | 83 | SMYD3^{E} | |
| MDM4 | 17 | 1q32.1 | chr1: 204367383-204548517 | 1.98E−29 | 3 | MDM4^{K} | |
| TERT | 18 | 5p15.33 | chr5: 1287704-1300024 | 9.34E−27 | 1 | TERT^{K} | |
| KDM5A | 19 | 12p13.33^{T} | chr12: 1-980639 | 1.59E−25 | 11 | KDM5A^{E} | |
| MYCL1 | 20 | 1p34.2 | chr1: 40317971-40417342 | 3.99E−25 | 2 | MYCL1^{K} | |
| IGF1R | 21 | 15q26.3 | chr15: 98667475-100292401 | 8.62E−25 | 9 | IGF1R^{K} | |
| PARP10 | 22 | 8q24.3 | chr8: 144925436-145219779 | 5.44E−20 | 15 | PARP10^{P, E}, | |
| | | | | | | CYC1^{M} | |
| G6PD | 23 | Xq28 | chrX: 153760870-153767853 | 3.66E−19 | 1 | G6PD | |
| PHF12 | 24 | 17q11.2 | chr17: 27032828-27327946 | 1.75E−16 | 21 | PHF12^{E}, | |
| | | | | | | ERAL1^{M} | |
| 20q13.33 | 25 | 20q13.33 | chr20: 62187847-62214354 | 2.96E−16 | 2 | | |
| PAF1 | 26 | 19q13.2 | chr19: 39699366-39945515 | 1.66E−15 | 13 | PAF1^{P, E} | IL28A = 0.021, |
| | | | | | | | SUPT5H = 0.084 |
| BCL2L1 | 27 | 20q11.21 | chr20: 30179028-30320705 | 2.85E−15 | 4 | BCL2L1^{K} | |
| TUBD1 | 28 | 17q23.1 | chr17: 57922443-57946458 | 7.19E−15 | 1 | TUBD1 | TUBD1 = 0.009 |
| [ZNF703] | 29 | 8p11.23 | chr8: 37492669-37527108 | 2.44E−14 | 0 | | |
| 1q23.3 | 30 | 1q23.3 | chr1: 160949115-161115281 | 7.73E−13 | 9 | | |
| 8q22.2 | 31 | 8q22.2 | chr8: 101324079-101652657 | 4.22E−11 | 3 | | SNX31 = 0.015 |
| BRD4 | 32 | 19p13.12 | chr19: 15310246-15428182 | 5.04E−10 | 3 | NOTCH3^{P}, | |
| | | | | | | BRD4^{P, E} | |
| KRAS | 33 | 12p12.1 | chr12: 24880663-25722878 | 9.47E−10 | 7 | KRAS^{K} | KRAS = 1.5e−14 |
| NKX2-1 | 34 | 14q13.2 | chr14: 35587755-37523513 | 1.33E−09 | 14 | NKX2-1^{K} | NFKBIA = 0.0098, |
| | | | | | | | RALGAPA1 = 0.027 |
| NFE2L2 | 35 | 2q31.2 | chr2: 178072322-178171101 | 5.48E−09 | 5 | NFE2L2 | NFE2L2 = 3.9e−14 |
| ZNF217 | 36 | 20q13.2 | chr20: 52148496-52442225 | 5.83E−08 | 1 | ZNF217^{K} | ZNF217 = 0.0082 |
| 13q34 | 37 | 13q34^{T} | chr13: 108818892-115169878 | 6.28E−08 | 45 | ING1^{E} | ING1 = 0.00026 |
| KAT6B | 38 | 10q22.2 | chr10: 76497097-77194071 | 1.41E−07 | 9 | KAT6B^{E}, | |
| | | | | | | VDAC2^{M} | |
| NSD1 | 39 | 5q35.3 | chr5: 176337344-177040112 | 1.75E−06 | 22 | NSD1^{E}, | NSD1 = 4.9e−10 |
| | | | | | | PRELID1^{M} | |
| FGFR3 | 40 | 4p16.3 | chr4: 1778797-1817427 | 2.14E−06 | 2 | FGFR3^{P}, | FGFR3 = 0.00018 |
| | | | | | | LETM1^{M} | |
| 9p13.3 | 41 | 9p13.3 | chr9: 35652385-35739486 | 2.55E−06 | 8 | | |
| COX18 | 42 | 4q13.3 | chr4: 73530210-74658151 | 2.68E−06 | 7 | COX18^{M} | |
| 7q36.3 | 43 | 7q36.3^{T} | chr7: 153768037-159138663 | 3.19E−06 | 30 | PTPRN2^{L}, | |
| | | | | | | DPP6^{L} | |
| 18q11.2 | 44 | 18q11.2 | chr18: 23857484-24119078 | 3.83E−06 | 2 | | |
| SOX17 | 45 | 8q11.23 | chr8: 55069781-55384342 | 2.02E−05 | 1 | SOX17 | SOX17 = 0.00092 |
| 11q22.2 | 46 | 11q22.2 | chr11: 102295593-102512085 | 0.00015337 | 3 | | |
| CBX8 | 47 | 17q25.3 | chr17: 77770110-77795534 | 0.00023029 | 1 | CBX8^{E} | |
| AKT1 | 48 | 14q32.33 | chr14: 105182581-105333748 | 0.00028451 | 7 | AKT1^{K} | AKT1 = 1.1e−14 |
| CDK6 | 49 | 7q21.2 | chr7: 92196092-92530348 | 0.00069831 | 3 | CDK6^{K} | |
| 6p21.1 | 50 | 6p21.1 | chr6: 41519930-44297771 | 0.0010459 | 70 | | |
| EHF | 51 | 11p13 | chr11: 34574296-34857324 | 0.0011002 | 1 | EHF | |
| 6q21 | 52 | 6q21 | chr6: 107098934-107359899 | 0.0011806 | 4 | | |
| 19q13.42 | 53 | 19q13.42^{T} | chr19: 55524376-59128983 | 0.0013319 | 138 | TRIM28^{E}, | ZNF471 = 5.4e−05 |
| | | | | | | SUV420H2^{E} | |
| 17q21.33 | 54 | 17q21.33 | chr17: 47346425-47509605 | 0.0025775 | 2 | | |
| BPTF | 55 | 17q24.2 | chr17: 65678858-66288612 | 0.0028375 | 11 | BPTF^{E} | |
| E2F3 | 56 | 6p22.3 | chr6: 19610794-22191922 | 0.0033658 | 7 | E2F3^{K} | |
| 19p13.2 | 57 | 19p13.2 | chr19: 10260457-10467501 | 0.0038041 | 12 | MRPL4^{M} | DNMT1 = 0.099 |
| 17q25.1 | 58 | 17q25.1 | chr17: 73568926-73594884 | 0.012337 | 2 | | |
| KDM2A | 59 | 11q13.2 | chr11: 67025375-67059633 | 0.012445 | 3 | KDM2A^{E} | |
| 8q21.13 | 60 | 8q21.13 | chr8: 80432552-81861219 | 0.020548 | 6 | MRPS28^{M} | |
| 2p15 | 61 | 2p15 | chr2: 59143237-63355557 | 0.021056 | 25 | | XPO1 = 1.1e−05 |
| 14q11.2 | 62 | 14q11.2^{T} | chr14: 1-21645085 | 0.027803 | 57 | | |
| NEDD9 | 63 | 6p24.2 | chr6: 11180426-11620845 | 0.082606 | 2 | NEDD9^{K} | |
| 5p13.1 | 64 | 5p13.1 | chr5: 35459650-50133375 | 0.094657 | 61 | | SLC1A3 = 0.0021, |
| | | | | | | | IL7R = 0.0021 |
| LINC00536 | 65 | 8q23.3 | chr8: 116891361-117360815 | 0.095294 | 1 | LINC00536 | |
| 10p15.1 | 66 | 10p15.1 | chr10: 4190059-6130004 | 0.10391 | 21 | | |
| 22q11.21 | 67 | 22q11.21 | chr22: 18613558-23816427 | 0.13213 | 105 | | |
| PHF3 | 68 | 6q12 | chr6: 63883156-64483307 | 0.17851 | 4 | PHF3^{E}, | PHF3 = 0.051 |
| | | | | | | EYS^{L} | |
| PAX8 | 69 | 2q13 | chr2: 113990138-114122826 | 0.19717 | 2 | PAX8^{K} | |
| 9p24.2 | 70 | 9p24.2^{T} | chr9: 1-7379570 | 0.20405 | 45 | SMARCA2^{E}, | |
| | | | | | | KDM4C^{E}, | |
| | | | | | | UHRF2^{E}, | |
| | | | | | | KIAA2026^{E} | |

TABLE 3
Exemplary genetic variations associated with tumors (Deletion of the gene)

| | | Genomic | | GISTIC | Gene | | Frequently |
|---|---|---|---|---|---|---|---|
| Peak Name | Rank | location | Peak region | q-value | count | Target(s) | mutated genes^{B} |
| CDKN2A | 1 | 9p21.3 | chr9: 21865498-22448737 | 0 | 4 | CDKN2A^{K} | CDKN2A = 4.4e−15 |
| STK11 | 2 | 19p13.3 | chr19: 1103715-1272039 | 1.46E−238 | 7 | STKU^{K} | STK11 = 2.5e−13 |
| PDE4D | 3 | 5q11.2 | chr5: 58260298-59787985 | 2.02E−143 | 3 | PDE4D^{L} | |
| PARK2 | 4 | 6q26 | chr6: 161693099-163153207 | 5.85E−137 | 1 | PARK2^{L, K} | |
| LRP1B | 5 | 2q22.1 | chr2: 139655617-143637838 | 4.25E−107 | 1 | LRP1B^{L} | |
| CSMD1 | 6 | 8p23.2 | chr8: 2079140-6262191 | 2.39E−96 | 1 | CSMD1^{L} | |
| 1p36.23 | 7 | 1p36.23 | chr1: 7829287-8925111 | 1.23E−93 | 8 | | |
| ARID1A | 8 | 1p36.11 | chr1: 26900639-27155421 | 5.74E−87 | 2 | ARID1A^{K} | ARID1A = 1.5e−14 |
| PTEN | 9 | 10q23.31 | chr10: 89615138-90034038 | 1.12E−79 | 2 | PTEN^{K} | PTEN = 2.2e−15 |
| WWOX | 10 | 16q23.1 | chr16: 78129058-79627770 | 8.14E−76 | 1 | WWOX^{L} | WWOX = 0.092 |
| RB1 | 11 | 13q14.2 | chr13: 48833767-49064807 | 3.88E−75 | 2 | RB1^{K} | RB1 = 1.7e−13 |
| FAM190A | 12 | 4q22.1 | chr4: 90844993-93240505 | 9.26E−75 | 1 | FAM190A^{L} | |
| 2q37.3 | 13 | 2q37.3^{T} | chr2: 241544527-243199373 | 1.77E−70 | 29 | ING5^{E} | |
| 22q13.32 | 14 | 22q13.32^{T} | chr22: 48026910-51304566 | 8.20E−65 | 45 | BRD1^{E}, | |
| | | | | | | HDAC10^{E} | |
| 11p15.5 | 15 | 11p15.5^{T} | chr11: 1-709860 | 1.02E−62 | 34 | SIRT3^{E}, | HRAS = 7.8e−13 |
| | | | | | | PHRF1^{E} | |
| LINC00290 | 16 | 4q34.3 | chr4: 178911874-183060693 | 1.21E−55 | 1 | LINC00290 | |
| FHIT | 17 | 3p14.2 | chr3: 59034763-61547330 | 3.01E−55 | 1 | FHIT^{L} | |
| RBFOX1 | 18 | 16p13.3 | chr16: 5144019-7771745 | 1.00E−45 | 1 | RBFOX1^{L} | |
| PTPRD | 19 | 9p24.1 | chr9: 8310705-12693402 | 3.24E−38 | 1 | PTPRD^{L} | |
| 18q23 | 20 | 18q23^{T} | chr18: 74979706-78077248 | 1.69E−37 | 12 | | |
| FAT1 | 21 | 4q35.2 | chr4: 187475875-188227950 | 6.81E−36 | 1 | FAT1^{K} | FAT1 = 2.4e−15 |
| MPHOSPH8 | 22 | 13q12.11^{T} | chr13: 1-20535070 | 2.57E−31 | 10 | MPHOSPH8^{E} | |
| 15q15.1 | 23 | 15q15.1 | chr15: 41795901-42068054 | 2.71E−29 | 4 | | MGA = 0.0083, |
| | | | | | | | RPAP1 = 0.035 |
| 11q25 | 24 | 11q25^{T} | chr11: 133400280-135006516 | 4.93E−26 | 14 | | |
| 1p13.2 | 25 | 1p13.2 | chr1: 110048528-117687124 | 1.69E−25 | 100 | TRIM33^{E} | NRAS = 1.8e−13, |
| | | | | | | | CD58 = 0.079 |
| NF1 | 26 | 17q11.2 | chr17: 29326736-29722618 | 6.59E−23 | 5 | NF1^{K} | NF1 = 3.3e−13 |
| MACROD2 | 27 | 20p12.1 | chr20: 14302876-16036135 | 9.00E−19 | 3 | MACROD2^{L} | |
| 7p22.3 | 28 | 7p22.3^{T} | chr7: 1-1496620 | 1.04E−17 | 18 | | |
| 6p25.3 | 29 | 6p25.3 | chr6: 1608837-2252425 | 3.01E−17 | 2 | | |
| 21q11.2 | 30 | 21q11.2^{T} | chr21: 1-15482604 | 2.34E−14 | 14 | | |
| 9p13.1 | 31 | 9p13.1 | chr9: 38619152-71152237 | 9.75E−14 | 48 | | |
| ZNF132 | 32 | 19q13.43^{T} | chr19: 58661582-59128983 | 3.77E−13 | 24 | TRIM28^{E}, | |
| | | | | | | ZNF132 | |
| 5q15 | 33 | 5q15 | chr5: 73236070-114508587 | 8.15E−13 | 156 | APC^{K}, | APC = 2.6e−13, |
| | | | | | | CHD1^{E} | RASA1 = 0.0029 |
| MLL3 | 34 | 7q36.1 | chr7: 151817415-152136074 | 9.26E−13 | 1 | MLL3^{K, E} | MLL3 = 1.1e−05 |
| 19q13.32 | 35 | 19q13.32 | chr19: 47332686-47763284 | 2.38E−12 | 10 | | |
| 15q12 | 36 | 15q12^{T} | chr15: 1-32929863 | 3.40E−11 | 155 | | OTUD7A = 0.027 |
| 12q24.33 | 37 | 12q24.33^{T} | chr12: 131692956-133851895 | 1.24E−10 | 27 | | POLE = 3.9e−05, |
| | | | | | | | PGAM5 = 0.038 |
| 10q26.3 | 38 | 10q26.3^{T} | chr10: 135190263-135534747 | 2.09E−10 | 14 | | |
| 6q21 | 39 | 6q21 | chr6: 86319089-117076132 | 4.56E−10 | 141 | PRDM1^{E}, | PRDM1 = 0.00054 |
| | | | | | | HDAC2^{E}, | |
| | | | | | | PRDM13^{E} | |
| PPP2R2A | 40 | 8p21.2 | chr8: 25896447-26250295 | 1.78E−09 | 1 | PPP2R2A | |
| IKZF2 | 41 | 2q34 | chr2: 211542637-214143899 | 3.24E−09 | 4 | IKZF2^{K}, | ERBB4 = 0.00058 |
| | | | | | | ERBB4^{L} | |
| CNTN4 | 42 | 3p26.3^{T} | chr3: 1-3100786 | 6.44E−09 | 3 | CNTN4^{L} | |
| 3p12.2 | 43 | 3p12.2 | chr3: 75363575-86988125 | 1.22E−07 | 12 | ROBO1^{E}, | |
| | | | | | | CADM2^{L} | |
| RAD51B | 44 | 14q24.1 | chr14: 68275375-69288431 | 1.38E−07 | 2 | RAD51B^{L} | ZFP36L1 = 0.0016 |
| 11q23.1 | 45 | 11q23.1 | chr11: 105849158-117024891 | 5.31E−07 | 84 | ATM^{K} | ATM = 1.4e−06, |
| | | | | | | | POU2AF1 = 0.082 |
| IMMP2L | 46 | 7q31.1 | chr7: 109599468-111366370 | 5.74E−07 | 2 | IMMP2L^{L} | |
| NEGR1 | 47 | 1p31.1 | chr1: 71699756-74522473 | 7.25E−07 | 2 | NEGR1^{L} | |
| BRCA1 | 48 | 17q21.31 | chr17: 41178765-41336147 | 7.25E−07 | 2 | BRCA1^{K} | BRCA1 = 3.5e−08 |
| 9q34.3 | 49 | 9q34.3 | chr9: 135441810-139646221 | 8.73E−06 | 94 | NOTCH1^{K}, | NOTCH1 = 1e−08, |
| | | | | | | BRD3^{E}, | RXRA = 2.1e−05, |
| | | | | | | GTF3C4^{E} | COL5A1 = 0.002, |
| | | | | | | | TSC1 = 0.012 |
| ANKS1B | 50 | 12q23.1 | chr12: 99124001-100431272 | 8.73E−06 | 2 | ANKS1B^{L} | |
| DMD | 51 | Xp21.2 | chrX: 30865118-34644819 | 5.15E−05 | 4 | DMD^{L} | |
| ZMYND11 | 52 | 10p15.3^{T} | chr10: 1-857150 | 7.12E−05 | 4 | ZMYND11^{E} | |
| PRKG1 | 53 | 10q11.23 | chr10: 52644085-54061437 | 9.79E−05 | 3 | PRKG1^{L} | |
| FOXK2 | 54 | 17q25.3 | chr17: 80443432-80574531 | 0.00019271 | 1 | FOXK2 | |
| AGBL4 | 55 | 1p33 | chr1: 48935280-50514967 | 0.000219 | 2 | AGBL4^{L} | |
| CDKN1B | 56 | 12p13.1 | chr12: 12710990-12966966 | 0.00035777 | 5 | CDKN1B^{K} | CDKN1B = 2.2e−06 |
| 14q32.33 | 57 | 14q32.33^{T} | chr14: 94381429-107349540 | 0.00074358 | 227 | SETD3^{E}, | AKT1 = 2.1e−13, |
| | | | | | | TDRD9^{E} | TRAF3 = 9.7e−05 |
| 14q11.2 | 58 | 14q11.2^{T} | chr14: 1-30047530 | 0.0010181 | 162 | PRMT5^{E}, | CHD8 = 0.034 |
| | | | | | | CHD8^{E} | |
| 2p25.3 | 59 | 2p25.3^{T} | chr2: 1-20072169 | 0.0011137 | 86 | MYCN^{K} | MYCN = 0.068 |
| 5q35.3 | 60 | 5q35.3^{T} | chr5: 153840473-180915260 | 0.0028515 | 212 | NSD1^{E}, | NPM1 = 3.5e−13, |
| | | | | | | ODZ2^{L} | NSD1 = 1.9e−09, |
| | | | | | | | ZNF454 = 0.0019, |
| | | | | | | | UBLCP1 = 0.03, |
| | | | | | | | GABRB2 = 0.07 |
| PTTG1IP | 61 | 21q22.3 | chr21: 46230687-46306160 | 0.012227 | 1 | PTTG1I^{P} | |
| 22q11.1 | 62 | 22q11.1^{T} | chr22: 1-17960585 | 0.020332 | 15 | | |
| SMAD4 | 63 | 18q21.2 | chr18: 48472083-48920689 | 0.036866 | 3 | SMAD4^{K} | SMAD4 = 6.6−15 |
| 17p13.3 | 64 | 17p13.3^{T} | chr17: 1-1180022 | 0.040814 | 16 | | |
| 4p16.3 | 65 | 4p16.3^{T} | chr4: 1-1243876 | 0.056345 | 27 | | |
| 9p21.2 | 66 | 9p21.2 | chr9: 27572512-28982153 | 0.091742 | 3 | | |
| 10q25.1 | 67 | 10q25.1 | chr10: 99340084-113910615 | 0.11879 | 137 | HPSE2^{L}, | SMC3 = 0.00031, |
| | | | | | | SMNDC1^{E} | GSTO2 = 0.086 |
| SMYD3 | 68 | 1q44 | chr1: 245282267-247110824 | 0.15417 | 8 | SMYD3^{E} | |
| 8p11.21 | 69 | 8p11.21 | chr8: 42883855-47753079 | 0.17382 | 4 | | |
| Xp22.33 | 70 | Xp22.33^{T} | chrX: 1-11137490 | 0.21462 | 52 | | MXRA5 = 0.031 |

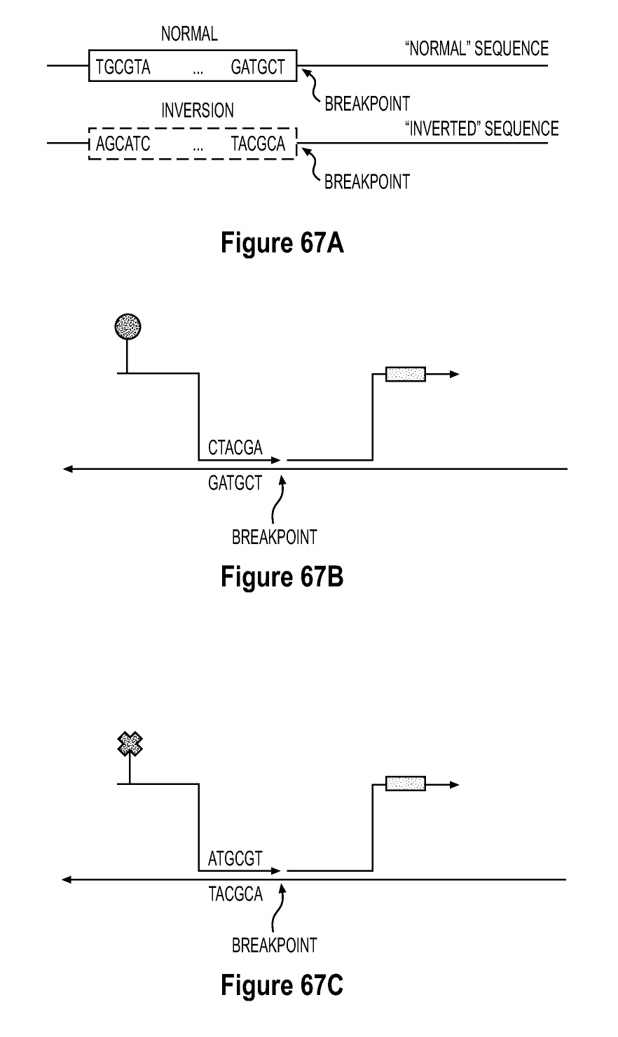

In the method of diagnosing cancer according to some embodiments, inversions that occur at known locations (FIG. 67A) may easily be targeted by designing probes that at least partially overlap the breakpoint in one probe arm. A first probe that binds the “normal” sequence targets non-inverted genomic material (FIG. 67B) and carries a first label type. A second probe that binds the “inverted” target carries a second label type (FIG. 67C). A common right probe arm binds native sequence that is not susceptible to inversion, immediately adjacent the first two probes. This right probe arm further carries a common pull-down tag that localizes the probe products to the same region of an imaging substrate. In this way, the probe pairs may hybridize to the genomic targets, ligate, and be imaged to yield relative counts of the two underlying species.

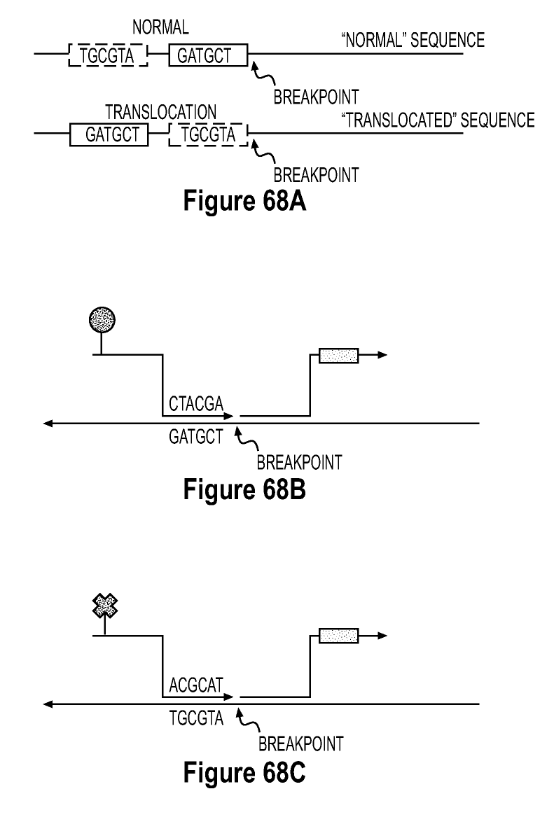

Similarly, translocations that have known breakpoints may also be assayed. FIG. 68A shows two genetic elements that are either in their native order or translocated. Probe arms that at least partially overlap these translocation breakpoints allow differentiation between normal and transposed orders of genetic material. As shown in FIGS. 68B and 68C, by choosing unique labels on the two left arms, the resulting ligated probe products may be distinguished and counted during imaging.

These methods for detecting copy neutral changes (e.g., inversions, translocation) may also be used to detect germline variants in cancer or in other disease or conditions.

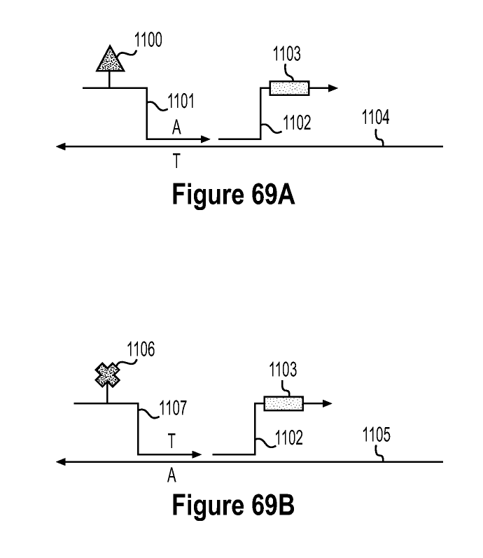

Mutations or SNPs are also implicated in numerous cancers, and are targeted in a similar manner to those that are interrogated in determining fetal fraction in the prenatal diagnostics application. In some embodiments shown in FIGS. 69A and 69B, left probe arms are designed to take advantage of an energetic imbalance caused by one or more mismatched SNPs. This causes one probe arm (1101, carrying one label) to bind more favorably than a second probe arm (1107, carrying a second type of label). Both designs ligate to the same right probe arm (1102) that carries the universal pull-down tag.

A given patient's blood may be probed by one method, or a hybrid of more than one method. Further, in some cases, customizing specific probes for a patient may be valuable. This would involve characterizing tumor features (SNPs, translocations, inversions, etc.) in a sample from the primary tumor (e.g., a biopsy) and creating one or more custom probe sets that is optimized to detect those patient-specific genetic variations in the patient's blood, providing a low-cost, non-invasive method for monitoring. This could have significant value in the case of relapse, where detecting low-level recurrence of a tumor type (identical or related to the original tumor) as early as possible is ideal.

For common disease progression pathways, additional panels may be designed to anticipate and monitor for disease advancement. For example, if mutations tend to accumulate in a given order, probes may be designed to monitor current status and progression “checkpoints,” and guide therapy options.

Early detection of cancer: For example, the ALK translocation has been associated with lung cancer. A probe designed to interrogate the ALK translocation may be used to detect tumors of this type via a blood sample. This would be highly advantageous, as the standard method for detecting lung tumors is via a chest x-ray an expensive procedure that may be deleterious to the patient's health and so is not standardly performed.

Detection of recurrence of the primary tumor type: For example, a HER2+ breast tumor is removed by surgery and the patient is in remission. A probe targeting the HER2 gene may be used to monitor for amplifications of the HER2 gene at one or more time points. If these are detected, the patient may have a second HER2+ tumor either at the primary site or elsewhere.

Detection of non-primary tumor types: For example, a HER2+ breast tumor is removed by surgery and the patient is in remission. A probe targeting the EGFR gene may be used to monitor for EGFR+ tumors. If these are detected, the patient may have a second EGFR+ tumor either at the primary site or elsewhere.

Detection of metastasis: For example, the patient has a HER2+ breast tumor. A probe designed to interrogate the ALK translocation may be used to detect tumors of this type via a blood sample. This tumor may not be in the breast and is more likely to be in the lung. If these are detected, the patient may have a metastatic tumor distal to the primary organ.

Determining tumor heterogeneity: Many tumors have multiple clonal populations characterized by different genetic variants. For example, a breast tumor may have one population of cells that are HER2+ and another population of cells that are EGFR+. Using probes designed to target both these variants would allow the identification of this underlying genetic heterogeneity.

Measurement of tumor load: In all the above examples, the quantity of tumor cfDNA may be measured and may be used to determine the size, growth rate, aggressiveness, stage, prognosis, diagnosis and other attributes of the tumor and the patient. Ideally, measurements are made at more than one time point to show changes in the quantity of tumor cfDNA.

Monitoring treatment: For example, a HER2+ breast tumor is treated with Herceptin. A probe targeting the HER2 gene may be used to monitor for quantity of tumor cfDNA, which may be a proxy for the size of the tumor. This may be used to determine if the tumor is changing in size and treatment may be modified to optimize the patient's outcome. This may include changing the dose, stopping treatment, changing to another therapy, combing multiple therapies.

Screening for tumor DNA: There is currently no universal screen for cancer. The present invention offers a way to detect tumors at some or all locations in the body. For example, a panel of probes is developed at a spacing of 100 kb across the genome. This panel may be used as a way to detect genetic variation across the genome. In one example, the panel detects copy number changes of a certain size across the genome. Such copy number changes are associated with tumor cells and so the test detects the presence of tumor cells. Different tumor types may produce different quantities of tumor cfDNA or may have variation in different parts of the genome. As such, the test may be able to identify which organ is affected. Further the quantity of tumor cfDNA measured may indicate the stage or size of the tumor or the location of the tumor. In this way, the test is a whole-genome screen for many or all tumor types.

For all the above tests, in order to mitigate false positives, a threshold may be used to determine the presence or certainty of a tumor. Further, the test may be repeat on multiple sample or at multiple time points to increase the certainty of the results. The results may also be combined with other information or symptoms to provide more information or more certain information on the tumor.

Exemplary probe sets and primers that may be used in the method described herein to measure copy number of nucleic acid regions of interest are listed in Table 4 below. Each of the exemplary probe sets in Table 4 comprises two probes. The first (tagging) probe has a structure including a forward priming site, tag, and homology 1. The second (labeling) probe has structure, including homology 2 and reverse primer site, which is used in labeling. The component sequences of the probes (tag, homology sequence etc.) are also shown.

TABLE 4
Exemplary probes and primers.

| | | Tagging Probe | Labeling Probe | | | | | |
|---|---|---|---|---|---|---|---|---|
| Chromo- | Locus | (Forward Primer + | (3′-Hop + | | | | | Reverse |
| some | ID | Tag + 5pHom) | Reverse Primer) | Forward primer | Tag | Hom 5p | Hom 3p | primer |
| 18 | 18-1 | GCCCTCATCTTC | CGTGCTAATAGTC | GCCCTCAT | GTTCTCA | GGAAGAA | CGTGCTA | TTCCTCCA |
| | | TTCCCTGCGTTC | TCAGGGCTTCCTC | CTTCTTCC | CCACCCT | GTGAGGG | ATAGTCT | CCGAACGT |
| | | TCACCACCCTCA | CACCGAACGTGT | CTGC (SEQ | CACCAA | CTTCTC | CAGGGC | GTCT (SEQ |
| | | CCAAGGAAGAA | CT (SEQ ID NO: 17) | ID NO: 33) | (SEQ ID | (SEQ ID | (SEQ ID | ID NO: 67) |
| | | GTGAGGGCTTCT | | | NO: 34) | NO: 35) | NO: 51) | |
| | | C (SEQ ID NO: 1) | | | | | | |
| 18 | 18-2 | GCCCTCATCTTC | CGACGCTTCATTG | GCCCTCAT | GTTCTCA | AAATCAA | CGACGC | TTCCTCCA |
| | | TTCCCTGCGTTC | CTTCATTTTCCTC | CTTCTTCC | CCACCCT | GGTGACC | TTCATTG | CCGAACGT |
| | | TCACCACCCTCA | CACCGAACGTGT | CTGC (SEQ | CACCAA | AGCTCC | CTTCATT | GTCT (SEQ |
| | | CCAAAAATCAAG | CT (SEQ ID NO: 18) | ID NO: 33) | (SEQ ID | (SEQ ID | (SEQ ID | ID NO: 67) |
| | | GTGACCAGCTCC | | | NO: 34) | NO: 36) | NO: 52) | |
| | | (SEQ ID NO: 2) | | | | | | |
| 18 | 18-3 | GCCCTCATCTTC | CTTGCGCCAAAC | GCCCTCAT | GTTCTCA | TCATCTG | CTTGCGC | TTCCTCCA |
| | | TTCCCTGCGTTC | AATTGTCCTTCCT | CTTCTTCC | CCACCCT | CCAAGAC | CAAACA | CCGAACGT |
| | | TCACCACCCTCA | CCACCGAACGTG | CTGC (SEQ | CACCAA | AGAAGTT | ATTGTCC | GTCT (SEQ |
| | | CCAATCATCTGC | TCT (SEQ ID NO: | ID NO: 33) | (SEQ ID | C (SEQ ID | (SEQ ID | ID NO: 67) |
| | | CAAGACAGAAG | 19) | | NO: 34) | NO: 37) | NO: 53) | |
| | | TTC (SEQ ID NO: | | | | | | |
| | | 3) | | | | | | |
| 18 | 18-4 | GCCCTCATCTTC | GCTGCAGAGTTTG | GCCCTCAT | GTTCTCA | GCAGGAG | GCTGCA | TTCCTCCA |
| | | TTCCCTGCGTTC | CATTCATTTCCTC | CTTCTTCC | CCACCCT | AGTCAAA | GAGTTTG | CCGAACGT |
| | | TCACCACCCTCA | CACCGAACGTGT | CTGC (SEQ | CACCAA | GGTCTG | CATTCAT | GTCT (SEQ |
| | | CCAAGCAGGAG | CT (SEQ ID NO: 20) | ID NO: 33) | (SEQ ID | (SEQ ID | (SEQ ID | ID NO: 67) |
| | | AGTCAAAGGTCT | | | NO: 34) | NO: 38) | NO: 54) | |
| | | G (SEQ ID NO: 4) | | | | | | |
| 18 | 18-5 | GCCCTCATCTTC | CATACACACAGA | GC CCTCAT | GTTCTCA | GTTGC CA | CATACA | TTCCTCCA |
| | | TTCCCTGCGTTC | CCGAGAGTCTTCC | CTTCTTCC | CCACCCT | TGGAGAT | CACAGA | CCGAACGT |
| | | TCACCACCCTCA | TCCACCGAACGT | CTGC (SEQ | CACCAA | TGTTGC | CCGAGA | GTCT (SEQ |
| | | CCAAGTTGCCAT | GTCT (SEQ ID NO: | ID NO: 33) | (SEQ ID | (SEQ ID | GTC (SEQ | ID NO: 67) |
| | | GGAGATTGTTGC | 21) | | NO: 34) | NO: 39) | ID NO: 55) | |
| | | (SEQ ID NO: 5) | | | | | | |
| 18 | 18-6 | GCCCTCATCTTC | GGATGTCAGCCA | GCCCTCAT | GTTCTCA | CAGCTCA | GGATGT | TTCCTCCA |
| | | TTCCCTGCGTTC | GCATAAGTTTCCT | CTTCTTCC | CCACCCT | GTGATGT | CAGCCA | CCGAACGT |
| | | TCACCACCCTCA | CCACCGAACGTG | CTGC (SEQ | CACCAA | CATTGC | GCATAA | GTCT (SEQ |
| | | CCAACAGCTCAG | TCT (SEQ ID NO: | ID NO: 33) | (SEQ ID | (SEQ ID | GT (SEQ | ID NO: 67) |
| | | TGATGTCATTGC | 22) | | NO: 34) | NO: 40) | ID NO: 56) | |
| | | (SEQ ID NO: 6) | | | | | | |
| 18 | 18-7 | GCCCTCATCTTC | GCAAGTGCCAAA | GCCCTCAT | GTTCTCA | CCTTGAC | GCAAGT | TTCCTCCA |
| | | TTCCCTGCGTTC | CAGTTCTCTTCCT | CTTCTTCC | CCACCCT | CTCTGCT | GCCAAA | CCGAACGT |
| | | TCACCACCCTCA | CCACCGAACGTG | CTGC (SEQ | CACCAA | AATGTGG | CAGTTCT | GTCT (SEQ |
| | | CCAACCTTGACC | TCT (SEQ ID NO: | ID NO: 33) | (SEQ ID | (SEQ ID | C (SEQ ID | ID NO: 67) |
| | | TCTGCTAATGTG | 23) | | NO: 34) | NO: 41) | NO: 57) | |
| | | G (SEQ ID NO: 7) | | | | | | |
| 18 | 18-8 | GCCCTCATCTTC | GATTCCAGCACA | GCCCTCAT | GTTCTCA | CACCTGT | GATTCCA | TTCCTCCA |
| | | TTCCCTGCGTTC | CTTGAGTCTTTCC | CTTCTTCC | CCACCCT | CCAACAG | GCACAC | CCGAACGT |
| | | TCACCACCCTCA | TCCACCGAACGT | CTGC (SEQ | CACCAA | CTACAG | TTGAGTC | GTCT (SEQ |
| | | CCAACACCTGTC | GTCT (SEQ ID NO: | ID NO: 33) | (SEQ ID | (SEQ ID | T (SEQ ID | ID NO: 67) |
| | | CAACAGCTACAG | 24) | | NO: 34) | NO: 42) | NO: 58) | |
| | | (SEQ ID NO: 8) | | | | | | |
| X | X-1 | GCCCTCATCTTC | CCGTTGCAGGTTT | GCCCTCAT | GTTCTCA | AGAATGT | CCGTTGC | GCCCTATT |
| | | TTCCCTGCGTTC | AAATGGCGCCCT | CTTCTTCC | CCACCCT | ATCTTCA | AGGTTTA | GCAAGCCC |
| | | TCACCACCCTCA | ATTGCAAGCCCTC | CTGC (SEQ | CACCAA | GGCCTGC | AATGGC | TCTT (SEQ |
| | | CCAAAGAATGTA | TT (SEQ ID NO: 25) | ID NO: 33) | (SEQ ID | (SEQ ID | (SEQ ID | ID NO: 68) |
| | | TCTTCAGGCCTG | | | NO: 34) | NO: 43) | NO: 59) | |
| | | C (SEQ ID NO: 9) | | | | | | |
| X | X-2 | GCCCTCATCTTC | CAAGAGTGCTTTA | GCCCTCAT | GTTCTCA | AAGTAAT | CAAGAG | GCCCTATT |
| | | TTCCCTGCGTTC | TGGGCCTGCCCTA | CTTCTTCC | CCACCCT | CACTCTG | TGCTTTA | GCAAGCCC |
| | | TCACCACCCTCA | TTGCAAGCCCTCT | CTGC (SEQ | CACCAA | GGTGGC | TGGGCCT | TCTT (SEQ |
| | | CCAAAAGTAATC | T (SEQ ID NO: 26) | ID NO: 33) | (SEQ ID | (SEQ ID | (SEQ ID | ID NO: 68) |
| | | ACTCTGGGTGGC | | | NO: 34) | NO: 44) | NO: 60) | |
| | | (SEQ ID NO: 10) | | | | | | |
| X | X-3 | GCCCTCATCTTC | GCACTCAAGGAG | GCCCTCAT | GTTCTCA | AGCTCAC | GCACTC | GCCCTATT |
| | | TTCCCTGCGTTC | ATCAGACTGGCC | CTTCTTCC | CCACCCT | AGACAAC | AAGGAG | GCAAGCCC |
| | | TCACCACCCTCA | CTATTGCAAGCCC | CTGC (SEQ | CACCAA | CTTGTG | ATCAGA | TCTT (SEQ |
| | | CCAAAGCTCACA | TCTT (SEQ ID NO: | ID NO: 33) | (SEQ ID | (SEQ ID | CTG (SEQ | ID NO: 68) |
| | | GACAACCTTGTG | 27) | | NO: 34) | NO: 45) | ID NO: 61) | |
| | | (SEQ ID NO: 11) | | | | | | |
| X | X-4 | GCCCTCATCTTC | GGCTATCGAACT | GCCCTCAT | GTTCTCA | GCAATAG | GGCTATC | GCCCTATT |
| | | TTCCCTGCGTTC | ACAACCACAGCC | CTTCTTCC | CCACCCT | ACACCTA | GAACTA | GCAAGCCC |
| | | TCACCACCCTCA | CTATTGCAAGCCC | CTGC (SEQ | CACCAA | CAGGCG | CAACCA | TCTT (SEQ |
| | | CCAAGCAATAGA | TCTT (SEQ ID NO: | ID NO: 33) | (SEQ ID | (SEQ ID | CA (SEQ | ID NO: 68) |
| | | CACCTACAGGCG | 28) | | NO: 34) | NO: 46) | ID NO: 62) | |
| | | (SEQ ID NO: 12) | | | | | | |
| X | X-5 | GCCCTCATCTTC | GTAGCTGTCTGTG | GCCCTCAT | GTTCTCA | GCACATT | GTAGCT | GCCCTATT |
| | | TTCCCTGCGTTC | GTGTGATCGCCCT | CTTCTTCC | CCACCCT | ATCAAAG | GTCTGTG | GCAAGCCC |
| | | TCACCACCCTCA | ATTGCAAGCCCTC | CTGC (SEQ | CACCAA | GCCACG | GTGTGAT | TCTT (SEQ |
| | | CCAAGCACATTA | TT (SEQ ID NO: 29) | ID NO: 33) | (SEQ ID | (SEQ ID | C (SEQ ID | ID NO: 68) |
| | | TCAAAGGCCACG | | | NO: 34) | NO: 47) | NO: 63) | |
| | | (SEQ ID NO: 13) | | | | | | |
| X | X-6 | GCCCTCATCTTC | CAAGAAACTTCG | GCCCTCAT | GTTCTCA | CAACGAC | CAAGAA | GCCCTATT |
| | | TTCCCTGCGTTC | AGCCTTAGCAGC | CTTCTTCC | CCACCCT | CTAAAGC | ACTTCGA | GCAAGCCC |
| | | TCACCACCCTCA | CCTATTGCAAGCC | CTGC (SEQ | CACCAA | ATGTGC | GCCTTAG | TCTT (SEQ |
| | | CCAACAACGACC | CTCTT (SEQ ID | ID NO: 33) | (SEQ ID | (SEQ ID | CA (SEQ | ID NO: 68) |
| | | TAAAGCATGTGC | NO: 30) | | NO: 34) | NO: 48) | ID NO: 64) | |
| | | (SEQ ID NO: 14) | | | | | | |
| X | X-7 | GCCCTCATCTTC | GTGAACCAGTCC | GCCCTCAT | GTTCTCA | GACATAC | GTGAAC | GCCCTATT |
| | | TTCCCTGCGTTC | GAGTGAAAGCCC | CTTCTTCC | CCACCCT | ATGGCTT | CAGTCC | GCAAGCCC |
| | | TCACCACCCTCA | TATTGCAAGCCCT | CTGC (SEQ | CACCAA | TGGCAG | GAGTGA | TCTT (SEQ |
| | | CCAAGACATACA | CTT (SEQ ID NO: | ID NO: 33) | (SEQ ID | (SEQ ID | AA (SEQ | ID NO: 68) |
| | | TGGCTTTGGCAG | 31) | | NO: 34) | NO: 49) | ID NO: 65) | |
| | | (SEQ ID NO: 15) | | | | | | |
| X | X-8 | GCCCTCATCTTC | GCAAATGATGTTC | GCCCTCAT | GTTCTCA | GAGATAC | GCAAAT | GCCCTATT |
| | | TTCCCTGCGTTC | AGCACCACGCCC | CTTCTTCC | CCACCCT | TGCCACT | GATGTTC | GCAAGCCC |
| | | TCACCACCCTCA | TATTGCAAGCCCT | CTGC (SEQ | CACCAA | TATGCAC | AGCACC | TCTT (SEQ |
| | | CCAAGAGATACT | CTT (SEQ ID NO: | ID NO: 33) | (SEQ ID | G (SEQ ID | AC (SEQ | ID NO: 68) |
| | | GCCACTTATGCA | 32) | | NO: 34) | NO: 50) | ID NO: 66) | |
| | | CG (SEQ ID NO: | | | | | | |
| | | 16) | | | | | | |

Exemplary probe sets and primers that may be used in the method described herein to detect a polymorphism at a SNP site are listed in Table 5 below. Each of the exemplary probe sets in Table 5 comprises three probes, two allele specific probes (that are used for labeling) and a tagging probe. In these examples, the two allele specific probes have homology sequences that are different at one or more nucleotides. The structure of the first allelic probe includes a Forward Primer Site Allele 1 and Homology Allele 1; and the structure of the second allelic probe includes a Forward Primer Site Allele 2 and Homology Allele 2. In practice, labeled primers may be used with different labels on the two primers (so the labels are allele specific). In these examples, there also is a universal 3′ probe which includes a homology region (without any SNP), the tagging sequence and a reverse primer site. The component sequences of the probes (tag, homology sequence etc.) are also shown.

TABLE 5
Exemplary probes and primers.

| | Labeling Probe- | Labeling Probe- | | | | | | | | |
|---|---|---|---|---|---|---|---|---|---|---|
| | Allele 1 | Allele 2 | Tagging Probe | | | | | | | |
| | (Forward Primer | (Forward Primer | (Hom 3p + | Forward | Forward | | | | | |
| Chromo- | Allele 1 + Hom | Allele 2 + Hom | Tag + | Primer- | Primer- | Hom 5p- | Hom 5p- | | | Reverse |
| some | 5p Allele 1) | 5p Allele 2) | Reverse Primer) | Allele 1 | Allele 2 | Allele 1 | Allele 2 | Hom 3p | Tag | Primer |
| chr21 | TTCCTCCACC | GCCCTATTGCA | CACTTGACAA | TTCCTC | GCCCTAT | AGACCA | AGACC | CACTT | GCCGA | GCCCT |
| | GAACGTGTCT | AGCCCTCTTAG | AGTTCTCACG | CACCGA | TGCAAG | GCACAA | AGCAC | GACA | AGTTC | CATCT |
| | AGACCAGCAC | ACCAGCACAAC | CGCCGAAGTT | ACGTGT | CCCTCTT | CTTACTc | AACTT | AAGTT | TCCGA | TCTTC |
| | AACTTACTcg | TTACTta (SEQ ID | CTCCGAAGGA | CT (SEQ | (SEQ ID | g (SEQ ID | ACTta | CTCAC | AGGAT | CCTGC |
| | (SEQ ID NO: 69) | NO: 112) | TGCCCTCATC | ID NO: | NO: 68) | NO: 198) | (SEQ ID | GC | (SEQ | (SEQ |
| | | | TTCTTCCCTGC | 67) | | | NO: 241) | (SEQ | ID NO: | ID NO: |
| | | | (SEQ ID NO: | | | | | ID NO: | 327) | 33) |
| | | | 155) | | | | | 284) | | |
| chr3 | TTCCTCCACC | GCCCTATTGCA | CATTAGGGAT | TTCCTC | GCCCTAT | CCAAAT | CCAAA | CATTA | GACA | GCCCT |
| | GAACGTGTCT | AGCCCTCTTCCA | TAACGGCTTG | CACCGA | TGCAAG | gCACCT | TtCACC | GGGAT | GACTG | CATCT |
| | CCAAATgCAC | AATtCACCTGCC | GGACAGACTG | ACGTGT | CCCTCTT | GCCtg | TGCCca | TAACG | ACGG | TCTTC |
| | CTGCCtg (SEQ | ca (SEQ ID NO: | ACGGAGCTTC | CT (SEQ | (SEQ ID | (SEQ ID | (SEQ ID | GCTTG | AGCTT | CCTGC |
| | ID NO: 70) | 113) | AGCCCTCATC | ID NO: | NO: 68) | NO: 199) | NO: 242) | G (SEQ | CA | (SEQ |
| | | | TTCTTCCCTGC | 67) | | | | ID NO: | (SEQ | ID NO: |
| | | | (SEQ ID NO: | | | | | 285) | ID NO: | 33) |
| | | | 156) | | | | | | 328) | |
| chr13 | TTCCTCCACC | GCCCTATTGCA | CACACGTTAA | TTCCTC | GCCCTAT | AGTTTG | AGTTT | CACAC | TGACT | GCCCT |
| | GAACGTGTCT | AGCCCTCTTAGT | GAAGACTTTC | CACCGA | TGCAAG | GACAAA | GGACA | GTTAA | CTGCC | CATCT |
| | AGTTTGGACA | TTGGACAAAGG | TGCTGACTCT | ACGTGT | CCCTCTT | GGCaAT | AAGGC | GAAG | GCACA | TCTTC |
| | AAGGCaATTcg | CgATTta (SEQ ID | GCCGCACATG | CT (SEQ | (SEQ ID | Tcg (SEQ | gATTta | ACTTT | TGATC | CCTGC |
| | (SEQ ID NO: 71) | NO: 114) | ATCGCCCTCA | ID NO: | NO: 68) | ID NO: | (SEQ ID | CTGC | (SEQ | (SEQ |
| | | | TCTTCTTCCCT | 67) | | 200) | NO: 243) | (SEQ | ID NO: | ID NO: |
| | | | GC (SEQ ID NO: | | | | | ID NO: | 329) | 33) |
| | | | 157) | | | | | 286) | | |
| chr3 | TTCCTCCACC | GCCCTATTGCA | CTAAGTGCCC | TTCCTC | GCCCTAT | TGAGCT | TGAGC | CTAAG | GATCC | GCCCT |
| | GAACGTGTCT | AGCCCTCTTTGA | TCCATGAGAA | CACCGA | TGCAAG | TAGCCA | TTAGC | TGCCC | GATAG | CATCT |
| | TGAGCTTAGC | GCTTAGCCAAT | AGGATCCGAT | ACGTGT | CCCTCTT | ATATCA | CAATA | TCCAT | CCCTC | TCTTC |
| | CAATATCAAg | ATCAAcAAGa | AGCCCTCTGC | CT (SEQ | (SEQ ID | AgAAGg | TCAAcA | GAGA | TGCAG | CCTGC |
| | AAGg (SEQ ID | (SEQ ID NO: 115) | AGGCCCTCAT | ID NO: | NO: 68) | (SEQ ID | AGa | AAG | (SEQ | (SEQ |
| | NO: 72) | | CTTCTTCCCTG | 67) | | NO: 201) | (SEQ ID | (SEQ | ID NO: | ID NO: |
| | | | C (SEQ ID NO: | | | | NO: 244) | ID NO: | 330) | 33) |
| | | | 158) | | | | | 287) | | |
| chr9 | TTCCTCCACC | GCCCTATTGCA | GCACAGATTT | TTCCTC | GCCCTAT | ACGTGA | ACGTG | GCACA | CAACA | GCCCT |
| | GAACGTGTCT | AGCCCTCTTAC | CCCACACTCT | CACCGA | TGCAAG | ACTTTC | AACTT | GATTT | GGCCT | CATCT |
| | ACGTGAACTT | GTGAACTTTCCT | CAACAGGCCT | ACGTGT | CCCTCTT | CTTGGT | TCCTTG | CCCAC | GCTAA | TCTTC |
| | TCCTTGGTAcA | TGGTAaAt (SEQ | GCTAAACACC | CT (SEQ | (SEQ ID | AcAc | GTAaAt | ACTCT | ACACC | CCTGC |
| | c (SEQ ID NO: | ID NO: 116) | GCCCTCATCT | ID NO: | NO: 68) | (SEQ ID | (SEQ ID | (SEQ | (SEQ | (SEQ |
| | 73) | | TCTTCCCTGC | 67) | | NO: 202) | NO: 245) | ID NO: | ID NO: | ID NO: |
| | | | (SEQ ID NO: | | | | | 288) | 331) | 33) |
| | | | 159) | | | | | | | |
| chr3 | TTCCTCCACC | GCCCTATTGCA | CTTACAGGAG | TTCCTC | GCCCTAT | TGAAGA | TGAAG | CTTAC | GGTCA | GCCCT |
| | GAACGTGTCT | AGCCCTCTTTGA | GTCTGGCATC | CACCGA | TGCAAG | TGTTCT | ATGTT | AGGA | ACAAC | CATCT |
| | TGAAGATGTT | AGATGTTCTAA | AGGTCAACAA | ACGTGT | CCCTCTT | AATACC | CTAAT | GGTCT | CGAG | TCTTC |
| | CTAATACCTT | TACCTTGCta | CCGAGGGACT | CT (SEQ | (SEQ ID | TTGCcg | ACCTT | GGCAT | GGACT | CCTGC |
| | GCcg (SEQ ID | (SEQ ID NO: 117) | CGCCCTCATC | ID NO: | NO: 68) | (SEQ ID | GCta | CA | C (SEQ | (SEQ |
| | NO: 74) | | TTCTTCCCTGC | 67) | | NO: 203) | (SEQ ID | (SEQ | ID NO: | ID NO: |
| | | | (SEQ ID NO: | | | | NO: 246) | ID NO: | 332) | 33) |
| | | | 160) | | | | | 289) | | |
| chr17 | TTCCTCCACC | GCCCTATTGCA | CCACAATGAG | TTCCTC | GCCCTAT | CAGTGT | CAGTG | CCACA | TTGTC | GCCCT |
| | GAACGTGTCT | AGCCCTCTTCA | AAGGCAGAGT | CACCGA | TGCAAG | GGAGAC | TGGAG | ATGAG | ATTAA | CATCT |
| | CAGTGTGGAG | GTGTGGAGACc | TGTCATTAAT | ACGTGT | CCCTCTT | tGAACg | ACcGA | AAGG | TGCTG | TCTTC |
| | ACtGAACg | GAACa (SEQ ID | GCTGGCGGCG | CT (SEQ | (SEQ ID | (SEQ ID | ACa | CAGA | GCGGC | CCTGC |
| | (SEQ ID NO: 75) | NO: 118) | CCCTCATCTTC | ID NO: | NO: 68) | NO: 204) | (SEQ ID | G (SEQ | (SEQ | (SEQ |
| | | | TTCCCTGC | 67) | | | NO: 247) | ID NO: | ID NO: | ID NO: |
| | | | (SEQ ID NO: | | | | | 290) | 333) | 33) |
| | | | 161) | | | | | | | |
| chr16 | TTCCTCCACC | GCCCTATTGCA | GCTGTGGCAT | TTCCTC | GCCCTAT | AGGCAG | AGGCA | GCTGT | CGGTG | GCCCT |
| | GAACGTGTCT | AGCCCTCTTAG | AGCTACACTC | CACCGA | TGCAAG | GGTAAT | GGGTA | GGCAT | ACGGT | CATCT |
| | AGGCAGGGTA | GCAGGGTAATG | CGGTGACGGT | ACGTGT | CCCTCTT | GTCATG | ATGTC | AGCTA | TTGCA | TCTTC |
| | ATGTCATGAA | TCATGAAgTt | TTGCAACTTT | CT (SEQ | (SEQ ID | AAaTg | ATGAA | CACTC | ACTTT | CCTGC |
| | aTg (SEQ ID | (SEQ ID NO: 119) | GCCCTCATCT | ID NO: | NO: 68) | (SEQ ID | gTt (SEQ | (SEQ | (SEQ | (SEQ |
| | NO: 76) | | TCTTCCCTGC | 67) | | NO: 205) | ID NO: | ID NO: | ID NO: | ID NO: |
| | | | (SEQ ID NO: | | | | 248) | 291) | 334) | 33) |
| | | | 162) | | | | | | | |
| chr21 | TTCCTCCACC | GCCCTATTGCA | CAGGGTAATT | TTCCTC | GCCCTAT | GATTGT | GATTG | CAGG | GTCCG | GCCCT |
| | GAACGTGTCT | AGCCCTCTTGAT | TGTGGGTCTG | CACCGA | TGCAAG | CTGGAG | TCTGG | GTAAT | GCAGT | CATCT |
| | GATTGTCTGG | TGTCTGGAGgGC | GTCCGGCAGT | ACGTGT | CCCTCTT | cGCTg | AGgGC | TTGTG | TAAGG | TCTTC |
| | AGcGCTg (SEQ | Tc (SEQ ID NO: | TAAGGGTCTC | CT (SEQ | (SEQ ID | (SEQ ID | Tc (SEQ | GGTCT | GTCTC | CCTGC |
| | ID NO: 77) | 120) | GCCCTCATCT | ID NO: | NO: 68) | NO: 206) | ID NO: | G (SEQ | (SEQ | (SEQ |
| | | | TCTTCCCTGC | 67) | | | 249) | ID NO: | ID NO: | ID NO: |
| | | | (SEQ ID NO: | | | | | 292) | 335) | 33) |
| | | | 163) | | | | | | | |
| chr2 | TTCCTCCACC | GCCCTATTGCA | GGGCTATCCA | TTCCTC | GCCCTAT | AGGGAG | AGGGA | GGGCT | TACTC | GCCCT |
| | GAACGTGTCT | AGCCCTCTTAG | GAAAGATAAG | CACCGA | TGCAAG | CAATAG | GCAAT | ATCCA | ACAA | CATCT |
| | AGGGAGCAAT | GGAGCAATAGG | AATACTCACA | ACGTGT | CCCTCTT | GCcg | AGGCta | GAAA | ACGAC | TCTTC |
| | AGGCcg (SEQ | Cta (SEQ ID NO: | AACGACTGCG | CT (SEQ | (SEQ ID | (SEQ ID | (SEQ ID | GATAA | TGCGC | CCTGC |
| | ID NO: 78) | 121) | CAGCCCTCAT | ID NO: | NO: 68) | NO: 207) | NO: 250) | GAA | A (SEQ | (SEQ |
| | | | CTTCTTCCCTG | 67) | | | | (SEQ | ID NO: | ID NO: |
| | | | C (SEQ ID NO: | | | | | ID NO: | 336) | 33) |
| | | | 164) | | | | | 293) | | |
| chr2 | TTCCTCCACC | GCCCTATTGCA | CATAACTGGT | TTCCTC | GCCCTAT | CTGCAG | CTGCA | CATAA | CGTAT | GCCCT |
| | GAACGTGTCT | AGCCCTCTTCTG | GGAGTATTTC | CACCGA | TGCAAG | GGTACA | GGGTA | CTGGT | ATGGC | CATCT |
| | CTGCAGGGTA | CAGGGTACAAg | ACTCGTATAT | ACGTGT | CCCTCTT | AcACg | CAAgA | GGAGT | CGACT | TCTTC |
| | CAAcACg (SEQ | ACa (SEQ ID NO: | GGCCGACTGG | CT (SEQ | (SEQ ID | (SEQ ID | Ca (SEQ | ATTTC | GGAG | CCTGC |
| | ID NO: 79) | 122) | AGGGCCCTCA | ID NO: | NO: 68) | NO: 208) | ID NO: | ACT | G (SEQ | (SEQ |
| | | | TCTTCTTCCCT | 67) | | | 251) | (SEQ | ID NO: | ID NO: |
| | | | GC (SEQ ID NO: | | | | | ID NO: | 337) | 33) |
| | | | 165) | | | | | 294) | | |
| chr19 | TTCCTCCACC | GCCCTATTGCA | CTTCAAGGAA | TTCCTC | GCCCTAT | CGTATC | CGTAT | CTTCA | TAGGG | GCCCT |
| | GAACGTGTCT | AGCCCTCTTCGT | GAAATTCAAC | CACCGA | TGCAAG | TGGGAA | CTGGG | AGGA | TTTGC | CATCT |
| | CGTATCTGGG | ATCTGGGAAGAt | AGGGTAGGGT | ACGTGT | CCCTCTT | GAcGGc | AAGAtG | AGAA | GGCG | TCTTC |
| | AAGAcGGc | GGg (SEQ ID NO: | TTGCGGCGAT | CT (SEQ | (SEQ ID | (SEQ ID | Gg (SEQ | ATTCA | ATAAG | CCTGC |
| | (SEQ ID NO: 80) | 123) | AAGGGCCCTC | ID NO: | NO: 68) | NO: 209) | ID NO: | ACAG | G (SEQ | (SEQ |
| | | | ATCTTCTTCCC | 67) | | | 252) | GG | ID NO: | ID NO: |
| | | | TGC (SEQ ID | | | | | (SEQ | 338) | 33) |
| | | | NO: 166) | | | | | ID NO: | | |
| | | | | | | | | 295) | | |
| chr9 | TTCCTCCACC | GCCCTATTGCA | CATGGATTCA | TTCCTC | GCCCTAT | CCTGTA | CCTGT | CATGG | CCAAG | GCCCT |
| | GAACGTGTCT | AGCCCTCTTCCT | ACACAGCAAA | CACCGA | TGCAAG | ATCCCT | AATCC | ATTCA | TCAAC | CATCT |
| | CCTGTAATCC | GTAATCCCTTGC | CACCAAGTCA | ACGTGT | CCCTCTT | TGCAAT | CTTGC | ACACA | CACCC | TCTTC |
| | CTTGCAATgc | AATaa (SEQ ID | ACCACCCGAG | CT (SEQ | (SEQ ID | gc (SEQ | AATaa | GCAA | GAGA | CCTGC |
| | (SEQ ID NO: 81) | NO: 124) | ACGCCCTCAT | ID NO: | NO: 68) | ID NO: | (SEQ ID | ACA | C (SEQ | (SEQ |
| | | | CTTCTTCCCTG | 67) | | 210) | NO: 253) | (SEQ | ID NO: | ID NO: |
| | | | C (SEQ ID NO: | | | | | ID NO: | 339) | 33) |
| | | | 167) | | | | | 296) | | |
| chr16 | TTCCTCCACC | GCCCTATTGCA | CTCTGACCTC | TTCCTC | GCCCTAT | GGTCTC | GGTCT | CTCTG | ACTTC | GCCCT |
| | GAACGTGTCT | AGCCCTCTTGGT | CTTCACTCTTA | CACCGA | TGCAAG | AGCACG | CAGCA | ACCTC | CCTGG | CATCT |
| | GGTCTCAGCA | CTCAGCACGGTc | CACTTCCCTG | ACGTGT | CCCTCTT | GTtCTg | CGGTcC | CTTCA | CCTTC | TCTTC |
| | CGGTtCTg | CTt (SEQ ID NO: | GCCTTCCTTCT | CT (SEQ | (SEQ ID | (SEQ ID | Tt (SEQ | CTCTT | CTTCT | CCTGC |
| | (SEQ ID NO: 82) | 125) | GCCCTCATCT | ID NO: | NO: 68) | NO: 211) | ID NO: | AC | (SEQ | (SEQ |
| | | | TCTTCCCTGC | 67) | | | 254) | (SEQ | ID NO: | ID NO: |
| | | | (SEQ ID NO: | | | | | ID NO: | 340) | 33) |
| | | | 168) | | | | | 297) | | |
| chr9 | TTCCTCCACC | GCCCTATTGCA | GCTTTCATTTG | TTCCTC | GCCCTAT | GCACCT | GCACC | GCTTT | GCTTG | GCCCT |
| | GAACGTGTCT | AGCCCTCTTGC | TGCTAAACCT | CACCGA | TGCAAG | CCCTAc | TCCCT | CATTT | GGTCC | CATCT |
| | GCACCTCCCT | ACCTCCCTAtCA | CGCTTGGGTC | ACGTGT | CCCTCTT | CACAc | AtCACA | GTGCT | TCTCC | TCTTC |
| | AcCACAc (SEQ | CAt (SEQ ID NO: | CTCTCCTGAA | CT (SEQ | (SEQ ID | (SEQ ID | t (SEQ | AAACC | TGAAC | CCTGC |
| | ID NO: 83) | 126) | CGCCCTCATC | ID NO: | NO: 68) | NO: 212) | ID NO: | TC | (SEQ | (SEQ |
| | | | TTCTTCCCTGC | 67) | | | 255) | (SEQ | ID NO: | ID NO: |
| | | | (SEQ ID NO: | | | | | ID NO: | 341) | 33) |
| | | | 169) | | | | | 298) | | |
| chr3 | TTCCTCCACC | GCCCTATTGCA | CATCCCAGAT | TTCCTC | GCCCTAT | GCCTCT | GCCTC | CATCC | AACGT | GCCCT |
| | GAACGTGTCT | AGCCCTCTTGCC | GCCCTCATAA | CACCGA | TGCAAG | AGCTAG | TAGCT | CAGAT | CCGAA | CATCT |
| | GCCTCTAGCT | TCTAGCTAGAG | CGTCCGAACC | ACGTGT | CCCTCTT | AGAGAA | AGAGA | GCCCT | CCACA | TCTTC |
| | AGAGAGAAGt | AGAAGcg (SEQ | ACAATGCTGC | CT (SEQ | (SEQ ID | Gtc (SEQ | GAAGcg | CAT | ATGCT | CCTGC |
| | c (SEQ ID NO: | ID NO: 127) | CCTCATCTTCT | ID NO: | NO: 68) | ID NO: | (SEQ ID | (SEQ | (SEQ | (SEQ |
| | 84) | | TCCCTGC (SEQ | 67) | | 213) | NO: 256) | ID NO: | ID NO: | ID NO: |
| | | | ID NO: 170) | | | | | 299) | 342) | 33) |
| chr20 | TTCCTCCACC | GCCCTATTGCA | GTAGAAATCC | TTCCTC | GCCCTAT | CTGGCA | CTGGC | GTAGA | CTCCT | GCCCT |
| | GAACGTGTCT | AGCCCTCTTCTG | CAAGGCAATC | CACCGA | TGCAAG | GTCTAG | AGTCT | AATCC | CGCAT | CATCT |
| | CTGGCAGTCT | GCAGTCTAGCCa | AGCTCCTCGC | ACGTGT | CCCTCTT | CCgTTAc | AGCCaT | CAAG | CCAAC | TCTTC |
| | AGCCgTTAc | TTAt (SEQ ID NO: | ATCCAACAGT | CT (SEQ | (SEQ ID | (SEQ ID | TAt | GCAAT | AGTCG | CCTGC |
| | (SEQ ID NO: 85) | 128) | CGGCCCTCAT | ID NO: | NO: 68) | NO: 214) | (SEQ ID | CAG | (SEQ | (SEQ |
| | | | CTTCTTCCCTG | 67) | | | NO: | (SEQ | ID NO: | ID NO: |
| | | | C (SEQ ID NO: | | | | NO: 257) | 300) | 343) | 33) |
| | | | 171) | | | | | | | |
| chrX | TTCCTCCACC | GCCCTATTGCA | GAACAACTAA | TTCCTC | GCCCTAT | TGTCTT | TGTCTT | GAAC | CCACC | GCCCT |
| | GAACGTGTCT | AGCCCTCTTTGT | CTCCACAGAA | CACCGA | TGCAAG | AGAATT | AGAAT | AACTA | GTAGC | CATCT |
| | TGTCTTAGAA | CTTAGAATTTG | CCCCCACCGT | ACGTGT | CCCTCTT | TGGCAA | TTGGC | ACTCC | ACTCC | TCTTC |
| | TTTGGCAACTg | GCAACTaGt | AGCACTCCTT | CT (SEQ | (SEQ ID | CTgGc | AACTaG | ACAG | TTCTT | CCTGC |
| | Gc (SEQ ID NO: | (SEQ ID NO: 129) | CTTGCCCTCA | ID NO: | NO: 68) | (SEQ ID | t (SEQ | AACCC | (SEQ | (SEQ |
| | 86) | | TCTTCTTCCCT | 67) | | NO: 215) | ID NO: | (SEQ | ID NO: | ID NO: |
| | | | GC (SEQ ID NO: | | | | 258) | ID NO: | 344) | 33) |
| | | | 172) | | | | | 301) | | |
| chr7 | TTCCTCCACC | GCCCTATTGCA | GTGCAGAGGA | TTCCTC | GCCCTAT | GCAGGA | GCAGG | GTGCA | CGGA | GCCCT |
| | GAACGTGTCT | AGCCCTCTTGC | CAGGAAGAAC | CACCGA | TGCAAG | AAGCCT | AAAGC | GAGG | GCGTC | CATCT |
| | GCAGGAAAGC | AGGAAAGCCTAt | GGAGCGTCGG | ACGTGT | CCCTCTT | AcTGAA | CTAtTG | ACAG | GGTAG | TCTTC |
| | CTAcTGAAc | TGAAt (SEQ ID | TAGTGTAAAG | CT (SEQ | (SEQ ID | c (SEQ ID | AAt | GAAG | TGTAA | CCTGC |
| | (SEQ ID NO: 87) | NO: 130) | CCCTCATCTTC | ID NO: | NO: 68) | NO: 216) | (SEQ ID | AA | A (SEQ | (SEQ |
| | | | TTCCCTGC | 67) | | | NO: 259) | (SEQ | ID NO: | ID NO: |
| | | | (SEQ ID NO: | | | | | ID NO: | 345) | 33) |
| | | | 173) | | | | | 302) | | |
| chr3 | TTCCTCCACC | GCCCTATTGCA | GGTGCTTCAA | TTCCTC | GCCCTAT | GGGAGC | GGGAG | GGTGC | ACAAC | GCCCT |
| | GAACGTGTCT | AGCCCTCTTGG | GACATACACC | CACCGA | TGCAAG | CAGAGA | CCAGA | TTCAA | TCGAC | CATCT |
| | GGGAGCCAGA | GAGCCAGAGAA | TTAACAACTC | ACGTGT | CCCTCTT | AATgTCc | GAAATt | GACAT | GAACC | TCTTC |
| | GAAATgTCc | ATtTCt (SEQ ID | GACGAACCTA | CT (SEQ | (SEQ ID | (SEQ ID | TCt | ACACC | TACCG | CCTGC |
| | (SEQ ID NO: 88) | NO: 131) | CCGGCCCTCA | ID NO: | NO: 68) | NO: 217) | (SEQ ID | TTA | (SEQ | (SEQ |
| | | | TCTTCTTCCCT | 67) | | | NO: 260) | (SEQ | ID NO: | ID NO: |
| | | | GC (SEQ ID NO: | | | | | ID NO: | 346) | 33) |
| | | | 174) | | | | | 303) | | |
| chr2 | TTCCTCCACC | GCCCTATTGCA | GGAACCTCTG | TTCCTC | GCCCTAT | TGTCTC | TGTCTC | GGAA | TGGCC | GCCCT |
| | GAACGTGTCT | AGCCCTCTTTGT | TGACCTTGGA | CACCGA | TGCAAG | CAGTTC | CAGTT | CCTCT | CATCC | CATCT |
| | TGTCTCCAGT | CTCCAGTTCCAC | TGGCCCATCC | ACGTGT | CCCTCTT | CACTTC | CCACT | GTGAC | TTATG | TCTTC |
| | TCCACTTCATt | TTCATgTAa (SEQ | TTATGTGCTG | CT (SEQ | (SEQ ID | ATtTAg | TCATgT | CTTGG | TGCTG | CCTGC |
| | TAg (SEQ ID | ID NO: 132) | GCCCTCATCT | ID NO: | NO: 68) | (SEQ ID | Aa (SEQ | A (SEQ | (SEQ | (SEQ |
| | NO: 89) | | TCTTCCCTGC | 67) | | NO: 218) | ID NO: | ID NO: | ID NO: | ID NO: |
| | | | (SEQ ID NO: | | | | 261) | 304) | 347) | 33) |
| | | | 175) | | | | | | | |
| chr15 | TTCCTCCACC | GCCCTATTGCA | CCCAGTGGTA | TTCCTC | GCCCTAT | CCCGTT | CCCGT | CCCAG | GGTCG | GCCCT |
| | GAACGTGTCT | AGCCCTCTTCCC | CCTTCTGAAG | CACCGA | TGCAAG | AATTGC | TAATT | TGGTA | TTATT | CATCT |
| | CCCGTTAATT | GTTAATTGCCTA | GTCGTTATTG | ACGTGT | CCCTCTT | CTAcTcg | GCCTAt | CCTTC | GCTCA | TCTTC |
| | GCCTAcTcg | tTta (SEQ ID NO: | CTCAAGCCCG | CT (SEQ | (SEQ ID | (SEQ ID | Tta (SEQ | TGAA | AGCCC | CCTGC |
| | (SEQ ID NO: 90) | 133) | CCCTCATCTTC | ID NO: | NO: 68) | NO: 219) | ID NO: | (SEQ | (SEQ | (SEQ |
| | | | TTCCCTGC | 67) | | | 262) | ID NO: | ID NO: | ID NO: |
| | | | (SEQ ID NO: | | | | | 305) | 348) | 33) |
| | | | 176) | | | | | | | |
| chr15 | TTCCTCCACC | GCCCTATTGCA | CTTCTGTTGCT | TTCCTC | GCCCTAT | CTCGGT | CTCGG | CTTCT | TTGAT | GCCCT |
| | GAACGTGTCT | AGCCCTCTTCTC | TATTTGGGTA | CACCGA | TGCAAG | CCCACT | TCCCA | GTTGC | TCTGG | CATCT |
| | CTCGGTCCCA | GGTCCCACTGGg | ACTTGATTCT | ACGTGT | CCCTCTT | GGaAAg | CTGGg | TTATT | CCCTC | TCTTC |
| | CTGGaAAg | AAa (SEQ ID NO: | GGCCCTCCCA | CT (SEQ | (SEQ ID | (SEQ ID | AAa | TGGGT | CCATC | CCTGC |
| | (SEQ ID NO: 91) | 134) | TCGCCCTCAT | ID NO: | NO: 68) | NO: 220) | (SEQ ID | AAC | (SEQ | (SEQ |
| | | | CTTCTTCCCTG | 67) | | | NO: 263) | (SEQ | ID NO: | ID NO: |
| | | | C (SEQ ID NO: | | | | | ID NO: | 349) | 33) |
| | | | 177) | | | | | 306) | | |
| chr2 | TTCCTCCACC | GCCCTATTGCA | CCCACTGGAT | TTCCTC | GCCCTAT | ACACCC | ACACC | CCCAC | CTCAC | GCCCT |
| | GAACGTGTCT | AGCCCTCTTAC | GCCTCCCTCA | CACCGA | TGCAAG | ATGATT | CATGA | TGGAT | GCCGG | CATCT |
| | ACACCCATGA | ACCCATGATTC | CGCCGGCTAT | ACGTGT | CCCTCTT | CAGTTA | TTCAG | GCCTC | CTATT | TCTTC |
| | TTCAGTTACtg | AGTTACca (SEQ | TTAGGTGCCC | CT (SEQ | (SEQ ID | Ctg (SEQ | TTACca | C (SEQ | TAGGT | CCTGC |
| | (SEQ ID NO: 92) | ID NO: 135) | TCATCTTCTTC | ID NO: | NO: 68) | ID NO: | (SEQ ID | ID NO: | (SEQ | (SEQ |
| | | | CCTGC (SEQ ID | 67) | | 221) | NO: 264) | 307) | ID NO: | ID NO: |
| | | | NO: 178) | | | | | | 350) | 33) |
| chr9 | TTCCTCCACC | GCCCTATTGCA | CGGAGAGACG | TTCCTC | GCCCTAT | GCTAGT | GCTAG | CGGA | AGTCT | GCCCT |
| | GAACGTGTCT | AGCCCTCTTGCT | CATCTGAAAG | CACCGA | TGCAAG | ATGAAC | TATGA | GAGA | GGGTA | CATCT |
| | GCTAGTATGA | AGTATGAACAT | TCTGGGTAGG | ACGTGT | CCCTCTT | ATCACA | ACATC | CGCAT | GGTGG | TCTTC |
| | ACATCACAgGc | CACAaGt (SEQ ID | TGGAGGACGC | CT (SEQ | (SEQ ID | gGc (SEQ | ACAaGt | CTGAA | AGGA | CCTGC |
| | (SEQ ID NO: 93) | NO: 136) | CCTCATCTTCT | ID NO: | NO: 68) | ID NO: | (SEQ ID | (SEQ | C (SEQ | (SEQ |
| | | | TCCCTGC (SEQ | 67) | | 222) | NO: 265) | ID NO: | ID NO: | ID NO: |
| | | | ID NO: 179) | | | | | 308) | 351) | 33) |
| chr7 | TTCCTCCACC | GCCCTATTGCA | CAGGATTTCC | TTCCTC | GCCCTAT | ACAAAT | ACAAA | CAGG | CGACT | GCCCT |
| | GAACGTGTCT | AGCCCTCTTAC | AGCTTACAGG | CACCGA | TGCAAG | GAGTAA | TGAGT | ATTTC | GAGCC | CATCT |
| | ACAAATGAGT | AAATGAGTAAG | GCGACTGAGC | ACGTGT | CCCTCTT | GAAGCG | AAGAA | CAGCT | ACATC | TCTTC |
| | AAGAAGCGAG | AAGCGAGTta | CACATCCAAC | CT (SEQ | (SEQ ID | AGTcg | GCGAG | TACAG | CAACT | CCTGC |
| | Tcg (SEQ ID | (SEQ ID NO: 137) | TGCCCTCATC | ID NO: | NO: 68) | (SEQ ID | Tta (SEQ | GG | (SEQ | (SEQ |
| | NO: 94) | | TTCTTCCCTGC | 67) | | NO: 223) | ID NO: | (SEQ | ID NO: | ID NO: |
| | | | (SEQ ID NO: | | | | 266) | ID NO: | 352) | 33) |
| | | | 180) | | | | | 309) | | |
| chr20 | TTCCTCCACC | GCCCTATTGCA | CTTGCAAGAT | TTCCTC | GCCCTAT | GATAAG | GATAA | CTTGC | GAGCC | GCCCT |
| | GAACGTGTCT | AGCCCTCTTGAT | GTGCCTCTTA | CACCGA | TGCAAG | GGTTGC | GGGTT | AAGAT | TCAGC | CATCT |
| | GATAAGGGTT | AAGGGTTGCTC | GAGCCTCAGC | ACGTGT | CCCTCTT | TCTgCg | GCTCTa | GTGCC | CGGA | TCTTC |
| | GCTCTgCg | TaCa (SEQ ID NO: | CGGAATTGAA | CT (SEQ | (SEQ ID | (SEQ ID | Ca (SEQ | TCTTA | ATTGA | CCTGC |
| | (SEQ ID NO: 95) | 138) | GCCCTCATCT | ID NO: | NO: 68) | NO: 224) | ID NO: | (SEQ | A (SEQ | (SEQ |
| | | | TCTTCCCTGC | 67) | | | 267) | ID NO: | ID NO: | ID NO: |
| | | | (SEQ ID NO: | | | | | 310) | 353) | 33) |
| | | | 181) | | | | | | | |
| chr20 | TTCCTCCACC | GCCCTATTGCA | GGGTGGTTTC | TTCCTC | GCCCTAT | CCATGC | CCATG | GGGTG | TTGCC | GCCCT |
| | GAACGTGTCT | AGCCCTCTTCCA | TCTAAACACA | CACCGA | TGCAAG | ACCAGC | CACCA | GTTTC | ATTCT | CATCT |
| | CCATGCACCA | TGCACCAGCTA | AATTGCCATT | ACGTGT | CCCTCTT | TACcc | GCTACt | TCTAA | GCACC | TCTTC |
| | GCTACcc (SEQ | Cta (SEQ ID NO: | CTGCACCAAT | CT (SEQ | (SEQ ID | (SEQ ID | a (SEQ | ACACA | AATGC | CCTGC |
| | ID NO: 96) | 139) | GCGCCCTCAT | ID NO: | NO: 68) | NO: 225) | ID NO: | AA | (SEQ | (SEQ |
| | | | CTTCTTCCCTG | 67) | | | 268) | (SEQ | ID NO: | ID NO: |
| | | | C (SEQ ID NO: | | | | | ID NO: | 354) | 33) |
| | | | 182) | | | | | 311) | | |
| chr1 | TTCCTCCACC | GCCCTATTGCA | GCAGGGTATT | TTCCTC | GCCCTAT | AACTGT | AACTG | GCAG | TATTG | GCCCT |
| | GAACGTGTCT | AGCCCTCTTAA | GAGAGAAGG | CACCGA | TGCAAG | ACCCTA | TACCC | GGTAT | GTGTT | CATCT |
| | AACTGTACCC | CTGTACCCTACT | ATCTATTGGT | ACGTGT | CCCTCTT | CTCCCA | TACTC | TGAGA | CGCGG | TCTTC |
| | TACTCCCAgc | CCCAat (SEQ ID | GTTCGCGGCT | CT (SEQ | (SEQ ID | gc (SEQ | CCAat | GAAG | CTGAT | CCTGC |
| | (SEQ ID NO: 97) | NO: 140) | GATGCCCTCA | ID NO: | NO: 68) | ID NO: | (SEQ ID | GATC | (SEQ | (SEQ |
| | | | TCTTCTTCCCT | 67) | | 226) | NO: 269) | (SEQ | ID NO: | ID NO: |
| | | | GC (SEQ ID NO: | | | | | ID NO: | 355) | 33) |
| | | | 183) | | | | | 312) | | |
| chr2 | TTCCTCCACC | GCCCTATTGCA | GTGCACATTT | TTCCTC | GCCCTAT | AGGACC | AGGAC | GTGCA | ATGGG | GCCCT |
| | GAACGTGTCT | AGCCCTCTTAG | CTTGATGAAG | CACCGA | TGCAAG | AAGGGA | CAAGG | CATTT | CGTAA | CATCT |
| | AGGACCAAGG | GACCAAGGGAC | GGATGGGCGT | ACGTGT | CCCTCTT | CCAGTTt | GACCA | CTTGA | CAGG | TCTTC |
| | GACCAGTTtAg | CAGTTcAc (SEQ | AACAGGAGGA | CT (SEQ | (SEQ ID | Ag (SEQ | GTTcAc | TGAAG | AGGA | CCTGC |
| | (SEQ ID NO: 98) | ID NO: 141) | CTGCCCTCAT | ID NO: | NO: 68) | ID NO: | (SEQ ID | GG | CT | (SEQ |
| | | | CTTCTTCCCTG | 67) | | 227) | NO: 270) | (SEQ | (SEQ | ID NO: |
| | | | C (SEQ ID NO: | | | | | ID NO: | ID NO: | 33) |
| | | | 184) | | | | | 313) | 356) | |
| chr7 | TTCCTCCACC | GCCCTATTGCA | GAGCAATGCC | TTCCTC | GCCCTAT | AGAGTT | AGAGT | GAGC | GGAAT | GCCCT |
| | GAACGTGTCT | AGCCCTCTTAG | TGTTTCATGA | CACCGA | TGCAAG | CCTCCA | TCCTCC | AATGC | GGCCT | CATCT |
| | AGAGTTCCTC | AGTTCCTCCAA | GAGGAATGGC | ACGTGT | CCCTCTT | AGAAAT | AAGAA | CTGTT | ACCTG | TCTTC |
| | CAAGAAATTG | GAAATTGta | CTACCTGCAT | CT (SEQ | (SEQ ID | TGcg | ATTGta | TCATG | CATCA | CCTGC |
| | cg (SEQ ID NO: | (SEQ ID NO: 142) | CAGCCCTCAT | ID NO: | NO: 68) | (SEQ ID | (SEQ ID | AGA | (SEQ | (SEQ |
| | 99) | | CTTCTTCCCTG | 67) | | NO: 228) | NO: 271) | (SEQ | ID NO: | ID NO: |
| | | | C (SEQ ID NO: | | | | | ID NO: | 357) | 33) |
| | | | 185) | | | | | 314) | | |
| chr5 | TTCCTCCACC | GCCCTATTGCA | GTTAACATTA | TTCCTC | GCCCTAT | ACATTA | ACATT | GTTAA | CCCGT | GCCCT |
| | GAACGTGTCT | AGCCCTCTTAC | TACAGCATGG | CACCGA | TGCAAG | TACAGC | ATACA | CATTA | TGTTG | CATCT |
| | ACATTATACA | ATTATACAGCA | TGGCCCCGTT | ACGTGT | CCCTCTT | ATGCTG | GCATG | TACAG | TCATC | TCTTC |
| | GCATGCTGGc | TGCTGGtTAga | GTTGTCATCG | CT (SEQ | (SEQ ID | GcTAtc | CTGGtT | CATGG | GCATC | CCTGC |
| | TAtc (SEQ ID | (SEQ ID NO: 143) | CATCGCCCTC | ID NO: | NO: 68) | (SEQ ID | Aga | TGGC | (SEQ | (SEQ |
| | NO: 100) | | ATCTTCTTCCC | 67) | | NO: 229) | (SEQ ID | (SEQ | ID NO: | ID NO: |
| | | | TGC (SEQ ID | | | | NO: 272) | ID NO: | 358) | 33) |
| | | | NO: 186) | | | | | 315) | | |
| chr2 | TTCCTCCACC | GCCCTATTGCA | GCAGAACATG | TTCCTC | GCCCTAT | GAGGAA | GAGGA | GCAG | GTTCG | GCCCT |
| | GAACGTGTCT | AGCCCTCTTGA | TCCTGAAGCG | CACCGA | TGCAAG | GAAAGT | AGAAA | AACAT | ATGCG | CATCT |
| | GAGGAAGAA | GGAAGAAAGTG | TTCGATGCGT | ACGTGT | CCCTCTT | GAGgTT | GTGAG | GTCCT | TCCCA | TCTTC |
| | AGTGAGgTTT | AGaTTTGt (SEQ | CCCATGAGTG | CT (SEQ | (SEQ ID | TGc (SEQ | aTTTGt | GAAG | TGAGT | CCTGC |
| | Gc (SEQ ID NO: | ID NO: 144) | CCCTCATCTTC | ID NO: | NO: 68) | ID NO: | (SEQ ID | C (SEQ | (SEQ | (SEQ |
| | 101) | | TTCCCTGC | 67) | | 230) | NO: 273) | ID NO: | ID NO: | ID NO: |
| | | | (SEQ ID NO: | | | | | 316) | 359) | 33) |
| | | | 187) | | | | | | | |
| chr15 | TTCCTCCACC | GCCCTATTGCA | CAGCTTGTTC | TTCCTC | GCCCTAT | CTGAAT | CTGAA | CAGCT | CAACC | GCCCT |
| | GAACGTGTCT | AGCCCTCTTCTG | CCAAACCCAT | CACCGA | TGCAAG | TATGTG | TTATGT | TGTTC | CGCGT | CATCT |
| | CTGAATTATG | AATTATGTGCTT | CAACCCGCGT | ACGTGT | CCCTCTT | CTTACC | GCTTA | CCAAA | AGATG | TCTTC |
| | TGCTTACCAaG | ACCAgGAGt | AGATGTTCCT | CT (SEQ | (SEQ ID | AaGAGc | CCAgG | CCCAT | TTCCT | CCTGC |
| | AGc (SEQ ID | (SEQ ID NO: 145) | GCCCTCATCT | ID NO: | NO: 68) | (SEQ ID | AGt | (SEQ | (SEQ | (SEQ |
| | NO: 102) | | TCTTCCCTGC | 67) | | NO: 231) | (SEQ ID | ID NO: | ID NO: | ID NO: |
| | | | (SEQ ID NO: | | | | NO: 274) | 317) | 360) | 33) |
| | | | 188) | | | | | | | |
| chr9 | TTCCTCCACC | GCCCTATTGCA | CAAAGTGTGG | TTCCTC | GCCCTAT | TGGGTT | TGGGT | CAAA | GCCAG | GCCCT |
| | GAACGTGTCT | AGCCCTCTTTGG | AAGTTGCTTC | CACCGA | TGCAAG | CTGATA | TCTGA | GTGTG | CTCAA | CATCT |
| | TGGGTTCTGA | GTTCTGATAAC | CGCCAGCTCA | ACGTGT | CCCTCTT | ACCTTA | TAACC | GAAGT | GAGTG | TCTTC |
| | TAACCTTATC | CTTATCAAct | AGAGTGTAGC | CT (SEQ | (SEQ ID | TCAAgc | TTATC | TGCTT | TAGCC | CCTGC |
| | AAgc (SEQ ID | (SEQ ID NO: 146) | CGCCCTCATC | ID NO: | NO: 68) | (SEQ ID | AAct | CC | (SEQ | (SEQ |
| | NO: 103) | | TTCTTCCCTGC | 67) | | NO: 232) | (SEQ ID | (SEQ | ID NO: | ID NO: |
| | | | (SEQ ID NO: | | | | NO: 275) | ID NO: | 361) | 33) |
| | | | 189) | | | | | 318) | | |
| chr2 | TTCCTCCACC | GCCCTATTGCA | GGTCGACTTT | TTCCTC | GCCCTAT | GGTTAG | GGTTA | GGTCG | TTCTT | GCCCT |
| | GAACGTGTCT | AGCCCTCTTGGT | GTCCATCCTT | CACCGA | TGCAAG | TCAAAC | GTCAA | ACTTT | GATCC | CATCT |
| | GGTTAGTCAA | TAGTCAAACAT | CTTGATCCTG | ACGTGT | CCCTCTT | ATGcTGc | ACATGt | GTCCA | TGCGC | TCTTC |
| | ACATGcTGc | GtTGt (SEQ ID | CGCGATGTGC | CT (SEQ | (SEQ ID | (SEQ ID | TGt | TCC | GATGT | CCTGC |
| | (SEQ ID NO: | NO: 147) | CCTCATCTTCT | ID NO: | NO: 68) | NO: 233) | (SEQ ID | (SEQ | (SEQ | (SEQ |
| | 104) | | TCCCTGC (SEQ | 67) | | | NO: 276) | ID NO: | ID NO: | ID NO: |
| | | | ID NO: 190) | | | | | 319) | 362) | 33) |
| chr17 | TTCCTCCACC | GCCCTATTGCA | CTCTGTTGCCT | TTCCTC | GCCCTAT | GACACT | GACAC | CTCTG | ATCGC | GCCCT |
| | GAACGTGTCT | AGCCCTCTTGA | GTGGACTCAT | CACCGA | TGCAAG | GGCAGA | TGGCA | TTGCC | AGGC | CATCT |
| | GACACTGGCA | CACTGGCAGAA | CGCAGGCGTT | ACGTGT | CCCTCTT | ATCAAA | GAATC | TGTGG | GTTCC | TCTTC |
| | GAATCAAAtC | TCAAAcCAa | CCCTATACGC | CT (SEQ | (SEQ ID | tCAc | AAAcC | ACTC | CTATA | CCTGC |
| | Ac (SEQ ID NO: | (SEQ ID NO: 148) | CCTCATCTTCT | ID NO: | NO: 68) | (SEQ ID | Aa (SEQ | (SEQ | C (SEQ | (SEQ |
| | 105) | | TCCCTGC (SEQ | 67) | | NO: 234) | ID NO: | ID NO: | ID NO: | ID NO: |
| | | | ID NO: 191) | | | | 277) | 320) | 363) | 33) |
| chr6 | TTCCTCCACC | GCCCTATTGCA | CTAACTAGAA | TTCCTC | GCCCTAT | AGAGTT | AGAGT | CTAAC | TATTG | GCCCT |
| | GAACGTGTCT | AGCCCTCTTAG | TTAGTCTGCC | CACCGA | TGCAAG | ACACCT | TACAC | TAGAA | GACCT | CATCT |
| | AGAGTTACAC | AGTTACACCTTT | TGCCTATTGG | ACGTGT | CCCTCTT | TTAGCT | CTTTA | TTAGT | CCGAC | TCTTC |
| | CTTTAGCTAA | AGCTAACtAg | ACCTCCGACC | CT (SEQ | (SEQ ID | AACcAc | GCTAA | CTGCC | CACGA | CCTGC |
| | CcAc (SEQ ID | (SEQ ID NO: 149) | ACGAGCCCTC | ID NO: | NO: 68) | (SEQ ID | CtAg | TGCC | (SEQ | (SEQ |
| | NO: 106) | | ATCTTCTTCCC | 67) | | NO: 235) | (SEQ ID | (SEQ | ID NO: | ID NO: |
| | | | TGC (SEQ ID | | | | NO: 278) | ID NO: | 364) | 33) |
| | | | NO: 192) | | | | | 321) | | |
| chr7 | TTCCTCCACC | GCCCTATTGCA | GTGAGCCATA | TTCCTC | GCCCTAT | CCAGGA | CCAGG | GTGAG | AGCCA | GCCCT |
| | GAACGTGTCT | AGCCCTCTTCCA | ATCGTGTCAA | CACCGA | TGCAAG | GTTCAA | AGTTC | CCATA | CCATT | CATCT |
| | CCAGGAGTTC | GGAGTTCAAGg | GCCACCATTT | ACGTGT | CCCTCTT | GaAGCg | AAGgA | ATCGT | TAGAT | TCTTC |
| | AAGaAGCg | AGCa (SEQ ID | AGATCCGCGG | CT (SEQ | (SEQ ID | (SEQ ID | GCa | GTCA | CCGCG | CCTGC |
| | (SEQ ID NO: | NO: 150) | CCCTCATCTTC | ID NO: | NO: 68) | NO: 236) | (SEQ ID | (SEQ | (SEQ | (SEQ |
| | 107) | | TTCCCTGC | 67) | | | NO: 279) | ID NO: | ID NO: | ID NO: |
| | | | (SEQ ID NO: | | | | | 322) | 365) | 33) |
| | | | 193) | | | | | | | |
| chr4 | TTCCTCCACC | GCCCTATTGCA | GAGAATTAAT | TTCCTC | GCCCTAT | ACCACT | ACCAC | GAGA | GACCA | GCCCT |
| | GAACGTGTCT | AGCCCTCTTACC | GCTCCCTCTC | CACCGA | TGCAAG | CCTTTC | TCCTTT | ATTAA | GTAGA | CATCT |
| | ACCACTCCTT | ACTCCTTTCTCC | CTGGACCAGT | ACGTGT | CCCTCTT | TCCCaTC | CTCCCg | TGCTC | AGTCT | TCTTC |
| | TCTCCCaTCTc | CgTCTt (SEQ ID | AGAAGTCTGC | CT (SEQ | (SEQ ID | Tc (SEQ | TCTt | CCTCT | GCCCG | CCTGC |
| | (SEQ ID NO: | NO: 151) | CCGGCCCTCA | ID NO: | NO: 68) | ID NO: | (SEQ ID | CCTG | (SEQ | (SEQ |
| | 108) | | TCTTCTTCCCT | 67) | | 237) | NO: 280) | (SEQ | ID NO: | ID NO: |
| | | | GC (SEQ ID NO: | | | | | ID NO: | 366) | 33) |
| | | | 194) | | | | | 323) | | |
| chr2 | TTCCTCCACC | GCCCTATTGCA | GTGGTCTGCT | TTCCTC | GCCCTAT | GTCTTA | GTCTT | GTGGT | TTTCA | GCCCT |
| | GAACGTGTCT | AGCCCTCTTGTC | GTTGACCAAT | CACCGA | TGCAAG | TGGGAC | ATGGG | CTGCT | GAATG | CATCT |
| | GTCTTATGGG | TTATGGGACAA | TTCAGAATGG | ACGTGT | CCCTCTT | AATGGT | ACAAT | GTTGA | GCCGA | TCTTC |
| | ACAATGGTtG | TGGTcGATAt | CCGAGCTGTG | CT (SEQ | (SEQ ID | tGATAg | GGTcG | CCAA | GCTGT | CCTGC |
| | ATAg (SEQ ID | (SEQ ID NO: 152) | CCCTCATCTTC | ID NO: | NO: 68) | (SEQ ID | ATAt | (SEQ | (SEQ | (SEQ |
| | NO: 109) | | TTCCCTGC | 67) | | NO: 238) | (SEQ ID | ID NO: | ID NO: | ID NO: |
| | | | (SEQ ID NO: | | | | NO: 281) | 324) | 367) | 33) |
| | | | 195) | | | | | | | |
| chr17 | TTCCTCCACC | GCCCTATTGCA | GGTTGCAACT | TTCCTC | GCCCTAT | CTACCC | CTACC | GGTTG | AGGTG | GCCCT |
| | GAACGTGTCT | AGCCCTCTTCTA | GCTGATCTAT | CACCGA | TGCAAG | TCAACC | CTCAA | CAACT | ACCTT | CATCT |
| | CTACCCTCAA | CCCTCAACCCTC | AGGTGACCTT | ACGTGT | CCCTCTT | CTCgTc | CCCTCa | GCTGA | CTTGT | TCTTC |
| | CCCTCgTc | aTt (SEQ ID NO: | CTTGTACGCC | CT (SEQ | (SEQ ID | (SEQ ID | Tt (SEQ | TCTAT | ACGCC | CCTGC |
| | (SEQ ID NO: | 153) | GCCCTCATCT | ID NO: | NO: 68) | NO: 239) | ID NO: | (SEQ | (SEQ | (SEQ |
| | 110) | | TCTTCCCTGC | 67) | | | 282) | ID NO: | ID NO: | ID NO: |
| | | | (SEQ ID NO: | | | | | 325) | 368) | 33) |
| | | | 196) | | | | | | | |
| chr7 | TTCCTCCACC | GCCCTATTGCA | CTTTCCCAGT | TTCCTC | GCCCTAT | CCAAGA | CCAAG | CTTTC | GGCGC | GCCCT |
| | GAACGTGTCT | AGCCCTCTTCCA | CAAGGCAGGG | CACCGA | TGCAAG | CTGATC | ACTGA | CCAGT | GTCCT | CATCT |
| | CCAAGACTGA | AGACTGATCAT | CGCGTCCTTA | ACGTGT | CCCTCTT | ATGCcg | TCATG | CAAG | TATTT | TCTTC |
| | TCATGCcg | GCta (SEQ ID NO: | TTTCCATCGC | CT (SEQ | (SEQ ID | (SEQ ID | Cta (SEQ | GCAG | CCATC | CCTGC |
| | (SEQ ID NO: | 154) | CCTCATCTTCT | ID NO: | NO: 68) | NO: 240) | ID NO: | (SEQ | (SEQ | (SEQ |
| | 111) | | TCCCTGC (SEQ | 67) | | | 283) | ID NO: | ID NO: | ID NO: |
| | | | ID NO: 197) | | | | | 326) | 369) | 33) |

### EXAMPLES

The following protocol describes the processing of up to 24 cell-free DNA samples through hybridization-ligation, purification, amplification, microarray target preparation, microarray hybridization and microarray washing.

### Example 1

The following materials were prepared or obtained: Cell-free DNA (cfDNA) in a volume of 20 L water; Probe Mix: mixture of all Tagging and Labeling probe oligonucleotides at a concentration of 2 nM each; Taq Ligase (40 U/μL); Magnetic Beads: MyOne Streptavidin C1 Dynabeads; Bead Binding and Washing Buffer, 1× and 2× concentrations; Forward amplification primer, 5′ phosphate modified; Reverse amplification primer, labeled; AmpliTaq Gold Enzyme (5 U/μL); dNTP Mix; Lambda Exonuclease (5 U/μL); Hybridization Buffer, 1.25×; Hybridization control oligonucleotides; Microarray Wash Buffer A; Microarray Wash Buffer B; Microarray Wash Buffer C

Hybridization-Ligation Reaction:

The cfDNA samples (20 μL) were added to wells A1-H3 of a 96-well reaction plate. The following reagents were added to each cfDNA sample for a total reaction volume of 50 μL, and mixed by pipetting up and down 5-8 times.

| Component | Volume |
|---|---|
| H₂O | 19.33 μL |
| Probe Mix | 5 μL |
| 10X Taq Ligase Buffer | 5 μL |
| Taq Ligase | 0.67 μL |

The plate was placed in a thermal cycler and ligate using the following cycling profile: (i) 95° C. for 5 minutes; (ii) 95° C. for 30 seconds; (iii) 45° C. for 25 minutes; (iv) Repeat steps b to c 4 times; and (v) 4° C. hold.

Hybridization-Ligation Product Purification:

Wash Dynabeads: a vial of Dynabeads was vortexted at highest setting for 30 seconds. 260 μL beads were transferred to a 1.5 mL tube. 900 μL of 2× Bead Binding and Washing Buffer and mix beads were mixed by pipetting up and down 5-8 times. The tube was placed on a magnetic stand for 1 min, and the supernatant was discarded. The tube from the magnetic stand was removed and resuspended the washed magnetic beads in 900 μL of 2× Bead Binding and Washing Buffer by pipetting up and down 5-8 times. The tube was placed on the magnetic stand for 1 min and discard the supernatant. The tube was removed from the magnetic stand and add 1,230 μL of 2× Bead Binding and Washing Buffer. The beads were resuspended by pipetting up and down 5-8 times.

Immobilize HL Products: 50 μL of washed beads was transferred to each hybridization-ligation reaction product in the 96-well reaction plate and mix by pipetting up and down 8 times, was incubated for 15 min at room temperature, mixed on a plate magnet twice during the incubation time. The beads were separated with on a plate magnet for 3 min and then remove and discard the supernatant. The plate was removed from the plate magnet, 200 μL 1× Bead Binding and Washing Buffer were added, and the beads were resuspended by pipetting up and down 5-8 times. The plate was placed on the plate magnet for 1 min, and the supernatant was discarded. The plate was removed from the plate magnet, 180 μL 1×SSC was added, and the beads were resuspended by pipetting up and down 5-8 times. The plate was placed on the plate magnet for 1 min, and the supernatant was discarded.

Purify Hyb-Ligation Products: 50 μL of freshly prepared 0.15 M NaOH was added to each well and, the beads were resuspended by pipetting up and down 5-8 times, and incubated at room temperature for 10 minutes. The plate was placed on the plate magnet for 2 minutes and then was removed, and the supernatant was discarded. The plate was removed from the plate magnet, 200 μL of freshly prepared 0.1 M NaOH was added, and the beads were resuspended by pipetting up and down 5-8 times. The plate was placed on the plate magnet for 1 min, and the supernatant was discarded. The plate was removed from the plate magnet, and 180 μL 0.1 M NaOH was added, and the beads were resuspended by pipetting up and down 5-8 times. The plate was placed on the plate magnet for 1 min, and the supernatant was discarded. The plate was removed from the plate magnet, 200 μL of 1× Binding and Wash Buffer were added, and the beads were resuspended by pipetting up and down 5-8 times. Place the plate on the plate magnet for 1 min and discard the supernatant. Remove the plate from the plate magnet, add 180 μL TE, and the beads were resuspended by pipetting up and down 5-8 times. The plate was placed on the plate magnet for 1 min, and the supernatant was discarded. 20 μL water was added to each well and the beads were resuspended by pipetting up and down 5-8 times. The plate was sealed and store at 4° C. until used in subsequent steps.

Amplification:

The following reagents were added to each hybridization-ligation reaction product in the 96-well reaction plate for a total reaction volume of 50 μL.

| Component | Volume |
|---|---|
| H2O | 17.25 μL |
| Forward Primer, 10 μM | 2.5 μL |
| Reverse Primer, 10 μM | 2.5 μL |
| 4 mM dNTP Mix (L/N 052114) | 2.5 μL |
| 10X AmpliTaq Gold Buffer | 5 μL |
| AmpliTaq Gold Enzyme | 0.25 μL |

The plate was placed in a thermal cycler, and the probes were ligated using the following cycling profile: (i) 95° C. for 5 minutes; (ii) 95° C. for 30 seconds; (iii) 45° C. for 25 minutes; (iv) Repeat steps b to c 4 times; and (v) 4° C. hold.

Hybridization-ligation Product Purification: the reagents were mixed by pipetting up and down 5-8 times. The plate was placed in a thermal cycler, and the probes were amplified using the following cycling profile: (i) 95° C. for 5 minutes; (ii) 95° C. for 30 seconds; (iii) 54° C. for 30 seconds; (iv) 72° C. for 60 seconds, (v) Repeat steps b to d 29 times; (vi) 72° C. for 5 minutes; (vii) Repeat steps b to c 4 times; and (v) 4° C. hold.

Microarray Target Preparation

Single strand digestion: the following reagents were added to each amplified reaction product in the 96-well reaction plate for a total reaction volume of 60 μL.

| Component | Volume |
|---|---|
| H₂O | 3 μL |
| 10X Lambda Exonuclease Buffer | 6 μL |
| Lambda Exonuclease Enzyme | 1 μL |

The reagents were mixed by pipetting up and down 5-8 times. The plate was placed in a thermal cycler, and the probes were digested using the following cycling profile: (i) 37° C. for 60 minutes; (ii) 80° C. for 30 minutes; (iii) 4° C. hold. The plate was placed in Speed-vac and dry down samples using medium heat setting for about 60 minutes or until all liquid has evaporated. Samples were stored at 4° C. in the dark until used in subsequent steps.

Microarray Hybridization:

the following reagents were added to each dried Microarray Target in the 96-well reaction plate for a total reaction volume of 20 μL.

| Component | Volume |
|---|---|
| H₂O | 3 μL |
| 1.25X Hybridization Buffer | 16 μL |
| Hybridization control oligonucleotides | 1 μL |

The reagents were mixed by pipetting up and down 10-20 times to be resuspended and were spun briefly to bring contents to the bottoms of the plate wells. The plate was placed in a thermal cycler, and the probes were denatured using the following cycling profile: (i) 70° C. for 3 minutes; (ii) 42° C. hold. The barcode of the microarray to be used was recorded for each sample in the Tracking Sheet. A hybridization chamber containing a Lifter Slip for each microarray to be processed is prepared. For each sample, 15 μL of Microarray Target was added to the center of a Lifter Slip in a hybridization chamber, and the appropriate microarray was immediately placed onto the target fluid by placing the top edge down onto the lifter slip and slowly letting it fall down flat. The hybridization chambers were closed and incubated them at 42° C. for 60 minutes. The hybridization chambers were opened, and each microarray was removed from the Lifter Slips and placed into a rack immersed in Microarray Wash Buffer A. Once all the microarrays were in the rack, the rack was stirred at 650 rpm for 5 minutes. The rack of microarrays was removed from Microarray Wash Buffer A, excess liquid on a clean room wipe was tapped off, and the rack were quickly placed into Microarray Wash Buffer B. The rack was stirred at 650 rpm for 5 minutes. The rack of microarrays was removed from Microarray Wash Buffer B, excess liquid was tapped off on a clean room wipe, and the rack was quickly placed into Microarray Wash Buffer C. The rack was stirred at 650 rpm for 5 minutes. Immediately upon completion of the 5 minute wash in Microarray Wash Buffer C, the rack of microarrays was slowly removed from the buffer. This took 5-10 seconds to maximize the sheeting of the wash buffer from the cover slip surface. Excess liquid was tapped off on a clean room wipe. A vacuum aspirator was used to remove any remaining buffer droplets present on either surface of each microarray. The microarrays were stored in a slide rack under nitrogen and in the dark until the microarrays were analyzed.

### Example 2

The following materials were prepared or obtained: Cell-free DNA (cfDNA) in a volume of 20 μL water; Probe Mix: mixture of all Tagging and Labeling probe oligonucleotides at a concentration of 50 nM each; Taq Ligase (40 U/μL); 10× Taq Ligase Buffer; Spermidine (1 M); EDTA (0.5 M); Magnetic Beads (MyOne Streptavidin C1 Dynabeads); Bead Binding and Washing Buffer (1× and 2× concentrations); Forward amplification primer; Reverse amplification primer A, 5′ end-labeled with dye A; Reverse amplification primer B, 5′ end-labeled with dye B; AmpliTaq Gold Enzyme (5 U/μL); 10× AmpliTaq Buffer I; dNTP Mix; Exonuclease I; Exonuclease I 10× Reaction Buffer; Hybridization Buffer, 1.25×; Hybridization control oligonucleotides; Microarray Wash Buffer A; Microarray Wash Buffer B; Microarray Wash Buffer C.

Hybridization-Ligation Reaction:

The cfDNA samples (20 μL) were added to wells A3-H3 of a 96-well reaction plate. The following reagents were added to each cfDNA sample for a total reaction volume of 50 μL, and mixed by pipetting up and down 5-8 times.

| Component | Volume |
|---|---|
| H₂O | 22.3 μL |
| Probe Mix | 2.5 μL |
| 10X Taq Ligase Buffer | 5 μL |
| Spermidine (1M) | 0.2 μL |

An Enzyme Master Mix was prepared by mixing the following reagents in the order indicated in a 1.5 mL tube and was mixed by pipetting and spin briefly to bring contents to the bottom of the tube.

| Component | Volume (30X) |
|---|---|
| H₂O | 250 μL |
| 10X Taq Ligase Buffer | 30 μL |
| Taq Ligase | 20 μL |

After mixing, 35 μL of the Mix was transferred to wells A5-H5 of the reaction plate. The plate was placed in a thermal cycler and processed using the following cycling profile: (i) 95° C. for 1 minutes; (ii) 95° C. for 30 seconds; (iii) pause at 60° C. (during this pause, 10 μL of Enzyme Master Mix from wells A5-H5 was added to each of wells A1-H3 and mixed); (iv) 60° C. for 5 minutes; (v) 95° C. for 30 seconds; (vi) 60° C. for 5 minutes; (vii) Repeat steps (ii) and (iii) 3 times each; (viii) 60° C. HOLD. Within 1 minute of initiation of 60° C. HOLD step, 5 μL 0.5 M EDTA was added to each reaction before it is removed from the thermal cycler and mixed by pipetting up and down 5-8 times. The plate was sealed before removal from the thermal cycler. The plate was removed, vortexted to mix and centrifuged briefly to bring contents to the well bottoms. The plate was stored at 4° C. until used in subsequent steps.

Hybridization-Ligation Product Purification:

Wash Dynabeads: a vial of Dynabeads was vortexted at highest setting for 30 seconds. 260 μL beads were transferred to a 1.5 mL tube. 900 μL of 2× Bead Binding and Washing Buffer and mix beads were mixed by pipetting up and down 5-8 times. The tube was placed on a magnetic stand for 1 min, and the supernatant was discarded. The tube from the magnetic stand was removed and resuspended the washed magnetic beads in 900 μL of 2× Bead Binding and Washing Buffer by pipetting up and down 5-8 times. The tube was placed on the magnetic stand for 1 min and discard the supernatant. The tube was removed from the magnetic stand and add 1,430 μL of 2× Bead Binding and Washing Buffer. The beads were resuspended by pipetting up and down 5-8 times.

Immobilize HL Products: 55 μL of washed beads was transferred to each hybridization-ligation reaction product in the 96-well reaction plate and mix by pipetting up and down 8 times, was incubated for 15 min at room temperature, mixed on a plate magnet twice during the incubation time. The beads were separated with on a plate magnet for 3 min and then remove and discard the supernatant.

Purify Hyb-Ligation Products: The plate was removed from the plate magnet, 200 μL 1× Bead Binding and Washing Buffer were added, and the beads were resuspended by pipetting up and down 5-8 times. The plate was placed on the plate magnet for 1 min, and the supernatant was discarded. The plate was removed from the plate magnet, 180 μL 1×SSC was added, and the beads were resuspended by pipetting up and down 5-8 times. The plate was placed on the plate magnet for 1 min, and the supernatant was discarded. The plate was removed from the magnet, and 180 μL TE was added. Beads were suspended by pipetting up and down 5-8 times. The plate was placed on the plate magnet for 1 min, the supernatant was discarded, and 20 μL TE buffer was added to each tube. Beads were resuspended by pipetting up and down 5-8 times. The plate was sealed and stored at 4° C. until used in subsequent steps.

Amplification:

2 μL of each purified hybridization-ligation reaction product was transferred to the corresponding well of a new 96-well reaction plate. The following reagents were added to each plate well for a total reaction volume of 100 μL.

| Component | Volume |
|---|---|
| H₂O | 72.5 μL |
| Forward Primer, 10 μM | 5 μL |
| Reverse end-labeled with dye A, 10 μM | 2.5 μL |
| Reverse end-labeled with dye B, 10 μM | 2.5 μL |
| 4 mM dNTP Mix (L/N 052114) | 15 μL |
| 10X AmpliTaq Gold Buffer | 10 μL |
| AmpliTaq Gold Enzyme | 0.5 μL |

The plate was placed in a thermal cycler, and the probes were ligated using the following cycling profile: (i) 95° C. for 5 minutes; (ii) 95° C. for 30 seconds; (iii) 54° C. for 30 seconds; (iv) 72° C. for 60 seconds; (v) Repeat steps (ii) to (iv) 29 times; (vi) 72° C. for 5 minutes; and (vi) 4° C. HOLD.

Exonuclease I Treatment:

The following reagents were added to each amplified reaction product in the 96-well reaction plate for a total reaction volume of 108 μL.

| Component | Volume |
|---|---|
| Exonuclease I 10X Reaction Buffer (L/N 0011410) | 6 μL |
| Exonuclease I (20 U/μL)(L/N 0191502) | 2 μL |

The plate was placed in a thermal cycler, and the probes were digested using the following cycling profile: (i) 37° C. for 60 minutes; (ii) 80° C. for 30 minutes; (iii) 4° C. hold.

Assay Product Purification and QC:

PCR products were purified using the GeneJET PCR Purification Kit (Thermo Scientific) according to the following protocol.
i. Transfer each Exonuclease I treated PCR product (108 μL) from the plate to a labeled 1.5 mL tube.
ii. Add 108 μL of Binding Buffer and 108 μL molecular biology grade isopropanol to each tube. Mix by vortexing and spin briefly to bring contents to tube bottom.
iii. Transfer the contents of each tube into a labeled spin column in a collection tube.
iv. Centrifuge columns for 1 minute and discard waste.
v. Add 700 μL of Wash Buffer to each column.
vi. Centrifuge columns for 1 minute and discard waste.
vii. Re-centrifuge columns for 2 minutes.
viii. Transfer each column to a new 1.5 mL labeled tube.
ix. Add 20 μL of Elution Buffer to each column.
x. Incubate columns at room temperature for 5 minutes.
xi. Centrifuge tubes for 1 minute and discard the columns.
xii. Mix by vortexing and spin briefly to bring contents to tube bottom.

The DNA concentration of each sample was determined using a NanoDrop 2000 spectrophotometer, and the dye fluorescence for each sample was determined using a NanoDrop 3300 Fluorospectrometer. The resulting values were determined to meet required specifications.

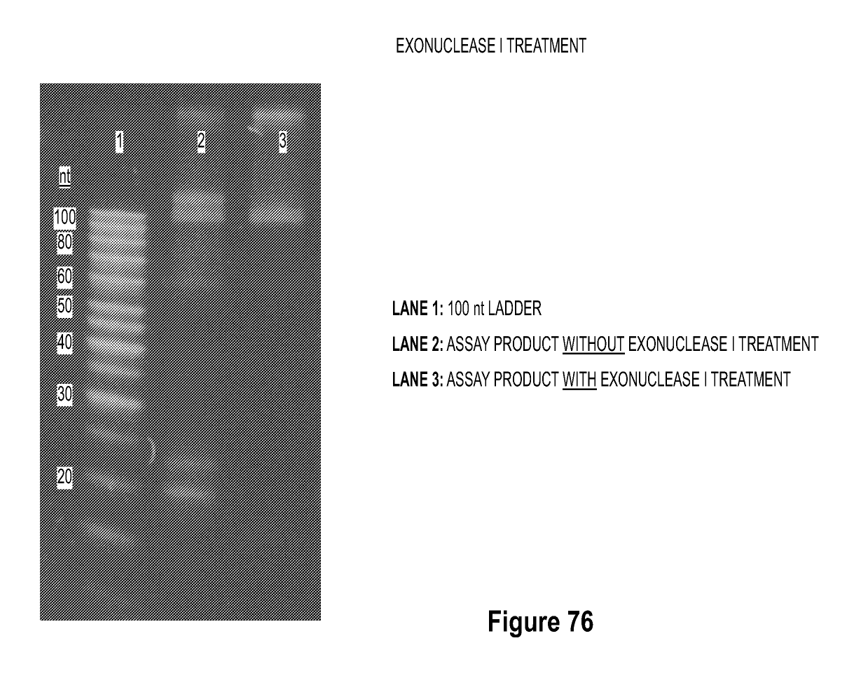

Aliquot of each assay product was run on a denaturing polyacrylamide gel to verify its fragment size. The results are shown in FIG. 76, supporting that Exonuclease I treatment eliminates the following single stranded DNA species resulting in a primary band at about 100 nt: Non-ligated tagging probe (left arm) at about 60 nt; Forward primer at about 20 nt; Reverse primer (dye-labeled) at about 24 nt; and Material at about 120 nt that likely represents 100 nt assay product in a complex with primer.

Microarray Hybridization:

The volume of each assay product required for a final concentration of 2 nM in the hybridization reaction was calculated, and this volume was transferred to a 96-well reaction plate for each. The following reagents were added to each plate well for a total reaction volume of 20 μL.

| Component | Volume |
|---|---|
| 2X Hybridization Buffer | 10 μL |
| Hybridization control oligonucleotides | 1 μL |
| H₂O | To 20 μL |

The reagents were mixed by pipetting up and down, and the plate was spun briefly to bring contents to the bottoms of the plate wells. The plate was placed in a thermal cycler, and the probes were denatured using the following cycling profile: (i) 95° C. for 10 minutes; (ii) 42° C. HOLD. The barcode of the microarray to be used was recorded for each sample in the Tracking Sheet. A hybridization chamber containing a Lifter Slip for each microarray to be processed is prepared. For each sample, 15 μL of Microarray Target was added to the center of a Lifter Slip in a hybridization chamber, and the appropriate microarray was immediately placed onto the target fluid by placing the top edge down onto the lifter slip and slowly letting it fall down flat. The hybridization chambers were closed and incubated them at 42° C. for 60 minutes. The hybridization chambers were opened, and each microarray was removed from the Lifter Slips and placed into a rack immersed in Microarray Wash Buffer A. Once all the microarrays were in the rack, the rack was stirred at 650 rpm for 5 minutes. The rack of microarrays was removed from Microarray Wash Buffer A, excess liquid on a clean room wipe was tapped off, and the rack were quickly placed into Microarray Wash Buffer B. The rack was stirred at 650 rpm for 5 minutes. The rack of microarrays was removed from Microarray Wash Buffer B, excess liquid was tapped off on a clean room wipe, and the rack was quickly placed into Microarray Wash Buffer C. The rack was stirred at 650 rpm for 5 minutes. Immediately upon completion of the 5 minute wash in Microarray Wash Buffer C, the rack of microarrays was slowly removed from the buffer. This took 5-10 seconds to maximize the sheeting of the wash buffer from the cover slip surface. Excess liquid was tapped off on a clean room wipe. A vacuum aspirator was used to remove any remaining buffer droplets present on either surface of each microarray. The microarrays were stored in a slide rack under nitrogen and in the dark until the microarrays were analyzed.

1. A method of isolating a ligated probe set hybridized to a genetic sample, comprising
contacting probe sets to a genetic sample under a condition effective to hybridize the probe sets to a nucleic acid region of interest in nucleotide molecules of the genetic sample, wherein
each of the probe sets comprises a first oligonucleotide probe at the 5′ end of the probe set and a second oligonucleotide probe at the 3′ end of the probe set,
each of the oligonucleotide probes is configured to hybridize to a part of the nucleic acid region of interest,
the first oligonucleotide probe comprises phosphorothioate bonds at four or more nucleotide bonds from the 5′ end, and
the second oligonucleotide probe comprises phosphorothioate bonds at four or more nucleotide bonds from the 3′ end,
ligating the first and second oligonucleotide probes that are hybridized to the nucleotide molecules,
digesting terminal phosphodiester bonds in non-hybridized oligonucleotide probes and/or partially hybridized nucleic acid molecules with one or more exonuclease, and
isolating ligated oligonucleotide probes hybridized to digested nucleic acid molecules from the genetic sample.

2. The method according to claim 1, wherein the one or more exonuclease comprises a mixture of exonuclease enzymes that digest both double and single-stranded oligonucleotide molecules from 5′ and 3′-end directions.

3. The method according to any one of claims 1-2, wherein the one or more exonuclease comprises one or more of Exonuclease I, Exonuclease III, Exonuclease VII, Lambda Exonuclease, and T7 Gene 6 Exonuclease.

4. The method according to any one of claims 1-3, wherein the first and/or second oligonucleotide probe comprises a label.

5. The method according to claim 4, wherein the label is a fluorescent dye.

6. The method according to any one of claims 1-5, wherein the first or second oligonucleotide probes comprise a tag.

7. The method according to any one of claims 1-6, wherein the first or second oligonucleotide probe comprises biotin.

8. The method according to any one of claims 1-7, wherein the length of the first and second oligonucleotide probes are from 5 to 150 nucleotides.

9. The method according to any one of claims 1-8, wherein
the probe set further comprises a third oligonucleotide probe that is configured to hybridize to a nucleic acid region in the nucleotide molecules between the nucleic acid regions to which the first and second oligonucleotide probes hybridize, and
the ligating step comprises ligating the first, second and third oligonucleotide probes that are hybridized to the nucleotide molecules.

10. The method according to claim 9, wherein each of the first, second, and third oligonucleotide probes comprise a label.

11. The method according to any one of claims 1-10, wherein the genetic sample is isolated from plasma.

12. The method according to any one of claims 1-11, wherein the first and second oligonucleotide probes comprise first and second cap structures at the 5′ terminus of the first oligonucleotide probe and 3′ terminus of the second oligonucleotide probe, respectively.

13. The method according to any one of claims 1-12, wherein
the ligating comprises (i) raising temperature of the probe sets and the nucleotide molecules, and/or (ii) contacting a stabilizing agent to the probe sets and the nucleotide molecules.

14. The method according to any one of claims 1-12, wherein the ligating comprises raising temperature of the probe sets and the nucleotide molecules to an increased temperature, and contacting a ligation agent to the probe sets at the increased temperature.

15. The method according to any one of claims 1-12, wherein the ligating comprises raising temperature of the probe sets and the nucleotide molecules to an increased temperature, contacting a stabilizing agent to the probe sets and the nucleotide molecules, and contacting a ligation agent to the probe sets at the increased temperature in the presence of the stabilizing agent.

16. A method of detecting a genetic variation in a genetic sample from a subject, comprising
contacting probe sets to a genetic sample under a condition effective to hybridize the probe sets to a nucleic acid region of interest in nucleotide molecules of the genetic sample, wherein
each of the probe sets comprises a first oligonucleotide probe at the 5′ end of the probe set and a second oligonucleotide probe at the 3′ end of the probe set,
each of the oligonucleotide probes is configured to hybridize to a part of the nucleic acid region of interest,
the first oligonucleotide probe comprises phosphorothioate bonds at four or more nucleotide bonds from the 5′ end, and
the second oligonucleotide probe comprises phosphorothioate bonds at four or more nucleotide bonds from the 3′ end,
ligating the first and second oligonucleotide probes that are hybridized to the nucleotide molecules,
digesting terminal phosphodiester bonds in non-hybridized oligonucleotide probes and/or partially hybridized nucleic acid molecules with one or more exonuclease,
detecting non-digested and ligated probe sets to determine the presence or absence of the genetic variation in a genetic sample from a subject.

17. The method according to claim 16, wherein the one or more exonuclease comprises a mixture of exonuclease enzymes that digest both double and single-stranded oligonucleotide molecules from 5′ and 3′-end directions.

18. The method according to any one of claims 16-17, wherein the one or more exonuclease comprises one or more of Exonuclease I, Exonuclease III, Exonuclease VII, Lambda Exonuclease, and T7 Gene 6 Exonuclease.

19. The method according to any one of claims 16-18, wherein the method comprises amplifying the ligated probe sets before the digesting.

20. The method according to any one of claims 16-19, wherein the first oligonucleotide probe comprises a label and the second oligonucleotide comprises a tag.

21. The method according to any one of claims 16-20, wherein the first oligonucleotide probe comprises a tag and the second oligonucleotide comprises a label.

22. The method according to claim 21, wherein the label is a fluorescent dye.

23. The method according to any one of claims 16-22, wherein the genetic variation is polymorphism.

24. The method according to any one of claims 16-23, wherein the first or second oligonucleotide probe comprises biotin.

25. The method according to any one of claims 16-24, wherein the length of the first and second oligonucleotide probe is from 5 to 150 nucleotides.

26. The method according to any one of claims 16-25, wherein
the probe set further comprises a third oligonucleotide probe that is configured to hybridize to a nucleic acid region in the nucleic acid molecules between the nucleic acid regions to which the first and second oligonucleotide probes hybridize, and
the ligating step comprises ligating the first, second and third oligonucleotide probes that are hybridized to the nucleotide molecule.

27. The method according to claim 26, wherein each of the first, second, and third oligonucleotide probes comprises a label.

28. The method according to any one of claims 16-27, wherein the genetic sample is isolated from plasma.

29. The method according to any one of claims 16-28, wherein the first and second oligonucleotide probes comprise first and second cap structures at the 5′ terminus of the first oligonucleotide probe and 3′ terminus of the second oligonucleotide probe, respectively.

30. The method according to any one of claims 16-29, wherein
the ligating comprises (i) raising temperature of the probe sets and the nucleotide molecules, and/or (ii) contacting a stabilizing agent to the probe sets and the nucleotide molecules.

31. The method according to any one of claims 16-29, wherein the ligating comprises raising temperature of the probe sets and the nucleotide molecules to an increased temperature, and contacting a ligation agent to the probe sets at the increased temperature.

32. The method according to any one of claims 16-29, wherein the ligating comprises raising temperature of the probe sets and the nucleotide molecules to an increased temperature, contacting a stabilizing agent to the probe sets and the nucleotide molecules, and contacting a ligation agent to the probe sets at the increased temperature in the presence of the stabilizing agent.

33. A method of detecting a genetic variation in a genetic sample from a subject, comprising
contacting first and second probe sets to the genetic sample under a condition effective to hybridize the probe sets to a nucleic acid region of interest in nucleotide molecules of the genetic sample, wherein
the first probe set comprises a first labeling probe and a first tagging probe, and the second probe set comprises a second labeling probe and a second tagging probe, and
each of the probes is configured to hybridize to a part of the nucleic acid region of interest in the nucleic acid molecules and comprises phosphorothioate bonds at four or more nucleotide bonds from the 5′ or 3′ end,
hybridizing at least parts of the first and second probe sets to first and second nucleic acid regions of interest in nucleotide molecules of the genetic sample, respectively,
ligating the first probe set by ligating the first labeling probe and the first tagging probe to produce a first ligated probe set comprising the phosphorothioate bonds at the 5′ and 3′ ends,
ligating the second probe set by ligating the second labeling probe and the second tagging probe to produce a second ligated probe set comprising the phosphorothioate bonds at the 5′ and 3′ ends,
digesting terminal phosphodiester bonds in non-hybridized probe sets and/or partially hybridized nucleic acid molecules with one or more exonuclease,
immobilizing the tagging probes to a pre-determined location on a substrate, wherein
the first and second labeling probes and/or the amplified labeling probes thereof ligated to the immobilized tagging probes comprise first and second labels, respectively,
the first and second labels are different,
the immobilized labels are optically resolvable, and
the immobilized first and second tagging probes and/or the amplified tagging probes thereof comprise first and second tags, respectively,
counting (i) a first number of the first label immobilized to the substrate, and (ii) a second number of the second label immobilized to the substrate, and
comparing the first and second numbers to determine the genetic variation in the genetic sample.

34. A method of detecting a genetic variation in a genetic sample from a subject, comprising
contacting probes or probe sets to the genetic sample,
hybridizing the probes or probe sets to a nucleic acid region of interest in nucleotide molecules of the genetic sample,
amplifying the probes or probe sets with forward and reverse primers, wherein the forward or reverse primers comprise one or more label,
digesting terminal phosphodiester bonds in single-stranded oligonucleotides of the probes or probe sets with a first exonuclease after the amplifying,
after the digesting with the first exonuclease, inactivating the first exonuclease,
after the inactivating, digesting an end of the amplified double-stranded probes or probe sets with a second exonuclease to produce an amplified single-stranded probe or probe sets, and
after the digesting with the second exonuclease, detecting single-stranded oligonucleotides of the probes or probe sets to determine the presence or absence of the genetic variation in a genetic sample from a subject.

35. The method according to claim 34, wherein
the probe set is contacted to the genetic sample,
the probe set comprises a labeling probe and a tagging probe, and
the method further comprises ligating the labeling probe and the tagging probe prior to the amplifying.

36. The method according to any one of claims 34-35, wherein the first exonuclease is Exonuclease I, and the second exonuclease is lambda Exonuclease.

37. The method according to any one of claims 34-36, wherein the first exonuclease is inactivated by heat.

38. The method according to any one of claims 34-37, wherein
the probe set is contacted to the genetic sample,
the probe set comprises a labeling probe and a tagging probe,
the method further comprises ligating the labeling probe and the tagging probe prior to the amplifying.
the ligated probe set comprises the labeling probe at the 3′-end and the tagging probe at the 5′-end,
the labeling probe hybridizes to the reverse primer,
the tagging probe comprises the isolating tag,
the tagging probe hybridizes to the forward primer, and
the reverse primer comprises the label.

39. The method according to any one of claims 34-38, wherein the label is a fluorescent dye.

40. The method according to claim 35, wherein the lengths of the labeling and tagging probes are from 5 to 150 nucleotides.

41. The method according to any one of claims 34-40, wherein
the probe set is contacted to the genetic sample, and
the probe set further comprises a gap probe ligated between the labeling and tagging probes.

42. The method according to claim 41, wherein one or more of the labeling, tagging, and gap probes comprises a label.

43. A method of isolating amplified products of a ligated probe set comprising
immobilizing a composition comprising single-stranded ligated probe sets and second probes on a substrate, wherein
each of the single-stranded ligated probe set comprises a labeling probe and a tagging probe ligated to each other,
each of the second probes comprises the labeling probe or the tagging probe, and
the labeling probe or the tagging probe comprises an isolating tag configured to bind to the substrate,
amplifying one or more of the single-stranded ligated probe sets with forward and reverse primers after the immobilizing to form one or more double-stranded ligated probe set, wherein
the forward or reverse primer hybridizing to the labeling probe of the one or more of the single-stranded ligated probe sets comprises a label,
digesting terminal phosphodiester bonds in the second probes and/or the single-stranded ligated probe sets with one or more exonuclease after the amplifying, and
isolating the non-digested and ligated probe sets after the digesting.

44. The method according to claim 43, wherein the isolating tag is biotin, and the substrate comprises streptavidin.

45. The method according to any one of claims 43-44, wherein the substrate comprises a streptavidin magnetic bead.

46. The method according to any one of claims 43-45, wherein
each of the single-stranded ligated probe sets comprises the labeling probe at the 3′-end and the tagging probe at the 5′-end,
the second probes comprise the tagging probe,
the tagging probe comprises the isolating tag,
the labeling probe hybridizes to the reverse primer,
the tagging probe hybridizes to the forward primer, and
the reverse primer comprises the label.

47. The method according to any one of claims 43-46, wherein the label is a fluorescent dye.

48. The method according to any one of claims 43-47, wherein the lengths of the labeling and tagging probes are from 5 to 150 nucleotides.

49. The method according to any one of claims 43-48, wherein
the probe set further comprises a gap probe ligated between the labeling and tagging probes.

50. The method according to claim 49, wherein one or more of the labeling, tagging, and gap probes comprises a label.

51. A method of detecting a genetic variation in a genetic sample from a subject, comprising
contacting single-stranded probe sets to the genetic sample, wherein each of the single-stranded probe sets comprises a labeling probe and a tagging probe, and the labeling probe or the tagging probe comprises an isolating tag configured to bind to the substrate,
hybridizing the single-stranded probe sets to a nucleic acid region of interest in nucleotide molecules of the genetic sample,
ligating the single-stranded probe sets at least by ligating the labeling probe and the tagging probe to produce first single-stranded ligated probe sets,
immobilizing the first single-stranded ligated probe sets on a substrate,
amplifying the first single-stranded ligated probe sets with forward and reverse primers after the immobilizing to form double-stranded ligated probe set, wherein
the forward or reverse primer hybridizing to the labeling probe of the first single-stranded ligated probe set comprises one or more label,
digesting terminal phosphodiester bonds in the single-stranded probe sets with a first exonuclease after the amplifying,
after the digesting with the first exonuclease, inactivating the first exonuclease,
after the inactivating, digesting an end of the amplified double-stranded ligated probe sets with a second exonuclease to produce second single-stranded ligated probe set, and
after the digesting with the second exonuclease, detecting the second single-stranded ligated probe sets to determine the presence or absence of the genetic variation in a genetic sample from a subject.

52. The method according to claim 51, wherein the first exonuclease is Exonuclease I, and the second exonuclease is lambda Exonuclease.

53. The method according to any one of claims 51-52, wherein the first exonuclease is inactivated by heat.

54. The method according to any one of claims 51-53, wherein the isolating tag is biotin, and the substrate comprises streptavidin.

55. The method according to claim 54, wherein the substrate comprises a streptavidin magnetic bead.

56. The method according to any one of claims 51-55, wherein
the first single-stranded ligated probe set comprises the labeling probe at the 3′-end and the tagging probe at the 5′-end,
the labeling probe hybridizes to the reverse primer,
the tagging probe comprises the isolating tag,
the tagging probe hybridizes to the forward primer, and
the reverse primer comprises the label.

57. The method according to any one of claims 51-56, wherein the label is a fluorescent dye.

58. The method according to any one of claims 51-57, wherein the lengths of the labeling and tagging probes are from 5 to 150 nucleotides.

59. The method according to any one of claims 51-58, wherein
the probe set further comprises a gap probe ligated between the labeling and tagging probes.

60. The method according to claim 59, wherein one or more of the labeling, tagging, and gap probes comprises a label.

61. A method of detecting a genetic variation in a genetic sample from a subject, comprising
contacting non-ligated first and second probe sets to the genetic sample, wherein each of the first probe sets comprises a first labeling probe and a first tagging probe, and each of the second probe sets comprises a second labeling probe and a second tagging probe,
hybridizing one or more of the first probe set and one or more of the second probe set to first and second nucleic acid regions of interest in nucleotide molecules of the genetic sample, respectively, to produce hybridized first and second probe set,
ligating the hybridized first probe set at least by ligating the first labeling probe and the first tagging probe to produce a ligated first probe set,
ligating the hybridized second probe set at least by ligating the second labeling probe and the second tagging probe to produce a ligated second probe set,
immobilizing one or more of the tagging probes or the labeling probes of each of the non-ligated and ligated probe sets on one or more bead,
amplifying the ligated probe set with forward and reverse primers after the immobilizing, wherein
the forward or reverse primer hybridizing to the labeling probe of the ligated probe set comprises a label,
digesting terminal phosphodiester bonds in non-ligated probes with one or more exonuclease after the amplifying,
immobilizing the tagging probes to a pre-determined location on a substrate, wherein
the first and second labeling probes and/or the amplified labeling probes thereof ligated to the immobilized tagging probes comprise first and second labels, respectively,
the first and second labels are different,
the immobilized labels are optically resolvable, and
the immobilized first and second tagging probes and/or the amplified tagging probes thereof comprise first and second tags, respectively,
counting (i) a first number of the first label immobilized to the substrate, and (ii) a second number of the second label immobilized to the substrate, and
comparing the first and second numbers to determine the genetic variation in the genetic sample.

62. A method of isolating a ligated probe set hybridized to a genetic sample, comprising
contacting a probe set to a genetic sample under a condition effective to hybridize the probe set to a nucleic acid region of interest in nucleotide molecules of the genetic sample, wherein the probe set comprises first and second oligonucleotide probes,
ligating the first and second oligonucleotide probes to form a ligated probe set,
denaturing the ligated probe set from the genetic sample,
hybridizing at least a part of a junction capture probe to the ligated probe set, wherein the junction capture probe is hybridized to (i) at least a part of the first oligonucleotide probe and (ii) at least a part of the second oligonucleotide probe, and
isolating the ligated probe set hybridized to the junction capture probe.

63. The method according to claim 62, wherein the junction capture probe comprises a tag, and the isolating comprises immobilizing the tag on a substrate and washing the substrate.

64. The method according to claim 63, wherein the tag is biotin, and the substrate comprises streptavidin.

65. The method according to claim 64, wherein the substrate comprises a streptavidin magnetic bead.

66. The method according to any of claims 63-65, wherein the immobilizing the tag is performed prior to the hybridizing the at least a part of a junction capture probe to the ligated probe set, and the washing is performed after the hybridizing.

67. The method according to any of claims 62-66, wherein the isolating comprises separating the ligated probe set from the substrate.

68. The method according to claim 63, wherein the substrate comprises an anchor tag, and the immobilizing comprises immobilizing the tag to the anchor tag of the substrate.

69. The method according to claim 68, wherein the tag of the junction capture probe and the anchor tag of the substrate comprise complementary oligonucleotide sequences, and the immobilizing comprises hybridizing the tag to the anchor tag of the substrate.

70. The method according to any of claims 62-69, wherein the isolating comprises isolating the ligated probe set hybridized to the junction capture probe from the genetic sample.

71. The method according to any of claims 62-70, wherein (i) said at least a part of the first oligonucleotide probe hybridized to the junction capture probe and (ii) said at least a part of the second oligonucleotide probe hybridized to the junction capture probe are adjacent to each other.

72. The method according to any one of claims 62-71, wherein
the ligating comprises (i) raising temperature of the probe set and the nucleotide molecules, and/or (ii) contacting a stabilizing agent to the probe set and the nucleotide molecules.

73. The method according to any one of claims 62-71, wherein the ligating comprises raising temperature of the probe set and the nucleotide molecules to an increased temperature, and contacting a ligation agent to the probe set at the increased temperature.

74. The method according to any one of claims 62-71, wherein the ligating comprises raising temperature of the probe set and the nucleotide molecules to an increased temperature, contacting a stabilizing agent to the probe set and the nucleotide molecules, and contacting a ligation agent to the probe set at the increased temperature in the presence of the stabilizing agent.

75. A method of detecting a genetic variation in a genetic sample from a subject, comprising
contacting a probe set to a genetic sample under a condition effective to hybridize the probe set to a nucleic acid region of interest in nucleotide molecules of the genetic sample, wherein
the probe set comprises first and second oligonucleotide probes, and
each of the oligonucleotide probes is configured to hybridize to a part of the nucleic acid region of interest,
ligating the first and second oligonucleotide probes that are hybridized to the nucleotide molecules to form a ligated probe set,
denaturing the ligated probe set from the genetic sample,
hybridizing at least a part of a junction capture probe to the ligated probe set, wherein the junction capture probe is hybridized to (i) at least a part of the first oligonucleotide and (ii) at least a part of the second oligonucleotide,
isolating the ligated probe set hybridized to the junction capture probe,
amplifying the ligated probe set to form amplified ligated probe sets, and
detecting the amplified ligated probe sets to determine the presence or absence of the genetic variation in a genetic sample from a subject.

76. The method according to claim 75, wherein the junction capture probe comprises a tag, and the isolating comprises immobilizing the tag on a substrate and washing the substrate.

77. The method according to claim 76, wherein the tag is biotin, and the substrate comprises streptavidin.

78. The method according to claim 77, wherein the substrate comprises a streptavidin magnetic bead.

79. The method according to any of claims 76-78, wherein the immobilizing the tag is performed prior to the hybridizing the at least a part of a junction capture probe to the ligated probe set, and the washing is performed after the hybridizing.

80. The method according to any of claims 75-79, wherein the isolating comprises separating the ligated probe set from the substrate.

81. The method according to claim 76, wherein the substrate comprises an anchor tag, and the immobilizing comprises immobilizing the tag to the anchor tag of the substrate.

82. The method according to claim 81, wherein the tag of the junction capture probe and the anchor tag of the substrate comprise complementary oligonucleotide sequences, and the immobilizing comprises hybridizing the tag to the anchor tag of the substrate.

83. The method according to any of claims 75-82, wherein the isolating comprises isolating the ligated probe set hybridized to the junction capture probe from the genetic sample.

84. The method according to any of claims 75-83, wherein (i) said at least a part of the first oligonucleotide probe hybridized to the junction capture probe and (ii) said at least a part of the second oligonucleotide probe hybridized to the junction capture probe are adjacent to each other.

85. The method according to any one of claims 75-84, wherein
the ligating comprises (i) raising temperature of the probe set and the nucleotide molecules, and/or (ii) contacting a stabilizing agent to the probe set and the nucleotide molecules.

86. The method according to any one of claims 75-84, wherein the ligating comprises raising temperature of the probe set and the nucleotide molecules to an increased temperature, and contacting a ligation agent to the probe set at the increased temperature.

87. The method according to any one of claims 75-84, wherein the ligating comprises raising temperature of the probe set and the nucleotide molecules to an increased temperature, contacting a stabilizing agent to the probe set and the nucleotide molecules, and contacting a ligation agent to the probe set at the increased temperature in the presence of the stabilizing agent.

88. A method of detecting a genetic variation in a genetic sample from a subject, comprising
contacting first and second probe sets to the genetic sample under a condition effective to hybridize the probe sets to a nucleic acid region of interest in nucleotide molecules of the genetic sample, wherein
the first probe set comprises a first labeling probe and a first tagging probe, and
the second probe set comprises a second labeling probe and a second tagging probe,
denaturing the ligated probe set from the genetic sample,
ligating at least parts of the first probe set at least by ligating the first labeling probe and the first tagging probe to form a first ligated probe set,
ligating at least parts of the second probe set at least by ligating the second labeling probe and the second tagging probe to form a second ligated probe set,
hybridizing at least a part of each of first and second junction capture probes to the first and second ligated probe sets, respectively, wherein the first junction capture probe is hybridized to at least a part of each of the first labeling probe and the first tagging probe, and the second junction capture probe is hybridized to at least a part of each of the second labeling probe and the second tagging probe,
isolating at least a part of the first and second ligated probe sets that are hybridized to the first and second junction capture probes, respectively, to form first and second isolated ligated probe sets,
amplifying (i) the first isolated ligated probe set with first forward and reverse primers, wherein at least one of the first forward and reverse primers comprises a first label, and (ii) the second isolated ligated probe set with second forward and reverse primers, wherein at least one of the second forward and reverse primers comprises a second label, to form amplified first and second ligated probe sets comprising the first and second labels, respectively, wherein the first and second labels are different,
immobilizing at least parts of the amplified first and second ligated probe sets on a substrate, wherein the first and second labels of the amplified first and second ligated probe sets are optically resolvable after immobilization,
counting (i) a first number of the first label in the amplified first probe set immobilized to the substrate, and (ii) a second number of the second label in the amplified second probe set immobilized to the substrate, and
comparing the first and second numbers to determine the presence or absence of the genetic variation in a genetic sample from a subject.

89. The method according to claim 88, wherein the junction capture probe comprises a tag, and the isolating comprises immobilizing the tag on a substrate and washing the substrate.

90. The method according to claim 89, wherein the tag is biotin, and the substrate comprises streptavidin.

91. The method according to claim 90, wherein the substrate comprises a streptavidin magnetic bead.

92. The method according to any of claims 89-91, wherein the immobilizing the tag is performed prior to the hybridizing the at least a part of a junction capture probe to the ligated probe set, and the washing is performed after the hybridizing.

93. The method according to any of claims 88-92, wherein the isolating comprises separating the ligated probe set from the substrate.

94. The method according to claim 89, wherein the substrate comprises an anchor tag, and the immobilizing comprises immobilizing the tag to the anchor tag of the substrate.

95. The method according to claim 94, wherein
the tag of the junction capture probe and the anchor tag of the substrate comprise complementary oligonucleotide sequences, and
the immobilizing comprises hybridizing the tag to the anchor tag of the substrate.

96. The method according to any of claims 88-95, wherein the isolating comprises isolating the ligated probe set hybridized to the junction capture probe from the genetic sample.

97. The method according to any of claims 88-96, wherein (i) said at least a part of the first oligonucleotide probe hybridized to the junction capture probe and (ii) said at least a part of the second oligonucleotide probe hybridized to the junction capture probe are adjacent to each other.

98. The method according to any one of claims 88-97, wherein
the ligating comprises (i) raising temperature of the first and second probe sets and the nucleotide molecules, and/or (ii) contacting a stabilizing agent to the first and second probe sets and the nucleotide molecules.

99. The method according to any one of claims 88-97, wherein the ligating comprises raising temperature of the first and second probe sets and the nucleotide molecules to an increased temperature, and contacting a ligation agent to the first and second probe sets at the increased temperature.

100. The method according to any one of claims 88-97, wherein the ligating comprises raising temperature of the first and second probe sets and the nucleotide molecules to an increased temperature, contacting a stabilizing agent to the first and second probe sets and the nucleotide molecules, and contacting a ligation agent to the first and second probe sets at the increased temperature in the presence of the stabilizing agent.

101. A method of isolating a ligated probe set hybridized to a genetic sample, comprising
contacting a probe set to a genetic sample under a condition effective to hybridize the probe set to a nucleic acid region of interest in nucleotide molecules of the genetic sample, wherein the probe set comprises first and second oligonucleotide probes,
ligating the first and second oligonucleotide probes to form a ligated probe set,
hybridizing at least a part of a junction capture probe to the ligated probe set, wherein the junction capture probe is hybridized to (i) at least a part of the first oligonucleotide probe and (ii) at least a part of the second oligonucleotide probe, and
isolating the ligated probe set hybridized to the junction capture probe.

102. A method of detecting a genetic variation in a genetic sample from a subject, comprising
contacting a probe set to a genetic sample under a condition effective to hybridize the probe set to a nucleic acid region of interest in nucleotide molecules of the genetic sample, wherein
the probe set comprises first and second oligonucleotide probes, and
each of the oligonucleotide probes is configured to hybridize to a part of the nucleic acid region of interest,
ligating the first and second oligonucleotide probes that are hybridized to the nucleotide molecules to form a ligated probe set,
hybridizing at least a part of a junction capture probe to the ligated probe set, wherein the junction capture probe is hybridized to (i) at least a part of the first oligonucleotide and (ii) at least a part of the second oligonucleotide,
isolating the ligated probe set hybridized to the junction capture probe,
amplifying the ligated probe set to form amplified ligated probe sets, and
detecting the amplified ligated probe sets to determine the presence or absence of the genetic variation in a genetic sample from a subject.

103. A method of detecting a genetic variation in a genetic sample from a subject, comprising
contacting first and second probe sets to the genetic sample under a condition effective to hybridize the probe sets to a nucleic acid region of interest in nucleotide molecules of the genetic sample, wherein
the first probe set comprises a first labeling probe and a first tagging probe, and
the second probe set comprises a second labeling probe and a second tagging probe,
ligating at least parts of the first probe set at least by ligating the first labeling probe and the first tagging probe to form a first ligated probe set,
ligating at least parts of the second probe set at least by ligating the second labeling probe and the second tagging probe to form a second ligated probe set,
hybridizing at least a part of each of first and second junction capture probes to the first and second ligated probe sets, respectively, wherein the first junction capture probe is hybridized to at least a part of each of the first labeling probe and the first tagging probe, and the second junction capture probe is hybridized to at least a part of each of the second labeling probe and the second tagging probe,
isolating at least a part of the first and second ligated probe sets that are hybridized to the first and second junction capture probes, respectively, to form first and second isolated ligated probe sets,
amplifying (i) the first isolated ligated probe set with first forward and reverse primers, wherein at least one of the first forward and reverse primers comprises a first label, and (ii) the second isolated ligated probe set with second forward and reverse primers, wherein at least one of the second forward and reverse primers comprises a second label, to form amplified first and second ligated probe sets comprising the first and second labels, respectively, wherein the first and second labels are different,
immobilizing at least parts of the amplified first and second ligated probe sets on a substrate, wherein the first and second labels of the amplified first and second ligated probe sets are optically resolvable after immobilization,
counting (i) a first number of the first label in the amplified first probe set immobilized to the substrate, and (ii) a second number of the second label in the amplified second probe set immobilized to the substrate, and
comparing the first and second numbers to determine the presence or absence of the genetic variation in a genetic sample from a subject.
